SEQUENCE LISTING (91 sequences)

SEQ ID NO: 1 - protein, 1111 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSTLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

SEQ ID NO: 2 - protein, 174 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 3 - protein, 168 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNC

SEQ ID NO: 4 - protein, 138 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRA

SEQ ID NO: 5 - protein, 133 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPA

SEQ ID NO: 6 - protein, 116 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYC

SEQ ID NO: 7 - protein, 100 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRL

SEQ ID NO: 8 - protein, 147 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 9 - protein, 141 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNC

SEQ ID NO: 10 - protein, 129 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGT

SEQ ID NO: 11 - protein, 111 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRA

SEQ ID NO: 12 - protein, 106 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPA

SEQ ID NO: 13 - protein, 89 aa, Homo sapiens
CPCHNGFSCSVIPETEEVVCNNCPPGVTGARCELCADGYFGDPFGEHGPVRPCQPCQCNSNVDPSASGNCDRLTGRCLKCIHNTAGIYC

SEQ ID NO: 14 - protein, 73 aa, Homo sapiens
CPCHNGFSCSVIPETEEVVCNNCPPGVTGARCELCADGYFGDPFGEHGPVRPCQPCQCNSNVDPSASGNCDRL

SEQ ID NO: 15 - protein, 114 aa, Homo sapiens
LCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 16 - protein, 108 aa, Homo sapiens
LCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGTCVCKPGFGGPNC

SEQ ID NO: 17 - protein, 96 aa, Homo sapiens
LCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGT

SEQ ID NO: 18 - protein, 78 aa, Homo sapiens
LCADGYFGDPFGEHGPVRPCQPCQCNSNVDPSASGNCDRLTGRCLKCIHNTAGIYCDQCKAGYFGDPLAPNPADKCRA

SEQ ID NO: 19 - protein, 73 aa, Homo sapiens
LCADGYFGDPFGEHGPVRPCQPCQCNSNVDPSASGNCDRLTGRCLKCIHNTAGIYCDQCKAGYFGDPLAPNPA

SEQ ID NO: 20 - protein, 56 aa, Homo sapiens
LCADGYFGDPFGEHGPVRPCQPCQCNSNVDPSASGNCDRLTGRCLKCIHNTAGIYC

SEQ ID NO: 21 - protein, 40 aa, Homo sapiens
LCADGYFGDPFGEHGPVRPCQPCQCNSNVDPSASGNCDRL

SEQ ID NO: 22 - protein, 409 aa, Artificial
  Synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPEFIVTD

SEQ ID NO: 23 - protein, 227 aa, Homo sapiens
CICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 24 - protein, 20 aa, Murine Antibodies
CRSSKSLLHNIGITYLFWYL

SEQ ID NO: 25 - protein, 20 aa, Murine Antibodies
CRSSQGLVHSNGNTYLHWYL

SEQ ID NO: 26 - protein, 14 aa, Murine Antibodies
CSASSSVSYIHWYQ

SEQ ID NO: 27 - protein, 20 aa, Artificial
  Synthetic
  misc_feature (5)..(5): Xaa can be any naturally occurring amino acid
  misc_feature (10)..(11): Xaa can be any naturally occurring amino acid
CRSSXSLLHXXGITYLHWYL

SEQ ID NO: 28 - protein, 12 aa, Murine Antibodies
IYQMSKLASGVP

SEQ ID NO: 29 - protein, 12 aa, Murine Antibodies
IYRVSNRFSGVP

SEQ ID NO: 30 - protein, 12 aa, Murine Antibodies
IYDTSKLASGVP

SEQ ID NO: 31 - protein, 12 aa, Artificial
  Synthetic
  misc_feature (3)..(3): Xaa can be any naturally occurring amino acid
IYXMSKLASGVP

SEQ ID NO: 32 - protein, 14 aa, Mus musculus
CAQNLELPPTFGSG

SEQ ID NO: 33 - protein, 14 aa, Mus musculus
CSQSTHVPWTFGGS

SEQ ID NO: 34 - protein, 14 aa, Murine Antibodies
CQQWSSSPPTFGGG

SEQ ID NO: 35 - protein, 14 aa, Artificial
  Synthetic
  misc_feature (4)..(4): Xaa can be any naturally occurring amino acid
  misc_feature (6)..(6): Xaa can be any naturally occurring amino acid
CAQXSXLPPTFGGG

SEQ ID NO: 36 - protein, 16 aa, Murine Antibodies
CVASGFTFSNFWMNWV

SEQ ID NO: 37 - protein, 16 aa, Murine Antibodies
CSVTGDSITSGYRNWI

SEQ ID NO: 38 - protein, 17 aa, Murine Antibodies
CTVTGYSITSDYAWNWI

SEQ ID NO: 39 - protein, 17 aa, Artificial
  Synthetic
  misc_feature (11)..(11): Xaa can be any naturally occurring amino acid
  misc_feature (13)..(13): Xaa can be any naturally occurring amino acid
  misc_feature (15)..(15): Xaa can be any naturally occurring amino acid
CSVTGFSITSXYXNXWI

SEQ ID NO: 40 - protein, 27 aa, Murine Antibodies
LEWVAEIRLKSNNYATHYAESLKGRFT

SEQ ID NO: 41 - protein, 24 aa, Murine Antibodies
LEYMGYISYRGTTYYNPSLKSRIS

SEQ ID NO: 42 - protein, 24 aa, Murine Antibodies
LEWMGYITYSGGTNYNPSLKSRIS

SEQ ID NO: 43 - protein, 27 aa, Artificial
  Synthetic
  misc_feature (12)..(14): Xaa can be any naturally occurring amino acid
LEWMGYISYKGXXXATHYNPSLKSRIS

SEQ ID NO: 44 - protein, 16 aa, Murine Antibodies
CTRDYYGSSFAYWGQG

SEQ ID NO: 45 - protein, 18 aa, Murine Antibodies
CAGPYDYDERTFAYWGQG

SEQ ID NO: 46 - protein, 16 aa, Murine Antibodies
CANNFYGNFFDYWGQG

SEQ ID NO: 47 - protein, 18 aa, Artificial
  Synthetic
  misc_feature (3)..(5): Xaa can be any naturally occurring amino acid
  misc_feature (10)..(10): Xaa can be any naturally occurring amino acid
CAXXXDYYGXSFAYWGQG

SEQ ID NO: 48 - protein, 20 aa, Artificial
  Synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(14): Xaa can be any naturally occurring amino acid
  misc_feature (16)..(17): Xaa can be any naturally occurring amino acid
  misc_feature (20)..(20): Xaa can be any naturally occurring amino acid
CXXSXXLXXXXGXXYXXWYX

SEQ ID NO: 49 - protein, 12 aa, Artificial
  Synthetic
  misc_feature (3)..(3): Xaa can be any naturally occurring amino acid
  misc_feature (5)..(5): Xaa can be any naturally occurring amino acid
  misc_feature (9)..(9): Xaa can be any naturally occurring amino acid
IYXMXKLAXGVP

SEQ ID NO: 50 - protein, 12 aa, Artificial
  Synthetic
  misc_feature (3)..(3): Xaa can be any naturally occurring amino acid
  misc_feature (5)..(5): Xaa can be any naturally occurring amino acid
  misc_feature (9)..(9): Xaa can be any naturally occurring amino acid
IYXMXKLAXGVP

SEQ ID NO: 51 - protein, 17 aa, Artificial
  Synthetic
  misc_feature (2)..(2): Xaa can be any naturally occurring amino acid
  misc_feature (5)..(6): Xaa can be any naturally occurring amino acid
  misc_feature (11)..(11): Xaa can be any naturally occurring amino acid
  misc_feature (13)..(13): Xaa can be any naturally occurring amino acid
  misc_feature (15)..(15): Xaa can be any naturally occurring amino acid
CXVTXXSITSXYXNXWI

SEQ ID NO: 52 - protein, 18 aa, Artificial
  Synthetic
  misc_feature (2)..(14): Xaa can be any naturally occurring amino acid
CXXXXXXXXXXXXXWGQG

SEQ ID NO: 53 - DNA, 32 bp, Artificial
  Synthetic
actagttttggctgaggagacggtgaccgtgg

SEQ ID NO: 54 - DNA, 32 bp, Artificial
  Synthetic
actagttttggctgaggagactgtgagagtgg

SEQ ID NO: 55 - DNA, 32 bp, Artificial
  Synthetic
actagttttggctgcagagacagtgaccagag

SEQ ID NO: 56 - DNA, 32 bp, Artificial
  Synthetic
actagttttggctgaggagacggtgactgagg

SEQ ID NO: 57 - DNA, 32 bp, Artificial
  Synthetic
tcatcaacactcattcctgttgaagctcttga

SEQ ID NO: 58 - DNA, 16 bp, Artificial
  Synthetic
gtaaaacgacggccag

SEQ ID NO: 59 - DNA, 17 bp, Artificial
  Synthetic
caggaaacagctatgac

SEQ ID NO: 60 - protein, 133 aa, Murine Antibodies
MRFSAQLLGLLVLWIPGSTADIVMTQAAFSNPVTLGTSASISCRSSKSLLHNIGITYLFWYLQKPGQSPQLLIYQMSKLASGVPDRFSGSGSGTDFTLRISRVEAEDVGIYYCAQNLELPPTFGSGTKLEIKR

SEQ ID NO: 61 - protein, 132 aa, Murine Antibodies
MKWPVRLLVLMFWIPASSSDVVMTQTPLSLSVSLGDQASISCRSSQGLVHSNGNTYLHWYLQKPGQSPKLLIYRVSNRFSGVPDRFSGSGSGTDFTLKISRVEAEDLGVYFCSQSTHVPWTFGGSTKLEIKR

SEQ ID NO: 62 - protein, 129 aa, Murine Antibodies
MDFQVQIFSFLLISASVIISRGQIVLTQSPAIMSASPGEKVTMTCSASSSVSYIHWYQQKSGTSPKRWIYDTSKLASGVPARFSGSGSGTSYSLTISSMQAEDAATYYCQQWSSSPPTFGGGTKLEIKR

SEQ ID NO: 63 - protein, 135 aa, Artificial
  Synthetic
  misc_feature (4)..(4): Xaa can be any naturally occurring amino acid
  misc_feature (9)..(9): Xaa can be any naturally occurring amino acid
  misc_feature (21)..(22): Xaa can be any naturally occurring amino acid
  misc_feature (31)..(31): Xaa can be any naturally occurring amino acid
  misc_feature (40)..(40): Xaa can be any naturally occurring amino acid
  misc_feature (49)..(49): Xaa can be any naturally occurring amino acid
  misc_feature (54)..(55): Xaa can be any naturally occurring amino acid
  misc_feature (77)..(77): Xaa can be any naturally occurring amino acid
  misc_feature (118)..(118): Xaa can be any naturally occurring amino acid
  misc_feature (120)..(120): Xaa can be any naturally occurring amino acid
MKFXVQLLXLLLIWIPASSSXXDIVMTQSPXSLSVSLGDXASISCRSSXSLLHXXGITYLHWYLQKPGQSPKLLIYXMSKLASGVPDRFSGSGSGTDFTLKISRVEAEDLGIYYCAQXSXLPPTFGGGTKLEIKR

SEQ ID NO: 64 - protein, 20 aa, Artificial
  Synthetic
  misc_feature (2)..(3): Xaa can be any naturally occurring amino acid
  misc_feature (5)..(14): Xaa can be any naturally occurring amino acid
  misc_feature (16)..(17): Xaa can be any naturally occurring amino acid
  misc_feature (20)..(20): Xaa can be any naturally occurring amino acid
CXXSXXXXXXXXXXYXXWYX

SEQ ID NO: 65 - protein, 9 aa, Murine Antibodies
IYQMSKLAS

SEQ ID NO: 66 - protein, 9 aa, Murine Antibodies
IYRVSNRFS

SEQ ID NO: 67 - protein, 9 aa, Murine Antibodies
IYDTSKLAS

SEQ ID NO: 68 - protein, 9 aa, Artificial
  Synthetic
  misc_feature (3)..(4): Xaa can be any naturally occurring amino acid
  misc_feature (6)..(8): Xaa can be any naturally occurring amino acid
IYXXSXXXS

SEQ ID NO: 69 - protein, 120 aa, Murine Antibodies
EVKLEESGGGLVQPGGSMKLSCVASGFTFSNFWMNWVRQSPEKGLEWVAEIRLKSNNYATHYAESLKGRFTISRDDSKSNVYLQMNNLRPEDTGIYYCTRDYYGSSFAYWGQGTLVTVSA

SEQ ID NO: 70 - protein, 119 aa, Murine Antibodies
EVQLQESGPSLVKPSQTLSLTCSVTGDSITSGYRNWIRKFPGNKLEYMGYISYRGTTYYNPSLKSRISITRDTSKNQYYLQLNSVTTEDTATYYCAGPYDYDERTFAYWGQGTLVTVSA

SEQ ID NO: 71 - protein, 118 aa, Murine Antibodies
DVHLQESGPGLVKPSQSLSLTCTVTGYSITSDYAWNWIRQFPGNRLEWMGYITYSGGTNYNPSLKSRISFTRDTSKNQFFLQLNSVTTEDTATYYCANNFYGNFFDYWGQGTTLTVSS

SEQ ID NO: 72 - protein, 123 aa, Artificial
  Synthetic
  misc_feature (1)..(1): Xaa can be any naturally occurring amino acid
  misc_feature (3)..(3): Xaa can be any naturally occurring amino acid
  misc_feature (5)..(5): Xaa can be any naturally occurring amino acid
  misc_feature (9)..(10): Xaa can be any naturally occurring amino acid
  misc_feature (13)..(13): Xaa can be any naturally occurring amino acid
  misc_feature (15)..(19): Xaa can be any naturally occurring amino acid
  misc_feature (21)..(21): Xaa can be any naturally occurring amino acid
  misc_feature (23)..(25): Xaa can be any naturally occurring amino acid
  misc_feature (27)..(35): Xaa can be any naturally occurring amino acid
  misc_feature (38)..(38): Xaa can be any naturally occurring amino acid
  misc_feature (40)..(41): Xaa can be any naturally occurring amino acid
  misc_feature (43)..(45): Xaa can be any naturally occurring amino acid
  misc_feature (48)..(51): Xaa can be any naturally occurring amino acid
  misc_feature (53)..(60): Xaa can be any naturally occurring amino acid
  misc_feature (62)..(62): Xaa can be any naturally occurring amino acid
  misc_feature (64)..(65): Xaa can be any naturally occurring amino acid
  misc_feature (69)..(69): Xaa can be any naturally occurring amino acid
  misc_feature (71)..(74): Xaa can be any naturally occurring amino acid
  misc_feature (77)..(77): Xaa can be any naturally occurring amino acid
  misc_feature (80)..(83): Xaa can be any naturally occurring amino acid
  misc_feature (86)..(86): Xaa can be any naturally occurring amino acid
  misc_feature (89)..(91): Xaa can be any naturally occurring amino acid
  misc_feature (95)..(96): Xaa can be any naturally occurring amino acid
  misc_feature (100)..(109): Xaa can be any naturally occurring amino acid
  misc_feature (111)..(111): Xaa can be any naturally occurring amino acid
  misc_feature (118)..(119): Xaa can be any naturally occurring amino acid
  misc_feature (123)..(123): Xaa can be any naturally occurring amino acid
XVXLXESGXXLVXPXXXXXLXCXXXGXXXXXXXXXNWXRXXPXXXLEXXXXIXXXXXXXXTXYXXSLKXRXXXXRDXSKXXXXLQXNSXXXEDTXXYYCXXXXXXXXXXFXYWGQGTXXYVSX

SEQ ID NO: 73 - protein, 16 aa, Murine Antibodies
CVASGFTFSNFWMNWV

SEQ ID NO: 74 - protein, 16 aa, Murine Antibodies
CSVTGDSITSGYRNWI

SEQ ID NO: 75 - protein, 17 aa, Murine Antibodies
CTVTGYSITSDYAWNWI

SEQ ID NO: 76 - protein, 17 aa, Artificial
  Synthetic
  misc_feature (11)..(11): Xaa can be any naturally occurring amino acid
  misc_feature (13)..(13): Xaa can be any naturally occurring amino acid
  misc_feature (15)..(15): Xaa can be any naturally occurring amino acid
CSVTGFSITSXYXNXWI

SEQ ID NO: 77 - protein, 14 aa, Artificial
  Synthetic
  misc_feature (2)..(2): Xaa can be any naturally occurring amino acid
  misc_feature (4)..(7): Xaa can be any naturally occurring amino acid
  misc_feature (9)..(9): Xaa can be any naturally occurring amino acid
  misc_feature (13)..(14): Xaa can be any naturally occurring amino acid
CXQXXXXPXTFGXX

SEQ ID NO: 78 - protein, 27 aa, Artificial
  Synthetic
  misc_feature (3)..(15): Xaa can be any naturally occurring amino acid
  misc_feature (17)..(17): Xaa can be any naturally occurring amino acid
  misc_feature (19)..(20): Xaa can be any naturally occurring amino acid
  misc_feature (24)..(24): Xaa can be any naturally occurring amino acid
  misc_feature (26)..(27): Xaa can be any naturally occurring amino acid
LEXXXXXXXXXXXXXTXYXXSLKXRXX

SEQ ID NO: 79 - protein, 18 aa, Artificial
  Synthetic
  misc_feature (2)..(11): Xaa can be any naturally occurring amino acid
  misc_feature (13)..(13): Xaa can be any naturally occurring amino acid
CXXXXXXXXXXFXYWGQG

SEQ ID NO: 80 - protein, 19 aa, Artificial
  Synthetic
  misc_feature (2)..(15): Xaa can be any naturally occurring amino acid
CXXXXXXXXXXXXXXWGQG

SEQ ID NO: 81 - protein, 156 aa, Homo sapiens
DENPDIECADCPIGFYNDPHDPRSCKPCPCHNGFSCSVIPETEEVVCNNCPPGVTGARCELCADGYFGDPFGEHGPVRPCQPCQCNSNVDPSASGNCDRLTGRCLKCIHNTAGIYCDQCKAGYFGDPLAPNPADKCRACNCNPMGSEPVGCRSDGT

SEQ ID NO: 82 - protein, 14 aa, Artificial
  Synthetic
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
CAQXSXLXPXFGGG

SEQ ID NO: 83 - protein, 174 aa, Homo sapiens
DENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 84 - protein, 138 aa, Murine Antibodies
MYLGLNCVFIVFLLKGVQSEVKLEESGGGLVQPGGSMKLSCVASGFTFSNFWMNWVRQSPEKGLEWVAEIRLKSNNYATHYAESLKGRFTISRDDSKSNVYLQMNNLRPEDTGIYYCTRDYYGSSFAYWGQGTLVTVS

SEQ ID NO: 85 - protein, 137 aa, Murine Antibodies
MMVLSLLYLLTALPGILSEVQLQESGPSLVKPSQTLSLTCSVTGDSITSGYRNWIRKFPGNKLEYMGYISYRGTTYYNPSLKSRISITRDTSKNQYYLQLNSVTTEDTATYYCAGPYDYDERTFAYWGQGTLVTVSA

SEQ ID NO: 86 - protein, 136 aa, Murine Antibodies
MRVLILLWLFTAFPGILSDVHLQESGPGLVKPSQSLSLTCTVTGYSITSDYAWNWIRQFPGNRLEWMGYITYSGGTNYNPSLKSRISFTRDTSKNQFFLQLNSVTTEDTATYYCANNFYGNFFDYWGQGTTLTVSS

SEQ ID NO: 87 - protein, 23 aa, Artificial
  Synthetic
QDCASGYKRDSARLGPFGTCIPR

SEQ ID NO: 88 - protein, 20 aa, Artificial
  Synthetic
DPDTGDCYSGDENPDIECAD

SEQ ID NO: 89 - protein, 24 aa, Artificial
  Synthetic
ELCADGYFGDPFGEHGPVRPCQPE

SEQ ID NO: 90 - protein, 21 aa, Artificial
  Synthetic
RQCNNNVDPSASGNCDRLTGR

SEQ ID NO: 91 - protein, 15 aa, Artificial
  Synthetic
LKCIHNTAGIYCDQR

(19) United States
(12) Patent Application Publication
(10) Pub. No.: US 2007/0065447 A1
(43) Pub. Date: Mar. 22, 2007

(54) Laminin-5 gamma2-binding peptides, related compositions, and use thereof

(75) Inventors: Karl Tryggvason, Djursholm (SE); Ida Stenfeldt Mathiasen, Kgs. Lyngby (DK); Soren Berg Padkaer, Vaerlose (DK); Svetlana Tarabykina, Frederiksberg (DK); Sirpa Salo, Oulu (FI); Ariel Boutaud, Cary, NC (US)

Correspondence Address: NOVO NORDISK, INC.;PATENT DEPARTMENT, 100 COLLEGE ROAD WEST, PRINCETON, NJ, 08540, US

(73) Assignees: Novo Nordisk A/S, Bagsvaerd (DK); BioStratum Incorporated, Durham, NC (US)

(21) Appl. No.: 11/413,663

(22) Filed: Apr. 28, 2006

Related U.S. Application Data
(63) Continuation of application No. PCT/DK04/00744, filed on Oct. 28, 2004.

(30) Foreign Application Priority Data
Oct. 29, 2003 (WO) PCT/EP03/12012

(51) Int. Cl. A61K 39/395; C07K 16/30

(52) U.S. Cl. 424/155.1; 530/388.8; 530/391.1

(57) ABSTRACT

Novel peptides that specifically bind the γ2 chain of laminin-5 and other γ2-associated proteins; related compositions (e.g., derivatives and variants of such peptides; nucleic acids comprising sequences encoding such peptides; pharmaceutical compositions comprising either of such molecules; etc.); methods of using the same for diagnostic, prophylactic, and therapeutic purposes; and additional new and useful related compositions and methods are provided.

### CROSS-REFERENCE TO RELATED PATENT APPLICATIONS

This patent application is a continuation of co-pending International Patent Application PCT/DK2004/000744 (published as WO 2005/040219), filed Oct. 28, 2004, which designated the US, and U.S. patent application Ser. No. 10/695,559, filed Oct. 28, 2003, and further claims priority to U.S. Provisional Patent Application 60/571656, filed May 13, 2004; U.S. Provisional Patent Application 60/523895, filed Nov. 20, 2003; and International Patent Application WO 2003EP12012, filed Oct. 29, 2003, the entirety of each of which is hereby incorporated by reference.

### FIELD OF THE INVENTION

This invention pertains to peptides that are capable of specifically and/or selectively binding a portion of the γ2 chain of laminin-5, related compositions, and various uses of such peptides and compositions.

### BACKGROUND OF THE INVENTION

The laminins are a family of basement membrane glycoproteins having a heterotrimeric chain composition of alpha, beta, and gamma subunits (“chains”). Laminin-5 (“Ln-5”), one member of the laminin protein family, is a component of epithelial basement membranes and has a chain composition of α3β3γ2 (Kallunki, et al., J. Cell Biol. 119:679-93, 1992; see also Matsui et al., J Biol Chem. 270(40):23496-503 (1995)). Ln-5 γ2 (or simply “γ2”) is approximately 1193 amino acids in length with a potential signal sequence of 21 amino acids (“AA”) and a mass of about 130 kD that in Ln-5 often is processed to about 100 kDa. Ln-5 γ2 is believed to be composed of five domains corresponding approximately to the following regions: Domain V: amino acid residues 22-196; Domain IV: amino acid residues 197-381; Domain III: amino acid residues 382-602; and Domain I & 2: amino acid residues 603-1193. Domain I & II appear to comprise a coiled coil domain structure. Between the coiled coil region of Domain I & II and Domain III is a flexible hinge region.

The γ2 chain has been shown to be strongly expressed in malignant cells located at the invasive front of several human carcinomas, as determined by in situ hybridization and immunohistochemical staining. More recently, it has been demonstrated that antibodies directed against Domain III, but not domains I-II of laminin-5, are able to reduce migration of epithelial derived cells, including epithelial-derived cancer cells (see Salo et al., Matrix Biol. 18(2):197-210 (1999) and US Patent Application 20020052307).

### BRIEF SUMMARY OF THE INVENTION

The invention provides novel peptides that bind to a portion of the human γ2 chain of Ln-5 (which also may be simply referred to herein as “γ2”), in most aspects particularly to Domain III of γ2 (“γ2 DIII” or simply “DIII”) and fragments thereof, related compositions (e.g., compositions that are structurally and/or functionally related to such Ln-5 binding proteins, such as peptides that induce an immune response including the production of anti-γ2 DIII antibodies (which may alternatively be referred to as “anti-L5G2D3 antibodies” or simply anti-L5G2D3 Abs), nucleic acids coding for expression of such Ln-5 binding peptides or immunogenic peptides, and the like), and new and useful methods of making, selling, and using such novel peptides, previously known γ2-binding peptides, and compositions related to either thereof.

In a particular exemplary aspect, the invention provides new and useful Ln-5 γ2 domain III (DIII) binding peptides (“L5G2D3BPs”), compositions comprising such L5G2D3BPs, and methods of using such peptides and/or compositions. L5G2D3BPs provided by the invention may be useful in, among other things, the detectable induction, promotion, enhancement, and/or other modulation of physiological and/or cellular responses related to the inhibition of cancer progression (e.g., the reduction of epithelial cell-derived neoplastic and/or preneoplastic cell migration) and/or diagnosis thereof in chordates (e.g., mammals), such as in a human afflicted with cancer or diagnosed as being at substantial risk of development of a cancer-related malignancy. In another sense, the invention relates to the use of a LGG2D3BP, such as one or more of the particular anti-γ2 DIII antibodies described herein, in the preparation of medicaments for the treatment of a cancer or pre-cancer condition in a human patient.

### BRIEF DESCRIPTION OF THE DRAWINGS

FIG. 1 shows the design of model of portion of γ2 DIII (residues 460-567) for epitope validation. Differences between LMG2_HUMAN and LMG2_MOUSE in C Terminal are highlighted.

FIG. 2 shows an alignment of laminin γ2 DIII and γ1 DIII sequences. At the top the amino acid sequence from human laminin gamma 2 domain III chain (382-602) is aligned to the gamma 1 chain domain III. The positions of cysteines are highlighted. The pattern of the cysteine bridges are known from the crystal structure of the gamma 1 chain and with this alignment of the gamma 2 domain III the pattern of the EGF domains in the domain III of gamma 2 can be seen to be conserved. Moreover, the organization of the cysteine bridges is depicted as pairs with bars connecting them. In addition, the reported cleavage site for BMP-1 is highlighted. The sequence used for generating the antibodies is also indicated by highlighting.

FIG. 3 shows a model of a portion of γ2 DIII (residues 460-567), showing predicted human Ln-5 specificity-determining amino acid residues.

FIG. 4 shows data obtained in an exemplary competition assay involving anti-γ2 Dill mAbs 5D5, 6C12, and 4G1.

FIG. 5 shows additional binding competition data for anti-γ2 DIII mAbs 5D5, 6C12, and 4G 1.

FIG. 6 shows the results of exemplary epitope mapping experiments involving γ2 DIII mAbs and peptides comprising particular portions of γ2 DIII.

FIG. 7 shows additional results of epitope mapping experiments involving anti-γ2 DIII mAbs 4G1, 5D5, and 6C12.

FIG. 8 shows further results of exemplary epitope mapping experiments involving mAbs 4G1, 5D5, and 6C12.

FIG. 9 illustrates the mapping of an ADR for mAb 4G1 (Ln-5 394-414) by direct ELISA performed as described in experimental procedures below. The results show binding of the 4G1 antibody to the epitope located at amino acid residues 394-414. Two other antibodies, 6C12 and 5D5, do not recognize the same epitope. BSA or a blocking buffer was used as a negative control for unspecific binding of the antibodies. The recombinant GST-LN5 γIII protein was used as a positive control. Mean value of three independent measurement ± SD is shown.

FIG. 10 shows the results of a competition ELISA performed as described in experimental procedures. The data show that both, mAb 5D5 and mAb 6C12, recognize a complex structural epitope, since all three peptides can to some degree inhibit binding of the antibody to GST-LN5 γIII. However, the epitopes are somewhat different for 5D5 and 6C12, as the peptide 518-537 does not interfere with binding of 5D5, while it inhibits 6C12 binding. Increasing concentrations of peptides ranging from 0 to 50 μg are indicated with a triangle.

FIG. 11 shows sequence alignments of sequences including CDRs of the light variable regions for mAbs 4G1, 5D5, and 6C12.

FIG. 12 shows sequence alignments of sequences including CDRs of the heavy variable in regions for mAbs 4G1, 5D5, and 6C12.

FIG. 13 shows a sequence alignment comparison of Fab light chain sequences of mAbs 4G1, 5D5, and 6C12.

FIG. 14 shows a sequence alignment comparison of Fab heavy chain sequences of mAbs 4G1, 5D5, and 6C12.

FIG. 15 provides views of the alignments of light and heavy Fab sequences for mAbs 4G1, 5D5, and 6C12.

FIG. 16 reflects the results of a dry down ELISA of selected tumor cells performed with mAb 5D5.

FIG. 17 provides a graph of the results of in vivo metastasis assays performed with mAbs 5D5 and 6C12, demonstrating the ability of these antibodies to inhibit metastases in vivo.

FIG. 18 provides sequence alignments of CDR-H1 sequences for mAbs 4G1, 5D5, and 6C1 2 as defined by AbM and Kabat rules.

FIGS. 19A-19C show reflection/refractive index data obtained by surface plasmon resonance affinity assays of mAbs 4G1, 5D5, and 6C12 and a γ2-associated peptide.

### DETAILED DESCRIPTION OF THE INVENTION

As indicated above, the invention described herein provides, among other things, new and useful peptides that are capable of binding to Ln-5 γ2, such as antibodies against Ln-5 γ2 and related γ2-binding peptides (including antibody fragments; antibody-like polypeptides—e.g., bispecific antibodies, diabodies, and single chain antibodies; and derivatives of any thereof, etc., examples of which are described in further detail below). The invention also provides nucleic acid molecules comprising sequences coding for production of such peptides; vectors comprising such nucleic acids; cells comprising such nucleic acids and/or vectors (e.g., bacterial cell vectors comprising such nucleic acids). The invention further provides compositions comprising such peptides, antibodies, nucleic acids, vectors, and cells. The invention additionally provides various new and useful methods of using such peptides, related molecules, and compositions (e.g., to induce, promote, and/or enhance one or more physiological responses associated with interfering with γ2 interactions; to deliver a molecule to γ2-associated tissues; etc.). The invention further relates to the use of such peptides and/or related molecules (e.g., nucleic acids comprising sequences encoding such peptides) in the preparation of medicaments for the treatment of ailments wherein interference of γ2 interactions would be beneficial (e.g., in the treatment of γ2-associated carcinomas or pre-carcinomas). Related compositions, such as antigenic peptides and peptide-encoding nucleic acids, and other compositions that modulate Ln-5 γ2 peptides (e.g., antisense or siRNA molecules targeted against Ln-5 γ2 peptides) also are provided by the invention. These and various additional aspects and features of the invention are described in further detail herein.

In one aspect, the invention provides new and useful peptides that bind (typically specifically or selectively bind) to the γ2 domain of a laminin-5 protein.

Terms like laminin-5, Ln-5, Ln5, etc., used herein, refer to human laminin-5 protein, unless otherwise stated or clearly contradicted by context (Ln-5 also collectively refers to any γ2-comprising isoforms of Ln-5, such as Ln-5A and Ln-5B (see, e.g., Kariya et al., JBC e-published manuscript M400670200 (Mar. 23, 2004)—i.e., JBC Papers in Press doi:10.1074/jbc.M400670200)). Terms referring to Ln-5 subunits (e.g., γ2) and domains thereof (e.g., γ2 domain III) also should be read with reference to human Ln-5 unless otherwise stated or clearly indicated otherwise by context.

In the context of this invention, a peptide that binds γ2 (which also may be referred to as a “Ln-5 γ2-binding peptide”, L5G2BP, “γ2-binding peptide”, or the like) refers to a peptide that selectively and/or specifically binds and remains detectably associated with γ2 (a) under typical physiological conditions for a significant period of time, (b) can be detected as binding γ2 in an Enzyme-linked immunosorbent assay (ELISA or EIA), Western blot, or other suitable diagnostic assay of protein binding known in the art and/or described elsewhere herein, and/or (c) remains associated for a period sufficient to detectably induce, promote, enhance, and/or otherwise modulate a physiological activity associated with γ2 in a cellular and/or physiological environment.

L5G2BPs typically bind to DIII or a portion of γ2 near DIII (e.g., the hinge region). Generally, where a L5G2BP binds to a region of γ2 near DIII the region is sufficient close such that upon binding access to DIII is at least partially blocked by the binding of the L5G2BP and/or one or more biological activities/functions associated with DIII are reduced or otherwise modulated. Typically, a L5G2BP is capable of binding its target in any suitable context in which it may occur. For example, a L5G2BP may bind to γ2 in the context of various heterotrimeric forms of Ln-5 (typically including soluble form(s)); a heterodimeric γ2/β3; a free form of γ2; a form of γ2 covalently bound to laminin-6, laminin-7, or other matrix protein (see, e.g. Champliaud M F et al., J. Cell Biol. (1996) vol. 132 (6) 1189-1198)); and/or a fragment of γ2, such as γ2 fragments originating from in vivo processing (see, e.g., Schenk and Quarantas, TRENDS cell biol. (2003) vol. 13 (7) p. 366-375), etc. (additional targets for L5G2BPs are described elsewhere herein and/or may be known in the art). A L5G2BP also typically can bind to a γ2-associated target where such target appears in the context of a larger protein (e.g., a fusion protein). A L5G2BP also or alternatively can bind its target in free form, fragment form, and/or in one or more forms associated with other proteins, structures, etc. From the foregoing, it also should be understood that, unless otherwise stated or clearly contradicted by context, a γ2-binding peptide (such as a γ2 DIII-binding peptide) can bind to a portion of γ2 (e.g., γ2 DIII) in any suitable context (e.g., in the context of a γ2/β3 heterodimer, in the context of a free fragment of γ2, in the context of a heterotrimeric Ln-5 molecule, in the context of any of these in association with an interacting biomolecule, etc.).

Typical physiological conditions include a temperature of about 37° C., such as at a temperature of from about 20-40° C. (for example at room temperature), and a pH of about 7-8 (e.g., about 7.5), or other suitable combination of temperature, pH, and other conditions.

A significant period of time with respect to L5G2BP binding of a target typically refers to a period of at least about 30 minutes, at least about 45 minutes, at least about 1 hour, at least about 4 hours, at least about 8 hours, or longer such as about 1-12 hours, about 1-24 hours, about 1-36 hours, about 1-48 hours, about 1-72 hours, etc.

One exemplary aspect of the invention is embodied in a γ2-binding peptide that binds free γ2 and/or a related molecule comprising γ2, and/or heterotrimeric Ln-5 (optionally being or including a fragment of heterotrimeric Ln-5) under conditions such that epithelial cell motility is detectably reduced as compared to substantially similar cells not contacted with the antibody.

Terms such as “peptide,” “protein,” and “polypeptide” are to be understood to provide support for one another herein and to be amenable to interchangeable use generally, unless otherwise stated or contradicted by context; provided that the reader recognize that each type of respective amino acid polymer-containing molecule can be associated with significant differences and thereby form individual aspects of the invention (for example, an antibody, which is composed of multiple polypeptide chains, or similar polypeptide/protein may be significantly different from, for example, a single chain antibody, a peptide immunoadhesin, single chain immunogenic peptide, or other small “peptide” (single chain amino acid polymer molecule comprising less than about 100 amino acid residues) with respect to, for example, delivery, avidity, and/or stability). Furthermore, terms like “peptide” and “protein” used herein should generally be understood as referring to any suitable peptide of any suitable size and composition (e.g., with respect to the number of amino acids, number of associated chains in a protein molecule, overall size, etc.). Moreover, peptides in the context of the inventive methods and compositions described herein can comprise non-naturally occurring and/or non-L amino acid residues, unless otherwise stated or contradicted by context.

Unless otherwise stated or clearly contradicted by context, the term peptide (and if discussed as individual aspects the term(s) polypeptide and/or protein) also generally encompasses derivatized peptide molecules (“derivatives”). A “derivative” is a peptide in which one or more of the amino acid residues of the peptide have been chemically modified (e.g. by alkylation, acylation, ester formation, or amide formation) or associated with one or more non-amino acid organic and/or inorganic atomic or molecular substituents (e.g., a polyethylene glycol (PEG) group, a lipophilic substituent (which optionally may be linked to the amino acid sequence of the peptide by a spacer residue or group such as -alanine, gamma-aminobutyric acid (GABA), L/D-glutamic acid, succinic acid, and the like), a fluorophore, biotin, a radionuclide, etc.) and also or alternatively can comprise non-essential, non-naturally occurring, and/or non-L amino acid residues, unless otherwise stated or contradicted by context (however, it should be recognized that such derivatives can, in and of themselves, be considered independent features of the invention and inclusion of such molecules within the meaning of peptide is done for the sake of convenience in describing the invention rather than to imply any sort of equivalence between “naked” peptides and such derivatives). Non-limiting examples of unusual amino acid residues that can be comprised in a derivative include, for example, 2-aminoadipic acid; 3-Aminoadipic acid; β-Alanine; β-aminopropionic acid; 2-Aminobutyric acid; 4-Aminobutyric acid; 6-Aminocaproic acid; 2-Aminoheptanoic acid; 2-Aminoisobutyric acid; 3-Aminoisobutyric acid, 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; N-Methylisoleucine; 6-N-Methyllysine; N-Methylvaline; Norvaline; Norleucine; Ornithine; and the like. Derivatives of L5G2BPs provided by the invention are described in further detail elsewhere herein.

L5G2BPs include antibodies, antibody fragments, and antibody-like molecules. In one aspect, the invention provides L5G2BPs that can be characterized as a peptide that comprises one or more anti-γ2, and typically one or more anti-γ2 DIII, antibody CDRs or biologically functional variants thereof. Such a CDR-containing peptide typically includes at least one region of H chain and L chain CDRS. Plural CDRs can be bound directly or via an appropriate peptide linker.

The invention also provides non-antibody L5G2BPs, such as anti-γ2 DIII antibody fragments and other peptides that comprise portions of L5G2BPs of the invention.

To better illustrate the invention, a number of exemplary types of L5G2BPs (including particular examples thereof) are described in detail here.

Particularly useful types of L5G2BPs are antibodies that bind to γ2. Typically L5G2BP antibodies bind to γ2 DIII or a portion of γ2 located near DIII (e.g., in the hinge region). An antibody against γ2 (or “anti-γ2 antibody” or simply “γ2 antibody”), herein, typically refers to an antibody that specifically binds to one or more portions of γ2, typically portions of γ2 DIII, under cellular and/or physiological conditions. Often, an anti-γ2 antibody binds to γ2 for an amount of time sufficient to induce, promote, enhance, and/or otherwise modulate a physiological effect associated with Ln-5, γ2, or a γ2-associated molecule/structure. In another aspect, an anti-γ2 antibody also or alternatively can be characterized as an antibody that binds and remains associated with γ2 so as to allow detection of the bound γ2 portion/molecule or associated molecule/structure in a composition by ELISA, Western blot, or other similarly suitable protein binding technique described herein and/or known in the art and/or to otherwise be detectably bound thereto after a relevant period of time (e.g., at least about 15 minutes, at least about 30 minutes, at least about 45 minutes, at least about 1 hour, at least about 2 hours, at least about 4 hours, at least about 6 hours, at least about 12 hours, about 1-24 hours, about 1-36 hours, about 1-48 hours, about 1-72 hours, about one week, or longer). A γ2 antibody can be an antibody that specifically binds γ2 or a γ2 fragment/portion solely (in a context free from the α3 and/or β3 chains of Ln-5) and/or in the context of a heterotrimeric or otherwise multimeric Ln-5 protein—and in either case in free form or in a form associated with other structures and/or biomolecules, such as an Ln-5-binding protein, the basal lamina of a cell, etc.

Anti-γ2 antibodies, in the context of this invention, include antibodies that bind to other portions of Ln-5 or even other non-Ln-5 molecules. For example, an anti-γ2 antibody can also bind to the β3 chain of Ln-5 or α3 chain of Ln-5 in the context of an epitope presented by a heterotrimeric Ln-5 protein (e.g., a form of Ln-5 that is naturally occurring or a multiple chain fragment thereof) or a heterodimeric molecule, such as in the context of a conformational epitope (i.e., structural, noncontiguous, or nonlinear epitope). Other anti-γ2 antibodies can bind to non-similar/non-homologous targets in addition to γ2. For example, multispecific antibodies provided by the invention (discussed further elsewhere herein) can bind any suitable additional target(s) in addition to a portion of γ2 (e.g., a tumor-associated antigen such as carcinoembryonic antigen (CEA), Ep-CAM (i.e., KSA/GA733-2), 67 kDa laminin receptor (elastin receptor), gp 100, mucin (MUC-1), A33 antigen, etc.).

The term antibody generally refers to an immunoglobulin molecule, a “fragment” of an immunoglobulin molecule that at least partially retains a tetrameric antibody-like structure and comprises at least one heavy chain-light chain pair that is of essentially similar length as a wild-type antibody molecule (whether produced by actual fragmentation of an antibody or by recombinant/synthetic production), or a derivative of either thereof, which has the ability to specifically bind to one or more antigens under typical physiological conditions for significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (e.g., a time sufficient to induce, promote, enhance, and/or modulate a physiological response associated with antibody binding to Ln-5, γ2, or both).

The term immunoglobulin refers to a class of structurally related proteins consisting of two pairs of polypeptide chains, one pair of light (L) low molecular weight chains and one pair of heavy (H) chains, all four inter-connected by disulfide bonds. The structure of immunoglobulins has been well characterized. See, e.g., FUNDAMENTAL IMMUNOLOGY (Paul, W., ed., 2nd ed. Raven Press, N.Y. (1989)). Briefly, each heavy chain typically is comprised of a heavy chain variable region (abbreviated herein as HCVR or VH) and a heavy chain constant region. The heavy chain constant region typically is comprised of three domains, CH1, CH2, and CH3. Each light chain typically is comprised of a light chain variable region (abbreviated herein as LCVR or VL) and a light chain constant region. The light chain constant region typically is comprised of one domain, CL. The VH and VL regions can be further subdivided into regions of hypervariability (or hypervariable regions, which can be hypervariable in sequence and/or form of structurally defined loops), also termed complementarity determining regions (CDRs), interspersed with regions that are more conserved, termed framework regions (FR). In full length, naturally produced antibodies, each VH and VL typically is composed of three CDRs and four FRs, arranged from amino-terminus to carboxy-terminus in the following order: FR1, CDR1, FR2, CDR2, FR3, CDR3, FR4 (which also may be referred to as FR L1, CDR L1, etc. or loop L1, L2, L3 in the light chain variable domain and loop H1, H2, and H3 in the heavy chain domain in the case of hypervariable loop regions (see, e.g., Chothia and Lesk J. Mol. Biol. 196:901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases such as “variable domain residue numbering as in Kabat” and “according to Kabat” herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The carboxy-terminal portion of each antibody chain typically defines a constant region primarily responsible for effector function. Human light chains typically are classified as kappa and lambda light chains. Heavy chains typically are classified as mu, delta, gamma, alpha, or epsilon, and typically define an antibody's isotype as IgM, IgD, IgG, IgA, and IgE, respectively.

In describing the invention, it may be useful to refer to the antigen binding region of an anti-γ2 antibody. The antigen binding region refers to the portion of an antibody (or corresponding region in an antibody fragment or antibody-like molecule (e.g., a L5G2D3BP fusion protein that comprises antibody or antibody-like sequences/domains)) that contains the amino acid residues that interact with an antigen and confer on the antibody its specificity and affinity for the antigen. An antibody binding region typically includes the FR regions necessary to maintain the proper conformation of the antigen-binding residues (or suitable variants of such sequences).

It also should be understood that the term antibody also generally includes polyclonal antibodies; monoclonal antibodies (mAbs); and antibody-like proteins (antibody-like molecules) having an antibody structure (at least partially comprising a tetrameric heavy chain-light chain, heavy chain-light chain structure) such as chimeric antibodies and humanized antibodies.

Unless otherwise stated or clearly contradicted by context, an antibody can generally possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. No. 5,916,771), and other suitable techniques known in the art. This principle also applies for antibody “fragments” and other antibody-like molecules provided by this invention, examples of which are described further elsewhere herein.

An antibody can be characterized on its ability to specifically and/or selectively bind to an antigenic determinant region (ADR) or, more particularly, an epitope, on a particular molecule (e.g., γ2 DIII). An epitope is the area or region on an antigen to which a specifically-binding peptide (such as an antibody) binds. An epitope of γ2 may comprise amino acid residues directly involved in the binding of the antibody (the immunodominant component of the epitope) and other amino acid residues, which are not directly involved in the binding, such as amino acid residues which are effectively blocked by the specifically antigen binding peptide (in other words, the amino acid residue is within the “footprint” of the specific antigen binding peptide). Terms such as antigenic determinant, “antigenic determinant region,” antigenic determining region, and the like refer to any portion of a peptide, either in a single chain or across multiple chains that comprises one or more epitopes.

An antigenic determinant in the context of this invention generally includes any peptide or peptide-derivative determinant capable of being specifically bound by an immunoglobulin. Antigenic determinant regions and epitopes can comprise any suitable number of amino acids, in any suitable position (with respect to the linear sequence of Ln-5) orientation (with respect to folded Ln-5, γ22, DIII, or a fragment thereof), and amino acid composition (and consequently, at least in part, charge). Thus, for example, an epitope may be composed of about 3-10 amino acids, typically 3-8 amino acids, in one or more contiguous or noncontiguous locations with respect to the primary sequence of Ln-5 (e.g., an epitope can consist essentially of 2, 3, 4, 5, 6, 7, or 8 amino acid residues distributed in 1, 2, 3, 4, or 5 noncontiguous locations in γ2 DIII). Alternatively, for example, an antigenic determinant region can be considered to be defined by a region of about 5-40 contiguous amino acid residues (e.g., about 7-30 amino acid residues, about 5-20 amino acid residues, or about 3-15 amino acid residues) in γ2 DIII (solely or in combination with a portion of an adjacent Ln-5 domain). In some epitopes it may be the case that just one amino acid residue or only a few amino acid residues are critical to recognition by a CDR, CDR(s), or paratope (and thereby most important to L5G2D3BP:γ2 DIII antigen affinity and avidity). As such, an epitope can be characterized on the basis of one or more of such critical residues, with the recognition that other residues may also make some lesser contribution to the function of the epitope. In the case of an epitope defined by a region of amino acids, it may be that one or more amino acids in the region make only a minor contribution or even negligible contribution to antibody binding, such that the residue can be subject to substitution with an appropriate different residue without resulting in “a loss” of the epitope to at least some L5G2D3BPs specific for it.

An antibody specific for a particular epitope or antigenic determinant may yet cross-react with other epitopes or antigenic determinants on other Ln-5 chains or, more typically, other biomolecules that may be present in some biological context with Ln-5 (e.g., near Ln-5 and/or γ2 secreting cells). More typically, Ln-5 γ2 binding peptides will cross-react with Ln-5 homologues from other species. In either or both contexts, typically such cross-reactive antibodies are selective for human γ2 (in the context of heterotrimeric human Ln-5, free human γ2, a fragment of γ2, or a combination of any thereof) with respect to relevant structure and/or environmental factors (e.g., in the context of the lamina lucida, the invasive front of a cancer cell population or tumor cell mass, etc.).

Terms such as “selective,” “selectively,” and “selectivity” herein refer to the preferential binding of a γ2 binding peptide, such as an anti-γ2 antibody, for a particular region, target, or peptide; typically a region or epitope in γ2 (commonly in DIII), as opposed to one or more other biological molecules, structures, cells, tissues, etc. In one aspect, anti-γ2 antibodies provided by the invention may also or alternatively be characterized as being selective for a portion of γ2 or a peptide comprising a portion of γ2 in the context of a human epithelial cell and/or the context of a physiological environment in which γ2, Ln-5, and/or other γ2-associated peptides are secreted from cells (e.g., the anti-γ2 antibody will selectively bind to the portion of γ2 it targets or a peptide comprising the portion of γ2 over other components of an epithelial cell; the anti-γ2 antibody will selectively bind the targeted portion of γ2 or a peptide comprising the portion over other molecules present at the invasive front of a tumor). In another aspect, γ2 antibodies provided by the invention may be characterized as being selective for a portion of γ2 with respect to the basal lamina of a mammal, such as a human being (either individually or in a population of humans, such as a population of human patients with breast cancer, colorectal cancer, lung cancer, skin cancer, ovary cancer, cervical cancer, pancreatic cancer, vaginal cancer, head cancer, or neck cancer and having other characteristics selected according to standard principles for pharmaceutical clinical development, e.g., as set forth by current US FDA guidelines, regulations, and associated practices). In another aspect, an anti-γ2 antibody of the invention may be characterized as being selective for a peptide comprising a portion of γ2 DIII (such as a γ2/,3 heterodimer or γ2 fragment associated with cell migration) in the context of the invasive front of a cancer cell population or tumor growth or other γ2-sensitive cancer-associated or preneoplastic tissue(s). L5G2D3BP compositions of the invention, combination compositions, and related compositions (described further elsewhere herein) typically can be used to promote the treatment of any one or any combination of these types of cancers and/or modulate the activity of preneoplastic cells in such precancerous tissues.

In yet another aspect, the invention provides a Ln-5 γ2 binding peptide, such as an anti-γ2 antibody, that also or alternatively is selective for a particular region of γ2 over other regions of γ2. Thus, for example, the invention provides an anti-γ2 antibody that is selective for a region of γ2 DIII defined by one or more amino acids located within the region of about residue 494 to about residue 515 of Ln-5 γ2 DIII (e.g., γ2 chain residues 494-516) and/or one or more amino acid residues located within from about residue 540 to about residue 550 (such as residues 539-552 of Ln-5 γ2) as compared to intact and unprocessed (with respect to cleavage by proteolytic enzymes) γ2 domain III, a peptide comprising intact and unprocessed γ2 DIII, or a peptide comprising a larger or distinct fragment thereof (e.g., about residues 390-570, such as residues 392-597 of Ln-5, or about residues 395-490, etc.). Selectivity in this context can be determined by any suitable technique. For example, selectivity can be determined by competitive ELISA assays as described in the Examples provided herein.

Unless otherwise stated, all references to amino acid residue numbers/numbering (positions) herein, with respect to Ln-5 and portions thereof, is made with respect to full-length human Ln-5 γ2 (e.g., SEQ ID NO:1).

Ln-5 γ2 binding peptides of the invention are typically used in and provided in an at least substantially isolated form. A substantially isolated molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition comprising an anti-γ2 antibody or other L5G2BP provided in accordance with this invention will exhibit at least about 98%, 98%, or 99% homogeneity for the Ln-5 γ2 binding peptide or antibody in the context of all present peptide species in the composition or at least with respect to substantially active peptide species in the context of proposed use (in the context of combination compositions provided by the invention, such percentages may refer to the percentage of species that the combination makes up in the composition). For example, a peptide stabilizer/buffer such as an albumin may be intentionally included in a final pharmaceutical formulation, without impeding the activity of L5G2BPs.

An isolated molecule typically refers to a molecule that is not associated with significant amounts (e.g., more than about 1%, more than about 2%, more than about 3%, or more than about 5%) of extraneous and undesirable biological molecules, such as non-Ln-5 γ2 binding biomolecules (or γ2 binding molecules that may interfere with the binding and/or activity of a Ln-5 γ2 binding peptide) contained within a cell, cell culture, chemical media, or animal in which the Ln-5 γ2 binding peptide is produced. An isolated molecule also or alternatively can refer to a molecule that has passed through such a stage of purity due to non-natural (human) intervention (whether automatic, manual, or both) for a significant amount of time (e.g., at least about 10 minutes, at least about 20 minutes, at least an hour, or longer).

In many of the various compositions provided by the invention, such as in a composition comprising one or more pharmaceutically acceptable carriers, a Ln-5 γ2 binding peptide can be present in relatively small amounts in terms of numbers of total molecular species in the composition (e.g., in the case of a composition comprising a large amount of a pharmaceutically acceptable carrier, stabilizer, and/or preservative). In some cases additional peptides, such as BSA, can be included in such a composition with a previously purified Ln-5 γ2 binding peptide. However, provided that such additional constituents of the composition are acceptable for the intended application of the Ln-5 γ2 binding peptide, such a composition may still be described as comprising an “isolated” Ln-5 γ2 binding peptide, unless otherwise indicated or clearly contradicted by context.

In one aspect, the invention provides Ln-5 γ2 binding peptides that can be characterized as being substantially free of other Ln-5 γ2 binding peptides, such as γ2 binding antibodies having different antigenic specificities (e.g., an isolated antibody that specifically binds human γ2 DIII is substantially free of antibodies that specifically bind antigens other than Ln-5, particularly in a relevant context for use of such a γ2 binding peptide, such as in the lamina lucida, lamina densa, the invasive front of an epithelial-derived cancer cell population, a cancerous or precancerous tissue associated with poorly defined hemidesmosomes and/or promigratory γ2 DIII-associated peptides, etc.). However, in another aspect, the invention provides a composition comprising a number of L5G2D3BPs with different specificities and characteristics (e.g., the invention provides in one aspect a “cocktail” of anti-γ2 DIII antibodies having different specificity and/or selectivity characteristics).

Anti-γ2 DIII antibodies provided by the invention include polyclonal antibodies against Ln-5, γ2/β3 heterodimers, and/or free γ2 peptides comprising DIII (and suitable fragments thereof). Methods for producing polyclonal Abs against Ln-5 γ2 are described in, e.g., U.S. patent application Ser. No. 10/695,559 and U.S. Pat. No. Application Publication No. 20020062307. In general, peptides comprising any of the specifically identified regions of DIII described herein can be used to immunize an animal to produce a pool of anti-γ2 DIII polyclonal antibodies.

Typically, an anti-γ2 DIII“antibody” in the context of this invention refers to a monoclonal antibody. In the context of this invention, a “monoclonal antibody” refers to a composition comprising a homogeneous antibody population having a uniform structure and specificity. Typically a monoclonal antibody is an antibody obtained from a population of substantially homogeneous antibodies; i.e., the individual antibodies comprised in the population are identical except for possible naturally occurring mutations that may be present in minor amounts. Monoclonal antibodies commonly are highly specific, typically being directed against a single antigenic site. Furthermore, in contrast to conventional (polyclonal) antibody preparations which typically include different antibodies directed against different antigenic determinants, each monoclonal antibody typically is directed against a single determinant on the antigen. The modifier “monoclonal” indicates the character of the antibody as being obtained from a substantially homogeneous population of antibodies, and is not to be construed as requiring production of the antibody by any particular method unless specifically stated. For example, monoclonal antibodies to be used in accordance with various aspects of the present invention may be made by the hybridoma method first described by Kohler et al., Nature 256:495 (1975), or may be made by recombinant DNA methods (see, e.g., U.S. Pat. No. 4,816,567). Monoclonal antibodies may also be isolated from phage antibody libraries using the techniques described in, for example, Clackson et al., Nature 352:624-628 (1991) and Marks et al., J. Mol. Biol. 222:581-597 (1991).

Monoclonal antibodies herein specifically include “chimeric” antibodies. The term “chimeric antibody” refers to an antibody that contains one or more regions from one antibody and one or more regions from one or more other antibodies (typically of antibodies of different species). Chimeric antibodies include monovalent, divalent, and polyvalent antibodies. A monovalent chimeric antibody typically is a dimer (HL) formed by a chimeric H chain associated through disulfide bridges with a chimeric L chain. A divalent chimeric antibody typically is a tetramer (H2L2) formed by two HL dimers associated through at least one disulfide bridge. A polyvalent chimeric antibody can also be produced, for example, by employing a CH region that aggregates (e.g., from an IgM H chain, or μ chain). Typically, a chimeric antibody refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as the fragments exhibit the desired biological activity (see, e.g., U.S. Pat. No. 4,816,567; and Morrison et al., Proc. Natl. Acad. Sci. USA 81:6851-6855 (1984)).

Chimeric antibodies may be produced by recombinant processes well known in the art (see, e.g., Cabilly et al, Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); European Patent Application 125023; Neuberger et al., Nature 314:268-270 (1985); European Patent Application 171496; European Patent Application 173494; WO 86/01533; European Patent Application 184187; Sahagan et al., J. Immunol. 137:1066-1074 (1986); Robinson et al., International Patent Publication #PCT/US86/02269 (published May 7, 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane ANTIBODIES: A LABORATORY MANUAL, cited elsewhere herein). The production of L5G2BPs generally is discussed in further detail elsewhere herein.

Humanized monoclonal anti-γ2 antibodies (e.g., anti-γ2 DIII antibodies) also are provided by the invention. A “humanized” antibody is an antibody that is derived from a non-human species, in which certain amino acids in the framework and constant domains of the heavy and light chains have been mutated so as to avoid or abrogate an immune response in humans. Humanized forms of non-human (e.g., murine) antibodies are thus chimeric antibodies which contain at least (and usually only or little more than) minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications typically are made to further refine antibody functionality and/or physiochemical properties. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. A humanized antibody optionally also will comprise at least a portion of an immunoglobulin constant region (Fc), typically that of a human immunoglobulin. For further details regarding the characteristics and production of typical humanized antibodies, see, e.g., Jones et al., Nature 321:522-525 (1986); Riechmann et al., Nature 332:323-329 (1988); and Presta, Curr. Op. Struct. Biol. 2:593-596 (1992).

Alternatively, a humanized antibody may be produced by fusing the constant domains from a human antibody to the variable domains of a non-human species. Examples of methods that can be used to make humanized antibodies may be found in, e.g., U.S. Pat. Nos. 6,054,297, 5,886,152, and 5,877,293. As indicated above, a humanized antibody is designed to have greater homology to a human immunoglobulin than animal-derived monoclonal antibodies. Non-human amino acid residues from an “import” (non-human chordate, typically mammalian) variable domain typically are transfected into a human “backbone”. Humanization can be essentially performed following the method of Winter and co-workers (see, e.g., Jones et al., Nature 321:522-525 (1986); Riechmann et al., Nature 332:323-327 (1988); Verhoeyen, et al., Science, 239:1534-1536 (1988)), by substituting rodent complementarity determining regions (“CDRs”) or CDR sequences for the corresponding sequences of a human antibody. Accordingly, in such humanized antibodies, the CDR portions of the human variable domain are substituted by the corresponding sequence from a non-human species. Thus, humanized antibodies are typically human antibodies in which some CDR residues and possibly some framework residues are substituted by residues from analogous sites in rodent antibodies. The choice of human variable domains, both light and heavy, to be used in making the humanized antibodies is important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (see, e.g., Sims et al., J. Immunol., 151:2296 (1993) and Chothia et al., J. Mol. Biol., 196:901 (1987) for a description of such methods and related principles). Another method uses a particular framework derived from the consensus sequence of all human antibodies of a particular subgroup of light or heavy chains. The same framework may be used for several different humanized antibodies (see, e.g., Carter et al., Proc. Natl. Acad. Sci. USA, 89:4285 (1992) and Presta et al., J. Immunol., 151:2623 (1993)).

It typically also is important that humanized antibodies retain high affinity and/or high avidity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. In general, the construction of such models involves building a framework by reference to an antibody crystal structure, such as those that are available from the Protein Databank (http://www.rcsb.org/), applying least squares structure fitting (for example with ProFit—http://www.bioinf.org.uk/software/profit/); and optionally analyzing sidechain replacement using molecular graphics software or CONGEN (http://www.congen.com/). Canonical conformations for CDRs, which also are useful in modeling, can be identified using known canonicals for CDRs (e.g., Kabat, Abm, etc.). Computer programs also are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is maximized, although it is the CDR residues that directly and most substantially influence antigen binding.

Murine antibodies or antibodies from other species can be humanized or primatized using any suitable techniques, a number of suitable techniques being already well known in the art (see e.g., Winter and Harris Immunol Today 14:43-46 (1993) and Wright et al. Crit. Reviews in Immunol. 12125-168 (1992)). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (see, e.g., Liu et al. P.N.A.S. 84:3439 (1987) and J. Immunol. 139:3521 (1987)). mRNA can be isolated from a hybridoma or other cell producing the antibody and used to produce cDNA. The cDNA of interest may be amplified by the polymerase chain reaction (PCR) using specific primers (U.S. Pat. Nos. 4,683,195 and 4,683,202). Alternatively, a library can be made and screened to isolate a sequence of interest. The nucleic acid sequence encoding the variable region of the antibody can then fused to human constant region sequences. Sequences of human constant regions (as well as variable regions) may be found in Kabat et al. (1991) Sequences of Proteins of Immunological Interest, N.I.H. publication no. 91-3242 and more recent and related data can be accessed at http://www.biochem.ucl.ac.uk/˜martin/abs/GeneralInfo.html. The choice of isotype for a designed antibody typically can be guided by the desired effector functions, such as complement fixation, or activity in antibody-dependent cellular cytotoxicity. Exemplary isotypes are IgG1, IgG2, IgG3, and lgG4. Either of the human light chain constant regions, kappa or lambda, may be used. A humanized antibody encoded by such a nucleic acid can then be expressed by conventional methods.

Another important feature of the invention is anti-γ2 DIII antibodies that are “fully human.” The terms “human antibody”, “human antibodies”, “human Ln-5 γ2 antibody”, and “human Ln-5 γ2 antibodies” refer to antibodies having both variable and constant regions derived from human germ line immunoglobulin sequences. Such “human” antibodies may include amino acid residues not encoded by human germ line immunoglobulin sequences (e.g., mutations introduced by random or site-specific mutagenesis in vitro or by somatic mutation in vivo), for example in the CDRs, such as in CDR3. However, the term “human antibody”, as used herein, is not intended to include antibodies in which CDR sequences derived from the germ line of another mammalian species, such as a mouse, have been grafted onto human framework sequences, e.g., humanized antibodies or human/mouse chimeric antibodies.

Human anti-γ2 DIII antibodies can also be generated in humanized transgenic animals (e.g., mice, rats, sheep, pigs, goats, cattle, horses, etc.) comprising human immunoglobulin loci and native immunoglobulin gene deletions, such as in a XenoMouse™ (Abgenix—Fremont, Calif., USA) (see, e.g., Green et al. Nature Genetics 7:13-21 (1994); Mendez et al. Nature Genetics 15:146-156 (1997); Green and Jakobovits J. Exp. Med. 188:483-495 (1998); European Patent No., EP 0 463 151 B1; International Patent Application Nos. WO 94/02602, WO 96/34096; WO 98/24893, WO 99/45031, WO 99/53049, and WO 00/037504; and U.S. Pat. Nos. 5,916,771, 5,939,598, 5,985,615, 5,998,209, 5,994,619, 6,075,181, 6,091,001, 6,114,598 and 6,130,364) or transgenic vertebrates comprising a minilocus of human Ig-encoding genes. Splenocytes from these transgenic mice or other vertebrates can be used to produce hybridomas that secrete human monoclonal antibodies according to well known techniques. Such transgenic vertebrates, vertebrates comprising an operable nucleic acid sequence coding for expression of an L5G2D3BP, vertebrates stably transfected with one or more L5G2D3BP-encoding nucleic acid sequences, and the like, are additional features of the invention. Typically, such vertebrates are non-human mammals.

Non-limiting examples of antibodies and antibody-like molecules provided by this invention include (a) a complete functional, immunoglobulin molecule comprising: (i) two identical chimeric heavy chains comprising a variable region with a human B cell surface antigen specificity and human constant region and (ii) two identical all human (i.e. non-chimeric) light chains; (b) a complete, functional, immunoglobulin molecule comprising: (i) two identical chimeric heavy chains comprising a variable region as indicated, and a human constant region, and (ii) two identical all non-human (i.e. non-chimeric) light chains; and (c) a monovalent antibody, i.e., a complete, functional immunoglobulin molecule comprising: (i) two identical chimeric heavy chains comprising a variable region as indicated, and a human constant region, and (ii) two different light chains, only one of which has the same specificity as the variable region of the heavy chains.

In addition to such antibody-like molecules and full sized antibodies, the invention also provides “fragments” related to the anti-γ2 antibodies specifically described herein. Antibody “fragments” that retain/exhibit the ability to specifically bind to a γ2-associated peptide typically can be suitably used in various methods of the invention and/or incorporated in various compositions of the invention (sometimes advantageously so).

It should be generally understood that any suitable antibody fragment can be used as a surrogate for an antibody in inventive compositions, methods, and uses described herein, and visa versa, unless otherwise stated or clearly contradicted by context (in situations where the size of the L5G2BP is relevant fragments may be suitable whereas full-sized antibodies may not). Thus, in one exemplary aspect the invention provides functional fragments of any of the antibodies provided by the invention, which may generally be obtained by any known technique, such as, but not limited to, enzymatic cleavage, peptide synthesis, and recombinant protein production techniques. Such antibody “fragments” can be characterized by possessing any one or combination of the features described herein as being associated with anti-γ2 antibodies, to the extent appropriate (e.g., many fragments lack an Fc domain and, accordingly, do not induce or promote antibody-associated complement functions).

An antibody fragment also or alternatively can be characterized as a peptide that comprises a portion of a full length antibody. In one aspect, an antibody fragment refers to a peptide that consists essentially or consists only of a portion of an antibody molecule. Thus, for example, in one aspect the invention provides an antibody fragment comprising at least a portion of a heavy chain variable domain containing any of the VH CDRs described herein and optionally also a light chain-variable domain comprising any of the light chain CDRs described herein, wherein the heavy chain variable domain, and optionally the light chain variable domain, optionally is (are) fused to an additional moiety, such as an immunoglobulin constant domain. Constant domain sequences can be added to the heavy chain and/or light chain sequence(s) to form species with partial length heavy and/or light chain(s). Constant regions, or portions thereof, of any antibody isotype can be used for this purpose, including IgG, IgM, IgA, IgD, and IgE constant regions.

Examples of antibody fragments include (i) a Fab fragment, a monovalent fragment consisting essentially of the VL, VH, CL and CH I domains; (ii) F(ab)2 and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., (1989) Nature 341:544-546), which consists essentially of a VH domain; and (vi) one or more isolated CDRs or a functional paratope. Additional antibody fragments include Fab′ fragments, dsFv molecules, diabodies, and the like. In one exemplary aspect, the invention provides an antibody fragment comprising a first polypeptide chain that comprises any of the heavy chain CDRs described herein and a second polypeptide chain that comprises any of the light chain CDRs described herein, wherein the two polypeptide chains are covalently linked by one or more interchain disulfide bonds. In a more particular aspect, the invention provides a two-chain antibody fragment having such features wherein the antibody fragment is selected from Fab, Fab′, Fab′—SH, Fv, and/or F(ab′)2 fragments. Other antibody “fragments” include “kappa bodies” (see, e.g., III et al., Protein Eng 10: 949-57 (1997)) and “janusins” (described further elsewhere herein).

Antibodies can be fragmented using conventional techniques, and the fragments screened for utility in the same manner as described above for whole antibodies. For example, F(ab′)2 fragments can be generated by treating antibody with pepsin. The resulting F(ab′)2 fragment can be treated to reduce disulfide bridges to produce Fab′ fragments. Fab fragments can be obtained by treating an IgG antibody with papain; F(ab′) fragments can be obtained with pepsin digestion of IgG antibody. A F(ab′) fragment also can be produced by binding Fab′ described below via a thioether bond or a disulfide bond. A Fab′ fragment is an antibody fragment obtained by cutting a disulfide bond of the hinge region of the F(ab′)2. A Fab′ fragment can be obtained by treating a F(ab′)2 fragment with a reducing agent, such as dithiothreitol. Antibody fragment peptides can also be generated by expression of nucleic acids encoding such peptides in recombinant cells (see, e.g., Evans et al., J. Immunol. Meth. 184: 123-38 (1995)). For example, a chimeric gene encoding a portion of a F(ab′)2 fragment can include DNA sequences encoding the CH1 domain and hinge region of the H chain, followed by a translational stop codon to yield such a truncated antibody fragment molecule.

Any suitable antibody fragment can be used in the context of the inventive methods described herein, or incorporated in the compositions of the invention (in addition to or in place of, for example, an anti-γ2 DIII antibody) provided that it retains/exhibits at least a substantial proportion of the antigen-binding properties of the corresponding complete antibody. Typically, antibody fragments are associated with lower antigen-binding affinity than “full length” or “complete” antibodies, but can offer other advantageous features that may offset for any such loss in affinity (e.g., ease of delivery, ease of production, improved stability, reduced undesirable side effects in a patient, etc.).

Although having similar binding properties as full-length antibodies, the various types of antibody fragments described herein, collectively and each independently, is/are unique features of the invention, exhibiting different biological and/or physiochemical properties than antibodies.

Antibody fragments can be derived from any suitable type of antibody, having, for example, any suitable isotype. For example, bispecific F(ab′)2 mu fragments, derived from IgM antibodies, can be used in the methods and incorporated in the compositions of the invention (see, e.g., Morimoto et al., J Immunol Methods. Apr. 22, 1999;224(1-2):43-50 for a description of such antibody fragments).

To better illustrate this feature of the invention, a number of exemplary types of antibody fragments are discussed in further detail here.

In one exemplary aspect, the invention provides scFv molecule antibody fragments. Although the two domains of the Fv fragment, VL and VH, are coded for by separate genes, they can be joined, e.g., using recombinant methods, by a synthetic and typically flexible linker that enables them to be made as a single protein chain in which the VL and VH regions (typically the heavy and light chains in the Fv region of an antibody) pair to form monovalent molecules (known as single chain antibodies or single chain Fv (scFv) molecules—see e.g., Bird et al. (1988) Science 242:423-426: and Huston et al. (1988) Proc. Natl. Acad. Sci. USA 85:5879-5883). Usually the flexible linker is of about 10, 12, 15, or more amino acid residues in length. Methods of producing such antibodies are described in, e.g., U.S. Pat. No. 4,946,778; THE PHARMACOLOGY OF MONOCLONAL ANTIBODIES, vol.113, Rosenburg and Moore eds. Springer-Verlag, New York, pp. 269-315 (1994), Bird et al. (1988) Science 242:423-426; Huston et al. (1988) Proc. Natl. Acad. Sci. USA 85:5879-5883; and McCafferty et al., Nature (1990) 348:552-554. A single chain antibody may be monovalent, if only a single VH and VL are used, bivalent, if two VH and VL are used, or polyvalent, if more than two VH and VL are used to form the antibody.

Other forms of single chain antibody molecules, such as diabodies also are encompassed by the terms “antibody fragment” and “antibody-like peptide” (or “antibody-like molecule”) unless otherwise stated or clearly contradicted by context. Diabodies are bivalent, bispecific antibodies in which VH and VL domains are expressed on a single polypeptide chain, but using a linker that typically is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poijak, R. J., et al. (1994) Structure 2:1121-1123). A diabody can be considered an antibody fragment in which scFvs having the same or different antigen binding specificity form a dimer and, accordingly, is a molecule that has a divalent antigen binding activity to the same antigen or to two different antigens. Diabodies are described more fully in, for example, EP 404,097 and WO 93/11161.

In another aspect, the invention provides a L5G2D3BP dsFV molecules. A dsFV molecule can be obtained by binding polypeptides in which one amino acid residue of each of VH and VL is substituted with a cysteine residue via a disulfide bond between the cysteine residues. The amino acid residue which is substituted with a cysteine residue can be selected based on a three-dimensional structure estimation of the antibody, e.g., in accordance with the method described by Reiter et al. (Protein Engineering, 7, 697 (1994)). In a further aspect, the invention provides L5G2D3BP linear antibodies, which comprise a pair of tandem Fd segments that form a pair of antigen binding regions (such antibodies can be bispecific or monospecific). Linear antibodies are more fully described in, e.g., Zapata et al. Protein Eng. 8(10):1057-1062 (1995).

In addition to antibodies produced against γ2 by antibody producing cells, non-wild-type antibodies comprising suitable variants of the antibody sequences produced by such cells may be used in the various methods of the invention and represent an additional facet of the invention.

Variants of antibody sequences can be produced by any suitable method, several of which are commonly employed by those of ordinary skill in the art.

For example, to improve the quality and/or diversity of antibodies against γ2, the VL and VH segments of VL/VH pair(s) (or portions thereof) can be randomly mutated, typically at least within the CDR3 region of VH and/or VL, in a process analogous to the in vivo somatic mutation process responsible for affinity maturation of antibodies during a natural immune response. Such in vitro affinity maturation can be accomplished by, e.g., amplifying VH and VL regions using PCR primers complimentary to VH CDR3 or VL CDR3 encoding sequences, respectively, which primers typically are “spiked” with a random mixture of the four nucleotide bases at certain positions, such that the resultant PCR products encode VH and VL segments into which random mutations have been introduced thereby resulting (at least in some cases) in the introduction of sequence variations in the VH and/or VL CDR3 regions. Such randomly mutated VH and VL segments can thereafter be re-screened by phage display or other suitable technique for binding to γ2 DIII-containing peptides and advantageous variants analyzed and used to prepare novel L5G2BPs. Following screening, nucleic acid encoding a selected antibody or other L5G2BP, where appropriate, can be recovered from a display package (e.g., from a phage genome) and subcloned into an appropriate vector by standard recombinant techniques. If desired, such an antibody-encoding nucleic acid can be further manipulated to create other antibody forms or L5G2BPs. To express a recombinant human antibody isolated by screening of a combinatorial library, typically a nucleic acid comprising a sequence encoding the antibody is cloned into a recombinant expression vector and introduced into appropriate host cells (mammalian cells, yeast cells, etc.) under conditions suitable for expression of the nucleic acid and production of the antibody.

High-affinity antibody peptides, such as human single-chain Fv (ScFv) and Fab antibody fragments, also can be isolated from such display libraries using, e.g., a panning technique in which the antigen of interest is immobilized on a solid surface, such as microtiter plates or beads (see, e.g., Barbas and Burton, Trends. Biotechnol. 1996, 14:230-234 and Aujame et al, Hum. Antibodies 1997, 8:155-68). Phage display of large naive libraries, for example, also makes it possible to isolate human antibodies directly without immunization (see, e.g., DeHaard et al., J. Biol. Chem. 1999, 18218-18230).

Other potentially suitable techniques for preparing novel anti-γ2 DIII antibodies and other L5G2BPs include CDR walking mutagenesis, antibody chain shuffling, “parsimonious mutagenesis” (Balint and Larrick Gene 137:109-118 (1993)), and other affinity maturation techniques (see, e.g., Wu et al. PNAS (USA) 95: 6037-6-42 (1998)). Repertoire cloning procedures also can be useful in the production of variant antibodies (see, e.g., International Patent Application WO 96/33279).

If desired, the class of an anti-γ2 DIII antibody obtained by antibody producing cells may be “switched” by known methods. For example, an anti-γ2 DIII antibody that was originally produced as an IgM molecule may be class switched to an IgG anti-γ2 DIII antibody. Class switching techniques also may be used to convert one IgG subclass to another, e.g., from IgG1 to IgG2. Thus, the effector function of the antibodies of the invention may be changed by isotype switching to, e.g., an IgG1, IgG2, IgG3, IgG4, IgD, IgA, IgE, or IgM antibody for various therapeutic uses.

By way of the above-described techniques, other techniques provided herein, and related methods known in the art, the invention provides a number of methods for generating useful antibody and antibody-like peptide variants that are similar to one or more “parent” anti-γ2 DIII antibodies produced by mammals or other chordates in response to immunization with an antigen comprising one or more γ2 DIII peptide sequences (such as, for example, mAb 5D5 and/or mAb 6C12). A “variant” anti-γ2 DIII antibody is an antibody that differs from a parent antibody by one or more suitable amino acid residue substitutions, deletions, insertions, or terminal sequence additions in at least the CDRs or other VH and/or VL sequences (provided that at least a substantial amount of the epitope binding characteristics of the parent antibody are retained, if not improved upon, by such changes). Thus, for example, in an antibody variant or antibody-like peptide variant, one or more amino acid residues can be introduced or inserted in or adjacent to one or more of the hypervariable regions of a parent antibody, such as in one or more CDRs. For example, an anti-γ2 DIII antibody variant can comprise about 1-30 inserted amino acid residues, but about 2-10 inserted amino acid residues is more typically suitable.

It should be understood that fragments of antibody variants are also a feature of the invention. Thus, although the discussion herein focuses primarily on antibodies, it should be understood that the aspects and features of such antibody variants can equally be applied to antibody fragments, antibody-like peptides (e.g., immunoadhesins, diabodies, etc.), and other L5G2BPs, as appropriate. However, such non-antibody variants should be understood to be individual aspects of the invention with often significantly different biological properties from similar antibody variants. Examples of such differences include, without limitation, significantly smaller size for improved formulation, multivalency, avoidance of inducing complement-related functions, lack of steric hindrance from potential competing molecules, and/or easier production.

Amino acid sequence variants of the antibody can be obtained by, for example, introducing appropriate nucleotide changes into an antibody-encoding nucleic acid (e.g., by site directed mutagenesis), by chemical peptide synthesis, or any other suitable technique. Such variants include, for example, variants differing by deletions from, and/or insertions into and/or substitutions of, residues within the amino acid sequences of known anti-γ2 DIII antibodies (such as, e.g., mAb 4G1, mAb 5D5, and/or mAb 6C12). Any combination of deletions, insertions, and substitutions can be made to arrive at a desired variant, provided that the variant possesses suitable characteristics for practice in the methods of the invention (e.g., a retention of at least a substantial proportion of the parent antibodies affinity, specificity, and/or selectivity with respect to one or more γ2 DIII epitopes or antigenic determinant regions). Amino acid sequence changes, with respect to a parent antibody, also may alter post-translational processes of the variant antibody with respect to a parent antibody, such as by changing the number or position of glycosylation sites.

Typical lengths of the hypervariable regions of antibodies are known, and such information can be used to produce variant antibodies that include hypervariable region insertions. For example, for the first hypervariable region of a light chain variable domain, insertions can be introduced into the CDR L1 sequence of a parent antibody while retaining a substantially similar and thereby expected appropriate size, which according to Kabat et al., supra, e.g., typically has an overall of about 9-20 (e.g., about 10-17) residues. Similarly, CDR L2 typically has an overall length from about 5-10 residues; CDR L3 typically has a length of about 7-20 residues; CDR H1 typically has a length of about 10-15 residues; CDR H2 typically has a length of about 15-20 residues; and CDR H3 typically has a length of about 6-30 residues (e.g., 3-25 residues). Insertions in the VH region typically are made in CDR H3 and typically near the C-terminal of the domain, such as about residues 97-102 of the parent CDR H3 (e.g., adjacent to, and preferably C-terminal in sequence to, residue number 100 of the parent CDR H3 sequence) using the alignment and numbering as described in Kabat. Antibody variants with inserted amino acid residue(s) in a hypervariable region thereof may be prepared randomly, especially where the starting binding affinity of the parent antibody for the target antigen is such that randomly produced antibody variants can be readily screened. For example, phage display provides a convenient method of screening such random variants.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other antibody insertion variants include the fusion to the N— or C-terminus of the antibody of an enzyme or a polypeptide or PEG which increases the serum half-life of the antibody. Such anti-γ2 antibody fusion proteins and similar fusion proteins comprising L5G2BP sequences are another advantageous feature of the invention.

For antibodies, the sites of greatest interest for substitution variations are the hypervariable regions (or particular CDRs), but variants also or alternatively characterized by one or more framework (FR) alterations also are within the scope of antibody variants provided by the invention and may be associated with advantageous properties. For example, a substitution or other modification (insertion, deletion, or combination of any thereof) in a framework region or constant domain can be associated with an increase in the half-life of the variant antibody with respect to the parent antibody. A variation in a framework region or constant domain may also be made to alter the immunogenicity of the variant antibody with respect to the parent antibody, to provide a site for covalent or non-covalent binding to another molecule, or to alter such properties as complement fixation. Variations in an antibody variant may be made in each of the framework regions, the constant domain, and/or the variable regions (or any one or more CDRs thereof) in a single variant antibody. Alternatively, variations may be made in only one of the framework regions, the variable regions (or single CDR thereof), or the constant domain in an antibody. Alanine scanning mutagenesis techniques, such as described by Cunningham and Wells (1989), Science 244:1081-1085, can be used to identify suitable residues for substitution or deletion in generating L5G2D3BPs comprising variant VL, VH, or particular CDR sequences, although other suitable mutagenesis techniques also can be applied. Multiple amino acid substitutions also can be made and tested using known methods of mutagenesis and screening, such as those disclosed by Reidhaar-Olson and Sauer, Science 241:53-57 (1988) or Bowie and Sauer Proc. Natl. Acad. Sci. USA 86:2152-2156 (1989). Additional techniques that can be used to generate variant antibodies include the directed evolution and other variant generation techniques described in, e.g., US 20040009498; Marks et al., Methods Mol Biol. 2004;248:327-43 (2004); Azriel-Rosenfeld et al., J Mol Biol. Jan. 2, 2004;335(1):177-92; Park et al., Biochem Biophys Res Commun. Aug. 28, 2000;275(2):553-7; Kang et al., Proc Natl Acad Sci USA. Dec. 15, 1991;88(24):11120-3; Zahnd al., J Biol Chem. Apr. 30, 2004;279(18):18870-7; Xu et al., Chem Biol. August 2002;9(8):933-42; Border et al., Proc Natl Acad Sci USA. Sep. 26, 2000;97(20):10701-5; Crameri et al., Nat Med. January 1996;2(1):100-2; and as more generally described in, e.g., International Patent Application WO 03/048185. Thus, the invention in one aspect provides a method of preparing a variant anti-γ2 DIII antibody by applying such techniques to the γ2 DIII antibodies described herein or nucleic acids encoding such antibodies, as appropriate.

In the design, construction, and/or evaluation of CDR variants attention can be paid to the fact that CDR regions can vary to enable a better binding to the epitope. Antibody CDRs typically operate by building a “pocket,” or other paratope structure, into which the epitope fits. If the epitope is not fitting tightly, the antibody may not offer the best affinity. However, as with epitopes, there often are a few key residues in a paratope structure that account for most of this binding. Thus, CDR sequences can vary in length and composition significantly between antibodies for the same peptide (for example, the CDR-H3 sequence of mAb 5D5 may be significantly longer than the CDR-H3 sequences of mAb 4G1 and mAb 6C12). The skilled artisan will recognize that certain residues, such as tyrosine residues (e.g., in the context of CDR-H3 sequences), that are often significant contributors to such epitope binding, are typically desirably retained in a CDR variant.

A convenient way for generating substitution variants is affinity maturation using phage using methods known in the art. In order to identify candidate hypervariable region sites for modification, alanine scanning mutagenesis also can be performed to identify hypervariable region residues contributing significantly to antigen binding. Alternatively or additionally, it may be beneficial to analyze a crystal structure of the antigen-antibody complex to identify contact points between the antibody and antigen. Such contact residues and neighboring residues are likely suitable candidates for substitution.

Useful methods for rational design of CDR sequence variants are described in, e.g., International Patent Applications WO 91/09967 and WO 93/16184. Additional considerations in the production/selection of peptide variants (e.g., conservation of amino acid residue functional characteristics, conservation of amino acid residues based on hydropathic characteristics, and/or conservation of amino acid residues on the basis of weight/size, are described elsewhere herein). Typically, amino acid sequence variations, such as conservative substitution variations, desirably do not substantially change the structural characteristics of the parent sequence (e.g., a replacement amino acid should not tend to disrupt secondary structure that characterizes the function of the parent sequence). Examples of art-recognized polypeptide secondary and tertiary structures are described in, e.g., Proteins, Structures and Molecular Principles (Creighton, Ed., W. H. Freeman and Company, New York (1984)); INTRODUCTION TO PROTEIN STRUCTURE (C. Branden and J. Tooze, eds., Garland Publishing, New York, N.Y. (1991)); and Thornton et at. Nature 354:105 (1991). Additional principles relevant to the design and construction of peptide variants are discussed in, e.g., Collinet et al., J Biol Chem Jun. 9, 2000;275(23): 17428-33.

Typically, advantageous sequence changes are those that (1) reduce susceptibility to proteolysis, (2) reduce susceptibility to oxidation, (3) alter binding affinity of the variant sequence (typically desirably increasing affinity), and/or (4) confer or modify other physicochemical or functional properties on the associated variant/analog peptide. In the context of CDR variants, particularly in the context of variant anti-γ2 DIII antibodies, it is typically desired that residues required to support and/or orientate the CDR structural loop structure(s) are retained; that residues which fall within about 10 angstroms of a CDR structural loop (but optionally only residues in this area that also possess a water solvent accessible surface of about 5 angstroms² or greater) are unmodified or modified only by conservative amino acid residue substitutions; and/or that the sequence is subject to only a limited number of insertions and/or deletions (if any), such that CDR structural loop-like structures are retained in the variant (a description of related techniques and relevant principles is provided in, e.g., Schiweck et al., J Mol Biol. May 23, 1997;268(5):934-51; Morea, Biophys Chem. October 1997;68(1-3):9-16; al., FEBS Lett. Dec. 9, 1996;399(1-2):1-8; Shirai et al., FEBS Lett. Jul. 16, 1999;455(1-2): 188-97; Reckzo et al., Protein Eng. April 1995;8(4):389-95; and Eigenbrot et al., J Mol Biol. Feb. 20, 1993;229(4):969-95).

Amino acid sequence variations can result in an altered glycosylation pattern in the variant antibody with respect to a parent antibody. By “altering” it is meant deleting one or more carbohydrate moieties found in the parent antibody, and/or adding one or more glycosylation sites that are not present in the parent antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are common recognition sequences for enzymatic attachment of the carbohydrate moiety to the asparagine side chain. Thus, the presence of either of these tripeptide sequences in a polypeptide typically can create a potential glycosylation site. O-linked glycosylation refers to the attachment of sugars such as N-aceylgalactosamine, galactose, or xylose to a hydroxyamino acid, most commonly serine or threonine, although 5-hydroxyproline or 5-hydroxylysine may also be used. Addition of glycosylation sites to the antibody can be conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The phrase “potential amino acid interactions” can be used to refer to contacts or energetically favorable interactions between one or more amino acid residues present in an antigen and one or more amino acid residues which do not exist in a parent antibody but can be introduced therein so as to increase the amino acid contacts between the antigen and an antibody variant comprising those introduced amino acid residue(s). Desirably, antibody variants are associated with increased potential amino acid interactions with a portion of γ2, typically with a portion of γ2 DIII. Amino acid interactions of interest can be selected from hydrogen bonding interactions, van der Waals interactions, and/or ionic interactions.

The various CDR, VH, and VL sequences and variants provided herein can be combined in any suitable manner to produce new antibodies and antibody-like molecules. As one example of such an antibody variant, the invention provides a L5G2D3BP that comprises a CDR-L2 that is a fusion of CDR-L2 sequences from mAb 4G1 and mAb 6C12. In another context, the invention provides a variant anti-γ2 DIII antibody derived from mAb 4G1 or mAb 6C12, wherein the CDR-L2 sequences of the mAb are replaced with CDR-L2 sequences derived from the other mAb. In another aspect, the invention provides antibody and antibody like molecules derived from mAb 4G1 and/or mAb 5D5, wherein the CDR-L1 region of the derived antibody or antibody-like molecule is replaced with a CDR-L1 from the other molecule. In a further aspect, the invention provides a L5G2D3BP that comprises a CDR-L1 variant that corresponds to a fusion of CDR-L1 sequences from mAb 4G1 and/or mAb5D5. In another aspect, the invention provides a variant antibody derived from mAb 5D5 or mAb 4G1, wherein the light chain of the antibody comprises one or more substitutions, insertions, or deletions that correspond to the sequence of the other antibody (e.g., the invention provides a variant of mAb 4G1 wherein one or more residue changes are introduced into the light chain of the derivative based upon the residues found at corresponding positions in the light chain of mAb 5D5). In another aspect, the invention provides an antibody derived from mAb 5D5 or mAb 6C12, wherein the heavy chain sequence of the derived antibody varies from the parent antibody by one or more changes that correspond to the structure of the other antibodies heavy chain (e.g., the invention provides an anti-γ2 DIII antibody variant derived from mAb 6C12 wherein one or more of the heavy chain residues of mAb 6C1 2 are replaced with corresponding residues found in the mAb 5D5 heavy chain). Other changes to the VH, VL, and CDRs (e.g., the CDR-L1 and/or CDR-L2 regions) of anti-γ2 DIII variant antibodies provided by this invention also can be suitably introduced into variant antibodies and antibody-like molecules.

L5G2BPs of the invention can generally be in any suitable form with respect to multimerization. Anti-γ2 DIII antibodies and antibody fragments will typically be at least in heterotrimeric form if not in higher multimeric forms such as those associated with IgM antibodies. In other aspects, a L5G2BP may be presented as a dimer or monomer. Monomeric L5G2BPs of the invention can be, for example, modified by any suitable technique so as to form multimeric peptide compositions.

In one particular exemplary aspect, the invention provides an anti-γ2 DIII antibody variant wherein less than about 10, such as less than about 5, such as 3 or less amino acid variations are present in either the VH or VL regions of the variant antibody with respect to a parent anti-γ2 DIII antibody. In another exemplary aspect, the invention provides an anti-γ2 DIII antibody variant wherein less than about 15, such as less than about 10, such as less than about 5 amino acid variations exist in the constant domains of the variant antibody with respect to a parent anti-γ2 DIII antibody.

In another nonlimiting exemplary aspect, the invention provides a structural variant of an anti-γ2 DIII parent antibody. Structural determinations with respect to L5G2D3BPs can be made by any suitable technique, such as nuclear magnetic resonance (NMR) spectroscopic structure determination techniques, which are well-known in the art (See, e.g., Wuthrich, NMR of Proteins and Nucleic Acids, Wiley, New York, 1986; Wuthrich, K. Science 243:45-50 (1989); Clore et al., Crit. Rev. Bioch. Molec. Biol. 24:479-564 (1989); Cooke et. al. Bioassays 8:52-56 (1988)), typically in combination with computer modeling methods (e.g., by use of programs such as MACROMODEL™, INSIGHT™, and DISCOVER™, to obtain spatial and orientation requirements for structural analogs. Using information obtained by these and other suitable known techniques, structural analogs of L5G2D3BPs can be designed and produced through rationally-based amino acid substitutions, insertions, and/or deletions. Such structural variants may be useful in practicing methods of the invention. It also is possible and often desirable that such structural information be used in concert with parent antibody sequence information to design useful antibody variants.

Generated antibody variants can be subjected to any suitable screening technique and antibodies with suitable and desirably superior properties in one or more relevant assays may be selected for further development.

Examples of suitable amino acid changes in the context of CDRs obtained from mAbs 4G1, 5D5, and 6C12 are described elsewhere herein.

Another feature of the invention is the provision of multispecific and/or multivalent L5G2BPs, such as multispecific and multivalent anti-γ2 antibodies (which typically can be characterized as multispecific anti-γ2 DIII antibodies) and antibody-like molecules (e.g., a multispecific antibody fragment, antibody derivative, or antibody fragment derivative).

In general, anti-γ2 antibodies can have any suitable number of valencies and specificities. As mentioned elsewhere herein, for example, an anti-γ2 DIII antibody can be a univalent antibody. Antibodies with more than two valencies also can be prepared using known techniques and used in the context of the inventive methods described herein and incorporated into the compositions of this invention. For example, trispecific antibodies that are partially specific for γ2 DIII can be prepared by methods known in the art (see, e.g., Tutt et al. J. Immunol. 147: 60 (1991)).

In one exemplary aspect, the invention provides a bispecific antibody comprising at least one pair of VH sequence and VL sequence chains specific for an epitope comprised at least in part in γ2 DIII and a second at least one pair of VH and VL sequence chains specific for a second (i.e., different) epitope. The VH and VL sequences in such a bispecific antibody can comprise complete VH and VL sequences corresponding to anti-γ2 DIII antibody VH and VL region sequences, variant VH and/or VL sequences, and/or suitable portions of VH and/or VL regions, such as a combination of CDR sequences and other sequences (e.g., framework sequences/residues) sufficient to provide binding to an epitope or epitopes of interest.

Bispecific antibodies can be produced by a variety of known methods including fusion of hybridomas or linking of Fab′ fragments (see, e.g., Songsivilai & Lachmann Clin. Exp. Immunol. 79: 315-321 (1990) and Kostelny et al. J. Immunol. 148:1547-1553 (1992)). Traditionally, the recombinant production of bispecific antibodies is based on the co-expression of two immunoglobulin heavy chain-light chain pairs, where the two heavy chains have different specificities (see, e.g., Milstein and Cuello, Nature, 305: 537 (1983)). Because of the typical random assortment of immunoglobulin heavy and light chains, these hybridomas (quadromas) produce a potential mixture of 10 different antibody molecules, of which only one typically has the desired bispecific structure. The purification of the correct molecule, which is usually done by affinity chromatography, although suitable, can be rather cumbersome, and the product yields can be relatively low. Similar procedures are disclosed in WO 93/08829 and Traunecker et al., EMBO J., 10: 3655 (1991).

According to a different approach, antibody variable domains with the desired binding specificities (antibody-antigen combining sites) are fused to immunoglobulin constant domain sequences by recombinant or synthetic methods. The variable domain sequence is typically fused to an immunoglobulin heavy chain constant domain, comprising at least part of the hinge, CH2, and CH3 regions. A first heavy-chain constant region (CH1), containing the site necessary for light chain binding, also typically is present in at least one of the fusion peptides. In a more specific example of this type of approach, a bispecific antibody is produced comprising a hybrid immunoglobulin heavy chain with a first binding specificity in one arm, and a hybrid immunoglobulin heavy chain-light chain pair (providing a second binding specificity) in the other arm. Such an asymmetric structure can facilitate the separation of the desired bispecific compound from unwanted immunoglobulin chain combinations (such an approach is described in WO 94/04690). For further description of related methods for generating bispecific antibodies see, for example, Suresh et al., Methods in Enzymology, 121:210 (1986).

In yet another approach, the interface between a pair of antibody molecules can be engineered to maximize the percentage of heterodimers that are recovered from recombinant cell culture so as to form a population of bispecific antibody molecules. Typically, such an interface comprises at least a part of the CH3 domain of an antibody constant region. Normally in such a method, one or more amino acid residues with smaller side chains from the interface of the first antibody molecule are replaced with amino acid residues with larger side chains (e.g. tyrosine or tryptophan). Compensatory “cavities” of identical or similar size to the large side chain amino acid residue(s) are created on the interface of the second antibody molecule by replacing large amino acid side chain residues with smaller ones (e.g., alanine or threonine). This technique provides a mechanism for increasing the yield of the heterodimer over other unwanted end-products such as homodimers.

Cross-linked or “heteroconjugate” antibodies are another type of bispecific antibody provided by the invention. Derivatives of such antibodies also can be advantageous for certain applications. For example, one of the antibodies in a heteroconjugate can be coupled to avidin and the other to biotin. Such antibodies have, for example, been proposed to target immune system cells to unwanted cells (see, e.g., U.S. Pat. No. 4,676,980). Heteroconjugate antibodies may be made using any convenient cross-linking methods. Suitable peptide cross-linking agents and techniques are well known in the art, and examples of such agents and techniques are disclosed in, e.g., U.S. Pat. No. 4,676,980.

Bispecific antibodies and antibody-like molecules (e.g., bispecific molecules generated from two antibody fragments) generally can be prepared using chemical linkage techniques. Brennan et al., Science, 229: 81 (1985), for example, describe a procedure wherein intact antibodies are proteolytically cleaved to generate F(ab′)2 fragments. These fragments may then be reduced in the presence of the dithiol complexing agent sodium arsenite to stabilize vicinal dithiols and prevent intermolecular disulfide formation. The Fab′ fragments generated can then converted to thionitrobenzoate (TNB) derivatives. One of the Fab′-TNB derivatives can then be reconverted to the Fab′-thiol by reduction with mercaptoethylamine and mixed with an equimolar amount of the other Fab′-TNB derivative to form a bispecific antibody.

Fab′-SH fragments also recovered from E. coli also can be chemically coupled to form bispecific antibodies. Shalaby et al., J. Exp. Med., 175: 217-225 (1992), for example, describe the production of a fully humanized bispecific antibody F(ab′)2 molecule, according to a related technique.

Various techniques for making and isolating bispecific antibody fragment molecules directly from recombinant cell culture have also been described. For example, bispecific antibodies have been produced using leucine zippers (see, e.g., Kostelny et al., J. Immunol., 148(5): 1547-1553 (1992)). Leucine zipper peptides from the Fos and Jun proteins can be linked to the Fab′ portions of two different antibodies by gene fusion and the resulting antibody homodimers reduced at the hinge region to form monomers that can be re-oxidized to form the antibody heterodimers. The “diabody” technology described by Hollinger et al., Proc. Natl. Acad. Sci. USA, 90:6444-6448 (1993) also has provided an alternative mechanism for making bispecific antibody fragment molecules (see also Alt et al., FEBS Letters, 454 (1990) 90-94 for a description of similar diabody-related techniques). Another strategy for making bispecific antibody fragment molecules by the use of single-chain Fv (sFv) dimers has also been reported. See, e.g., Gruber et al., J. Immunol., 152:5368 (1994). In addition, bispecific antibodies may be formed as “Janusins” (Traunecker et al., EMBO J 10:3655-3659 (1991) and Traunecker et al., Int J Cancer Suppl 7:51-52 (1992)). Additional methods relevant to the production of multispecific antibody molecules are disclosed in, e.g., Fanger et al., Immunol. Methods 4:72-81 (1994).

Exemplary bispecific antibody and antibody-like molecules comprise (i) two antibodies one with a specificity to γ2 DIII and another to a second target that are conjugated together, (ii) a single antibody that has one chain specific to γ2 DIII and a second chain specific to a second molecule, and (iii) a single chain antibody that has specificity to γ2 DIII and a second molecule. Typically, the second target/second molecule is a molecule other than Ln-5. In one aspect, the second molecule is a cancer antigen/tumor-associated antigen such as carcinoembryonic antigen (CEA), prostate specific antigen (PSA), RAGE (renal antigen), α-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), a cancer-associated ganglioside antigen, tyrosinase, gp75, C-myc, Mart1, MelanA, MUM-1, MUM-2, MUM-3, HLA-B7, or Ep-CAM. Additional cancer antigens/tumor antigens that can be targeted by multispecific L5G2BPs are described elsewhere herein.

In another aspect, the invention provides a multispecific antibody that specifically binds a portion of γ2 (typically a portion of DIII) and at least one second molecule that is a cancer-associated integrin, such as α5β3 integrin.

In another aspect, the invention provides a multispecific antibody that specifically binds to a portion of γ2 and at least one second molecule that is an angiogenic factor or other cancer-associated growth factor, such as a vascular endothelial growth factor (VEGF), a fibroblast growth factor (FGF), epidermal growth factor (EGF), epidermal growth factor receptor (EGF-R), angiogenin, and receptors thereof, particularly receptors associated with cancer progression (e.g., one of the HER1-HER4 receptors).

In a further facet, the invention provides a multispecific antibody that specifically binds to a portion of γ2 and at least one second molecule that is a Ln-5-associated modulation of cell migration, such as integrin α3β1; one or more other Ln-5 associated integrins (e.g., alpha6beta1 (α6β1), and alpha6beta4 (α6β4) integrins); type VII collagen; fibulin-2; IL-7, heat shock protein 27; BP180; syndecan-4; dystroglycan; nidogen-1; Ln-5/cancer-associated matrix metalloproteinases (e.g., MMP-1, MMP-2, MMP-3, MMP-8, MMP-9, MMP-12, MMP-13, MMP-20, and MMP-14 (also known as MT1-MMP) (see, e.g., Pirila et al., Biochem Biophys Res Commun. Apr. 18, 2003;303(4):1012-7 with respect to examples of MMP processing of Ln-5)); EWI (see, e.g., Stipp et al., J Cell Biol. Dec. 8, 2003;163(5):1167-77); tissue inhibitor of matrix metalloproteinase-1 (TIMP-1) and TIMP-2; BB94 (see, e.g., Giles et al., J Cell Sci. August 2001;114(Pt 16):2967-76); tetraspanin D6.1A (see, e.g., Herlevsen et al., J Cell Sci. Nov. 1, 2003;116(Pt 21):4373-90); and bone morphogenic protein-1 (BMP-1—see, e.g., US Patent Application US 20020076736 with respect to methods involving BMP-1 and related molecules).

Other cancer progression-associated proteins discussed herein also or alternative can be suitable second molecules targeted by multispecific antibodies and antibody fragments of the invention (see, for example, the discussion of combination therapies provided elsewhere herein).

In other aspects, the invention provides a multispecific antibody or antibody fragment specific for a portion of γ2 (e.g., a portion of DIII) and a second target that is present in a heterotrimeric Ln-5 (e.g., a processed form of Ln-5—see, e.g., Hintermann et al., Matrix Biol. May 2004;23(2):75-85 and Ogawa et al., J Cell Biochem. Jul. 1, 2004;92(4):701-14), one or more Ln-5 monomers (such as β3 or γ2), γ2/β3 heterodimers, or another region of γ2 (e.g., a different region of DIII).

As indicated above, the invention provides multispecific antibody fragments as well as multispecific full size antibody molecules (in this respect, each of these terms should generally be considered as providing support for one another herein where applicable). Specific examples of multispecific antibody fragments include 60-65 kDa amphipathic helix-based scFv dimers (see, e.g., Pack et al. (1993) Bio/Technology 11: 1271-1277; Pack (1992) Biochemistry 31: 1579-1584 for related discussion), and 80 kDa (scFv-C_{H3})₂ LD minibodies and Flex minibodies (see, e.g., Hu et al. (1996) Cancer Res. 56: 3055-3061 for related discussion). While each of these proteins is capable of binding two antigen molecules, they differ in the orientation, flexibility and the span of their binding sites. At least some of these antibody fragments and similar molecules will be described in further detail elsewhere herein.

Minibodies are single chain antibody fragments that retain the antigen binding region, the CH3 domain to permit assembly into a bivalent molecule, and the antibody hinge to accommodate dimerization by disulfide linkages. Typical minibodies are comprised of the variable heavy and variable light chain domains of a native antibody fused to the hinge region and to the CH3 domain of the immunoglobulin molecule (the CH2 domain is typically deleted as the CH3 domain is sufficient for proper assembly). The heavy and light chain variable domains are typically linked by way of a short linker. The size and affinity of minibodies is particularly suited for in vivo administration. Expression in bacterial or mammalian cells is simplified because minibodies are single chains. Classic methods of preparing minibodies are provided in, e.g., U.S. Pat. No. 5,837,821.

In a further aspect, the invention provides a multispecific antibody formed by the inclusion of one or more multimerization domains, such as in a first single chain antibody construct specific for an AMR and a second single chain antibody construct specific for a STM. The multimerization may, inter alia, be obtained via heterodimerization. For example, the heterodimerization region of constant immunoglobulin domains may be employed. Other multidimerization and/or heterodimerization domains are known in the art, such as those based on leucine zippers, α- and β-chains of T-cell receptors, or MHC-class II molecules. Furthermore, jun- and fos-based domains may be employed as multimerization domains (see, e.g., de Kuif (1996) J. Biol. Chem. 271:7630-7634; Kostelny (1992), J. Immunol. 148,1547-1553). Additional examples of multimerization domains are p53- and MNT-domains as described, e.g., in Sakamoto (1994) Proc. Natl. Acad. Sci. USA 91, 8974-8978; Lee (1994) Nat. Struct. Biol. 1, 877-890; Jeffrey (1995) Science 267, 1498-5102 or Nooren (1999) Nat. Struct. Biol. 6, 755-759. The use of multimerization domains is not limited to forming L5G2BPs from single chain antibody constructs. For example, multimerization domains can be used to form multimers of non-antibody L5G2BPs, mutli-chain antibodies, etc.

In another aspect, the invention provides a heterominibody construct comprising at least one portion that binds to γ2 and a second portion that binds to at least one secondary (non-γ2) target (e.g., one of the targets described above). Heterominibodies are known in the art and their production is described, inter alia, in WO 00/06605. Briefly, a heterominibody is a heterodimer of two chains, the first of which comprises the CH1-domain (as the only constant antibody domain therein) and the second of which comprises the CL domain of an antibody light chain, wherein at least two peptides that lack intrinsic affinity for one another, at least one of which having specificity for at least one AMR and the second of which having specificity for at least one second target, are fused to the constant region domains. Typically, the domains are in the format of a scFv-fragment. Additional guidance in terms of methods and principles relevant to the production of similar single chain antibodies is provided in, e.g., Dreier et al., Int. J. Cancer: 100, 690-697 (2002); Dreier et al., J Immunol, 2003, 170(8): 4397-402; and Löffler et al., Leukemia, 2003, 17, 900-907, as well as U.S. Pat. No. 6,723,538. Additional exemplary bispecific antibody fragments are described in, e.g., Peipp and Valerus, Biochemical Society Transactions, 30(4):507-511 (2002).

The invention also provides domain-deleted anti-γ2 antibodies, such as CH₂domain-deleted bispecific antibodies (although such antibodies may be monospecific). The deletion of such domains in certain contexts has been found to not impair target antigen binding.

As described above, several of the multi-domain antibody fragment molecules provided by the invention comprise one or more linking structures or sequences between the various domains derived from the same or different antibodies. Such linkage can be provided by recombinant expression techniques or can be performed by, e.g., chemical cross-linking as described in, e.g., WO 94/04686. Typically, an amino acid sequence linker, which typically is a flexible linker, is used to link such domains. Such a linker typically will comprise a number of flexible residues (e.g., about 3-20 residues, such as 5-15 residues), and more typically will be predominantly comprised of Gly and/or Ser residues. For formation of scFv dimers, short linkers, such as linkers of about 3-12 residues, which do not permit formation of a functional Fv domain, are used, and even shorter linkers are used for formation of triabodies and tetrabodies, as is described in above-cited references (see, e.g., Kortt et al., Biomol Eng. Oct. 15, 2001;18(3):95-1 08).

In yet another aspect, the invention provides variant L5G2BP antibodies and antibody fragments wherein potential B cell epitopes and/or T cell epitopes in the antibody sequences have been reduced or eliminated through rationale design, mutagenesis, or other suitable technique. Thus, for example, in one aspect the invention provides a “deimmunized” anti-γ2 DIII antibody in which the potential T cell epitopes in the parent antibody (such as in a murine anti-γ2 DIII antibody like mAb 5D5) have been eliminated. The design and construction of deimmunized L5G2D3BPs can be accomplished by any suitable known technique (see, e.g., International Patent Application PCT/GB98/01473 with respect to exemplary methods for preparing deimmunized antibodies). Immunogenicity is expected to be eliminated or substantially reduced when such L5G2BPs are administered or otherwise delivered to a human as compared to a related (unmodified) “parent” antibody molecule. Such techniques can be used independently or as an adjunct to humanization (thus, for example, a humanized antibody comprising one or more pairs of CDRs from mAb 4G1, mAb 5D5, or mAb 6C12, which also is modified by substitution, removal, and/or insertion of one or more amino acid residues in the murine sequences and/or at the junction of the murine and human sequences so as to reduce the immunogenicity of the humanized antibody can be prepared in accordance with this invention).

Anti-γ2 antibodies that exhibit one or more superantibody functions due to incorporation and/or conjugation of superantibody domains to an anti-γ2 antibody are another feature of the invention.

The term “superantibody” was recently coined to signify antibodies with high potency and was based upon a discovery of self-binding or autophilic antibodies which exhibit higher potency. Such antibodies form homologous complexes not in solution but only after binding to their antigen targets. One such autophilic antibody, directed to phosphorylcholine, has been shown to be several fold more potent than traditional therapeutic antibody in protecting immune-deficient mice against bacterial infection. In this respect, the function of superantibodies can be characterized as operating as part of the innate immunity, in an antigen-specific manner but not as part of the antibody maturation process. Thus, natural superantibodies exhibit a synergism between the innate and acquired immune systems.

It also has been demonstrated that short synthetic peptides (e.g., of about 15-25 residues) can be conjugated to antibodies (including antibody fragments), so as to provide such autophilic properties and improved potency without loss of specificity. These artificial superantibodies form self-complexes when bound to a target antigen but remain soluble in solution. See, e.g., Kohler et al., Appl Biochem Biotechnol. January-March 2000;83(1-3):1-9; discussion 10-2, 145-53. The term superantibody also has come to be associated with other antibody fusion proteins/conjugates wherein a fusion partner or included domain imparts unconventional binding functionality to the antibody, which is not directly associated with the antibody antigen-binding site. Additional features now associated with artificially generated “superantibodies” include receptor or antigen cross-linking by interaction of antibodies through cross-linking-facilitating domains, which may result in improved signaling, etc.; other protein binding; the ability to cleave bound peptide portions upon antigen binding for the delivery of fusion partner payloads, such as immunogenic peptides, to a target cell bound by the superantibody; and the ability to penetrate cell membranes to access intracellular targets. Cell-penetrating superantibodies are described in, e.g., Zhao et al., J. Immunological Methods 254:137-145. Briefly, such antibodies are engineered to contain a membrane-translocating sequence (MTS), which facilitates cell membrane transport. This technique also can be applied to other proteins, such as fusion proteins (see, e.g., Rojas et al., Nature Biotech., 16:370-375 (1998) with respect to the application of such techniques to a non-antibody protein). Such superantibodies can be used to target internal cellular proteins that turn cells cancerous. Superantibody technology is further described in, e.g., Kohler, Immunol Today. May 1998;19(5):221-7; Zhao et al., J Immunother. January-February 2002;25(1):57-62; and International Patent Application WO 02/097041.

As indicated above, superantibody domains, specifically MTSs, can be used to impart internalizing functionality to antibodies. In another aspect, the invention provides internalizing non-superantibody/non-MTS-comprising anti-γ2 antibodies.

Many full length antibodies are internalized by cells (see, e.g., Liu et al., Cancer Research 57, 3629-3634 (1997)). Certain antibodies and antibody fragments of the invention may be internalized, particularly in the context of multispecific antibodies. The efficiency with which antibodies mediate internalization differs depending on the type of the antibody (e.g., whole antibody, fragment, single chain, monomeric, dimeric, etc.) and on the epitope recognized (Yarden (1990) Proc. Nati. Acad. Sci. USA 87: 2569-2573; Hurwitz et al (1995) Proc. Natl. Acad. Sci. USA 92: 3353-3357). Where a recombinant antibody is generated from vertebrate antibodies that normally are not internalized, a recombinant antibody comprising sequences from an antibody that is internalized can be generated using known techniques to impart internalizing functionality. Examples of such methods are described in, e.g., U.S. Pat. No. Application 20010008759. Antibodies selected and/or modified in terms of size or other features so as exhibit internalization-promoting properties can be identified through screening of phage display libraries. See, e.g., Becerill et al., Biochem Biophys Res Commun. Feb. 16, 1999;255(2):386-93; Poul et al., J Mol Biol. Sep 1, 2000;301(5):1149-61; Gao et al., J Immun Methods. Mar. 1, 2003;274(1-2):185-97; Nielsen et al., Pharm. Sci. Technol. Today. August 2000;3(8):282-291; Gao et al., J Immunol Methods. 2003 Mar 1;274(1-2):185-97; and Marks et al., Methods Mol Biol. 2004;248:201-8.

In another aspect, the invention provides anti-γ2 antibodies that are capable of catalyzing one or more enzymatic reactions. Such catalytic antibodies are typically referred to as abzymes.

Abzyme “variants” of antibodies or antibody fragments can be produced by any suitable combination of γ2-binding portion(s) and one or more abzyme portions created by the modification of a pre-existing anti-γ2 antibody or antibody fragment (e.g., by chemical mutation/derivation) or, more typically, through the inclusion of suitable catalysis-facilitating sequences obtained from an abzyme in an anti-γ2 antibody, antibody fragment, or other L5G2BP (e.g., in the context of a fusion protien comprising a complete antibody or antibody fragment portion, or in the context of introducing such sequences as an insertion or substitution of part of an antibody, antibody fragment, or other protein).

Abzymes can be generated by a number of known techniques, which are only briefly described here. Traditionally, abzymes were generated by immunizing a suitable chordate in vivo or chordate cells in vitro (e.g., nonhuman mammalian splenocytes) with a hapten that mimics the transition state of an enzyme-catalyzed reaction (i.e., a transition state analog). In vitro methods offer a number of advantages including the ability to use less hapten analog in the production of the abzyme. Resulting antibodies, which can be identified by, e.g., phage display or other suitable display techniques, should be specific for (complementary to) the transition state structure. Such antibodies by binding to the appropriate substrate can promote the formation of the transition state and thereby lower the energy barrier to the formation of the transition state (i.e., decrease the free energy of the transition state), similar to the action of enzymes. To increase the antibody specificity, immunogens in which no more than about 8 amino acids linked to the hapten typically are used for the generation of abzymes by in vivo or in vitro methods.

Evolutionary dynamics methods, which involve the use of phage display libraries derived from a first abzyme and selected against a second transition state analog (TSA) can be useful in maximize the differential affinity for the transition state relative to the ground state so as to provide abzymes with improved reaction rates (kcat).

Electrostatic complementarity can be used to generate catalytic antibodies with a precisely positioned negatively charged residue in the combining site. “Bait and switch” hapten design has proved a successful technique for catalytic antibody production and it offers, in conjunction with transition state stabilization, the potential to yield catalysts of heightened activity. In a bait-and-switch approach, charge complementarity between hapten and antibody is exploited to induce appropriately positioned acids, bases, and nucleophiles. Alternatively, catalytic residues can be selected directly by irreversible chemical modification when mechanism-based inhibitors are employed as haptens. The latter strategy, dubbed reactive immunization, has the virtue of allowing rational engineering of covalent catalysis.

Mutagenesis by typical recombinant technologies and/or chemical modification techniques (derivativization and/or mutation) can be used to generate abzymes or to modify the functionality of abzymes produced by other methods. For somatic mutagenesis, antibody structures can be elucidated, e.g. by X-ray crystallography, and the residues important in the catalytic process determined by docking modeling. Mutagenesis at these potential target sites can identify the residues that are important to catalyze the target reaction. Once identified, substitutions can be made and analyzed to identify advantageous variations in terms of catalysis. In another aspect, abzymes can be generated by the addition of suspected catalytic residues to the binding site of an antibody. Abzymes also can be selectively altered by cofactors to give modified activity through chemical derivation and abzymes can be generated by chemical mutation techniques which introduce moieties, such as modified amino acid residues, to a binding site. Another abzyme generation approach uses the properties of anti-idiotypic Abs to generate internal images of enzyme active sites.

Abzymes can catalyze many diverse types of reactions including, e.g., pericyclic processes; group transfer reactions; various additions and eliminations; oxidations and reductions; aldol reactions; cleavage and condensation reactions; and cofactor-dependent transformations, such as, for example, acyl transfer reactions; Diels-Alder condensation; Claisen rearrangement; Michael reactions; phosphodiester hydrolysis; aryl ester hydrolysis; aldol condensations; aldol additions; crossed aldol reactions; self aldol reactions; retro aldol reactions; Robinson anulation; kinetic resolutions; retro-aldol-retro-Michael reactions; and a variety of decarboxylation reactions (e.g., β-keto acid decarboxylations).

The rates of reactions catalyzed with abzymes, as measured by kinetic parameters such as KM and Vₘₐₓ , currently are up to a million-fold greater than the corresponding uncatalyzed reactions, but often less than exhibited in similar enzymes. Nonetheless, such molecules can be very useful for diagnostic and pharmaceutical applications.

In one particular aspect, the invention provides an anti-γ2 abzyme that is capable of catalyzing the synthesis/formation of a drug, such as an anti-cancer chemotherapeutic agent from a prodrug, zymogen, or the like. For example, anti-cancer chemotherapeutic agents such as doxorubicin and camptothecin can be provided to a patient in masked form (as a prodrug) by combination with a group comprising a hapten that the abzyme portion of the anti-γ2 abzyme is specific for, after pre-administration of the anti-γ2 abzyme. In such a method, the abzyme binds to target cells and the later-administered masked chemotherapeutic agent is able to bind to the abzyme bound to the target cells, resulting in catalysis of the prodrug to active drug reaction, and formation of the chemotherapeutic agent at the site of the target cancer cells. Undesirable toxic side effects in non-target cells, tissues, etc. are avoided in the patient, thereby improving the health of the patient and improving the likelihood of survival. An abzyme-catalyzed reaction desirably is a reaction that is not catalyzed readily by human enzymes. For example, abzymes can be generated that catalyze tandem retro-aldol, retro-Michael reactions with respect to such masked agents that are not readily catalyzed by native human enzymes, thereby avoiding undesirable formation of the chemotherapeutic agent outside the context of target cells to which the MNKAMRBP abzyme is bound. Abzymes also can be used to facilitate the cleavage of proteins at target cells, such as cytotoxic proteins, apoptosis-promoting proteins, immunogenic peptides, etc. In addition to therapeutic agents, abzymes can be used to release diagnostic reporters from a comprising molecule or support composition, wherein release from the support or comprising molecule is required for detection of the reporter. Such a method can be used to, for example, identify the site of cancer cells for directing operative methods (e.g., biopsy and/or targeted application of radiotherapy).

Additional detailed methods and principles relevant to the production of abzymes are provided in, e.g., Hilvert, Annual Review of Biochemistry, 2000 69:751-793; Partridge, Biochem Soc Trans. November 1993;21(4):1096-8; Wentworth et al., Cell Biochem Biophys. 2001 35(1):63-87; Tramontano et al., Science, 1986 234(4783):1566-70; Pollack et al., Science, 1986 234(4783):1570-3; Nishi, Curr Pharm Des. 2003;9(26):2113-30; Wentworth, Science, Vol 296, Issue 5576, 2247-2249, 2002; Janda et al., J. Am. Chem. Soc. 112,1274 (1990); Lerner et al., Science,1991, 252(5006):659-67; Fletcher et al., Nat. Biotech. 16(11):1065-1067 (1998); Barbas et al., Proc. Natl. Acad. Sci USA 96, 6925-6930, 1999; Shabat et al., Proc. Natl. Acad. Sci. USA 1999, 96, 6925-6930; Wentworth et al., Proc Natl Acad Sci USA Jan. 23, 1996;93(2):799-803; Frbioulet et al., Appl Biochem Biotechnol. May-June 1994;47(2-3):229-39; Luo et al., Biochem Biophys Res Commun. Feb. 15, 1994;198(3):1240-7; Takahashi, et al. Nat. Biotech., 19(6):563-567 (2001); International Patent Application No. PCT/AU97/00194; US Patent Application 20030148484; and U.S. Pat. Nos. 5,258,289; 4,792,446; 4,888,281; 5,229,272; 5,156,965; 5,126,258; 6,387,674; and 6,590,080; and Keinan and Ehud (Eds.) CATALYTIC ANTIBODIES (1st Ed. 2004) Wiley-VCH, Weinheim.

In another aspect, the invention provides an anti-γ2 antibody variant/fusion protein comprising an additional portion capable of binding antibodies native to the serum of patients to which the anti-γ2 antibody is administered under conditions such that native antibodies bound by the anti-γ2 variant/fusion protein are able to mediate their effector function. Related compositions and techniques are described in, e.g., U.S. Pat. No. 6,589,527.

In another aspect, the invention provides an anti-γ2 antibody comprising only V-domains. Camelids (camels, llamas, and alpacas) and sharks produce antibodies that display single chain high-affinity VH-domain-only antibodies, rather than an Fv module, and that therefore bind to their target antigens using just three CDR loops. These VH domains are typically referred to as VHHS. A relatively enlarged hypervariable region allows such molecules to exhibit a broad antigen-binding repertoire, despite the lack of combinatorial diversity associated with regular antibody variable regions. Human V-like domain proteins (“camelized” human VH antibodies) have been successfully adapted as a scaffold for display and selection using large CDR loops that can penetrate clefts in the target antigen. Such humanized V-like domains can be a suitable framework for targeting “clefts” and/or “canyons” in target proteins. Bispecific VHH antibodies have been generated by tethering two single-domain antibody fragments with the structural upper hinge of a typical antibody and other VHH antibodies have been successfully targeted to cancer-associated targets. Such VHH, camelized, and VHH-associated bispecific antibodies are described in, e.g., Muyldermans, J Biotechnol. June 2001 ;74(4):277-302; Riechmann and Muyidermans, J Immunol Methods. Dec. 10, 1999;231(1-2):25-38; Conrath et al., J Biol Chem. Mar. 9, 2001;276(10):7346-50; and Cortez-Retamozo et al., Int J Cancer. Mar. 20, 2002;98(3):456-62.

Ln-5 γ2 binding peptides provided by this invention and/or that are suitable for use in the context of this invention also include immunoadhesins, which are molecules wherein one or more CDRs of an anti-γ2 antibody are covalently or noncovalently associated with a molecule (immunoadhesins, however, lack the more complicated structure of antibodies and antibody fragments). An immunoadhesin may incorporate the CDR(s) as part of a larger polypeptide chain (as a fusion protein), may covalently link the CDR(s) to another polypeptide chain, or may incorporate the CDR(s) noncovalently. The included CDRs (and typically supporting sequences/residues, such as FRs) permit the immunoadhesin to specifically bind to a particular portion of an antigen of interest (as defined by an epitope or, more generally, an antigenic determinant region). A number of similar antibody domain/fragment molecules have been described and/or proposed, which also may be suitably adapted to the methods, uses, and compositions of this invention (see, e.g., U.S. Pat. No. Application 20040033561).

As suggested above, the invention also provides new and useful L5G2BP derivatives. As described elsewhere herein, a derivative is any peptide in which one or more of the amino acid residues of the peptide have been chemically modified (e.g., by alkylation, acylation, ester formation, amide formation, or other similar type of modification) or covalently associated with one or more heterologous substituents (e.g., a lipophilic substituent, a PEG moiety, a peptide side chain linked by a suitable organic moiety linker, etc.). The second type of derivative can separately be described as a conjugate.

In general, L5G2BPs described herein can be modified by inclusion of any suitable number of such modified amino acids and/or associations with such conjugated substituents. Suitability in this context general is determined by the ability to at least substantially retain γ2 DIII selectivity and/or specificity associated with the non-derivatized parent L5G2D3BP (and therefore also implies retention of a suitable level of affinity and/or avidity). The inclusion of one or more modified amino acids may be advantageous in, for example, (a) increasing polypeptide serum half-life, (b) reducing polypeptide antigenicity, or (c) increasing polypeptide storage stability. Amino acid (s) are modified, for example, co-translationally or post-translationally during recombinant production (e.g., N-linked glycosylation at N—X—S/T motifs during expression in mammalian cells) or modified by synthetic means.

Non-limiting examples of modified amino acids that may make up part of a derivative include glycosylated amino acids, sulfated amino acids, prenlyated (e.g., farnesylated, geranylgeranylated) amino acids, acetylated amino acids, acylated amino acids, PEGylated amino acids, biotinylated amino acids, carboxylated amino acids, phosphorylated amino acids, and the like. References adequate to guide one of skill in the modification of amino acids are replete throughout the literature. Exemplary protocols are found in, e.g., Walker (1998) PROTEIN PROTOCOLS ON CD-ROM Humana Press, Towata, N.J. Typically, a modified amino acid residue in a derivative is a glycosylated amino acid, a PEGylated amino acid, a farnesylated amino acid, an acetylated amino acid, a biotinylated amino acid, a lipid moiety-conjugated amino acid residue, or an organic derivatizing agent-conjugated residue.

Additionally, antibodies and antibody fragments can be chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties (e.g., a L5G2D3BP can be conjugated to a PEG with a molecular weight of between about 1,000 and about 40,000, such as between about 2000 and about 20,000, e.g., about 3,000-12,000).

In another exemplary aspect, the invention provides a L5G2BP that is conjugated to a second molecule that is selected from a radionuclide, an enzyme, an enzyme substrate, a cofactor, a fluorescent marker, a chemiluminescent marker, a peptide tag, a magnetic particle, a toxin, or other drug. Another exemplary feature of the invention is a L5G2BP that is conjugated to one or more antibody fragments, nucleic acids (oligonucleotides), nucleases, hormones, immunomodulators, chelators, boron compounds, photoactive agents, dyes, and the like. These and other suitable agents can be coupled either directly or indirectly to L5G2BPs of the invention. One example of indirect coupling of a second agent is coupling by a spacer moiety. These spacers, in turn, can be either insoluble or soluble (see, e.g., Diener, et al., Science, 231:148,1986) and can be selected to enable drug release from the L5G2D3BP at a target site and/or under particular conditions. Additional examples of therapeutic agents that can be coupled to L5G2BPs include lectins and fluorescent peptides.

In another aspect, the invention provides crosslinked L5G2BP derivatives. For example, a L5G2BP derivative can be produced by crosslinking two or more antibodies, at least one of which is specific/selective for γ2 DIII (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Co., Rockford, Ill.

L5G2D3BPs also can be conjugated with any suitable type of chemical group, such as polyethylene glycol (PEG), a methyl or ethyl group, or a carbohydrate group. These and other suitable conjugated groups may be used to improve the biological characteristics of the L5G2D3BP, e.g., to increase serum half-life, solubility, and/or tissue binding.

L5G2D3BP derivatives can be produced by chemically conjugating a radioisotope, protein, or other agent/moiety/compound to, for example, (a) the N-terminal side or C-terminal side of the L5G2D3BP or subunit thereof (e.g., an anti-γ2 DIII antibody H chain, L chain, or anti-γ2 DIII specific/selective fragment thereof), (b) an appropriate substituent group or side chain or (c) a sugar chain associated with the L5G2D3BP (see, e.g., ANTIBODY ENGINEERING HANDBOOK, edited by Osamu Kanemitsu, published by Chijin Shokan (1994)). Derivatives also can be generated by conjugation at internal residues or sugars, where appropriate and available.

In one aspect, a derivatizing agent is a low molecular weight compound. Examples include anticancer agents, such as alkylating agents (e.g., nitrogen mustard, cyclophosphamide); metabolic antagonists (e.g., 5-fluorouracil, methotrexate); plant alkaloids (e.g., vincristine, vinblastine, vindesine); hormone drugs (e.g., tarnoxifen, dexamethasone), and the like (see, e.g., CLINICAL ONCOLOGY, edited by Japanese Society of Clinical Oncology, published by Cancer and Chemotherapy (1996)).

It will be recognized that although the description of derivatives herein primarily focuses on antibodies (and to a lesser extent antibody fragments), these principles also may be applied to other L5G2BPs of the invention (e.g., L5G2D3BP antibody mimetics, proteins and fusion proteins comprising γ2 DIII-binding sequences from non-antibody L5G2D3BPs, etc.). Accordingly, derivatives of these types of L5G2BPs are also a feature of this invention.

To better illustrate these aspects of the invention, particular examples of types of derivatives are discussed in further detail here.

In one aspect, L5G2BP derivatives comprising one or more radiolabeled amino acids are provided. A radiolabeled L5G2D3BP may, for example, be used for both diagnostic and therapeutic purposes (conjugation to separate radiolabeled molecules is another possible feature). Nonlimiting examples of labels for conjugation to peptides and/or incorporation in amino acid residues include, but are not limited to ³H, ¹⁴C, ¹⁵N, 35S, ⁹⁰Y, ⁹⁹Tc, and ¹²⁵I, ¹³¹I, and ¹⁸⁶Re. Methods for preparing radiolabeled amino acids and related peptide derivatives are known in the art (see, e.g., Junghans et al. in CANCER CHEMOTHERAPY AND BIOTHERAPY 655-686 (2d edition, Chafner and Longo, eds., Lippincott Raven (1996)) and U.S. Pat. Nos. 4,681,581, 4,735,210, 5,101,827, 5,102,990 (U.S. Re. Pat. No. 35,500), 5,648,471, and 5,697,902. For example, a radioisotope can be conjugated by a chloramine T method.

Advantageous radionuclides in diagnostic contexts typically include indium isotopes and in the context of therapeutic applications advantageous radionuclides typically include yttrium isotopes, which are commonly cytotoxic. Photon-emitting radioisotopes, in general, are advantageous in diagnostic (e.g., radioimmunoscintigraphy (RIS)) methods. Auger electrons have a very short path length (about 5-10 nm) and normally need to be internalized to be cytotoxic (see, e.g., Adelstein, et al., Nucl. Med. Biol. 14:165-169 (1987)). Accordingly, peptides conjugated to such isotopes can be useful in diagnostic methods, but generally only peptides that are internalized should be considered for radioisotopes that emit Auger electrons in therapeutic contexts. Alpha particles need to be close to a cell (within 3-4 cell diameters) to be effective as therapeutic agents (Vriesendorp, et al., “Radioimmunoglobulin therapy,” in HIGH DOSE CANCER THERAPY, Armitage, et al. (eds). (Williams & Wilkins, Baltimore, Md. 1992)). Both Auger electrons and alpha emitters can be considered to have high selectivity because their short-range emission typically will not irradiate neighboring normal cells. Thus, in one aspect, the invention provides a L5G2D3BP conjugated to and/or comprising one or more radionuclide molecules that is highly selective for targeted cells and/or tissues (such as the invasive front of a carcinoma cell population in a human patient).

The radiometals ¹¹¹In and ⁹⁰Y are, respectively, a pure γ-emitter and a pure β-emitter. Iodine-125, the most commonly used emitter of Auger electrons, has a half-life of about 60 days and frequently is released by immunoconjugates in vivo (due to dehalogenation). The most commonly considered alpha emitters for clinical use, astatine-211 and bismuth-212, have relatively short half-lives (7.2 h and 1.0 h, respectively) and decay into radioactive isotopes that may not be retained by the immunoconjugate after the first alpha emission (see, e.g., Wilbur, Antibiot. Immunoconjug. Radiopharm. 4:85-97 (1991)). For diagnostic applications, L5G2D3BPs labeled with indium-111 or technetium-99 m can be advantageously used. Both of these isotopes emit gamma rays within an appropriate energy range for imaging, i.e., about 100-250 keV. Energies below this range typically are not penetrating enough to reach an external imaging device. Higher energy levels are difficult to collimate and provide diagnostic images with poor resolution. The short-half life of ⁹⁹Tc typically restricts its use to immunoconjugates with rapid tumor uptake.

In another aspect of the invention, first and second L5G2BPs conjugated with first and second radioisotopes are provided. In another facet, a single L5G2BP conjugated with two radioisotopes is provided. An advantage of using two separate radioisotopes, e.g., one for imaging and one for therapy, is that it can facilitate outpatient treatment. In such a method, the low amount of radioactivity used diagnostically typically does not represent a radiation hazard, while the radiation emitted by a therapeutic isotope, such as a pure beta-emitter, typically will largely be absorbed in the vicinity of the targeted cells.

Radioisotopes can be attached directly or indirectly to a L5G2BP. The radioisotopes ¹²⁵I, ¹³¹I, ⁹⁹Tc, ¹⁸⁶Re, and ¹⁸⁸Re can be, for example, covalently bound to proteins (including antibodies) through amino acid functional groups. For radioactive iodine it is usually through the phenolic group found on tyrosine. There are numerous methods to accomplish this including, e.g., methods involving chloramine-T (see, e.g., Greenwood, et al. Biochem J. 89: 114-123 (1963) and lodogen (Salacinski, et al. Anal. Biochem. 117: 136-146 (1981)). Tc and Re isotopes can be covalently bound through the sulfhydryl group of cysteine residues (see, e.g., Griffiths, et al. Cancer Res. 51: 4594-4602 (1991)). However, such compositions may be relatively better suited for diagnostic purposes as the body often can break these covalent bonds, releasing the radioisotopes to the circulatory system.

L5G2BPs that are linked to cytotoxins are another useful feature of the invention. Cytotoxic drugs which can be conjugated to L5G2BPs, such as anti-γ2 DIII antibodies, and used for in vivo therapy include, but are not limited to, daunorubicin, mercaptopurine, adriamycin, doxorubicin, methotrexate, and Mitomycin C. Cytotoxic drugs can interfere with critical cellular processes including DNA, RNA, and protein synthesis. For a description of these classes of drugs which are well known in the art, and their mechanisms of action, see Goodman, et al., GOODMAN AND GILMAN'S THE PHARMACOLOGICAL BASIS OF THERAPEUTICS, 8th Ed., Macmillan Publishing Co., 1990. Additional techniques relevant to the preparation of antibody immunotoxins are provided in, e.g., Vitetta, Immunol. Today 14:252 (1993) and U.S. Pat. No. 5,194,594. Cytotoxic proteins, such as pseudomonas exotoxin, also can be conjugated or linked to a L5G2BP (several examples of such proteins are described elsewhere herein with reference to L5G2BP fusion proteins).

Additional examples of toxic molecules that can be conjugated to L5G2BPs include diphtheria toxin (e.g., diphtheria A chain and active fragments thereof) and related molecules (e.g., hybrid molecules) (see, e.g., U.S. Pat. No. 4,675,382), ricin toxin (e.g., a deglycosylated ricin A chain toxin) (see, e.g., Vitetta et al., Science 238, 1098 (1987) and U.S. Pat. No. 4,643,895), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin toxins. Conjugates of the monoclonal antibody and such cytotoxic moieties can be made using a variety of bifunctional protein coupling agents. Examples of such reagents include SPDP, IT, bifunctional derivatives of imidoesters such a dimethyl adipimidate HCI, active esters such as disuccinimidyl suberate, aldehydes such as glutaraldehyde, bis-azido compounds such as bis (p-azidobenzoyl)hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin typically may be readily joined to the Fab fragment/portion of an antibody or antibody fragment. Other suitable conjugated molecules include ribonuclease (RNase), DNase I, Staphylococcal enterotoxin-A, pokeweed antiviral protein, diphtherin toxin, and Pseudomonas endotoxin. See, for example, Pastan et al., Cell 47:641 (1986), and Goldenberg, Calif.—A Cancer Journal for Clinicians 44:43 (1994). Additional toxins suitable for use in the present invention are known (see, e.g., U.S. Pat. No. 6,077,499).

In one aspect, the invention provides a L5G2BP that is conjugated to a mixed toxin. A mixed toxin molecule is a molecule derived from two different (typically polypeptide) toxins. Generally, peptide toxins comprise one or more domains responsible for generalized eukaryotic cell binding, at least one enzymatically active domain, and at least one translocation domain. The binding and translocation domains are required for cell recognition and toxin entry respectively. Naturally-occurring proteins which are known to have a translocation domain include diphtheria toxin, Pseudomonas exotoxin A, and possibly other peptide toxins. The translocation domains of diphtheria toxin and Pseudomonas exotoxin A are well characterized (see, e.g., Hoch et al., Proc. Natl. Acad. Sci. USA 82:1692, 1985; Colombatti et al., J. Biol. Chem. 261:3030, 1986; and Deleers et al., FEBS Lett. 160:82, 1983), and the existence and location of such a domain in other molecules may be determined by methods such as those employed by Hwang et al. (Cell 48:129, 1987); and Gray et al. (Proc. Natl. Acad. Sci. USA 81:2645, 1984). In view of these techniques, a useful mixed toxin hybrid molecule can be formed, for example, by fusing the enzymatically active A subunit of E. coli Shiga-like toxin (Calderwood et al., Proc. Natl. Acad. Sci. USA 84:4364,1987) to the translocation domain (amino acid residues 202 through 460) of diphtheria toxin, and to a molecule targeting a particular cell type, as described in U.S. Pat. No. 5,906,820. The targeting portion of the three-part hybrid can cause the molecule to attach specifically to the targeted cells, and the diphtheria toxin translocation portion can act to insert the enzymatically active A subunit of the Shiga-like toxin into a targeted cell. The enzymatically active portion of Shiga-like toxin, like diphtheria toxin, acts on the protein synthesis machinery of the cell to prevent protein synthesis, thus killing the targeted cell.

Additionally useful conjugate substituents include anti-cancer retinoids, taxane conjugates (see, e.g., Jaime et al., Anticancer Res. March-April 2001;21(2A):1119-28), cisplatin conjugates, thapsigargin conjugates, linoleic acid conjugates, calicheamicin conjugates (see, e.g., Damle et al., Curr Opin Pharmacol. August 2003;3(4):386-90), doxorubicin conjugates, geldanamycin conjugates, and the like, also may be useful in promoting the treatment of cancer (see, generally, Trail et al., Cancer Immunol Immunother. May 2003;52(5):328-37).

In another aspect, a L5G2BP is conjugated to a tumor targeting domain peptide or molecule. In one example, a L5G2BP is conjugated to a tumor targeting factor VII sequence.

In another aspect, the invention provides L5G2BPs conjugated to or otherwise stably associated with one or more detection-facilitating agents (i.e., detection agents, tags, or labeling moieties). Useful detection agents with which a L5G2BP may be derivatized include fluorescent compounds, including fluorescein, fluorescein isothiocyanate, rhodamine, 5-dimethylamine-1-napthalenesulfonyl chloride, lanthanide phosphors, and the like. Additional examples of suitable fluorescent labels include a ¹²⁵Eu label, an isothiocyanate label, a phycoerythrin label, a phycocyanin label, an allophycocyanin label, an o-phthaldehyde label, a fluorescamine label, etc. Examples of chemiluminescent labels include luminal labels, isoluminal labels, aromatic acridinium ester labels, imidazole labels, acridinium salt labels, oxalate ester labels, a luciferin labels, luciferase labels, aequorin labels, etc.

A L5G2BP also can be labeled with enzymes or enzyme substrates that are useful for detection, such as horseradish peroxidase, β-galactosidase, luciferase, alkaline phosphatase, glucose oxidase, and the like. An L5G2BP also be labeled with biotin, and accordingly detected through indirect measurement of avidin or streptavidin binding. A L5G2BP may also be labeled with a predetermined polypeptide epitopes recognized by a secondary reporter (e.g., leucine zipper pair sequences, binding sites for secondary antibodies, metal binding domains, epitope tags, etc.). Additional examples of enzyme conjugate candidates include malate dehydrogenase, staphylococcal nuclease, delta-V-steroid isomerase, yeast alcohol dehydrogenase, α-glycerophosphate dehydrogenase, triose phosphate isomerase, asparaginase, glucose oxidase, ribonuclease, urease, catalase, glucose-6-phosphate dehydrogenase, glucoamylase, and acetylcholinesterase.

Additional exemplary labeling moieties generally include, but are not limited to spin-labeled molecules and other labeling moieties of diagnostic value (e.g., molecules that act as contrast agents in MRI diagnosis).

In another aspect, the invention provides a L5G2BP that is conjugated to an immunomodulator, such as an immunomodulating cytokine, stem cell growth factor, lymphotoxin (e.g., a TNF such as TNFα), or a hematopoietic factor. Examples of such molecules that may be useful as conjugates include IL-1, IL-2, IL-3, IL-6, IL-10, IL-12, IL-18, and IL-21, colony stimulating factors (e.g., granulocyte-colony stimulating factor (G-CSF) and granulocyte macrophage-colony stimulating factor (GM-CSF)), interferons (e.g., IFNα, IFNβ, and IFNγ), the stem cell growth factor designated “S1 factor,” erythropoietin, and thrombopoietin, active fragments thereof, derivatives thereof, variants thereof, or a combination of any thereof. Examples of immunomodulating agents (e.g., various cytokines, T cell activity modulators, NK cell activity modulators, etc.) are described elsewhere herein.

In another aspect, a L5G2BP derivative comprises a conjugated nucleic acid or nucleic acid-associated molecule. Typically, such derivatives are associated with an antibody, antibody fragment, antibody mimetic, or other type of L5G2BP that can be internalized by γ2-associated cells (e.g., cells actively secreting γ2 or γ2-associated peptides). In one such facet of the invention, the conjugated nucleic acid is a cytotoxic ribonuclease. In another facet, the conjugated nucleic acid is an antisense nucleic acid (e.g., a S100A10 targeted antisense molecule, which also can be an independent component in a combination composition or combination administration method of the invention—see, e.g., Zhang et al., J Biol Chem. Jan. 16, 2004;279(3):2053-62). In another facet, the conjugated nucleic acid is an inhibitory RNA molecule (e.g., a siRNA molecule). In another facet, the conjugated nucleic acid is an immunostimulatory nucleic acid (e.g., an immunostimulatory CpG motif-containing DNA molecule). In another aspect, the conjugated nucleic acid is an expression cassette coding for expression of a tumor suppressor gene, anti-cancer vaccine, anti-cancer cytokine, or apoptotic agent. Such derivatives also may comprise conjugation of a nucleic acid coding for expression of one or more cytotoxic proteins.

Thus, in one exemplary aspect, a L5G2D3BP or related compound/molecule (such as a L5G2D3BP-encoding nucleic acid, a L5G2D3BP related antigenic peptide, etc.) is conjugated to or otherwise associated with a functional nucleic acid molecule. Functional nucleic acids include antisense molecules, interfering nucleic acid molecules (e.g., siRNA molecules), aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

In one such aspect a L5G2D3BP or related molecule is conjugated to an antisense nucleic acid, such as an antisense nucleic acid targeted against Ln-5 γ2, against a Ln-5-interacting integrin, against beta-catenin, or other relevant target. A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

In another such aspect, a L5G2D3BP or a related molecule is conjugated to an aptamer. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No.5,631,146) and theophiline (U.S. Pat. No. 5,580,737), as well as large molecules, such as reverse transcriptase (U.S. Pat. No. 5,786,462) and thrombin (U.S. Pat. No. 5,543,293). Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130,6,028,186, 6,030,776, and 6,051,698. Thus, a L5G2D3BP can be conjugated to an aptamer that binds to Ln-5, an Ln-5 associated molecule, a component of the basement membrane associated with preneoplastic and neoplastic cells (e.g., invasive carcinoma cells), or other suitable target.

In a further aspect, the invention provides a L5G2D3BP or related molecule (such as an L5G2D3BP-encoding nucleic acid, an L5G2D3BP-related antigenic peptide, or a nucleic acid encoding such an antigenic peptide) which is conjugated to a ribozyme. Ribozymes are nucleic acid molecules that are capable of catalyzing a chemical reaction, either intramolecularly or intermolecularly. Ribozymes are thus catalytic nucleic acids. There are a number of different types of ribozymes that catalyze nuclease or nucleic acid polymerase type reactions which are based on ribozymes found in natural systems, such as (a) hammerhead ribozymes, (described in, for example, U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, and 5,998,203, and International Patent Applications WO 9858058, WO 9858057, and WO 9718312), (b) hairpin ribozymes (described in, e.g., U.S. Pat. Nos.: 5,631,115, 5,646,031, 5,683,902, 5,712,384, 5,856,188, 5,866,701, 5,869,339, and 6,022,962), and (c) tetrahymena ribozymes (described in, e.g., U.S. Pat. Nos.: 5,595,873 and 5,652,107). There are also a number of ribozymes that are not found in natural systems, but which have been engineered to catalyze specific reactions de novo (examples of which are described in, e.g., U.S. Pat. Nos.: 5,580,967, 5,688,670, 5,807,718, and 5,910,408). Ribozymes typically cleave RNA or DNA substrates, and more commonly cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756. L5G2BPs can be conjugated to ribozymes that target any suitable substrate.

In an additional facet, the invention provides a L5G2BP or related molecule that is conjugated to a triplex forming function nucleic acid. Such nucleic acid molecules can interact with either double-stranded or single-stranded nucleic acid. When triplex molecules interact with a target region, a structure called a triplex is formed, in which three strands of DNA form a complex dependant on both Watson-Crick and Hoogsteen base-pairing. Triplex molecules can bind target regions with high affinity and specificity. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

In another aspect, a L5G2D3BP or related molecule is conjugated to an external guide sequence. External guide sequences (EGSs) are molecules that bind a target nucleic acid molecule forming a complex that is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (see, e.g., WO 92/03566 and Forster and Altman, Science 238:407-409 (1990) for discussion). Representative examples of how to make and use EGS molecules to facilitate cleavage of a variety of different target molecules are provided in the following non-limiting list of U.S. Pat. Nos.: 5,168,053, 5,624,824, 5,683,873, 5,728,521, 5,869,248, and 5,877,162.

Another feature of the invention is a L5G2D3BP that is conjugated to an interfering nucleic acid molecule, such as a siRNA or other RNAi molecule (e.g., an inhibitory double stranded (ds) RNA molecule of about 20-25 nucleotides), which is targeted to interfere with the action of a target gene expression product, such as a gene expression product involved in Ln-5-associated cancer progression. Methods for the production and use of interfering nucleic acid molecules are provided in, e.g., Nishikura, Cell. Nov. 16, 2001;107(4):415-8; Fjose et al., Biotechnol Annu Rev. 2001;7:31-57; Hanon, Nature. Jul. 11, 2002;418(6894):244-51; Brantl, Biochim Biophys Acta. May 3, 2002;1575(1-3):15-25; Tuschl, Chembiochem. Apr. 6, 2001;2(4):239-45; Caplen, Expert Opin Biol Ther. July 2003;3(4):575-86; Lu et al., Curr Opin Ther. 2003 Jun;5(3):225-34; Shuey et al., Drug Discov Today. Oct. 15, 2002;7(20):1040-6; Shi, Trends Genet. January 2003;19(1):9-12; Kovar et al., Semin Cancer Biol. August 2003;13(4):275-81; Lavrey et al., Curr Opin Drug Discov Devel. July 2003;6(4):561-9; Clewey, Commun Dis Public Health. June 2003;6(2):162-3; Duxbury et al., J Surg Res. April 2004;117(2):339-44; Caplen et al., Ann N Y Acad Sci. December 2003;1002:56-62; International Patent Application WO 01/75164; U.S. Pat. No. 6,506,559; and U.S. Patent Applications 20040086884, 20040077574, 20040063654, 20040033602, 20030167490, 20030157030, 20030114409, 20030108923, 20040014113, and 20020132788.

In the case of functional nucleic acids, typically the L5G2BP may be engineered to deliver the nucleic acid to the interior of target cells by known and standard techniques, a L5G2BP may be selected for cellular uptake, or a related molecule that is normally taken up by cells can be used so as to deliver the functional nucleic acid to the interior of a cell wherein its activity is desired.

Any method known in the art for conjugating a L5G2BP or related molecule to the conjugated molecule(s) (substituent(s) or derivatizer(s)), such as those described above, may be employed, including those methods described by Hunter, et al., Nature 144:945 (1962); David, et al., Biochemistry 13:1014 (1974); Pain, et al., J. Immunol. Meth. 40:219 (1981); and Nygren, J. Histochem. and Cytochem. 30:407 (1982). Linkage/conjugation can be accomplished in any suitable way. For example, a covalent linkage may take the form of a disulfide bond (if necessary and suitable, a L5G2D3BP could be engineered to contain an extra cysteine codon, which desirably does not interfere with the γ2 DIII binding activity of the molecule). A toxin molecule, derivatized with a sulfhydryl group reactive with a cysteine of a modified L5G2BP or unmodified L5G2BP, can form an immunoconjugate with such a L5G2BP peptide. Alternatively, a sulfhydryl group can be introduced directly to a L5G2BP using solid phase polypeptide techniques. For example, the introduction of sulfhydryl groups into peptides is described by Hiskey (Peptides 3:137,1981). The introduction of sulfhydryl groups into proteins is described in, e.g., Maasen et al. Eur. J. Biochem. 134:32, (1983). Once the correct sulfhydryl groups are present, the cytotoxin and L5G2D3BP can be purified, both sulfur groups reduced; cytotoxin and ligand mixed; (e.g., in a ratio of about 1:5 to 1:20); and disulfide bond formation allowed to proceed to completion (generally in about 20 to 30 minutes) at a suitable temperature (e.g., room temperature). The mixture can then be dialyzed against phosphate buffered saline or chromatographed in a resin such as Sephadex to remove unreacted ligand and toxin molecules.

Numerous types of cytotoxic compounds and other derivatizers can be joined to proteins through the use of a reactive group on the cytotoxic compound or through the use of a cross-linking agent. A common reactive group that will form a stable covalent bond in vivo with an amine is isothiocyanate (Means, et al. Chemical Modifications of Proteins (Holden-Day, San Francisco 1971) pp.105-110). This group preferentially reacts with the i-amine group of lysine. Maleimide is a commonly used reactive group to form a stable in vivo covalent bond with the sulfhydryl group on cysteine (Ji, Methods Enzymol 91: 580-609 (1983)). Monoclonal antibodies typically are incapable of forming covalent bonds with radiometal ions, but they can be, if necessary, attached to the antibody indirectly through the use of chelating agents that are covalently linked to the antibodies. Chelating agents can be attached through amines (Meares, et al., Anal. Biochem. 142:68-78 (1984)) and sulfhydral groups (Koyama Chem. Abstr. 120:217262t (1994)) of amino acid residues and also through carbohydrate groups (Rodwell, et al., Proc. Natl. Acad. Sci. 83:2632-2636 (1986); Quadri, et al., Nucl. Med. Biol. 20:559-570 (1993)). Since these chelating agents contain two types of functional groups, one to bind metal ions and the other to join the chelate to the antibody, they are commonly referred as bifunctional chelating agents (Sundberg, et al., Nature 250:587-588 (1974)).

Crosslinking agents that have two reactive functional groups are classified as being homo or heterobifunctional. Examples of homobifunctional crosslinking agents include bismaleimidohexane (BMH), which is reactive with sulfhydryl groups (Chen, et al. J Biol Chem 266: 18237-18243 (1991)) and ethylene glycolbis[succinimidylsucciate] (EGS), which is reactive with amino groups (Browning, et al., J. Immunol. 143: 1859-1867 (1989)). An example of a heterobifunctional crosslinker is m-maleimidobenzoyl-N-hydroxysuccinimide ester (MBS) (Myers, et al. J. Immunol. Meth.121: 129-142 (1989)).

A therapeutic or diagnostic agent also or alternatively can be attached at the hinge region of a reduced antibody component via disulfide bond formation. As an alternative, such peptides can be attached to the antibody component using a heterobifunctional cross-linker, such as N-succinyl 3-(2-pyridyldithio)proprionate (SPDP). Yu et al., Int. J. Cancer 56: 244 (1994). General techniques for such conjugation are well known in the art. See, for example, Wong, Chemistry Of Protein Conjugation And Cross-Linking (CRC Press 1991); Upeslacis et al., “Modification of Antibodies by Chemical Methods,” in MONOCLONAL ANTIBODIES: PRINCIPLES AND APPLICATIONS, Birch et al. (eds.) (Wiley-Liss, Inc. 1995) (text incorporated in its entirety); and Price, “Production and Characterization of Synthetic Peptide-Derived Antibodies,” in MONOCLONAL ANTIBODIES:

PRODUCTION, ENGINEERING AND CLINICAL APPLICATION, Ritter et al. (eds.) (Cambridge University Press 1995) (text incorporated in its entirety).

In some aspects, labels or other conjugated substituents are attached to the L5G2D3BP amino acid sequence by spacer arms of various lengths to reduce potential steric hindrance.

Unlabeled L5G2BP(s) can be used in combination with other labeled antibodies (second antibodies) that are reactive with the L5G2BP(s), such as antibodies specific for human immunoglobulin constant regions of antibodies that bind to anti-γ2 DIII mAbs. Alternatively, a L5G2BP can be directly labeled by conjugation with a derivative. As indicated by the foregoing discussion of derivatives, a wide variety of labels may be employed for direct or indirect labeling of L5G2BPs, such as radionuclides, fluors, enzymes, enzyme substrates, enzyme cofactors, enzyme inhibitors, ligands (particularly haptens), etc.

Conjugated L5G2BPs and the use of such molecules in the inventive methods described herein represent distinguishable aspects of the invention from “naked” molecules and the use thereof. Thus, for example, in one illustrative aspect the invention provides a method of reducing cancer progression in a human patient in need thereof by a method that includes delivery of an effective amount of a conjugated L5G2BP to the patient. In contrast, another feature of the invention is directed to “naked” L5G2BP reduction of cancer progression in a patient, including the delivery of an effective amount of an unconjugated L5G2BP to the patient, such that cancer progression in the patient is reduced.

L5G2BPs include a number of types of molecules in addition to the antibody, antibody fragment, and antibody-like molecules explicitly described above. In one aspect, the invention provides new non-antibody L5G2BPs. In another aspect, non-antibody L5G2BPs, including known γ2 DIII binding proteins can be used in various inventive methods provided here. In one exemplary aspect, the invention provides an antibody mimetic, such as a peptide based on a high affinity peptide-binding scaffold, which specifically binds one or more of the γ2 ADRs described herein. In another aspect, the invention relates to the use of such mimetics or other non-antibody L5G2BPs in the practice of various inventive methods provided herein and/or in the use of such molecules for the preparation of medicaments to treat conditions associated with γ2 (e.g., a cancer or pre-cancer condition). To illustrate this facet of the invention further, various types of non-antibody L5G2BPs are described here.

In one aspect, the invention provides affibodies (or affybodies) that specifically bind DIII or a portion of γ2 near DIII (e.g., the hinge region). Affibodies are a class of small, highly specific, and robust affinity proteins, designed to bind desired target proteins (see, e.g., U.S. Pat. No. 5,831,012). Affibodies typically can be characterized as simple proteins composed of a three-helix bundle based on the scaffold of one of the IgG-binding domains of Protein A. Protein A is a well known surface protein from the bacterium Staphylococcus aureus. This scaffold has excellent features as an affinity ligand and can be designed to bind with high affinity to any given target protein. The domain consists of 58 amino acids, 13 of which are randomized to generate affibody libraries with a large number of ligand variants. Such libraries can consist of a multitude of protein ligands with an identical backbone and variable surface-binding properties. Current libraries (e.g., available through Affibody®, Teknikringen 30, floor 6, Box 700 04, Stockholm SE-10044, Sweden) contain billions of variants.

Affibodies are characterized as “robust” in that they typically are able to withstand a broad range of physical conditions, including pH and elevated temperature, as compared to e.g., antibodies. Affibodies typically have a molecular weight of about 6 kDa, compared to the molecular weight of antibodies, which typically is about 150 kDa.

In function, affibody molecules mimic antibodies. In spite of the small size of these molecules, the binding site of affibody molecules has been demonstrated to be very similar to that of an antibody. Affibodies advantageously can be produced in bacteria and by chemical synthesis (e.g., combinatorial protein engineering). They also can be effectively coupled to form multimeric constructs. Affibodies can further be conjugated to other molecules to form derivatives and fused to form fusion proteins.

The properties of affibodies make them suitable for a number of biotechnological and therapeutic applications, such as industrial separation of proteins for production of protein-based pharmaceuticals and identification/validation of potential drug targets.

Affibodies can be “engineered” to have desired properties (e.g., high specificity and affinity—typically nanomolar level affinity). A specific affibody will thus typically bind only to its target in a wide context of molecules. The small size (only about 60 amino acids), high solubility, ease of further engineering into multifunctional constructs, excellent folding, absence of cysteines, and stable scaffold that can be produced in large quantities using low cost bacterial expression systems, make affibodies powerful capture molecules.

Affibodies can be useful as diagnostic agents and receptor/ligand blocking agents. Systems based on affibodies labeled for fluorescence resonance energy transfer (FRET)-based detection have been developed to allow quantitative measurements of non-labeled target molecules, for applications in, for example, protein “chip” formats. So-called anti-idiotypic affibodies have also been developed and employed to produce Self-assembled Networks of Artificial Proteins (SNAPs) to allow the build-up of molecular micro structures. Selection systems for affibody libraries have also been developed both based on Gram positive bacteria, FACS-sorting of micro beads, and protein contact assays. Typically affibodies are selected from phage display libraries expressing a segment of the target protein of interest (e.g., a portion of DIII or a peptide comprising the same).

In one aspect, the invention provides affibodies that individually are specific for any one of the γ2 ADRs described herein. In another aspect, the invention provides a method of preparing an affibody that binds to one of the γ2 ADRs described herein. In a further aspect, the invention relates to the use of an affibody specific for a γ2 ADR described herein in the preparation of a medicament to treat cancer or a pre-cancerous condition in a patient.

Methods and principles relevant to the design (e.g., generation), production, and use of affibodies (including exemplary additional modifications to such molecules), can be found in, e.g., Graslund et al., J. Biotechnol. 99, 41; Nygren et al., Curr Opin Struct Biol 7, 463-469 (1997); Nord et al., Nature Biotechnol 15, 772-777 (1997); Nord et al., Protein Eng 8, 601-608 (1995); Högbom et al., Proc. Natl. Acad. Sci. U.S.A. 100, 3191-3196; Wahlberg et al., Proc. Natl. Acad. Sci. U.S.A. 100, 3185-3190; Ronnmark et al., J. Immunol. Meth. 261, 199-211; Ronnmark et al., J. Immunol. Meth. 281, 149-160; Karlström, et al., J. Anal. Biochem. 295, 22-30; Nord et al., J. Biotechnol. 80, 45-54; Eklund et al., Proteins 48, 454-462 (2002); Gunneriusson et al., Protein Eng 12, 873-878 (1999); Wikman et al., Protein Engineering, Design & Selection (advance access published Jun. 18, 2004); Sandstrom et al., Protein Engineering vol.16 no. 9 pp. 691-697, 2003; Högbom et al., Curr. Opin. Biotechnol., 15(4):364-373 (2004); and U.S. Pat.No. 6,740,734.

In a further aspect, the invention provides a γ2-binding trinectin, monobody, or other binding protein based on a fibronectin scaffold.

In one aspect, the invention provides trinectins that specifically bind to one or more of the γ2 ADRs described herein. In another aspect, inventive methods described herein (e.g., modulation of γ2-associated physiological activities, reduction of γ2-associated cancer progression, etc.) are performed with (possibly among other things) delivery of an effective amount of such a trinectin to a subject (e.g., a human host). In a further aspect, the invention relates to the use of an effective amount of such a trinectin in the preparation of a medicament for the treatment of cancer or a pre-cancer condition.

Trinectins comprise a protein binding scaffold that is based on a domain of fibronectin (the 10th fibronectin type III domain). Because these proteins are derived from naturally occurring, circulating human proteins, immune reactions that would otherwise interfere with therapeutic utility are expected to be minimized. In addition, the low molecular weight and compact structure of the molecule (as compared to antibodies) results in a highly stable structure that may enhance target antigen binding. Trinectins are described in, e.g., Xu et al., Chem. Biol. 9:933, 2002 and International Patent Application WO 02/32925. Such molecules are commercially available from Phylos, Inc. (USA) now owned by Compound Therapeutics (Waltham, Mass., USA).

In a further aspect, the invention provides fibronectin type III domain (Fn3) monobodies that specifically bind to one or more of the γ2 ADRs specifically described herein. In a related facet, inventive methods described herein are practiced with (possibly among other things) such a monobody. Another related feature of the invention is provided in the use of such a Fn3 monobody for the production of a medicament to treat γ2-associated ailments, such as cancer or a pre-cancer condition. An example of an Fn3 monobody is embodied in a polypeptide comprising least two Fn3 β-strand domain sequences with a loop region sequence linked between each Fn3 β-strand domain sequence, wherein the loop region comprises γ2-binding sequences. In a particular aspect, the Fn3 monobody loop region varies in one or more ways from the related wild-type Fn3 structure. Examples of such monobodies and related principles, compositions, and methods are described further in, e.g., U.S. Pat. Nos. 6,673,901; 6,703,199; and 6,462,189; Koide et al., (1998), J. Mol. Biol. 284,1141-1151; Batori et al., Protein Eng. 2002 Dec; 15(12):1015-20; Karatan et al., Chem Biol. 2004 Jun; 11(6):835-44; and Koide et al., (2001) Biochemistry 40, 10326-10333.

In another aspect, the invention provides anticalins that bind to one or more of the γ2 ADRs described herein. Various inventive methods described herein (e.g., modulation of γ2-associated physiological activities) also can be practiced with such anticalin molecules. In another facet, the invention relates to the use of such an anticalin for the preparation of a medicament to treat a γ2-associated disease. The invention also relates to the production of such anticalin molecules and to anticalins produced by such processes.

Anticalins, like trinectins, are relatively small proteins (as compared to antibodies) that can be engineered to bind specific targets. Anticalins are based on a lipocalin protein scaffold. Anticalins typically are obtained by modifying a protein of the lipocalin family by amino acid replacement in their natural ligand binding pocket, e.g., using genetic engineering methods. Anticalins typically are small monomeric proteins consisting of only 150 to 190 amino acids. Anticalins can exhibit highly specific binding of small molecules and can penetrate tissues such as solid tumors more efficiently. Anticalins are produced by an entirely in vitro process, making it possible to access targets that are either toxic or non-immunogenic. The pharmacokinetic properties of anticalins can be easily controlled by chemical modifications. Compared to monoclonal antibody therapeutics on the market, anticalins might offer better delivery options, such as enhanced topical, pulmonary, or nasal delivery. Anticalins have two potential fusion termini that can be modified without impacting the binding site, thus multispecific anticalins and/or other conjugates or fusion proteins such as, for example, immunotoxins can readily be generated.

The central element of the anticalin protein architecture is a beta-barrel structure of eight antiparallel strands, which supports four loops at its open end. These loops form the natural binding site of the lipocalins and can be reshaped in vitro by amino acid replacements and other modifications, thus creating novel binding specificities. Using bacterial phagemid display and colony screening techniques, anticalins can be selected from libraries of randomly generated molecules (typically molecules with affinities in the KD values in the low nanomolar range). Anticalins possess high affinity (e.g., low nanomolar or even in the range of about 100 picomolar) and specificity for their prescribed ligands as well as fast binding kinetics, so that their functional properties are similar to those of antibodies. However, anticalins comprise a simple set of four hypervariable loops that can be easily manipulated at the genetic level. Anticalins, related principles, methods, and the like are described further in, e.g., Skerra, A. (2000) Biochim. Biophys. Acta 1482, 337-350; Beste et al. (1999) Proc. Natl. Acad. Sci. USA 96, 1898-1903; Schlehuber et al. (2000) J. Mol. Biol. 297, 1105-1120; Schlehuber et al. (2001) Biol. Chem. 382, 1335-1342; Skerra, A. (2001) Rev. Mol. Biotechnol. 74, 257-275; Skerra, J Biotechnol. June 2001;74(4):257-75; Weiss et al., Chem Biol. August 2000;7(8):R177-84; WO 99016873; and EP1017814.

Additional suitable antibody mimetics generally can be used as surrogates for the antibodies and antibody fragments described herein. Such antibody mimetics may be associated with advantageous properties (e.g., they may be water soluble, resistant to proteolysis, and be nonimmunogenic). For example, peptides comprising a synthetic beta-loop structure that mimics the second complementarity-determining region (CDR) of MAbs have been proposed and generated. See, e.g., Saragovi et al., Science. Aug. 16, 1991;253(5021):792-5. Peptide Ab mimetics also have been generated by use of peptide mapping to determine ‘active’ antigen recognition residues, molecular modeling, and a molecular dynamics trajectory analysis, so as to design a peptide mimic containing antigen contact residues from multiple CDRs. See, e.g., Cassett et al., Biochem Biophys Res Commun. Jul. 18, 2003;307(1):198-205. Additional discussion of related principles, methods, etc., that may be applicable in the context of this invention are provided in, e.g., Fassina, Immunomethods. October 1994;5(2):121-9.

In general, any protein that suitably binds and remains associated with γ2 or a γ2-associated peptide so as to modulate γ2 activity and/or block access to γ2 by other molecules may be used in the methods of the invention. Amino acid sequences from such molecules also can be used in the generation of novel L5G2BPs in accordance with other various peptide production methods provided here (for example, γ2-binding domains of such peptides can be used in the formation of L5G2BP fusion proteins). Examples of known γ2-binding/interacting proteins specifically include α6β1 integrin, α3β1 integrin, α2β1 integrin, α6β1 integrin, laminin-6, laminin-7, EGF-R, type VII collagen, fibulin-1, fibulin-2, Rho GTPases, BP180, syndecan-4, nidogen-1, phosphorylated hsp-27, p300, a cytokeratin, and other matrix metalloproteinases (e.g., MMP-1, MMP-2, MMP-9, and MMP-14 (which also known as Membrane-type matrix metalloproteinase 1 (MT1)), tissue inhibitor of matrix metalloproteinase-1 (TIMP-1) and TIMP-2, E-cadherin, bone morphogenic protein-1 (BMP-1), and the 67 kDa laminin receptor.

L5G2BP fusion proteins represent another exemplary feature of the invention. In a particular exemplary aspect, the invention relates to the use of such a fusion protein in the preparation of medicaments for the treatment of conditions associated with γ2. In another aspect, such a fusion protein can be used in various inventive methods described herein.

L5G2BP fusion proteins typically comprise (a) any suitable sequence or combination of sequences specific and/or selective for γ2 (e.g., an anti-γ2 DIII antibody VH domain, VL domain, or particular CDRs thereof, or a non-antibody γ2-binding peptide sequence) and (b) at least one nonhomologous and typically substantially dissimilar amino acid sequence that imparts a detectable biological function and/or physiochemical characteristic to the fusion protein that cannot solely be attributed to the γ2-specific/selective sequence(s) (e.g., binding of a non-γ2-associated target, increased in vivo half-life, fluorescence, increased targeting to a particular type of cell, etc.). Such at least one substantially dissimilar sequence can be referred to as a “secondary sequence” or “fusion partner.” A substantially dissimilar sequence typically has less than about 40% amino acid sequence identity to the γ2-binding sequence(s), such as less than about 35%, less than about 30%, less than about 25%, or less than about 20% identity to the γ2 DIII-specific/selective sequence(s).

The functional sequences of a fusion protein can be separated by one or more linkers, which typically can be characterized as flexible linker(s).

Secondary sequence(s) can be derived from cytotoxic or apoptotic peptides (examples of peptides from which such sequences can be derived are described elsewhere herein). In this sense, similar to in the provision of various cytotoxic derivative molecules by the invention (discussed above), the invention provides a means for targeting a cytotoxic, apoptotic, or otherwise toxic payload to γ2-associated tissues and cells.

Secondary sequences also can confer diagnostic properties, such as fluorescence or enzymatic detection. Examples of such sequences include those derived from easily visualized enzymes, such as horseradish peroxidase. Various enzyme derivatizing agents described elsewhere herein also may be suitable fusion partners in L5G2BP fusion proteins.

L5G2BP fusion proteins also or alternatively can be characterized by comprising an epitope tag. An epitope tag is an amino acid sequence having enough residues to provide an epitope against which an antibody can be made, in the context of the L5G2BP, yet is short enough such that it does not substantially interfere with the activity (selectivity, specificity, affinity, and/or biological activity) of the L5G2BP (as compared to a “parent” L5G2BP lacking the epitope tag). An epitope tag desirably is sufficiently unique so that the anti-epitope tag antibody does not substantially cross-react with other epitopes. Suitable tag polypeptides generally have at least about 6 amino acid residues and usually between about 8-50 amino acid residues (e.g., about 9-30 residues). Examples of epitope tags include the flu HA tag polypeptide and its antibody 12CA5 (Field et al. (1988), Mol. Cell. Biol. 8:2159-2165); the c-myc tag and the 8F9, 3C7, 6E10, G4, B7 and 9E10 antibodies thereto (Evan et al. (1985), Mol. Cell. Biol. 5(12):3610- 3616); and the Herpes Simplex virus glycoprotein D (gD) tag and its antibody (Paborsky et al. (1990), Protein Engineering 3(6):547-553 (1990)). In certain embodiments, the epitope tag is a “salvage receptor binding epitope”. As used herein, the term “salvage receptor binding epitope” refers to an epitope of the Fc region of an IgG molecule (e.g., IgG₁, IgG₂, IgG₃, or lgG₄) that is responsible for increasing the in vivo serum half-life of the IgG molecule.

In another aspect, a L5G2BP fusion protein is provided wherein the fusion protein comprises a “fusion partner” sequence that corresponds to or consists essentially of a cytokine or biologically active fragment thereof (or a variant or derivative of either thereof), such as a cytokine or cytokine fragment that induces, promotes, and/or enhances immune cell activity (particularly anti-cancer immune cell activity) in vivo. Examples of such cytokines include interleukin 2 (IL-2), granulocyte macrophage colony-stimulating factor, interferon gammas (IFNγs), macrophage colony-stimulating factor, interleukin 12, and the like (additional immunomodulatory cytokines described elsewhere herein in the context of other aspects of the invention also or alternatively can be incorporated in such fusion proteins). A large number of cytokine variants and cytokine derivatives have been described in the art. Conjugate derivative L5G2BPs comprising linked cytokine or active cytokine fragment peptides are another feature of the invention.

In another exemplary aspect, the invention provides a L5G2BP that comprises a suitable leucine zipper sequence fusion partner that can increase the affinity and/or production efficiency of the L5G2BP fusion protein as compared to a protein consisting or consisting essentially of the L5G2BP sequence(s). Potentially suitable leucine zipper sequences include the jun and fos leucine zippers taught by Kostelney et al. (1992), J. Immunol., 148: 1547-1553, and the GCN4 leucine zipper. Cytokine fusion proteins are further described in, e.g., Helguera et al., Clin Immunol. December 2002;105(3):233-46 and Penichet et al., J Immunol Methods. Feb. 1, 2001;248(1-2):91-101.

To inhibit cancer and reduce the number of preneoplastic cells by killing such cells, a toxin such as ricin, diphtheria toxin, anthrax toxins (see, e.g., Frankel et al., Curr Protein Pept Sci. August 2002;3(4):399-407), eosinophil-derived neurotoxin, a tumor necrosis factor (e.g., TNFα), a mistletoe toxin (e.g., mistletoe lectin I A chain), abrin, saporin, Pseudomonas exotoxin, and the like, or a cytotoxic fragment thereof, or a combination of any thereof, can be incorporated in a L5G2BP fusion protein as a fusion partner (see generally, e.g., Frankel et al., Clinical Cancer Research Vol. 6, 326-334 (2000); Frankel, Clinical Cancer Research Vol. 8, 942-944 (2002); Brinkman et al., Expert Opin Biol Ther. July 2001;1(4):693-702; and Fitzgerald et al., Diagn. Ther. 7:447-62 (1992)). Apoptotic agents also can be suitable fusion partners, such as TNF-related apoptosis-inducing ligand (TRAIL/APO-2L), PML, apoptin, and the like (see, e.g., Wajant, Apoptosis. October 2002;7(5):449-59; see also generally, e.g., Thorbun et al., Apoptosis. January 2004;9(1):19-25). In another aspect, a cytotoxic RNase peptide fusion partner can be used (e.g., a fusion partner based on onconase or ribonuclease A). Fusion partner sequences also can be derived from cytotoxic or apoptotic peptides (e.g., can be cytotoxic fragments of such sequences or cytotoxic variants of such sequences, etc.), examples of peptides from which such sequences can be derived are described elsewhere herein (e.g., with respect to antibody conjugates). In general, any peptide conjugate described elsewhere herein can be used as a fusion partner in an antibody fusion protein or other L5G2BP fusion protein (and visa versa).

From the foregoing it should be clear there are a large number of fusion partner sequences that can be advantageously included in an antibody fusion protein. Fusion partner sequence can confer, for example, diagnostic properties, such as through facilitating fluorescence or enzymatic detection. Examples of such sequences include those derived from easily visualized enzymes, such as horseradish peroxidase, and fluorescent sequences, such as Green Fluorescent Protein (GFP) sequences. L5G2BP flourobodies are, for example, another aspect of the invention. Fluorobodies are molecules made by grafting a functional set of CDRs (and typically associated residues/sequences as framework therefore) onto a GFP sequence or other partner that emits a strong fluorescent signal.

In another exemplary aspect, the invention provides L5G2BP fusion proteins or conjugates (antibody and non-antibody) comprising a cyclic or multi-cyclic (e.g., double cyclic) partner, such as a cyclic tumor homing peptide, e.g., the cyclic CNGRC peptide or double-cyclic ACDCRGDCFC peptide (see, e.g., Ellersby et al., Nature Med., 5(9):1032-1038 (1999)). Similar cyclic homing peptides are known (see, e.g., Laakkonen, Nat Med. July 2002;8(7):751-5). L5G2BP fusion proteins (both antibody and non-antibody) can also comprise other homing/targeting domains, such as integrin-binding RGD domains and the like.

In a further aspect, the invention provides L5G2BP adzymes. Adzymes, are proteins containing a binding domain and a separate enzyme active site which together act on a therapeutic target. The protein binding domain attaches to the disease-causing target, allowing the enzyme domain to abolish the biological function of the target.

Fusion proteins can include any suitable number of any suitable types of linkers (between domains), such as a predominantly Gly and/or Ser flexible linker of about 5-20 amino acid residues, and may also or alternatively comprise a cleavable linker or cleavage site for proteinases, such as an enterokinase. Recombinant methods related to the insertion of linker sequences are well know (see, e.g., Sambrook et al., loc. cit., Ausubel, Current Protocols in Molecular Biology, Green Publishing Associates and Wiley lnterscience, N.Y. (1989)). Additional suitable linkers can comprise oligomerization domains.

Oligomerization/multimerization domains can facilitate the combination of two or several amino acid sequences. Non-limiting examples of oligomerization domains comprise leucine zippers (like jun-fos, GCN4, E/EBP; Kostelny, J. Immunol. 148 (1992), 1547-1553; Zeng, Proc. Natl. Acad. Sci. USA 94 (1997), 3673-3678, Williams, Genes Dev. 5 (1991), 1553-1563; Suter, PHAGE DISPLAY OF PEPTIDES AND PROTEINS, Chapter 11, (1996), Academic Press) (entire text incorporated), antibody-derived oligomerization domains, like constant domains CH1 and CL (Mueller, FEBS Letters 422 (1998), 259-264) and/or tetramerization domains like GCN4-LI (Zerangue, Proc. Natl. Acad. Sci. USA 97 (2000), 3591-3595). Such multimerization domains also can be used in the construction of anti-γ2 antibodies.

Superantibodies, which are a specialized type of antibody fusion protein/conjugate, and similar specialized antibody fusion proteins and antibody conjugates, are described separately elsewhere herein.

Fusion proteins can be produced by any suitable method (e.g., direct chemical synthesis on solid phase or in solution). For example, a fusion anti-γ2 DIII antibody with a protein can be produced by linking a cDNA encoding an antibody or antibody fragment to other cDNA encoding the protein, constructing DNA encoding the fusion antibody, and inserting the DNA into an expression vector for prokaryotic or eukaryotic cell expression which is expressed to produce the fusion protein. Conjugated derivative L5G2BPs wherein such toxins are linked to the amino acid sequence of the peptide also are features of the invention. Methods of generating fusion proteins, which can be applied to the production of antibody fusion proteins and other fusion proteins provided by the invention are well known in the art. Such methods and related principles are described in, e.g., U.S. Pat. Nos. 5,457,035, 5,563,046; 5,668,225; 5,698,679; 5,763,733; 5,908,626; 5,969,109; 6,008,319; 6,117,656; 6,121,424; 6,132,992; 6,207,804; 6,224,870; and Borrebaeck et al., ANTIBODY ENGINEERING (2^{nd} Ed., Oxford University Press 1995); W093/10151; MOLECULAR CLONING (Cold Spring Harbor Press) (cited elsewhere herein); Ashkenazi et al. (1991) PNAS 88, 10535; Byrn et al. (1990) Nature 344, 677; and Hollenbaugh et al. (1992) “Construction of Immunoglobulin Fusion Proteins,” in Current Protocols in Immunology, Suppl. 4, pp. 10.19.1 to 10.19.11.

As indicated above, the invention also provides non-antibody multispecific L5G2BPs that are fusion proteins comprising at least one sequence specific for γ2 binding and at least one sequence specific for at least one secondary target, and optionally one or more additional dissimilar sequences that impart additional biological and/or physiochemical properties to the fusion protein. As with other L5G2BPs, such a fusion protein can recognize and bind its two different targets at the same time or different times. Preferably, the fusion protein, as with other multispecific L5G2BPs, binds to both of its targets at the same time under appropriate conditions. An example of such a L5G2BP is a fusion protein comprising (a) a single chain γ2-binding HLA/MHC Class I portion (b) a secondary target-binding portion, such as a truncated cancer-associated receptor portion (e.g., a sequence that acts as a functional VEGF receptor) and, optionally, (c) a sequence that facilitates purification of the fusion protein (e.g., an epitope tag or hexa-histidine sequence), a sequence that enhances the stability of the fusion protein, or a sequence that facilitates detection of the fusion protein (e.g., a GFP sequence).

L5G2BPs can be produced by any suitable method and are generally not restricted in how they are produced (exception may be made for molecules typically produced according to particularly techniques, such as trinectins which are based upon a particular type of scaffold). Thus, for example, unless otherwise stated or clearly contradicted by context, antibody and antibody-like molecules provided by the invention can be prepared using any suitable technique or combinations thereof. Various antibody production and purification techniques are known in the art and include those described in, e.g., Harlow and Lane: ANTIBODIES; A LABORATORY MANUAL, infra; Harlow and Lane: USING ANTIBODIES: A LABORATORY MANUAL (Cold Spring Harbor Laboratory Press (1999)); U.S. Pat. No. 4,376,110; and Ausubel et al, eds., CURRENT PROTOCOLS IN MOLECULAR BIOLOGY, Greene Publishing Assoc. and Wiley Interscience, N.Y., (1987, 1992).

Monoclonal antibodies (mAbs), for example, can be obtained from any suitable source. Thus, for example, monoclonal antibodies can be obtained from hybridomas prepared from murine splenic B cells obtained from mice immunized with a γ2 DIII-containing peptide or γ2 DIII peptide-encoding nucleic acid. Monoclonal antibodies also can be obtained from hybridomas derived from antibody-expressing cells of other immunized non-human mammals such as rats, dogs, primates, etc. More particularly, monoclonal antibodies can be produced by the hybridoma method first described by Kohler et al., Nature, 256:495 (1975), or by other well-known, subsequently-developed methods (see, e.g., Goding, Monoclonal Antibodies: Principles and Practice, pp.59-103 (Academic Press, 1986)). Hybridomas useful in the production of any of the anti-γ2 DIII antibodies of the invention are another independent feature of the invention. Such hybridomas may be formed by chemical fusion, electrical fusion, or any other suitable technique, with any suitable type of myeloma, heteromyeloma, phoblastoid cell, plasmacytoma, or other equivalent thereof and any suitable type of antibody-expressing cell.

Transformed immortalized B cells also can be used to produce antibodies of the invention and also are an independent feature of the invention. Such cells can be produced by standard techniques, such as transformation with an Epstein Barr Virus, or a transforming gene. (See, e.g., “Continuously Proliferating Human Cell Lines Synthesizing Antibody of Predetermined Specificity,” Zurawaki, V. R. et al, in MONOCLONAL ANTIBODIES, ed. by Kennett R. H. et al, Plenum Press, N.Y. 1980, pp 19-33-text incorporated entirely). Thus, stable and continuous and/or immortalized anti-γ2 DIII expressing cells and cell lines are another feature of the invention. Eukaryotic and prokaryotic cells (e.g., yeast cells, continuous and/or immortalized mammalian cell lines (e.g., lymphoid antibody-producing cell derived cell lines), plant cells, insect cells, and bacterial cells such as E. coli cells, etc.) comprising L5G2D3BP-encoding or L5G2D3BP-fragment-encoding nucleic acids are features of the invention.

Transgenic cells and organisms, such as non-human primates, rodents (e.g., hamsters, guinea pigs, and rats—including modified strains thereof such as severe combined immunodeficient (SCID) mice and other immunocompromised animal strains), dogs, etc., expressing human anti-γ2 DIII antibodies of the invention also are provided by the invention. Antibodies of the invention can, for example, be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

Further, human antibodies or antibodies from other species can be generated through display-type technologies, including, without limitation, phage display, retroviral display, ribosomal display, and other related techniques, using methods well known in the art, and the resulting molecules can be subjected to additional maturation methods, such as affinity maturation, as such techniques also are well known (see, e.g., (Hoogenboom et al ., J. Mol. Biol. 227: 381 (1991) (phage display); Vaughan, et al., Nature Biotech 14:309 (1996) (phage display); Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743). If display technologies are utilized to produce antibodies that are not human, such antibodies can be humanized, e.g., as described elsewhere herein.

Anti-γ2 antibodies and antibody fragments also can be recovered from recombinant combinatorial antibody libraries, such as a scFv phage display library, which can be made with human VL and VH cDNAs prepared from mRNA derived from human lymphocytes. Methods for preparing and screening such libraries are known in the art. There are a number of commercially available kits for generating phage display libraries. There are also other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, and WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982). Suitable VL and VH nucleic acid sequences can be selected using any appropriate method. For example, VL and VH nucleic acids can be selected by employing the epitope imprinting methods described in International Patent Application WO 93/06213. Antibody libraries, such as scFv libraries can be prepared and screened using known and suitable methods (with human γ2 Dlil-containing peptides as antigen(s)), such as those described in, e.g., International Patent Application WO 92/01047, McCafferty et al., Nature (1990) 348:552-554; and Griffiths et al., (1993) EMBO J 12:725-734). Such antibody libraries and other combinations of L5G2BPs (libraries, pools, etc.) are features of the invention that can be used therapeutically to provide a more comprehensive immune response; as tools in screening methods for immunogenic peptides, small molecules, other anti-γ2 antibodies (e.g., by way of competition assays), and the like; and/or in diagnostic methods and compositions (e.g., an immunoassay chip comprising a panel of such antibodies optionally in association with other antibodies can be prepared by standard techniques). In the context of such methods, once initial human VL and VH segments are selected, “mix and match” experiments, in which different pairs of the initially selected VL and VH segments are screened for γ2 or γ2-associated peptide/structure binding, can be performed to select desirable VL/VH pair combinations. For example, reactivity of the peptides can be determined by ELISA or other suitable epitope analysis methods (see, e.g., Scott, J. K. and Smith, G. P. Science 249:386-390 (1990); Cwirla et al. PNAS USA 87:6378-6382 (1990); Felici et al. J. Mol. Biol. 222:301-310 (1991); and Kuwabara et al. Nature Biotechnology 15:74-78 (1997) for discussion of such techniques and principles). Antibodies can thereafter be selected by their affinity for antigen and/or by their kinetics of dissociation (off-rate) from antigen (see, e.g., Hawkins et al. J. Mol. Biol. 226:889-896 (1992)).

Recombinant cells comprising exogenous nucleic acids encoding L5G2BPs can be prepared by any suitable technique (e.g., transfection/transformation with a naked DNA plasmid vector, viral vector, invasive bacterial cell vector or other whole cell vector, etc., comprising an anti-γ2 DIII antibody-encoding sequence delivered into the cell by calcium phosphate-precipitation facilitated transfection, receptor-mediated targeting and transfection, biolistic delivery, electroporation, dextran-mediated transfection, liposome-mediated transformation, protoplast fusion, direct microinjection, etc.). Methods of transforming/transfecting cells are well known in the art (see, e.g., Sambrook et al. MOLECULAR CLONING: A LABORATORY MANUAL, Cold Spring Harbor Lab. Press (2d Ed., 1989 and 3rd Ed., 2001) and Ausubel, supra. Such cells are another feature of the invention.

Cell lines available as hosts for recombinant protein expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Other cell lines that may be used are insect cell lines, such as Sf9 cells. When nucleic acids (or nucleic acid-containing vectors) encoding antibody genes are introduced into mammalian host cells, antibodies can be produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Antibodies and other L5G2BPs also may be recovered from host cell lysates when directly expressed without a secretory signal.

The purification of antibodies and other L5G2BP forms from cell cultures, cell lysates, and animals (e.g., from the ascites fluid of a transgenic animal producing anti-γ2 DIII antibodies) can be achieved by application of any number of suitable techniques known in the art including, e.g., immunoaffinity column purification; sulfate precipitation; chromatofocusing; preparative SDS-PAGE; and the like.

Anti-γ2 antibodies, antibody fragments, and other L5G2BPs also can generally be produced in bacterial cells and eukaryotic unicellular microorganisms, such as yeast. Bacterial cell-produced peptides, such as antibodies, lack normal glycosylation and accordingly may be deficient in terms of biological functions, such as ADCC functions and other aspects of the immune response associated with anti-γ2 antibodies produced in mammalian cells and/or animals (e.g., the recruitment of NK cells). Yeast cell produced antibodies, for example, normally exhibit different types of glycosylation patterns than antibodies produced in mammalian cells. However, methods for producing antibodies with effective glycosylation in yeast are currently being developed by companies such as Glycofi, Inc. (Lebanon, NH, USA) (see, e.g., Hamilton et al., Science. 2003 Aug 29;301(5637):1244-6; Choi et al., Proc Natl Acad Sci U S A. Apr. 29, 2003;100(9):5022-7; and Gerngross et al., “Production of Complex Human Glycoproteins in Yeast” presented at “Antibody Engineering and Optimization,” available through Cambridge Health Institute Proceedings (presented on Apr. 28, 2004)). Glycosylation of proteins also can be modified using techniques such as are described in U.S. Patent Applications 20030124645, 20030180835, 20040063911, and U.S. Pat. No. 6,379,933. Antibodies and antibody fragments produced by any of these methods are additional features of this invention.

Anti-γ2 antibodies of any type, as well as other L5G2BPs, generally can be prepared by recombinant expression in any suitable type of cells or organisms, unless otherwise stated or clearly contradicted by context. Antibodies provided by the invention, for example, include human antibodies that are prepared, expressed, created, and/or isolated by recombinant means, such as antibodies expressed using a recombinant expression vector transfected into a host cell; antibodies isolated from a recombinant, combinatorial human antibody library; antibodies isolated from a transgenic animal; and antibodies prepared, expressed, created or isolated by any other means that involves splicing of human immunoglobulin-encoding nucleic acid sequences to other nucleic acid sequences exogenous to the human immunoglobulin-encoding nucleic acids and human immunoglobulin-encoding genes. Recombinant human antibodies typically have variable and constant regions derived from human germlne immunoglobulin sequences. In certain embodiments, however, such recombinant human antibodies are subjected to in vitro mutagenesis (or, when an animal transgenic for human immunoglobulin (Ig) sequences is used, in vivo somatic mutagenesis) and, thus, the amino acid sequences of the VH and VL regions of the recombinant antibodies can be sequences that, while derived from and related to human germlne VH and VL sequences, may not naturally exist within the human antibody germine repertoire in vivo. Both types of human antibodies are provided as separate aspects of the invention. Thus, in one aspect, the invention provides a recombinant human antibody that comprises VH, VL, and/or other antibody regions that are identical to a wild-type (naturally occurring) human antibody.

Exemplary methods for specifically producing Ln-5 γ2 mAbs are provided in, e.g., US patent application Ser. No. 10/695,559.

In addition to gross structural and physiochemical characteristics associated with particular types of L5G2BP molecules, L5G2BPs provided by and/or used in the context of the invention typically can be characterized on the basis of exhibiting one or more biological functions.

A γ2 binding peptide, such as an anti-γ2 antibody, in the context of the inventive methods and compositions described herein, specifically binds to a portion of Ln-5 γ2 and typically will bind to a particular portion of γ2 DIII.

In the context of this invention, the binding of a γ2-binding peptide to γ2 desirably at least partially and detectably inhibits (a) the functioning of Ln-5 γ2 DIII, (b) the functioning of other proteins that interact with γ2 DIII (in free form or in connection with a multimeric peptide) or a γ2 DIII fragment (alone or in association with other Ln-5 subunits or non-Ln-5 biomolecules), and/or (c) the activity of cells associated with Ln-5 (or at least a form of Ln-5 and/or fragment(s) thereof). Examples of such inhibiting activities include (a) interfering with Ln-5-associated, γ2-associated, γ2 complex-associated, and/or γ2 fragment-associated cell motility; (b) promoting one or more physiological effects associated with an anti-γ2 DIII humoral immune response (e.g., the opsonization of γ2 DIII peptides such as Ln-5 or γ2/p3 heterodimers, and/or Ln-5-associated structures and/or cells); (c) promoting the aggregation of Ln-5 peptides and/or Ln-5 associated cells (e.g., promotion of Ln-5 and/or γ2 crosslinking); (d) promoting activation of complement; (e) promoting the phagocytosis of γ2 peptides (e.g., γ2 chains or γ2-associated peptides, such as Ln-5 and Ln-5 fragments) or associated structures or cells; (f) promoting recruitment of cytotoxic immune cells, such as monocytes, NK cells, dendritic cells, and/or macrophages to Ln-5 associated-structures and/or cells (or promoting any other aspect of antibody-dependent cell mediated cytotoxicity (ADCC) and/or complement-dependent cytotoxicity (CDC)); and (g) promoting a combination of any or all thereof. Accordingly, antibodies, antibody fragments, and derivatives of either thereof, which typically exhibit one or more of such features, are advantageous γ2-binding peptides in many aspects of this invention.

As indicated above, L5G2BPs typically selectively or specifically bind at least a portion of γ2 (e.g., a portion of γ2 DIII). Terms like “specific” and “specificity” herein refer to the ability of a Ln-5 γ2 binding peptide, such as an anti-γ2 antibody, to bind a particular portion of a target (e.g., an epitope located at least partially within γ2) while only having little or no detectable reactivity with other portions of Ln-5 (including other epitopes that are bound by other anti-Ln-5 antibodies).

Specificity can be relatively determined by competition assays as described herein (see also, e.g., U.S. Pat. No. 5,660,827). Competition also can be assessed by a flow cytometry test. For example, γ2 or a γ2-associated peptide can be incubated first with the reference L5G2BP (e.g., mAb 5D5 or mAb 6C12) and then with the test antibody labeled with a fluorochrome or biotin. The test peptide typically is said to compete with the reference peptide if the binding obtained with preincubation with saturating amount of the reference peptide is about 80% or less, such as about 50% or less, such as about 40% or less than the binding (as measured by mean of fluorescence) obtained by the test antibody without preincubation with the reference antibody. Alternatively, an antibody is said to compete with a reference peptide if the binding obtained with a labeled reference peptide (e.g., a referenced peptide labeled by a fluorochrome or biotin) to γ2 or a γ2-associated peptide with saturating amount of test antibody is about 80% or less, such as about 50% or less, e.g., about 40%, or less than the binding of the reference peptide obtained without preincubation with the test peptide. Further examples of such assays are provided in Saunal and Regenmortel, (1995) J. Immunol. Methods 183: 33-41. Additional methods for determining mAb specificity by competitive inhibition are known in the art and useful examples of such techniques can be found in, e.g., Harlow, et al., ANTIBODIES: A LABORATORY MANUAL, Cold Spring Harbor Laboratory Press, Cold Spring Harbor, N.Y., 1988), Colligan et al., eds., Current Protocols in Immunology, Greene Publishing Assoc. and Wiley lnterscience, N.Y., (1992, 1993), and Muller, Meth. Enzymol. 92:589-601 (1983)). Specificity also can be determined, predicted, and/or confirmed by any of the epitope identification/characterization techniques described herein or their equivalents known in the art.

L5G2BPs typically are selective or specific for γ2 DIII in the context of epithelial cell or epithelial-derived cell populations and their environment and typically in the context of the basal lamina (basement membrane), in the context of a Ln-5 molecule, in the context of another γ2-associated molecule (such as a γ2/β3 heterodimer), and/or in the context of the γ2 chain or fragment thereof.

In one facet, the invention provides antibodies and other peptides that bind to γ2 domain III that may also bind to other portions of γ2. For example, the invention provides in one aspect an antibody that may be suitably used in methods provided here or included in compositions provided here can bind to a linear epitope that comprises a part of γ2 and a part of an adjacent sequence from Ln-5 or a bispecific antibody provided by the invention can bind γ2 and a sequence located in Ln-5 domain I and/or domain 11, for example, such that the bispecific antibody interferes with interchain coiled coil structure formation. In another exemplary aspect, the invention provides antibodies that are specific for regions only found within γ2 domain 111. In another aspect, the invention provides antibodies and other peptides selective or specific for γ2 DIII that do not detectably bind to the α3 chain of Ln-5. In yet another aspect, an antibody or other peptide that is selective or specific for γ2 DIII also or alternatively can be characterized as not detectably binding to the β3 chain of Ln-5.

L5G2BPs can be sufficiently specific and selective for γ2 DIII and γ2 DIII-associated peptides, such as free forms of γ2 and/or heterotrimeric Ln-5, which are associated with one or more of the disorders, diseases, or conditions described herein and/or cells involved in such disorders, diseases, and conditions (e.g., preneoplastic or neoplastic epithelial-derived cells, such as invasive carcinoma cells) that such L5G2BPs do not detectably modulate the activities of non-disorder, non-disease-associated cells in a human (e.g., the invention provides L5G2BPs that do not impede the migration of non-cancer associated epithelial cells). Thus, for example, the invention provides L5G2BPs that are specific for γ2 monomers and/or γ2/β3 heterodimers or at least selective for such peptides with respect to heterotrimeric Ln-5, such that the L5G2BP is directed to “target” cells secreting such monomers (e.g., cancer cells that co-express γ2 and ,3 chain peptides—see, e.g., Akimoto et al., Pathol Int. September 2004;54(9):688-92) and/or heterodimers, or associated tissues/spaces, as often are found in, e.g., invasive epithelial-derived cancer cells, but not healthy epithelial basement membrane-associated cells or associated tissues/spaces. The invention also provides L5G2BPs that also or alternatively are specific and/or selective for γ2 DIII-containing “processed” γ2 and γ2-associated peptides, as are associated with many invasive cancer cells but typically not with healthy epithelial basement membrane-associated cells. Such “processing” of γ2 is discussed in, e.g., Marinkovich, et al., JBC 267, 17900-17906; Amano et al., JBC 275, 22728-22735; and Pirila et al., Biochem Biophys Res Commun. Apr. 18, 2003;303(4):1012-7).

L5G2BPs can exhibit any suitable level of affinity and/or avidity for one or more epitopes or antigenic determinants contained at least partially in γ2 DIII. Affinity refers to the strength of binding of the L5G2BP to an epitope or antigenic determinant. Typically, affinity is measured in terms of a dissociation constant Kd, defined as [Ab]×[Ag]/[Ab-Ag] where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Suitable methods for determining binding peptide specificity and affinity by competitive inhibition, equilibrium dialysis, and the like can be found in, e.g., Harlow, et al., ANTIBODIES: A LABORATORY MANUAL, Cold Spring Harbor Laboratory Press, Cold Spring Harbor, N.Y., 1988); Colligan et al., eds., CURRENT PROTOCOLS IN IMMUNOLOGY, Greene Publishing Assoc. and Wiley Interscience, N.Y., (1992, 1993), and Muller, Meth. Enzymol. 92:589-601 (1983).

Typically a L5G2BP, such as a L5G2D3BP, and particularly an anti-y DIII antibody, provided by the invention, has an affinity for at least one epitope at least partially comprised in γ2 DIII in the range of about 10⁴ to about 10¹⁰ M-1 (e.g., about 10⁷ to about 10⁹ M⁻¹). The term immunoreact herein typically refers to binding of a L5G2BP to a γ2 peptide with a dissociation constant (Kd) that is about 10⁻⁴ M or less.

Desirably, a L5G2BP will have an affinity that is at least as great for γ2, a particular portion thereof (e.g., a particular portion of γ2 DIII), and/or a particular γ2-associated peptide as mAb GB3, mAB D4B5, and/or mAb B4-6, and in some aspects will also desirably have an affinity for such a peptide or peptides that is at least about as great as the affinity of mAb 4G1, mAb 5D5, and/or mAb 6C12. Affinity can be determined by any of the methods described elsewhere herein or their known equivalents in the art. An example of one method that can be used to determine affinity is provided in Scatchard analysis of Munson & Pollard, Anal. Biochem. 107:220 (1980). Binding affinity also may be determined by equilibrium methods (e.g. enzyme-linked immunoabsorbent assay (ELISA) or radioimmunoassay (RIA)) or kinetics analysis (e.g., BIACORE™ analysis).

Typically, the disassociation constant for anti-γ2 DIII antibodies of the invention is less than about 100 nM, less than about 50 nM, less than about 10 nM, about 5 nM or less, about 1 nM or less, about 0.5 nM or less, about 0.1 nM or less, about 0.01 nM or less, or even about 0.001 nM or less.

L5G2DBPs provided by this invention, and particularly anti-γ2 DIII antibodies provided by the invention, also or alternatively desirably exhibit at least as great, if not greater, avidity for γ2 DIII, or a portion of γ2 Dil, or for a peptide comprising at least a portion of γ2 DIII than mAb GB3, mAb D4B5, and/or mAb B4-6. A L5G2D3BP can desirably exhibit significantly greater avidity for such γ2 DIII-associated peptides than one or more of these mAbs (e.g., at least about 10% more, at least about 20% more, at least about 30% more, at least about 50% more, such as at least about 55-99% more avidity than one or more of these antibodies, or more than 2× of the avidity exhibited by one or more of these antibodies (e.g., at least about 150% more avidity, at least about 200% more avidity, etc.) for the target γ2 DIII peptide(s)). In another aspect, the invention provides L5G2D3BPs that also or alternatively exhibit greater avidity for γ2 Dil-associated peptides than mAb 4G1, mAb 5D5, and/or mAb 6C12. L5G2D3BPs having low affinity for one or more antigenic determinants can still possess a sufficiently high avidity for a γ2 DIII-associated peptide so as to be useful components of the methods and compositions of this invention. Likewise, a L5G2D3BP having low avidity for a γ2 DIII-associated peptide can have a sufficiently high affinity for a particular antigenic determinant to allow the binding peptide to be useful in the methods and compositions of this invention.

Avidity refers to the overall strength of the total interactions between a binding protein and antigen (e.g., the total strength of interactions between an anti-γ2 DIII antibody polymer and a γ2 DIII-associated peptide). Affinity is the strength of the total noncovalent interactions between a single antigen-binding site on an antibody or other binding peptide and a single epitope or antigenic determinant. Avidity typically is governed by three major factors: the intrinsic affinity of the binding protein for the epitope(s) or antigenic determinant(s) to which it binds, the valence of the antibody or binding protein and antigen (e.g., a multivalent antibody polymer will typically exhibit higher levels of avidity for an antigen than a bivalent antibody and a bivalent antibody can will have a higher avidity for an antigen than a univalent antibody, especially where there are repeated epitopes in the antigen), and/or the geometric arrangement of the interacting components

Anti-γ2 DIII variant antibodies and other L5G2D3BPs provided by the invention may desirably exhibit similar functional characteristics as parent γ2 DIII antibodies, such as may be determined by antibody-dependent cellular cytotoxicity (ADCC) and complement-mediated cytotoxicity (CDC) assays (see, e.g., U.S. Pat. No. 5,500,362).

L5G2D3BPs of the invention, and particularly anti-γ2 DIII antibodies, can be selected based on their ability to provide or not provide complement fixation and/or complement dependent cytotoxicity (CDC). There are a number of isotypes of antibodies that are capable of complement fixation and CDC, including, without limitation, the following: murine IgM, murine lgG2a, murine IgG2b, murine IgG3, human IgM, human IgG1, and human IgG3. Those isotypes that are not capable of complement fixation/CDC include, without limitation, human lgG2 and human IgG4. Isotype determination and other methods for modifying the complement fixation and CDC functional characteristics of antibodies are known in the art.

In one aspect, the invention provides L5G2D3BPs that may be characterized in possessing the ability to compete (competitively inhibit antigenic determinant region binding) or cross-compete (i.e., relatively partially inhibit antigenic determinant region binding) with one or more anti-γ2 antibodies (such as, for example, γ2 DIII mAb 4G1 (available from DAKO CYTOMATION—Stockholm, Sweden) or γ2 DIII mAbs 5D5 and/or 6C12 (deposited with DSMZ (Deutsche Sammlung von Mikroorganismen und Zellkulturen GmbH—Hanover, Germany) under deposit numbers DSM ACC2652 and DSM ACC2653, respectively).

In one such aspect of the invention, L5G2D3BPs that compete with mAb 5D5, mAb 6C12, or cross-competes with both 5D5 and 6C12, with respect to binding γ2 Dil, are provided. It is expected that antibodies (or other Ln-5 γ2 binding peptides) that compete with mAb 5D5 and/or mAb 6C12 in binding to γ2 (whether in a free γ2 chain context, a fragment thereof, in association with γ2-comprising heterodimers, in association with a heterotrimeric Ln-5 protein, or any combination thereof) will likely be useful in various therapeutic and diagnostic methods provided by the invention and particularly in those contexts where 5D5 and/or 6C1 2 exhibit significant physiological responses (e.g., in the reduction of epithelial-derived cancer cell invasiveness).

In another aspect, the invention provides a L5G2D3BP that is, corresponds to, or comprises a portion of a L5G2D3BP that competes with mAb 4G1, mAb 5D5, and/or mAb 6C12 for binding to one or more portions of γ2 DIII. Such a L5G2D3BP can be, for example, a Fab fragment, derived from an antibody that binds to an epitope that at least partially overlaps with an epitope bound by mAb 4G1, 5D5, and/or 6C12 or is located near enough to such an epitope so that such an antibody competes with mAb 4G1, 5D5, and/or 6C12 for binding to γ2 DIII or portions thereof due to steric hindrance. Such a Fab fragment, due to its relatively small size compared to the mAb molecules, may not significantly compete for binding to γ2 DIII although the antibody from which it derived does. Nonetheless, such a L5G2D3BP can be useful in similarly targeting nearby regions of γ2 DIII (e.g., in the context of targeting a cytotoxin, radionuclide, or the like in the context of an immunoconjugate L5G2D3BP). Therefore, such L5G2D3BPs can be useful in the context of the methods of this invention and, accordingly, also are to be considered useful features of this invention.

Competition for binding to Ln-5 γ2 DIII (in the context of heterotrimeric Ln-5, other γ2 associated peptide, or free γ2 or γ2 fragment), or a portion of Ln-5 (e.g., a portion of γ2 Dil, a fragment of Ln-5 comprising a portion of γ2 DIII, or a region of Ln-5 corresponding to a portion of γ2 DIII and an adjacent domain, such as the Ln-5 hinge region) by two or more Ln-5 γ2 binding peptides can be determined by any suitable technique. In one aspect, competition is determined by an ELISA assay as described, for example, in the Examples section of this document. Competition between particular portions of Ln-5, such as particular regions of γ2 Dil, with respect to a particular Ln-5 γ2 binding peptide, can be determined by a similar technique. In one aspect, competition is evaluated in the context of denatured or structurally modified γ2 DIII peptides, e.g., boiled γ2 DIII peptides, which can be particularly indicative of the presence of so-called linear epitopes. In most aspects, the evaluation of competition is made using free (e.g., substantially purified) γ2 DIII peptides in as near natural form as possible (e.g., non-denatured peptides) or for γ2 DIII peptides in association with cells, peptides, or other biomolecules.

Competition in the context of this invention refers to any detectably significant reduction in the propensity for a particular molecule to bind a particular binding partner in the presence of another molecule that binds the binding partner. Typically, competition means an at least about 10% reduction, such as an at least about 15%, or an at least about 20% reduction in binding (e.g., a reduction in binding of about 25% or more, about 30% or more, about 15-35%, etc.) between an Ln-5 γ2 binding peptide and (a) a form of Ln-5 (e.g., uncleaved Ln-5 (“unprocessed”, “not processed” or “immature” Ln-5)); (b) a form of free γ2 (e.g., a γ2 promigratory fragment and/or γ2 fragment produced by other in vivo processing); (c) another peptide associated with γ2, such as a γ2/β3 heterodimeric peptide; (d) a portion of γ2 such as DIII or a region comprising a portion of DIII and an adjacent N-terminal or C-terminal region of Ln-5 γ2; or (d) a portion of Dil, is caused by the presence of another Ln-5 γ2 binding peptide as determined by, e.g., ELISA analysis using sufficient amounts of the two or more competing L5G2D3BPs and a γ2 molecule (e.g., a γ2 DIII fragment or γ2 DIII fragment-comprising peptide) or a γ2-associated molecule. It also can be the case that the competition can exist between L5G2D3BPs with respect to more than one of Ln-5, γ22, Dil, and/or portion(s) of Dil, e.g. in a context where the binding properties of a particular region of Ln-5 are retained in fragments thereof, such as may be the case of a well-presented linear epitope located in various tested fragments or a conformational epitope that is presented in sufficiently large DIII fragments as well as in Dil, γ2, and Ln-5). In other aspects, L5G2D3BPs may compete only a certain levels of Ln-5 structure (e.g., competition may exist between two L5G2D3BPs for a region of γ2 DIII at the Ln-5 level, but not at the DIII level, due to conformational factors or due to contribution to the antigenic determinant region by a portion of the α3 and/or β3 chains).

Assessing competition typically involves an evaluation of relative inhibitory binding using a first amount of a first molecule; a second amount of a second molecule; and a third amount of a third molecule (or a standard determined by binding studies that can be reasonably compared to new binding data with respect to the first and second molecules as a surrogate for actual contemporaneous data), wherein the first, second, and third amounts all are sufficient to make a comparison that imparts information about the selectivity and/or specificity of the molecules at issue with respect to the other present molecules. The first, second, and third amounts can vary with the nature of the Ln-5 γ2 binding peptide and potential targets therefore at issue. Usually, for ELISA assessments, similar to those described in the Examples, about 5-50 μg (e.g., about 10-50 μg, about 20-50 μg, about 5-20 μg, about 10-20 μg, etc.) of Ln-5 binding peptide an Ln-5 targets are used to assess whether competition exists. Conditions also should be suitable for binding. Typically, physiological or near-physiological conditions (e.g., temperatures of about 20-40° C., pH of about 7-8, etc.) are suitable for Ln-5 γ2-binding peptide:γ2 DIII binding

Often competition is marked by a significantly greater relative inhibition than about 10% as determined by ELISA analysis. It can be desirable to set a higher threshold of relative inhibition as a criteria/determinant of what is a suitable level of competition in a particular context (e.g., where the competition analysis is used to select or screen for new antibodies designed with the intended function of blocking the binding of another peptide or molecule to Ln-5 (e.g., for blocking interaction of Ln-5 with an Ln-5-binding integrin, matrix metalloproteinase, heparin, or naturally occurring anti-Ln-5 antibody)). Thus, for example, it is possible to set a criteria for competitiveness wherein at least about 20% relative inhibition is detected; at least about 25% relative inhibition is detected; or at least about 35% relative inhibition is detected before an antibody is considered sufficiently competitive. In cases where epitopes belonging to competing antibodies are closely located in an antigen, competition can be marked by greater than about 40% relative inhibition of γ2 DIII binding (e.g., at least about 45% inhibition, such as about 40-95% inhibition), at least about 50% inhibition, at least about 55% inhibition, at least about 60% inhibition, at least about 75% inhibition, or a higher level of relative inhibition).

Competition can be considered the inverse of cross-reactivity between a molecule and two potential binding partners. Thus, in certain aspects of the invention, a Ln-5 γ2 binding peptide of the invention can be characterized as a peptide that specifically binds to one or more residues or regions in γ2 but also advantageously does not cross-react with other peptides, peptide regions, or molecules. For example, the invention provides an anti-γ2 DIII antibody that does not substantially cross-react with Ln-5 γ3; an anti-γ2 DIII antibody that does not cross-react with Ln-5 γ3; anti-γ2 DIII antibodies that do not cross-react with Ln-5 and/or fragments of Ln-5 in the context of non-neoplastic epithelial cells; and anti-γ2 DIII antibodies that do not cross-react with pre-processed γ2 and/or γ2-asociated peptides). Typically, a lack of cross-reactivity means less than about 10% relative competitive inhibition between the molecules when assessed by ELISA using sufficient amounts of the molecules under suitable assay conditions.

In one illustrative aspect, the invention provides a Ln-5 γ2 binding peptide that competes with mAb 5D5 for binding to Ln-5 γ2 DIII or a portion thereof. In another illustrative aspect, the invention provides a peptide comprising or corresponding to a fragment or portion of a Ln-5 γ2 binding peptide that competes with mAb 5D5 for binding to γ2 DIII.

A Ln-5 γ2 binding peptide that cross-competes with both mAb 5D5 and mAb 6C12 for binding to γ2 DIII or a portion thereof is another feature of the invention. As discussed elsewhere herein, unless otherwise stated or clearly contradicted by context, references to binding of a Ln-5 γ2 binding peptide herein to Ln-5, γ22, or DIII are intended to refer to binding of these molecules in any suitable context, such as in a conformational context where the native structure of DIII is present; in a linear epitope context; in the context of a free γ2 chain molecule, or in the context of naturally occurring heterotrimeric Ln-5). Of course, binding in a limited subset of such context(s) can be an important functional characteristic with respect to any Ln-5 γ2 binding peptide provided by the invention, such that antibodies differing by these binding profiles can be considered unique aspects of the invention. Cross-competition can be determined by any suitable method and criteria, but typically means the detection of competition among two molecules for binding to a third molecule in an ELISA or other suitable assay (at similar or different levels). Examples of the detection of cross-competition (with reference to mAb 5D5 and mAb 6C12) are illustrated in the Examples.

A Ln-5 γ2 binding peptide that competes with mAb 6C12 for binding to γ2 DIII or a portion thereof, but that does not substantially cross-compete with mAb 5D5 with respect to γ2 DIII is another important exemplary feature of the invention (e.g., a L5G2BP that does not cause more than about 40% inhibition of 5D5 binding to γ2 DIII as assessed by an ELISA as typified by the methods described in the Examples). In a more particular aspect, the invention provides a Ln-5 γ2 binding peptide that competes with mAb 6C12 for binding to a region of Ln-5 spanning from about residue 495 to about residue 555 of Ln-5 γ2 (e.g., residues 494-552 of Ln-5 γ2; residues 500-548 of Ln-5 γ2; residues 508-543 of Ln-5 γ2; residues 516-533 of Ln-

In a further aspect, the invention provides a Ln-5 γ2 binding peptide that competes with mAb 5D5 with respect to binding a region of Ln-5 γ2 defined by residues 518-537 or thereabouts (e.g., from a position located at about residue 515 or about residue 520, such as Pro516, to about residue 540, such as Arg533). In a different aspect, the invention provides a Ln-5 γ2 binding peptide that competes with 6C12 for binding to a region of Ln-5 γ2 consisting essentially of residues 494-516, residues 539-552, or both, but not with respect to binding a region of Ln-5 γ2 consisting essentially of residues 518-537.

Monoclonal antibodies specific for γ2 domain III have been developed, characterized, and demonstrated to be useful in modulating physiological events of interest, such as the reduction in epithelial cell and epithelial-derived cell migration. The use of these antibodies and antibodies having similar features thereto are important features of this invention. Antibodies having features similar to these antibodies are expected to often also be useful in modulating such physiological events.

In one facet, the invention provides L5G2BPs that are capable of binding particular regions of Ln-5 that are antigenic, potentially antigenic, otherwise immunogenic, and/or that are targets for modulating Ln-5 and/or γ2-associated biological activities (e.g., γ2 peptide-associated epithelial cell migration).

Human laminin-5 γ2 DIII comprises a number of different antigenic determinants (epitopes), which may include, without limitation, (1) peptide antigenic determinants that are comprised within the γ2 peptide chain, (2) conformational antigenic determinants that consist one or more noncontiguous amino acids on the γ2 chain and/or amino acids present on spatially contiguous but separate Ln-5 peptide chains or discontinuous portions of the γ2 chain that are located near to each other in a folded γ2-associated protein; and (3) post-translational antigenic determinants which consist, either in whole or part, of molecular structures covalently attached to human laminin-5 γ2 or an associated peptide, such as carbohydrate groups.

In one aspect, the present invention provides isolated antibodies and other L5G2D3BPs that specifically bind to one or more epitopes located at least partially within domain III of γ2 chain of human laminin-5 (Ln-5 residues 382-608). Such antibodies and other L5G2D3BPs can be used in various inventive methods described herein. In general, the identification of L5G2D3BPs that bind to such epitopes can be accomplished applying the principles provided herein in combination with techniques known in the art by ordinarily skilled researchers applying no more than routine experimentation.

Antigenic determinant regions and predicted epitopes for mAb 4G1, mAb 5D5, and mAb 6C12, for example, have been identified via standard mapping and characterization techniques, further refinement of which can be accomplished by application of any suitable technique, numerous examples of which are available to the skilled artisan (references to “epitope mapping” techniques, “epitope identification” techniques, and the like, herein, should be understood as describing techniques applicable to the identification and/or refinement of antigenic determinant regions as well as epitopes). These techniques also can be used to identify and/or characterize epitopes for other L5G2D3BPs generally. As one example of such mapping/characterization methods, an epitope for an anti-γ2 DIII antibody may be determined by epitope “footprinting” using chemical modification of the exposed amines/carboxyls in the Ln-5 γ2 DIII protein. One specific example of such a footprinting technique is the use of HXMS (hydrogen-deuterium exchange detected by mass spectrometry). Briefly, in HXMS, a hydrogen/deuterium exchange of receptor and ligand protein amide protons is instituted, followed by peptide-antigen binding, and back exchange of receptor and ligand protein amide protons. The backbone amide groups participating in protein binding are protected from back exchange and therefore remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) and/or Engen, J. R. and Smith, D. L. (2001) Anal. Chem. 73, 256A-265A.

Another example of a suitable epitope identification technique is nuclear magnetic resonance (NMR) epitope mapping, where typically the position of the signals in two-dimensional NMR spectres of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR-spectrum. Antigen signals originating from amino acids involved in the interaction with the antigen binding peptide typically will shift position in the spectres of the complex compared to the spectres of the free antigen, and the amino acids involved in the binding can accordingly be identified. See, e.g., Ernst, Schering Res Found Workshop. 2004;(44):149-67; Huang et al, Journal of Molecular Biology, Vol. 281 (1) pp. 61-67 (1998); and Saito and Patterson, Methods. 1996 Jun;9(3):516-24.

Epitope mapping/characterization also can be performed using other mass spectrometry methods. See, e.g., Downward, J Mass Spectrom. 2000 Apr;35(4):493-503 and Kiselar and Downard, Anal Chem. May 1, 1999;71(9):1792-801.

Protease digestion techniques also can be useful in the context of “epitope mapping” and identification of antigenic determinant regions. Antigenic determinant-relevant regions/sequences can be determined by protease digestion, e.g. by using trypsin in a ratio of about 1:50 to gamma 2 domain III in an overnight digestion at about 37° C. and about pH 7-8, followed by mass spectrometry (MS) analysis for peptide identification using standard techniques. Peptides protected from trypsin cleavage by the anti-gamma 2 domain III binder can subsequently be identified by comparison of samples directly subjected to trypsin digestion and samples incubated with antibody first and then subjected to digestion by e.g. trypsin (thereby revealing a foot print for a binder). Other enzymes like chymotrypsin, pepsin, etc., also or alternatively can be used in similar epitope characterization methods. Enzymatic digestion can provide a quick method for analyzing whether a potential antigenic determinant sequence is within a region of the γ2 chain or γ2 chain in the context of a Ln-5 polypeptide that is not surface exposed and, accordingly, most likely not relevant in terms of immunogenicity/antigenicity. For example, an antibody which gives the significantly same result as mAb 4G1 in these measurements can be deemed to be an antibody that binds to essentially the same antigenic determinant region (ADR) as mAb 4G1. See, e.g., Manca, Ann Ist Super Sanita. 1991;27(1):15-9 for a discussion of similar techniques.

Epitope mapping by competitive binding to the gamma 2 domain III with two antibodies where one is biotinylated or otherwise similarly labeled is another method for identifying relevant antigenic determinant regions. Examples of this technique are provided in the Examples section of this document.

Various phage display techniques also can be used to identify epitopes. See, e.g., Wang and Yu, Curr Drug Targets. January 2004;5(1):1-15; Burton, Immunotechnology. August 1995;1(2):87-94; Cortese et al., Immunotechnology. August 1995;1(2):87-94; and Irving et al., Opin Chem Biol. June 2001;5(3):314-24. Consensus epitopes also can be identified through modified phage display-related techniques (see, Mumey et al., J. Comput. Biol. 10:555-567 and Mumey, Proceedings of the Sixth Annual International Conference on Computational Molecular Biology (RECOMB-02), pp. 233-240 (ACM Press, New York)) for discussion (see also Bailey et al., Protein Science (2003), 12:2453-2475; Dromey et al., J Immunol. Apr. 1, 2004;172(7):4084-90; Parker et al., Mol Biotechnol. January 2002;20(1):49-62; and Czompoly et al., Biochem Biophys Res Commun. Aug. 8, 2003;307(4):791-6).

Other methods potentially helpful in mapping epitopes include crystallography techniques, X-ray diffraction techniques (such as the X-ray diffraction/sequence study techniques developed by Poljak and others in the 1970s-1980s), and the application of Multipin Peptide Synthesis Technology. Computer-based methods such as sequence analysis and three dimensional structure analysis and docking also can be used to identify antigenic determinants. For example, an epitope also can be determined by molecular modeling using a structure of gamma 2 domain III with docking of the structure of the Fab fragment of the individual mAb. These and other mapping methods are discussed in Epitope Mapping A Practical Approach (Westwood and Hay Eds.) 2001 Oxford University Press (see also, Cason, J Virol Methods. September 1994;49(2):209-19).

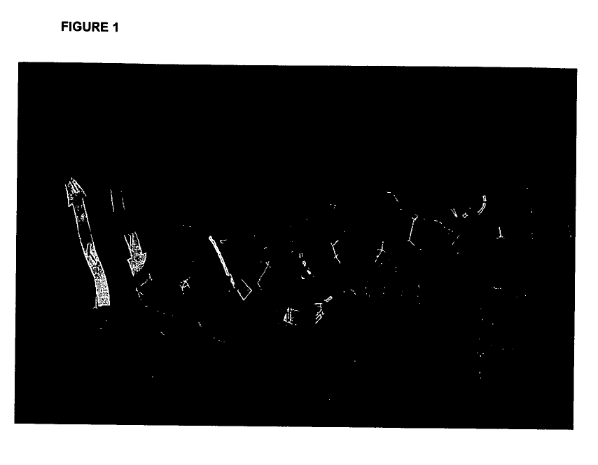

Amino acid residues in γ2 that are likely associated with antibody recognition by mAbs 5D5 and 6C12, for example, were identified by building a homology model using a template structure 1 NPE from PDB (Protein Data Bank—www.rcsb.org/pdb) using the program Moe 2003.02 (Molecular operating environment), which is commercially available from the Chemical Computing Group (Montreal, Quebec, Canada—www.chemcomp.com). The development and analysis of this model by standard techniques, in addition to the alignment of human and mouse gamma 2 domain III sequences, resulted in identification of a number of amino acids that are believed to confer selectivity and/or specificity for human γ2 DIII, as described elsewhere herein (see also, FIGS. 1-3).

If significant competition is observed for a peptide, such as a peptide comprising a portion of γ2 DIII or a fragment of γ2, in a competition assay with a known L5G2D3BP, that peptide typically is operationally identified as comprising at least a portion of an epitope, such as a sequential (linear) epitope of γ2. An epitope identified in this way may be incomplete, as there can be additional residues from the antigen which make contact with the antibody, but make little or no contribution to the binding energy. Often, a competition method relies on a very low level of contamination of the native antigen solution with denatured antigen. In addition, affinity of the L5G2D3BP for γ2 DIII and the concentration of the L5G2D3BP and γ2 can impact a competition assessment. It also may be the case that a peptide identified through such a technique comprises a conformational epitope, which is sufficiently recognized by the L5G2D3BPs due to sufficient retention of three dimensional structure.

In one aspect, the invention provides a method of identifying a L5G2D3BP that comprises performing one or more of these methods using a combination of one or more potential or known L5G2D3BPs and Ln-5 γ2 DIII or a related peptide. In particular aspects, the invention provides a method of identifying a peptide that competes with a peptide that binds to γ2, typically to γ2 DIII, such as one or more of mAbs 4G1, 5D5, and 6C12 by performing such methods with Ln-5 γ2 DIII or a peptide comprising γ2 DIII or a relevant portion thereof and one or more of such L5G2D3BPs.

In a particular exemplary aspect, the invention provides L5G2BPs, such as anti-γ2 antibodies, that specifically bind to a Ln-5 γ2 epitope or antigenic determinant that also is specifically bound by monoclonal antibody 4G1. For example, a chimeric or humanized antibody comprising 4G1 CDR sequences will likely exhibit the same specificity as mAb 4G1 with respect to a region of Ln-5 γ2 Dil. Other antibody-like proteins, such as immunoadhesins, comprising 4G1 CDR sequences, also can exhibit specificity for the same epitope as 4G1. However, it also is possible that L5G2D3BPs having one or more CDRs that differ from the CDRs of 4G1 can still be specific for the same epitope as 4G1. In some such cases, the 4G1 epitope-specific L5G2D3BP may better recognize or be more specific/selective for particular structures or regions of the 4G1 epitope than 4G1 (or recognize other residues located within the footprint of the 4G1 epitope).

In another specific exemplary aspect, the invention provides a Ln-5 γ2 binding peptide, e.g., a anti-γ2 DIII antibody, that binds to the same epitope or antigenic determinant region as monoclonal antibody 5D5 (as previously mentioned mAb 5D5 is under deposit with the DSMZ). A region in γ2 DIII that 5D5 is specific for has already been identified (see the Examples section herein). The epitope(s) or antigenic determinant(s) to which mAb 5D5 binds may be further refined using any one or more of the epitope mapping/identification methods described above. Antibodies with similar antigen-binding properties, such as specificity and/or selectivity, can be produced using any suitable technique, numerous examples of which are provided elsewhere herein.

In yet a further aspect, the invention provides a Ln-5 γ2 D III binding peptide, such as an antibody against γ2 Dil, which binds to the same epitope as monoclonal antibody 6C12. As previously mentioned, mAB 6C12 is under deposit with the DSMZ. A region of γ2 DIII to which 6C12 specifically binds has been determined by experimental method (see the Examples section provided herein). The epitope(s) or antigenic determinant(s) to which 6C12 binds and an antibody binding to the same epitope can be further refined using any one of the methods as described above with respect to the epitope of mAb 4G1.

A further facet of the invention is a Ln-5 γ2 DIII binding peptide having substantially the same specific binding characteristics of one or more mAbs selected from mAb 4G1, mAb 5D5, and mAb 6C12 (e.g., the subset thereof consisting of mAb 5D5 and mAb 6C12).

Advantageous L5G2D3BPs of the invention include “neutralizing” anti-γ2 DIII antibodies. The term “neutralizing antibody” refers to an antibody that is capable of substantially inhibiting or eliminating a biological activity of a γ2 DIII-associated peptide (e.g., a promigratory fragment of γ2, a promigratory γ2/β3 homodimer molecule, or a MMP-processed Ln-5 molecule). Typically, an anti-γ2 DIII neutralizing antibody will inhibit migration of epithelial cells in a degree that is about equal or greater than the inhibition of such cells occurring due to administration of an approximately equal amount of mAb 4G1, mAb 5D5, and/or mAb 6C12.

In some aspects, groups of L5G2BPs of the invention, particularly anti-γ2 DIII monoclonal antibodies provided by the invention, can be characterized as different from (i.e., as excluding) mAb D4B5 (see Koshikawa et al., Cancer Res. 1999 Nov 1;59(21):5596-601 and Mizushima et al., Horm Res. 1998;50 Suppl 2:7-14), mAb GB3 (Matsui et al. J Invest Dermatol. November 1995;105(5):648-52), and mAb B4-6 (Nguyen et al. (2000) JBC,275(41) p.31896-907). Thus, in some aspects the invention provides a L5G2D3BP and the use of a L5G2D3BPs having a certain set of characteristics, such as the ability to selectively and/or specifically bind γ2 Dil, with the proviso that the L5G2D3BP is not mAb D4B5, mAb GB3, or mAB B4-6. In other aspects, L5G2D3BPs of the invention, particularly anti-γ2 DIli antibodies of the invention, also or alternatively are distinguished from mAb 4G1, 5D5, and/or 6C12 (e.g., from mAb 4G1). Therefore, in some aspects, the invention provides a L5G2D3BP having certain characteristics, such as the ability to selectively and/or specifically bind γ2 DIII (and optionally further inhibit epithelial-derived cell migration) with the proviso that the L5G2D3BP is not mAb D4B5, mAb GB3, mAb B4-6, mAb 4G1, mAb 5D5, and/or mAb 6C12. In additional aspects, the invention provides a L5G2D3BP characterized by the inability to substantially (or in some cases even detectably) cross compete with mAb GB3, mAB D4B5, and/or mAb B4-6 with respect to the binding of at least one region of γ2 DIII.

To further illustrate the invention and describe particular aspects thereof, a number of ADRs identified/predicted by various experimental methods are described here. L5G2D3BPs that bind to, and typically are selective or specific for, such ADRs are features of this invention. The production of L5G2D3BPs, such as anti-γ2 DIII antibodies and antibody fragments, against such ADRs or portions of γ2 DIII comprising such ADRs, represent additional facets of the invention.

It will be appreciated by those knowledgeable in the field that apparent sequencing errors and/or other reasons have led to sequences for Ln-5 that differ from SEQ ID NO:1 being reported in the literature (see, e.g., Kallunki p. et al. J. Cell Biol. (1992) vol. 119 (3) 679-693). For every L5G2D3BP that binds to one or more ADRs defined by any portion of SEQ ID NO:1 recited herein, it should be understood that the invention provides a related L5G2D3BP that binds to an ADR defined by the portion of SEQ ID NO:1 but modified by changes that bring the sequence into compliance with other reported sequences for Ln-5. Thus, for example, L5G2D3BPs that bind to an ADR described herein that comprises Ln-5 residues 473 and/or 521 also should be deemed to provide support for a L5G2D3BP that binds to an essentially identical ADR with the modification that the Ilie residue at position 473 and/or the Ser residue at position 521 is/are substituted with Met and/or Asn, respectively. For example, in one aspect, the invention may describe a L5G2D3BP that selectively and/or specifically binds an ADR that consists essentially of residues 435-608 of Ln-5, having the following sequence Asp Glu Asn Pro Asp Ile Glu Cys Ala Asp Cys Pro Ile Gly Phe Tyr Asn Asp Pro His Asp Pro Arg Ser Cys Lys Pro Cys Pro Cys His Asn Gly Phe Ser Cys Ser Val Ile Pro Glu Thr Glu Glu Val Val Cys Asn Asn Cys Pro Pro Gly Val Thr Gly Ala Arg Cys Glu Leu Cys Ala Asp Gly Tyr Phe Gly Asp Pro Phe Gly Glu His Gly Pro Val Arg Pro Cys Gln Pro Cys Gln Cys Asn Ser Asn Val Asp Pro Ser Ala Ser Gly Asn Cys Asp Arg Leu Thr Gly Arg Cys Leu Lys Cys lIe His Asn Thr Ala Gly Ile Tyr Cys Asp Gln Cys Lys Ala Gly Tyr Phe Gly Asp Pro Leu Ala Pro Asn Pro Ala Asp Lys Cys Arg Ala Cys Asn Cys Asn Pro Met Gly Ser Glu Pro Val Gly Cys Arg Ser Asp Gly Thr Cys Val Cys Lys Pro Gly Phe Gly Gly Pro Asn Cys Glu His Gly Ala Phe Ser (SEQ ID NO:2), which means (in view of the foregoing) that the invention also provides a L5G2D3BP that selectively and/or specifically binds to an ADR that consists essentially of a portion of a Ln-5 protein having the sequence Asp Glu Asn Pro Asp lIe Glu Cys Ala Asp Cys Pro Ile Gly Phe Tyr Asn Asp Pro His Asp Pro Arg Ser Cys Lys Pro Cys Pro Cys His Asn Gly Phe Ser Cys Ser Val Met Pro Glu Thr Glu Glu Val Val Cys Asn Asn Cys Pro Pro Gly Val Thr Gly Ala Arg Cys Glu Leu Cys Ala Asp Gly Tyr Phe Gly Asp Pro Phe Gly Glu His Gly Pro Val Arg Pro Cys Gln Pro Cys Gln Cys Asn Asn Asn Val Asp Pro Ser Ala Ser Gly Asn Cys Asp Arg Leu Thr Gly Arg Cys Leu Lys Cys Ile His Asn Thr Ala Gly Ile Tyr Cys Asp Gln Cys Lys Ala Gly Tyr Phe Gly Asp Pro Leu Ala Pro Asn Pro Ala Asp Lys Cys Arg Ala Cys Asn Cys Asn Pro Met Gly Ser Glu Pro Val Gly Cys Arg Ser Asp Gly Thr Cys Val Cys Lys Pro Gly Phe Gly Gly Pro Asn Cys Glu His Gly Ala Phe Ser (SEQ ID NO:83). Other variations in the reported sequence of Ln-5 and SEQ ID NO:1 include substitution of the Gly residue at position 1110 with an Asp residue and substitution of the Met residue at position 1111 with a Gln residue. In cases where such residues are part of an ADR or target described herein described with reference to SEQ ID NO:1 it will be understood that the invention also provides an essentially identical L5G2D3BP or related composition that is defined by such an ADR or target that is identical except for such substitutions.

To identify more specific likely antigenic determinant regions in γ2, various predictive analytical methods were and/or can be applied, examples of which are described herein.

In a first analytical approach, γ2 DIII was analyzed for (1) highly hydropathic regions (using the Kyte-Doolittle method); (2) antigenicity as measured by the Protrusion Index method; (3) antigenicity as determined by the Parker method; (4) antigenicity as determined by the Hopp/Woods method; and (5) hydrophilicity as measured by the methods of Goldman, Engleman, and Steitz. Sequences ranging from 10-40 amino acids in length were selected based on exhibiting one or more of these properties. The regions of Ln-5 identified as likely antigenic determinants by this analysis are provided in Table 1. The rationale for this approach is the general consensus that many ideal B cell epitopes are hydrophilic, surface-oriented, and flexible sequences of about 8-10 amino acids in length.

TABLE 1

| | Ln-5 gamma2 domain III, | | | | | |
|---|---|---|---|---|---|---|
| Method | potential antigenic regions | | | | | |
| Kyte-Doolittle | | 400-420 | | 462-502 | 530-550 | |
| (Hydropathy) | | | | | | |
| Protrusion | 382-407 | 415-440 | | | 520-540 | |
| Index | | | | | | |
| (antigenicity) | | | | | | |
| Parker | | 420-440 | 450-460 | | 520-540 | 570-590 |
| (antigenicity) | | | | | | |
| Hopp/Woods | 390-400 | 425-440 | | 490-510 | | 560-600 |
| (antigenicity) | | | | | | |
| Goldman/Engelman/ | | | | | | |
| Steitz | | | | | | |
| (Hydrophilicity) | 385-395 | 425-440 | 445-460 | | 520-540 | 560-570 |
| hits | 3 | 5 | 2 | 2 | 4 | 3 |
| Most likely | 380-400 | 420-460 | | | 520-550 | 560-590 |
| antigenic | | | | | | |
| determinant | | | | | | |
| regions | | | | | | |

This sequence characterization information was further analyzed with respect to the previously predicted domain structure of γ2 DIII (see, e.g., UNIPRO record LMG2_HUMAN) and experimental evidence obtained with anti-γ2 DIII monoclonal antibodies (mAbs). Briefly, Ln-5 γ2 DIII reportedly contains five EGF-like domains (arranged one partial, three complete, one partial N-terminus to C-terminus) spanning essentially the entire gamma 2 III region. The approximate locations of these domains are residues 382-415; 416-461; 462-516; 517-572; and 573-602, respectively. Additionally, experimental evidence available prior to this analysis demonstrated that at least some γ2 DIII-specific mAbs bind to regions defined by about position 430 to about position 460 and separately to about position 460 to about position 540 of Ln-5. Thus, out of the sequences that the above-described sequence analysis justify as the most likely antigenic regions (i.e., regions defined by about residues 380-400, 420-460, 520-550, and 560-590 of Ln-5), the regions defined by about residue 420 to about residue 460, about residue 520 to about residue 550, and about residue 560 to about residue 590 of Ln-5 are particularly likely to define or comprise useful antigenic determinants. Given the proximity of two of the EGF-like domains, Ln-5 γ2 DIII binding peptides specific for residues 517-572 or residues 573-602 also are expected to be useful in the methods of the invention and provide additional inventive features. Ln-5 γ2 DIII binding peptides that specifically bind to any of these predicted antigenic determinant regions, and particularly that specifically bind a region defined by about residue 520 to about residue 550 and about residue 560 to about residue 590 of Ln-5, are exemplary features of this invention.

Additional analysis of the Ln-5 γ2 DIII sequence in view of these factors provided a set of exemplary likely antigenic determinant regions, specificity for which can characterize a Ln-5 γ2 DIII binding peptide of the invention. The regions of Ln-5 that correspond to these more particular exemplary likely antigenic determinants are provided in Table 2.

TABLE 2
Likely Ln-5 γ2 DIII Antigenic Determinant Regions Identified by
Multi-factored Sequence Analysis (numbers refer to γ2 residues
that approximately define the region)

| | | | |
|---|---|---|---|
| 382-567 | 391-567 | 420-460 | 435-608 |
| 435-602 | 435-590 | 435-550 | 435-534 |
| 462-608 | 462-602 | 462-572 | 462-567 |
| 462-550 | 462-534 | 494-608 | 494-602 |
| 494-590 | 494-572 | 494-567 | 494-550 |
| 494-534 | 520-550 | 560-590 | |

To further characterize the γ2 DIII with respect to the identification of antigenic determinants in terms of confirming the antigenic character of the above-identified regions and/or identifying additional likely antigenic determinants, the sequence of γ2 DIII was subjected to analysis by B cell epitope prediction software, such as the ANTIGENIC program currently publicly accessible at the Emboss web site—http://emboss.ch.embnet.org/Pise/antigenic.html (see Kolaskar et al., (1990) FEBS Letters 276: 172-174 and Parker et al., (1986) Biochemistry 25: 5425-5432) and the Prediction of Antigenic Determinants Program, which employs the methodology of Kolaskar and Tongaonkar, FEBS Lett. Dec. 10, 1990;276(1-2):172-4 and is currently publicly available through the Molecular Immunology Foundation at http://mif.dfci.harvard.edu/Tools/antigenic.pl. Through this further analysis the following regions of DIII were identified as expected antigenic determinants.

TABLE 3
Likely ADRs Identified By B Cell Epitope Prediction Software Analysis

| | | | |
|---|---|---|---|
| 385-399 | 386-400 | 409-418 | 411-420 |
| 439-447 | 441-449 | 457-464 | 476-498 |
| 478-500 | 507-521 | 509-523 | 533-555 |
| 535-557 | 568-575 | 570-577 | 587-595 |
| 589-597 | 602-618 | | |

Ln-5 γ2 DIII binding peptides specific for these regions of Ln-5 represent another feature of the invention. Moreover, the termini of these sequences may be compared to predicted antigenic determinant regions located through the other analyses described herein to provide additional specific likely antigenic determinant regions (e.g., the identification of residues 385-399 by computer epitope prediction methods and 380-400 by multi-pronged sequence characteristic analysis further suggests that a region defined by residues 385-400, a region defined by residues 380-399, or both may define suitable antigenic determinant regions within Ln-5 γ2 DIII). Other similar comparisons can readily be made to provide additional likely ADRs, which may be considered another feature of the invention.

Analysis of similarities between likely antigenic determinants identified by computer predicted antigenic sequence analysis and those identified by the multi-factor sequence characteristic analysis above suggests that regions of Ln-5 γ2 DIII corresponding to about residues 385-400, about residues 440-465, about residues 535-560, and about residues 560-580 of Ln-5 also are likely to contain or be antigenic determinants, such that Ln-5 γ2 binding peptides that bind specifically to such a region of Ln-5 are another feature of the invention.

Relative γ2 peptide binding inhibition studies (competition studies) can identify alternative antigenic regions and/or refine/complement the above-described predictive analysis. In this respect, the invention provides, for example, a anti-γ2 DIII antibody that binds to a portion of γ2 DIII that comprises residues 392-494 of γ2. In another aspect, the invention provides an anti-γ2 DIII antibody that binds to a portion of γ2 DIII that comprises residues 494-555 of γ2. In another aspect, the invention provides an anti-γ2 DIII antibody that binds to a portion of Dil comprising residues 461-494 of γ2. These and other ADRs have been identified through competition studies involving anti-γ2 DIII mAbs.

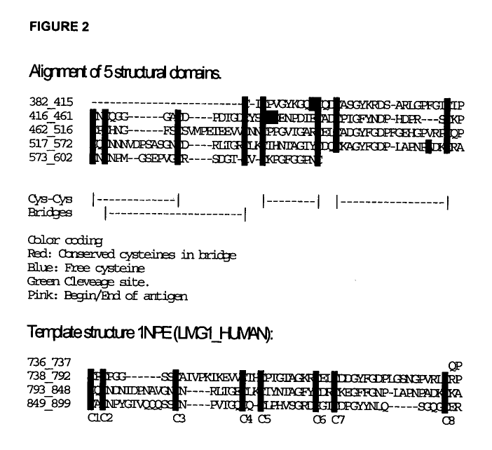

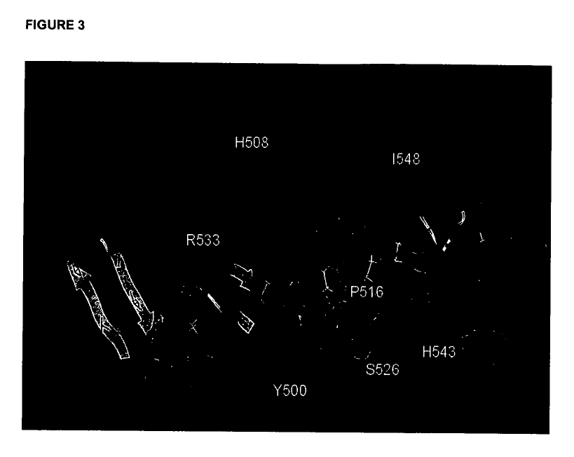

To further refine the identification/characterization of γ2 antigenic determinants, a three-dimensional model of Ln-5 γ2 was developed based on the known crystal structure of the laminin yl chain and comparisons between the structural domains of Ln-5 γ2 (see FIG. 2) and those of the laminin γ1 chain. High probability success models for γ2 can be readily developed in this manner because the cysteine bridges present in γ1 and γ2 have an essentially identical pattern (see FIG. 2). Using this information, a three dimensional model of a C-terminal portion of a γ2 DIII fragment, consisting of Ln-5 residues 392-567, which has been used successfully to immunize mice, was developed by computer modeling (see FIG. 1). The human gamma 2 domain III was thereafter aligned with murine Ln-5 gamma 2 domain III to identify possible epitopes. From these studies, the most C-terminal part of the peptide (residues 460-567 of Ln-5) was identified to have a number of amino acids as potential epitopes or epitope constituents (see FIG. 3). In particular, Tyr500 (i.e., Tyr₅₀₀), His508, Pro516, Ser526, Arg533, His543, and Ile548 were identified as residues within this portion of DIII that are highly likely to mark portions of antigenic determinants that confer selectivity for human Ln-5 γ2 DIII. In other words, murine to human sequence comparison combined with modeling led to a finding that L5G2D3BPs specific for epitopes comprising one or more of these residues will very likely exhibit selectivity for human Ln-5 γ2 DIII over murine Ln-5 γ2 DIII. Moreover, the position and nature of these residues suggest that in general they are potentially relevant components of γ2 DIII antigenic determinants. Accordingly, L5G2D3BPs comprising one or more of these residues are important features of this invention. In another facet, the invention provides a L5G2D3BP that specifically and/or selectively binds to an antigenic determinant comprising one or more of these residues and one or more (e.g., two, three, or four) residues located within about 15 angstroms therefrom (e.g., about 10-20 angstroms therefrom). In another aspect, the invention provides a L5G2D3BP that binds to one of these residues and one or more residues located within a typical epitope area (see, e.g., Graille M. et al, Structure (2001) vol. 9 (8) p. 679-87, for related discussion). As mentioned above, the “epitope area” for any L5G2D3BP encompasses both the actual contact site as well as the area where other binding molecules are subject to steric hindrance by the bound L5G2D3BP.

Taking information from experimental evidence (e.g., evidence that demonstrated that one or more likely antigenic determinants were located in a region defined by Ln-5 residues 391-461, 391-434, or 434-461 and one or more other likely antigenic determinants were located in a region defined by Ln-5 residues 494-555 or 494-567), sequence analysis, epitope prediction, and three-dimensional modeling and peptide homolog comparison studies, a number of regions of γ2 that are likely targets for L5G2BPs emerge.

Within these regions, certain amino acid residues are more likely than others to contribute to L5G2D3BP binding. Typically, charged amino acid residues (e.g., Lys and Glu) and hydrophobic amino acids (Ala, Leu, Tyr, Phe, and Val) are more likely to be important to antibody or other L5G2D3BP binding. In contrast, Cys residues are typically not critical components of epitopes. It is also important to note that although an epitope can be spread out over the surface of an antigen, a few residues, or single residue, can be critical to paratope/CDR binding of an epitope, whereas other residues may play a lesser, nonessential role in the affinity and/or avidity of an L5G2D3BP:epitope interaction.

With the foregoing in mind, in one aspect the invention provides a Ln-5 γ2 DIII binding peptide that specifically binds to a region of Ln-5 corresponding to about position 500 to about position 550 of Ln-5. Ln-5 γ2 DIII peptides that specifically bind such a region can be associated with a greater ability to reduce the migration of epithelial cell-derived cells, such as epithelial-derived cancer cells (epithelial cancer cells) than a Ln-5 γ2 DIII binding peptide that is specific for another region of γ2 DIII (e.g., mAb 4G1) or γ2 (e.g., mAb GB3 and/or mAb D4B5). A Ln-5 γ2 DIII binding peptide according to such an aspect also may be characterized as competing with mAb 5D5 and/or 6C1 2 at relative inhibition levels significantly higher than those posed by mAb 4G1 against this region of Ln-5. Exemplary sub-regions of this region of γ2 DIII that are likely to contain or correspond to more particular antigenic determinants are set forth in Table 4:

TABLE 4
Likely antigenic determinant regions in the portion of γ2 DIII defined
by about residue 500 to about residue 550 thereof

| | | | | | |
|---|---|---|---|---|---|
| 494-553 | 494-552 | 494-548 | 494-543 | 500-508 | 500-516 |
| 500-533 | 500-534 | 500-543 | 500-548 | 508-516 | 508-533 |
| 508-534 | 508-543 | 508-548 | 516-533 | 516-534 | 516-543 |
| 516-548 | 533-543 | 533-548 | 500-526 | 508-526 | 516-526 |

In a further aspect, the invention provides an antibody that specifically binds to a portion of Ln-5 according to any of these regions, wherein the region comprises Tyr500, His508, Pro516, Ser526, Arg533, His543, Ile548, or a combination of any contiguous amino acid residue sequences comprising two or more thereof. Likely antigenic residues located near these five specifically identified residues may be included in such an epitope or antigenic determinant region and may contribute to L5G2D3BP binding. These residues include Phe501 (with respect to Tyr500), Glu507 (with respect to His508), Ala527 (with respect to Ser526), Leu532 (with respect to Arg533), and Tyr549 (with respect to Ile548). Similar analysis of other nearby residues, taking into account the structure of γ2 DIII provided in FIGS. 1 and 2, may be used to identify additional nearby residues (within about 15 angstroms thereof) that may contribute to epitopes with these sequences.

In a particular aspect, the invention provides an L5G2D3BP that specifically binds to an epitope, such as a structural epitope, comprising Tyr500 and Pro516. In such an aspect, the epitope also may comprise, for example, Phe501.

In another particular aspect, the invention provides a L5G2D3BP that specifically and/or selectively binds to an epitope, such as a structural epitope, that comprises His508. Such an L5G2D3BP may specifically bind an epitope that also includes Arg533. Such an L5G2D3BP also may also bind an epitope that includes Glu507 and/or Leu532.

In a further particular aspect, the invention provides a L5G2D3BP that specifically binds a region of DIII that comprises Ser526. In a further aspect, the invention provides such an L5G2D3BP wherein the Ser526-containing epitope may further comprise Ala527.

The regions identified in the preceding paragraphs can include one or more epitopes specific for anti-γ2 DIII antibody/L5G2D3BP binding. Predictive data and/or experimental evidence points to the selection of these and other regions of Ln-5 as consisting essentially of, comprising, or corresponding to epitopes for anti-L5G2D3 Abs and other L5G2D3BPs.

Sequence alignment analysis and inter-sequence analysis (murine to human—see, e.g., FIG. 2), as described above with respect to the region of Ln-5 defined by residues 460-567, also was performed with respect to a more N-terminal portion of γ2 DIII, resulting in the identification of a number of additional amino acid residues that are likely to form part of antigenic determinant(s) and also may confer selectivity to human Ln-5 γ2 DIII over murine DIII (e.g., as might be determined by a competition assay, such as those described elsewhere herein, or by other suitable method such as direct affinity measurement comparisons). Specifically, the results of this analysis point to Asp395, Thr412, Ile414, and Cys442 (individually or collectively) as very likely components of Ln-5 γ2 DIII epitopes and selectivity determinants for human γ2 DIII in this region of DIII. Again, comparison with experimental data, sequence data based on the multi-featured analysis described above, predicted epitopes identified by sequence analysis, and structural components of Ln-5 γ2 DIII leads to the identification of a number of regions that likely comprise antigenic determinant sequences and/or structures in this region of Ln-5. Additional likely antigenicity-contributing or antigenicity-conferring residues located near these residues include Phe 410 (which is located near Thr412) and Glu441 and Ala443 (both near Cys442). L5G2D3BPs that are specific for regions of Ln-5 comprising any one or combination of such residues, or sequences comprising such residues, are another aspect of this invention. Exemplary aspects arising from this analysis include a L5G2D3BP that is specific for an epitope, such as a structural epitope, comprising Asp395 and Thr412; Asp395 and Ile414; Thr412 and Ile414; Asp395, Thr412, and Ile414; or any one of Asp395, Thr412, and Ile414, without other members of this group (e.g., without Cys442).

Similar strategies were employed to identify a number of regions of Ln-5 γ2 DIII, which may or may not overlap with the foregoing disclosure of antigenic determinants. For sake of further illuminating the invention through the description of various features and aspects, a number of exemplary L5G2D3BPs characterized by their specificity and/or selectivity for particular likely and/or confirmed antigenic determinant regions of Ln-5 γ2 DIII will be described in the following paragraphs.

In one such exemplary aspect, the invention provides a Ln-5 γ2 DIII binding peptide that specifically binds to a region of LN-5 defined, at least in part, by about residues 395-445 of γ2. Likely Ln-5 antigenic determinants comprised within this region include, but are not limited to, the regions defined by Ln-5 γ2 residues 395-442, 385-399, 385-400, 385-442, 390-460, 391-461, 391-460, 386-400, 382-407, 385-395, 400-440, 400-420, and 425-440.

In a particular exemplary aspect, the invention provides a L5G2D3BP that specifically binds to a region of Ln-5 γ2 defined, at least in part, by about residue 395 to about residue 414 (e.g., residues 394-414). The inventors have determined that such a region is related to an antigenic determinant for mAb 4G1, which has been demonstrated to bind γ2 DIII. Thus, the invention provides L5G2D3BPs, such as anti-γ2 DIII chimeric, humanized, or fully human monoclonal antibodies that specifically and/or selectively bind this region of Ln-5 γ2.

Other exemplary aspects of the invention include L5G2D3BPs that bind to a region in Ln-5 γ2 defined, at least in part, by a region from residue 412 to residue 442 of Ln-5 γ2. In one such aspect, the invention provides a L5G2D3BP that specifically binds to a region of Ln-5 γ2 defined by Ln-5 residues 415-440, 411-420, 409-418, 420-460, or 435-460.

In a further aspect, the invention provides a L5G2D3BP that binds to a Ln-5 epitope that comprises residue 412 and residue 414 of Ln-5 γ2.

In yet another aspect, the invention provides a L5G2D3BP that comprises an epitope that includes Cys442. In another aspect, the invention provides a L5G2D3BP that comprises a linear epitope that encompasses Cys442 or a structural epitope that when bound results in the blockage of access to Cys442 (such epitopes also may comprise Glu441 and/or Ala443). In one such aspect, the invention provides a L5G2D3BP that specifically binds to a region defined by about positions 439-447, 441-449, 442-460, 420-442, or 450-460 of Ln-5 β2. L5G2D3BPs according to such aspects may advantageously reduce the binding of Ln-5 to other peptides, small molecules, cells, structures, or materials, which involve Cys442.

In another facet, the invention provides an anti-γ2 DIII binding peptide that is specific for an amino acid sequence that comprises one or more residues in the hinge region of Ln-5 γ2. Exemplary L5G2D3BPs according to this aspect specifically bind a region defined by about position 600 to about position 620 of Ln-5 γ2 (e.g., positions 602-620, 608-620, 602-612, 602-611, 608-620, 608-612, 608-613, 607-612, 607-613, etc.).

In another aspect, the invention provides a Ln-5 γ2 DIII binding peptide that is specific for a region of Ln-5 γ2 that overlaps a γ2 DIII proteolytic cleavage site. Illustrative aspects include L5G2D3BPs that are specific for a region of Ln-5 γ2 defined by about position 410 to about position 445 thereof, such as a region defined by residues 412-442, residues 414-442, residues 420-442, residues 420-435, residues 414-435, residues 412-435, residues 433-442, residues 430-442, and residues 432:443 of Ln-5 γ2, etc.

However, experimental evidence demonstrates that antibodies specific for a region defined by residues 494-534 of Domain 1II, downstream of the processing site at residue 434 of Ln-5, can have a greater effect on inhibiting cell migration compared to an antibody which targets an epitope which spans the processing site (e.g., residues 391-461). This data supports the preferential targeting of sequences contained within a specific region of Domain III, which is downstream to the cleavage site at about residue 434, and remains as part of the Laminin 5 molecule after proteolytic processing has occurred, in connection with reducing cell migration (in other aspects, targeting a region spanning the processing site may be more advantageous). As is generally the case with antibodies described herein, such an antibody can be a polyclonal antibody or a monoclonal antibody, but typically is a monoclonal antibody. Such an antibody can be a humanized, fully human, or murine form of an antibody. A non-antibody L5G2D3BP specific for such regions also can be used in the methods of this invention and/or incorporated into a composition of this invention. As described elsewhere herein, combinations of any of the specifically described L5G2D3BPs of the invention also can be usefully included in the compositions and methods described herein, such that, for example, it can be possible to include an antibody specific for a region downstream of the processing site and an antibody specific for a region near or that spans the processing site in such methods or compositions.

Another feature of the invention is an antibody that specifically binds to a portion of γ2 defined by about position 495 to about position 590 (e.g., residues 494-590). In another aspect, the invention provides an anti-γ2 antibody that specifically binds to a portion of γ2, domain III, in a region defined by about position 495 to about position 570 of Ln-5 γ2 (e.g., residues 494-572, residues 494-567, etc.). Non-antibody L5G2D3BPs having such binding specificities also are provided.

In yet another aspect, the invention provides an anti-γ2 antibody that specifically binds to a portion of γ2 domain III, in a region defined by about position 495 to about position 550 of Ln-5 γ2 (e.g., a region defined by residues 494-550, such as a region defined by residues 494-549; a region defined by about position 530 to position 550 of γ2 (such as a region defined by residues 533-555 or residues 535-557 of γ2)). For example, the invention provides an anti-γ2 antibody that specifically binds to a portion of γ2, domain III, in a region defined by about position 495 to about position 545 of Ln-5 γ2. In an additional particular aspect, the invention provides an anti-γ2 antibody that specifically binds to a portion of γ2, domain III, in a region defined by about position 495 to about position 530 of Ln-5 γ2 (e.g., a region defined by residues 494-534 of Ln-5 γ2). In another exemplary aspect, the invention provides an anti-γ2 antibody that specifically binds to a portion of γ2, domain III, in a region defined by about position 500 to about position 550 of γ2 (e.g., a portion defined by residues 500-545, 500-548, or 500-549). Non-antibody L5G2D3BPs having such binding specificities also are provided by the invention.

The invention also provides, in one exemplary aspect, an anti-γ2 antibody that specifically binds to a portion of γ2, domain III, in a region defined by about position 510 to about position 545 of Ln-5 γ2. For example, the invention provides an anti-γ2 antibody that specifically binds to a portion of γ2, domain III, in a region defined by about position 510 to about position 535 of Ln-5 γ2. In a more particular aspect, the invention provides an anti-γ2 antibody that specifically binds to a portion of γ2, domain III, in a region defined by about position 510 to about position 520 of Ln-5 γ2. In yet another aspect, the invention provides an anti-γ2 antibody that specifically binds to a portion of γ2, domain III, in a region defined by about position 525 to about position 545 of Ln-5 γ2. An anti-γ2 antibody that specifically binds to a portion of γ2, domain III, in a region defined by about position 525 to about position 535 of Ln-5 γ2 is another exemplary feature of the invention. In still another aspect, the invention provides an anti-γ2 antibody that specifically binds to a portion of γ2, domain III, in a region defined by about position 535 to about position 545 of Ln-5 γ2. Where suitable non-antibody L5G2D3BPs having such binding specificities can serve as alternatives and thereby form additional features of the invention.

In still another aspect, the invention provides an anti-γ2 antibody that specifically binds to a portion of γ2, domain III, in a region defined by, at least in part, about position 560 to about position 600 of γ2 (e.g., about position 570 to about position 590, such as positions 568-575, 570-577, 560-579, about position 590 to about position 600, such as positions 587-595, 589-597, etc.). Non-antibody L5G2D3BPs having such specificities are additional features of the invention.

Anti-γ2 antibodies and L5G2D3BPs that specifically bind to a portion of γ2, domain III, in a region defined by, at least in part, about position 460 to about position 610 of Ln-5 γ2 (e.g., a region defined by about position 460 to about position 495, a region defined, at least in part, by residues 462-608, a region defined by residues 462-602, etc.) also may be useful in the practice of the inventive methods described herein. Accordingly, such antibodies (or other L5G2D3BPs) and compositions comprising such antibodies (or other L5G2D3BPs) are further features of this invention. In a more particular aspect, the invention provides an anti-γ2 antibody that specifically binds to a portion of γ2, domain III, in a region defined, at least in part, by about position 460 to about position 600 of Ln-5 γ2. In a further exemplary aspect, the invention provides an anti-γ2 antibody that specifically binds to a portion of γ2, domain III, in a region defined by about position 460 to about position 590 of Ln-5 (e.g., residues 462-590). An anti-γ2 antibody that specifically binds to a portion of γ2, domain III, in a region defined, at least in part, by about position 460 to about position 570 of Ln-5 (e.g., residues 462-572 or residues 462-567) is another feature of the invention. For example, the invention provides an anti-γ2 antibody that specifically binds to a portion of γ2, domain III, in a region defined, at least in part, by about position 460 (e.g., position 450, 455, 458, 461, 462, 465, etc.) to about position 550 (e.g., position 545, 547, 548, etc.) of Ln-5. Another exemplary aspect of the invention is an anti-γ2 antibody that specifically binds a portion of Ln-5 γ2 DIII in a region defined by, at least in part, about residues 460-530 thereof (e.g., residues 462-534); yet another aspect is an antibody that specifically binds a region defined by about residues 465-500 of γ2 (e.g., residues 462-502 or about residues 480-500, such as residues 476-498 or 478-500). In another aspect, the invention provides an antibody that specifically binds a region defined, at least in part, by about residues 455-465 (e.g., residues 457-464 of Ln-5 γ2, residues 445-460 of Ln-5 γ2, etc.). Where suitable other L5G2D3BPs can be used in place of antibodies having such features.

In a particular aspect, the invention provides an antibody or other L5G2D3BP that specifically binds to a portion of Ln-5 in a region defined, at least in part, by about position 460 to about position 525 of Ln-5 γ2, such as about position 460 to about position 515 of Ln-5 γ2 (e.g., a region defined by about residues 490-510 of Ln-5 or about residues 510-525, such as residues 507-521 or residues 509-523 of Ln-5 γ2). In a particular aspect, the invention provides an antibody or other L5G2D3BP that binds to a portion of Ln-5 in this region wherein the region comprises Tyr500 of Ln-5 γ2, Pro516 of Ln-5 γ2, or both. For example, the invention provides an antibody or other L5G2D3BP that binds to a portion of Ln-5 defined by, at least in part, residues 462-516 of Ln-5 γ2.

Antibodies and other L5G2D3BPs that specifically bind a portion of Ln-5 γ2 DIII in a region defined by, at least in part, about position 520 to about position 570 of γ2 (e.g., a portion of Ln-5 γ2 defined by positions 517-572), such as antibodies that bind to a portion of Ln-5 defined by, at least in part, about position 520 to about position 550 of γ2, also can be useful components in inventive methods and compositions described herein. Such antibodies, other L5G2D3BPs, and compositions comprising such antibodies and/or L5G2D3BPs are further features of the invention. In a more particular aspect, the invention provides an antibody or L5G2D3BP that binds to a portion of Ln-5 γ2 in a region defined by, at least in part, about position 525 to about position 545 of Ln-5 γ2. In further exemplary aspects, the invention provides antibodies or L5G2D3BP that specifically bind to a portion of Ln-5 in a region defined by, at least in part, about residues 525-535 or 535-545 of Ln-5 γ2. In another particular aspect, the invention provides an antibody that binds to a region of Ln-5 γ2 comprising Ser526, Arg533, His543, or a combination of any thereof (e.g., Ser526 and Arg 533; Arg533, and His543).

Antibodies and other L5G2D3BPs that specifically bind to a portion of γ2, domain III, in a region defined by about position 380 to about position 570 of Ln-5 γ2 (such as, for example, a region defined by positions 382-567 of Ln-5 γ2) or a region defined by about position 380 to about position 460 of Ln-5 γ2 (e.g., a region defined by about residues 382-407, about residues 385-395, about residues 390-570 (such as residues 391-567)), about residues 395-445 (such as residues 395-442), about residues 385-445 (such as residues 385-442, residues 395-442, etc.) about residues 385-400 (such as residues 386-400, 385-399, residues 386-399, etc.), about residues 395-445 (e.g., residues 395-442, residues 395-414, residues 395-412), or about residues 400-420 of γ2 (such as residues 409-418, 411-420, etc.)) may also or alternatively be useful in practicing the methods of the invention. Such antibodies and compositions comprising such antibodies are features of the invention.

In another exemplary aspect, the invention provides an anti-γ2 antibody that specifically binds to a portion of γ2, domain III, in a region defined by, at least in part, about position 380 to about position 570 of Ln-5 γ2. In another aspect, the invention provides an anti-γ2 antibody that specifically binds to a portion of γ2, domain III, in a region defined by, at least in part, about position 390 to about position 570 of Ln-5 γ2. In a further aspect, the invention provides an anti-γ2 antibody that specifically binds to a portion of γ2, domain III, in a region defined by, at least in part, about position 395 to about position 450 of Ln-5 γ2. In an alternative aspect, the invention provides a non-antibody L5G2D3BP that exhibits such specificity for Ln-5 γ2.

In another exemplary aspect, the invention provides an anti-γ2 antibody that specifically binds a portion of γ2, domain III, in a region defined by, at least in part, about position 395 to about position 445 of γ2. In a further aspect, the invention provides an anti-γ2 antibody that binds to a region defined by, at least in part, about position 395 to about position 415 of γ2 (e.g., a region defined by residues 395-414, 395-412, etc.). In another aspect, the invention provides an anti-γ2 antibody that binds to a portion of Ln-5 in a region defined by about position 410 to about position 445 of γ2 (e.g., a region defined by residues 412-442, 414-442, etc.). In one aspect, the invention provides an antibody that binds to such a region of Ln-5 wherein the region comprises Asp395 of Ln-5 γ2. In another aspect, the invention provides an antibody that binds to such a region wherein the region also or alternatively comprises Thr412, 11e414, or both. In a further aspect still, the invention provides such an antibody wherein the region also or alternatively comprises Cys442 of Ln-5 γ2. In another aspect, the invention provides an antibody that binds to an epitope which comprises residues 412-414 of Ln-5 γ2. Non-antibody L5G2D3BPs having such binding specificities, and the use of such L5G2D3BPs in the inventive methods described herein, are additional features of the invention.

Antibodies that specifically bind to a portion of Ln-5 γ2 in a region defined by about position 420 to about position 460 thereof also may be useful in the practice of inventive methods described herein. Such antibodies and compositions comprising such antibodies are further features of the invention. In a more particular aspect, the invention provides an antibody that specifically binds to a region of Ln-5 γ2 defined by, at least in part, about positions 420-460 thereof, wherein the region comprises Cys442 of Ln-5 γ2. In another aspect, the invention provides an antibody that specifically binds to a portion of Ln-5 γ2 defined by, at least in part, about position 430 to about position 460 thereof. In a more particular aspect, the invention provides such an antibody wherein the portion comprises Cys442 of Ln-5 γ2. Non-antibody L5G2D3BPs having such binding specificities are additional features of the invention.

Antibodies that specifically bind to a portion of Ln-5 γ2 in a region defined by about position 435 to about position 608 thereof (e.g., residues 435-602, 443-602, 442-602, 444-602, etc.) also can be useful components in compositions of the invention and in practicing the inventive methods described herein. Such antibodies and compositions comprising such antibodies are additional features of the invention. In an exemplary aspect, the invention provides an anti-γ2 antibody that specifically binds to a portion of γ2, domain III, in a region defined by, at least in part, about position 435 to about position 590 of Ln-5 γ2. In another exemplary aspect, the invention provides an anti-γ2 antibody that specifically binds to a portion of γ2, domain l1l, in a region defined by, at least in part, about position 435 to about position 550 of Ln-5. An antibody that specifically binds a portion of Ln-5 in a region defined by, at least in part, about position 435 to about position 535 of Ln-5 (e.g., positions 435-534, 442-533, 442-526, 442-516, 442-508, 442-500, etc.). In a more particular aspect, the invention provides an antibody that specifically binds to any of the portions described in this paragraph, wherein the portion comprises Cys442 of Ln-5. In a further aspect, the invention provides a non-antibody L5G2D3BP that has any of the binding specificities described here.

Antibodies that specifically bind to a portion of Ln-5 γ2 in a region defined by, at least in part, about position 435 to about position 455 (e.g., 435-450, 435-442, etc.) may be useful in inventive methods described herein. Such antibodies and compositions comprising such antibodies are yet another feature of the invention. In one aspect, the invention provides such an antibody wherein the antibody specifically binds to a portion of Ln-5 γ2 that comprises Cys442 thereof. An antibody that specifically binds to a portion of Ln-5 in a region defined by about position 435 to about position 455 of Ln-5 γ2 is another feature of the invention. In one aspect, such an antibody specifically binds a portion of Ln-5 that comprises Cys442 of Ln-5 γ2.

In a different aspect, the invention provides an antibody that specifically binds a portion of Ln-5 in a region defined by, at least in part, about position 380 to about position 420 of Ln-5 γ2 (e.g., a region defined by positions 382-415, a region defined by positions 394-414, a region defined by positions 395-412, etc.). In another aspect, the invention provides an antibody that specifically binds a portion of Ln-5 in a region defined by, at least in part, about position 380 to about position 400 of γ2 (e.g., a region defined by positions 382-395 of Ln-5 γ2). Non-antibody L5G2D3BPs having such binding specificities are also features of this invention.

In another aspect, the invention provides an antibody or other L5G2D3BP that specifically binds to a portion of Ln-5 in a region defined by, at least in part, about position 395 to about position 420 of Ln-5 γ2 (e.g., a region defined by position 395-415, 395-416, or 395-414 of Ln-5 γ2).

In another aspect, the invention provides an antibody or other L5G2D3BP that specifically binds to a portion of Ln-5 γ2 in a region defined by about position 550 to about position 590 thereof, such as a region defined by about position 560 to about position 590 thereof or a region defined by about position 550 to about position 570 thereof (e.g., a region defined by positions 548-570, 548-567, etc.), such as about position 560 to about position 570 thereof. In one aspect, the invention provides an antibody or other binding protein that binds to any such region wherein the N-terminus of the region is or is near Ile548 of Ln-5 γ2 (e.g., within 2-3 amino acid residues thereof).

In still a further aspect, the invention provides an antibody or other binding protein that binds to a portion of Ln-5 γ2 in a region defined by, at least in part, about position 415 to about position 460 thereof (e.g., a region defined by positions 412-462, 414-462, 416-461, 412-440, 414-440, 415-440, 420-460, 420-440, 425-440, 420-462, 435-462, 435-460, 412-444, 414-444, 411-444, 411-443, 411-442, 410-444, 410-443, 410-442, 420-445, 435-444, 435-443, 435-442, or about 440-450 thereof (such as 439-447 and 441-449), etc.). In another particular exemplary aspect, such an antibody or binding protein binds specifically to a region defined by, at least in part, about position 415 to about position 445 of Ln-5 γ2 (e.g., about positions 415-440 of Ln-5 γ2). In a more particular aspect, the invention provides an antibody or other binding protein that binds to such a portion of Ln-5 γ2 wherein the portion comprises Cys442 thereof.

In another additional aspect, the invention provides an antibody or other Ln-5 binding protein that binds to a portion of Ln-5 in a region defined by, at least in part, about position 575 to about position 600 of γ2 (e.g., a region defined by residues 573-602 of γ2).

In yet a further aspect, the invention provides a L5G2D3BP that specifically and/or selectively binds to a sequence that consists essentially of one of SEQ ID NOS:2-21 and 82 (the ability to specifically and/or selectively bind to each such sequence represent an individual aspect of the invention), which can be presented in any suitable form (e.g., as a cyclized peptide, a linear peptide fragment, a portion of a fusion protein, or a portion of γ2 alone or in association with other Ln-5 chains). Such an L5G2D3BP can be, for example, an immunoconjugate or other antibody derivative, a humanized antibody, a fully human antibody, or an antibody fragment.

It will be understood that the characterization of antibodies and other binding proteins of the invention based on particular regions of γ2 can apply to γ2 in any suitable context, such as in the context of free γ2, a fragment of γ2 comprising either DIII only or DIII and N-terminal and/or C-terminal adjacent sequences, a γ2 heterodimer or heterodimer fragment, or heterotrimeric Ln-5 or a fragment thereof. It also will be understood that such regions may define only part of an antigenic determinant, such as in the context of a conformational epitope presented by two or more Ln-5 chains and/or γ2 in combination with a heterologous protein (accordingly such antigenic determinant regions can be said to at least partially define a specific binding target). However, typically such regions will define an independent antigenic determinant region from other non-overlapping antigenic determinant regions specifically described herein, whether they comprise a linear epitope, conformational epitope, or both. Accordingly, in one aspect, the invention provides L5G2D3BPs that are specific for any of the specifically defined antigenic determinant regions of γ2 described herein, specifically (i.e., without inclusion of other noncontiguous regions or amino acids of other chains and/or heterologous proteins).

In another aspect, the invention relates to peptides, such as antibodies, antibody fragments, antibody-related peptides (e.g., diabodies), and derivatives of thereof, that specifically bind to one or more discontinuous epitopes, comprised at least in part (typically entirely) in Ln-5 γ2 DIII. A discontinuous epitope is an epitope defined by two or more amino acid residues that are separated from one another, typically by more than one or even more than several amino acid residues, in the primary sequence of the peptide (i.e., prior to folding of the peptide). Discontinuous epitopes also are often referred to as noncontiguous, noncontinuous, nonlinear, nonsequential, assembled, structural, conformational, or conformation-dependent epitopes. For example, the terms conformational epitope, conformation specific epitope, and discontinuous epitope are used synonymously herein.

One feature of the invention is a Ln-5 γ2 DIII binding peptide, such as an anti-γ2 Dil antibody, that comprises one or more complementarity determining regions (CDRs), a combination of CDRs, or a paratope that confers the ability to specifically bind a human laminin-5 γ2 discontinuous epitope comprising Tyr500 and Pro516 of Ln-5 γ2. In one aspect, such a peptide is provided wherein the epitope also comprises Arg533 of Ln-5 γ2. In another aspect, the epitope also or alternatively comprises Ser526 of Ln-5 γ2.

Another facet of the invention is a Ln-5 γ2 DIII binding peptide that specifically binds a human laminin-5 γ2 structural epitope comprising Tyr500 and His508 of Ln-5 γ2. In a more particular aspect, the epitope also comprises Arg533 of Ln-5, Ser526 of Ln-5 γ2, or both. In another aspect, the invention provides a L5G2D3BP that is specific for a conformational epitope at least partially formed on Ln-5 γ2 that comprises one or more of Tyr 500, Pro516, Arg533, Ser526, and His508.

In a further aspect, the invention provides a L5G2D3BP that binds to a conformation specific epitope that comprises Tyr500 of Ln-5 γ2 and one, two, three, four, five, six, or seven additional Ln-5 amino acids located within about 15 Å of Tyr500 (e.g., about 10-20 Å therefrom or about 5-10 Å therefrom on any side of this amino acid residue).

Additionally, the invention provides a L5G2D3BP that binds to a conformation specific epitope that comprises His508 of Ln-5 γ2 and one, two, three, four, five, six, or seven additional Ln-5 amino acids located within about 15 Å thereof.

The invention also provides a L5G2D3BP that binds to a conformation specific epitope that comprises Arg533 of Ln-5 γ2 and one, two, three, four, five, six, or seven additional Ln-5 amino acids located within about 15 Å thereof.

In an additional aspect, the invention provides a L5G2D3BP that binds to a conformation specific epitope that comprises His508 of Ln-5 and two, three, four, five, six, or seven additional amino acids located within about 15 Å thereof.

In a further aspect, the invention provides a L5G2D3BP that binds to a conformation specific epitope that comprises His543 of Ln-5 γ2 and one, two, three, four, five, six, or seven additional Ln-5 amino acids located within about 15 Å thereof.

In a further aspect, the invention provides a L5G2D3BP that binds to a conformation specific epitope that comprises Ile548 of Ln-5 γ2 and one, two, three, four, five, six, or seven additional Ln-5 amino acids located within about 15 Å thereof.

A L5G2D3BP that specifically binds a human laminin-5 γ2 discontinuous epitope comprising (I) Asp395 of Ln-5 γ2 and (II) (a) Thr412 of Ln-5 γ2, (b) Ile414 of Ln-5 γ2 Thr412 and Ile414 of Ln-5 γ2 also is provided by the invention. In a further aspect, such an L5G2D3BP is characterized by specifically binding a conformational epitope comprising Cys442. In another aspect, such an L5G2D3BP is characterized by binding to Cys442 or a residue in close proximity to Cys442 (e.g., Glu441, Ala443, or both) such that access to Cys442 is effectively blocked by the L5G2D3BP (in other words, Cys442 is within the footprint of the L5G2D3BP). In the latter case, it may be that the epitope comprises Cys442, but Cys442 does not significantly contribute to the avidity of the L5G2D3BP to the epitope, such that Cys442 can be considered not an immunodominant component of the epitope.

In a further aspect, the invention provides a L5G2D3BP that binds to a conformation specific epitope that comprises Asp395 of Ln-5 γ2 and one, two, three, four, five, six, or seven additional Ln-5 amino acids located within about 15 Å thereof.

The invention furthermore provides a L5G2D3BP that binds to a conformation specific epitope that comprises Ile414 of Ln-5 γ2 and one, two, three, four, five, six, or seven additional Ln-5 amino acids located within about 15 Å thereof.

In another aspect, the invention provides a L5G2D3BP that binds to a conformation specific epitope that comprises Thr412 of Ln-5 γ2 and one, two, three, four, five, six, or seven additional Ln-5 amino acids located within about 15 Å thereof.

In still a further aspect, the invention provides a L5G2D3BP that binds to a conformation specific epitope that comprises Cys442 of Ln-5 γ2 and one, two, three, four, five, six, or seven additional Ln-5 amino acids located within about 15 Å thereof.

An additional feature of the invention is a L5G2D3BP that specifically binds to a conformational epitope comprising Ile414 and/or Thr412 of Ln-5 γ2. In a more particular aspect, the invention provides such a L5G2D3BP wherein the epitope comprises one, two, three, four, five, six, or more Ln-5 amino acid residues located within about 15 Å of Ile414 and/or Thr412. In another aspect, the invention provides a L5G2D3BP that comprises a structural epitope comprising Cys442 and either Ile414, Thr412, or both Ile414 and Thr412.

Another inventive feature is a L5G2D3BP that specifically binds to a structural epitope defined by one or more amino acid residues located within a region defined by about position 415 to about position 516 of Ln-5 γ2 (e.g., residues 494-516) and one or more amino acid residues located within a region defined by about position 539 to about position 555 (e.g., residues 539-552) of Ln-5 γ2. In an aspect, the invention provides a L5G2D3BP that specifically binds to residues 494-516 and residues 539-552 with significantly higher affinity and/or avidity than residues 517-540.

As described further herein, the production of antibodies that bind to any of the antigenic determinants/epitopes described herein is another feature of the invention. Thus, for example, in one aspect the invention provides a method for producing a humanized or fully human antibody (or related antibody fragment or derivative of either thereof) that binds to an antigenic determinant in γ2 DIII and competes with mAb 5D5 and/or mAb 6C12 for binding to γ2 Dil, wherein the antigenic determinant comprises one of the above-described selectivity-conferring residues (e.g., Tyr 500, Pro516, Arg533, Ser526, and/or His508) and the antibody is more specific for the antigenic determinant than mAb 5D5 and/or mAb 6C12 and/or binds to the ADR with greater affinity than mAb 5D5 and/or mAb 6C12.

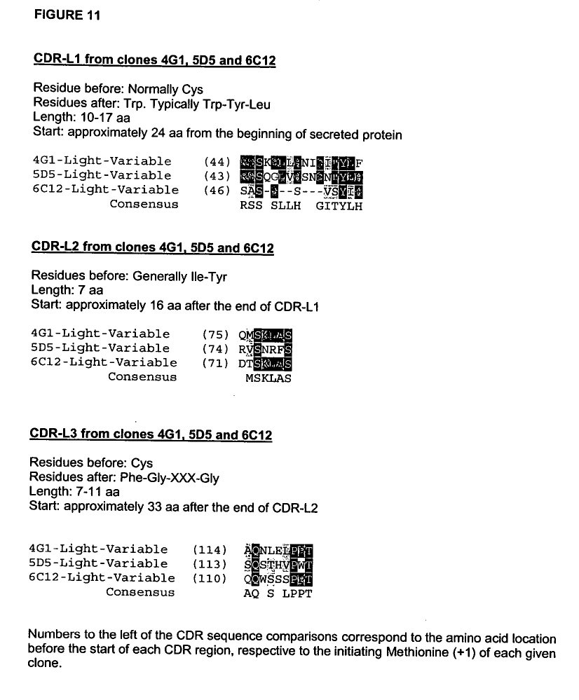

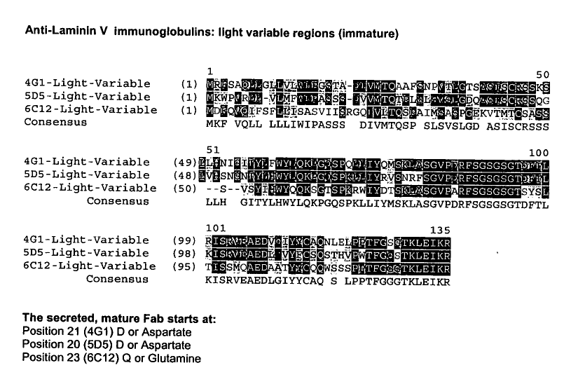

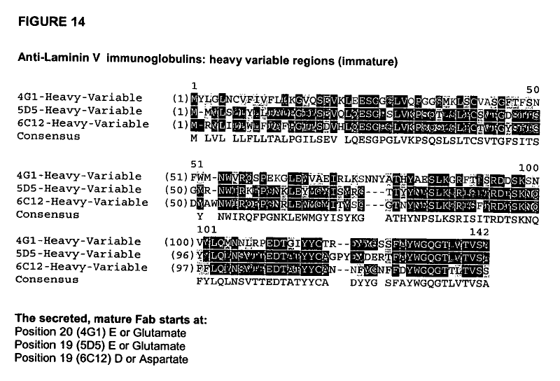

In another aspect of the invention, a L5G2D3BP that comprises one or more CDRs and optionally additional associated sequences that are associated with γ2 DIII binding is provided. In this respect, regions of murine anti-DIII mAbs 4G1, 5D5, and 6C12 that correspond (exactly or nearly exactly) to the CDRs for these antibodies have been identified and compared to identify additional CDR sequence candidates (see, e.g., FIGS. 11-15 and Example 6). Peptides comprising such CDRs and CDR variants are an advantageous feature of this invention. Desirably, such peptides possess sufficient CDR sequences and adequate structure (e.g., by retaining sufficient framework sequences/residues, which typically are variants of the framework sequences/residues normally associated with the CDR(s) or related CDRs (in the case of CDR variants)) to retain at least a significant proportion of the affinity, specificity, and/or selectivity for Ln-5 γ2 DIII as mAbs 4G1, 5D5, and 6C12, as applicable. It may be possible that L5G2D3BPs comprising sufficient amounts of functional CDR variants related to these “parent” CDR sequences actually exhibit greater specificity, selectivity and/or affinity for epitopes recognized by mAbs 4G1, 5D5, or 6C1 2, as applicable. It also can be the case that such L5G2D3BPs will induce less of an immune response in a human patient than these murine antibodies or chimeric antibodies comprising intact and unmodified parent CDR sequences. Moreover, these various CDRs and/or CDR variants can be combined in bispecific antibodies and other antibody-like peptides described herein to provide additional L5G2D3BPs having unique and useful features.

Thus in particularly exemplary aspects, the invention provides L5G2D3BPs that comprise a VL CDR-1 sequence consisting essentially of SEQ ID NO:24, SEQ ID NO:25, or SEQ ID NO:26; A VL CDR-2 sequence consisting essentially of SEQ ID NO:28, SEQ ID NO:29, SEQ ID NO:30, SEQ ID NO:65, SEQ ID NO:66, or SEQ ID NO:67; a VL CDR-3 sequence that consists essentially of SEQ ID NO:32, SEQ ID NO:33, or SEQ ID NO:34; a VH CDR-1 sequence that consists essentially of SEQ ID NO:36, SEQ ID NO:37, or SEQ ID NO:38; a VH CDR-2 sequence that consists essentially of SEQ ID NO:40, SEQ ID NO:41, or SEQ ID NO:42; and/or a VH CDR-3 sequence that consists essentially of SEQ ID NO:44, SEQ ID NO:45, and/or SEQ ID NO:46.

In another exemplary aspect, the invention provides a L5G2D3BP that comprises a VH sequence (comprising the VH CDRs and optionally associated sequences such as framework residues), a VL sequence (similarly comprising the VL CDRs and optional associated sequences), or both, that corresponds essentially to the VH and/or VL sequences of (i.e., consists essentially of the same sequence as the VH and/or VL sequences of) one or more of mAb 4G1, mAb 5D5, and mAb 6C12. In another aspect, the invention provides a L5G2D3BP that also or alternatively comprises a variant of such VL and/or VH sequences. Exemplary L5G2D3BPs comprising such sequences are described elsewhere herein.

In one aspect, the invention provides a L5G2D3BP comprising a complete set of VL CDRs (VL CDR1, CDR2, and CDR3) consisting essentially of SEQ ID NOS:24, 28, and 32, respectively. In another aspect, the invention provides a L5G2D3BP that comprises a complete set of VH CDRs (VH CDR1, CDR2, and CDR3) consisting essentially of SEQ ID NOS:36, 40 (or 65), and 44, respectively. A more particular feature of the invention is a LDBGP that comprises (a) three VL CDRs, which independently consist essentially of SEQ ID NOS:24, 28, and 32 in close proximity to one another (e.g., near the spacing of VL CDRs in a wild-type anti-γ2 DIII antibody) in the LDBGP and (b) three VH CDRs consisting essentially of SEQ ID NOS:36, 40 (or 65), and 44 in close proximity to one another. In one variation on such an aspect, the invention provides a L5G2D3BP that comprises a flexible linker positioned between the VL region and VH region (or included VL and VH CDRS) of the L5G2D3BP. In another variation, the invention provides a L5G2D3BP wherein the VL and VH regions are presented on separate chains in the context of an immunoglobulin fold protein and oriented such that the VL CDR1, CDR2, CDR3 and VH CDR1, CDR2, and CDR3 cooperatively associate to contribute in selectively and/or specifically bind an antigenic determinant on γ2 DIII. In a further aspect, the invention provides a L5G2D3BP that comprises two sets of variable domains (sets of associated VL and VH domains on associated separate chains), such that the L5G2D3BP comprises two identical antigenic determinant binding sites. Any of such L5G2D3BPs described in this paragraph are expected to, at least in part, have similar epitope specificity, selectivity, and/or share other biological/physiochemical characteristics with mAb 4G1, and, accordingly, can be useful in reducing the migration of epithelial-derived cancer cells such as in the context of reducing the invasiveness of such cells.

The basic and novel properties of such VL and VH domain sequences, or component CDRs, in this and other aspects of the invention is the ability to bind γ2 DIII peptides for periods of time sufficient to induce a desired physiological effect (e.g., reduction of epithelial cell migration; binding for sufficient period to deliver a conjugated toxic molecule; reduction of any aspect of cancer progression; etc.). Deletions, insertions, additions, and substitutions, into sequences identified as VL and VH domain sequences herein often may not detrimentally impact such novel and basic properties. It typically is advantageous that the binding is to a portion of γ2 DIII that is relatively similar to that of the binding of a peptide comprising a “parent” sequence from which the VL and/or VH sequence at issue is derived or otherwise similar (e.g., the binding of the VL and/or VH sequence at issue is associated with at least about 50% competition for binding to a γ2 DIII antigenic determinant bound by the parent) and marked by at least about as great affinity for the γ2 DIII antigenic determinant as the parent sequence. It often is even more advantageous where a variant sequence has essentially the same specificity and/or selectivity as the parent and/or greater affinity for a similar region of γ2 DIII.

In another particular aspect, the invention provides a L5G2D3BP comprising VL CDRs consisting essentially of SEQ ID NOS:25, 29, and 33, respectively. In another aspect, the invention provides a L5G2D3BP that comprises VH CDRs consisting essentially of SEQ ID NOS:37, 41 (or 66), and 45 respectively. A more particular feature of the invention is a LDBGP that comprises (a) three VL CDRs, which independently consist essentially of SEQ ID NOS:25, 29, and 33, respectively in close proximity to one another in the LDBGP and (b) three VH CDRs consisting essentially of SEQ ID NOS:37, 41 (or 66), and 45, respectively in close proximity to one another. In one variation on such an aspect, the invention provides a L5G2D3BP that comprises a flexible linker positioned between the VL region and VH region of the L5G2D3BP. In another variation, the invention provides a L5G2D3BP wherein the VL and VH regions are presented on separate chains in the context of an immunoglobulin fold protein and oriented such that the VL CDR1, CDR2, CDR3 and VH CDR1, CDR2, and CDR3 cooperatively associate to contribute in selectively and/or specifically bind an antigenic determinant on γ2 DIII. In a further aspect, the invention provides a L5G2D3BP that comprises two sets of variable domains (sets of associated VL and VH domains on associated separate chains), such that the L5G2D3BP comprises two identical antigenic determinant binding sites. Any of such L5G2D3BPs described in this paragraph are expected to, at least in part, have similar epitope specificity, selectivity, and other characteristics with mAb 5D5, and, accordingly, can be useful in reducing the migration of epithelial-derived cancer cells, such as in reducing the invasiveness of such cancer cells.

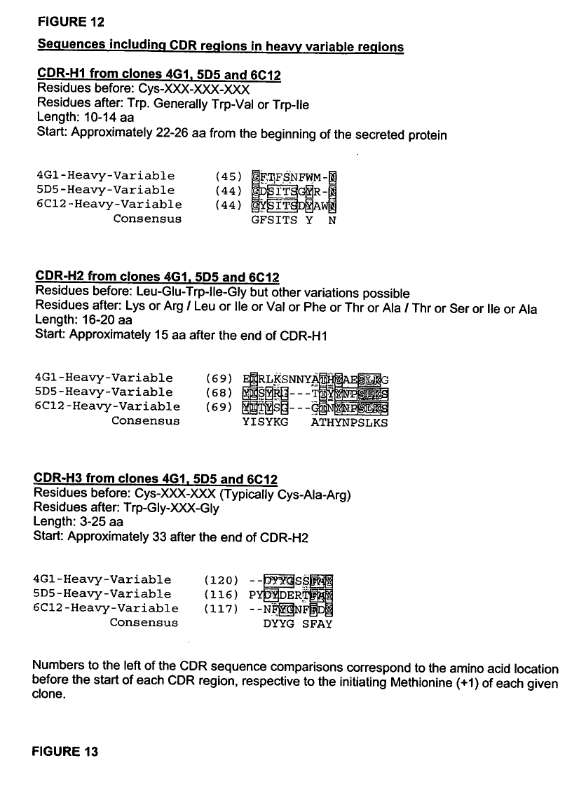

In one aspect, the invention provides a L5G2D3BP comprising VL CDRs consisting essentially of SEQ ID NOS:26, 30, and 34, respectively. In another aspect, the invention provides a L5G2D3BP that comprises VH CDRs consisting essentially of SEQ ID NOS:38, 42 (or 67), and 46, respectively. A more particular feature of the invention is a LDBGP that comprises (a) three VL CDRs, which independently consist essentially of SEQ ID NOS:26, 30, and 34 in close proximity to one another in the LDBGP and (b) three VH CDRs consisting essentially of SEQ ID NOS:38, 42 (or 67), and 46 in close (functionally related) proximity to one another (e.g., in a relationship essentially similar to that presented for CDRs in a wild-type antibody—see FIGS. 11 and 12 for a description of typical distances between CDRs, any near approximation of which is likely to be suitable). In one variation on such an aspect, the invention provides a L5G2D3BP that comprises a flexible linker positioned between the VL region and VH region of the L5G2D3BP. In another variation, the invention provides a L5G2D3BP wherein the VL and VH regions are presented on separate chains in the context of an immunoglobulin fold protein and oriented such that the VL CDR1, CDR2, CDR3 and VH CDR1, CDR2, and CDR3 cooperatively associate to contribute in selectively and/or specifically bind an antigenic determinant on γ2 DIII. In a further aspect, the invention provides a L5G2D3BP that comprises two sets of variable domains (sets of associated VL and VH domains on associated separate chains), such that the L5G2D3BP comprises two identical antigenic determinant binding sites. Any of such L5G2D3BPs described in this paragraph are expected to, at least in part, have similar epitope specificity, selectivity, and other characteristics with mAb 6C12, and, accordingly, can be useful in reducing the migration of epithelial-derived cancer cells such as in the context of reducing the invasiveness of such cells. A feature of some such L5G2D3BPs is the presence of a Tyr residue at the N-terminus of the VL CDR1 (CDR-L1).

L5G2D3BPs comprising CDR sequences having such features or variants thereof (as further described herein) can comprise any suitable number and combination of such VL and VH CDRs. In some cases, less than a full set of VL CDRs and/or VH CDRs can be present in a L5G2D3BP. However, in most cases all of the VL CDRs and VH CDRs will advantageously be present, as typically all of the six CDRs contribute to constructing a surface for epitope-specific binding.

The invention also provides L5G2D3BPs comprising functional variants of the VL region, VH region, or one or more CDRs of mAb 4G1, mAb 5D5, and mAb 6C12. A functional variant of a VL, VH, or CDR used in the context of a L5G2D3BP still allows the L5G2D3BP to retain at least a substantial proportion (at least about 50%, 60%, 70%, 80%, or more) of the affinity/avidity and specificity/selectivity of the parent mAb to which it most nearly relates and in some cases such a L5G2D3BP can be associated with greater affinity, selectivity, and/or specificity than the parent mAb to which it most nearly relates (as determined by overall and/or local amino acid sequence identity).

There are a number of techniques known for generating CDR variants, any suitable technique or combination of which can be used in the context of this invention. Examples of such techniques include the removal of nonessential residues as described in, e.g., Studnicka et al., Protein Engineering, Vol 7, 805-814 (1994) (see also Soderlind et al., Immunotechnology. March 1999;4(3-4):279-85), CDR walking mutagenesis and other artificial affinity maturation techniques (see, e.g., Journal of Molecular Biology, December 1995;254(3):392-403), CDR shuffling techniques wherein typically CDRs are amplified from a diverse set of gene templates optionally comprising synthetic oligonucleotides, the constant regions of the VL, VH, and/or CDRs are amplified, and the various fragments mixed (in single-stranded or double-stranded format) and assembled by polymerase chain reaction (PCR) to produce a set of antibody-fragment encoding gene products carrying shuffled CDR introduced into the master framework, which is amplified using external primers annealing to sites beyond inserted restriction sites to ensure production of full-length products, which are inserted into a vector of choice and used to expressed variant CDR-containing proteins. Appropriate structure can be determined by superimposition of the variant/mimetic structures and those of the parent sequences, e.g., by comparison of NMR solution structures. Additional examples of such methods are provided elsewhere herein.

CDR, VH, and VL sequence variants can exhibit any suitable level of identity to one or more “parent” CDRs, VH sequences, and VL sequences, respectively, such as the CDR, VH, and VL sequences of mAb 4G1, mAb 5D5, and/or mAb 6C12. Typically, a variant sequence that binds to an essentially identical antigenic determinant region as a parent will retain at least about 40% amino acid sequence identity to the parent sequence, such as about 50% or more, about 60% or more, about 70% or more, about 75% or more, about 80% or more, about 85% or more, about 90% or more, or at least about 95% (e.g., about 45-99%, about 55-99%, or about 65-99%) identity to the parent sequence. However, in some cases, particularly with respect to CDR sequences targeted to an essentially identical epitope, variants with even lower levels of identity can be suitable.

CDR, VH, and VL sequence variants that bind to different antigenic determinant regions or a different set (or “profile”) of antigenic determinant regions also can be generated by any of the techniques described elsewhere herein (rational design, mutagenesis, directed evolution, etc.). In such instances, significantly lower levels of amino acid sequence identity to a parent sequence can be expected. For example, in the context of a CDR-L1, CDR-H1, CDR-H2, or CDR H3 variant having a different epitope binding profile from a parent sequence, as little as about 20-30% amino acid sequence identity to a parent CDR sequence may be exhibited in variants that contribute to L5G2D3BP binding of γ2 DIII.

Typically, peptide variants differ from “parent” sequences mostly through conservative substitutions; e.g., at least about 35%, about 50% or more, about 60% or more, about 70% or more, about 75% or more, about 80% or more, about 85% or more, about 90% or more, about 95% or more (e.g., about 65-99%) of the substitutions in the variant are conservative amino acid residue replacements. In the context of this invention, conservative substitutions can be defined by substitutions within the classes of amino acids reflected in one or more of the following three tables:

TABLE 5
Amino Acid Residue Classes for Conservative Substitutions

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE 6
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE 7
Alternative Physical and Functional Classifications of
Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P, and T |
| formation | |
| Flexible residues | E, Q, T, K, S, G, P, D, E, and R |

More conservative substitutions groupings include: valine-leucine-isoleucine, phenylalanine-tyrosine, lysine-arginine, alanine-valine, and asparagine-glutamine. Additional groups of amino acids can also be formulated using the principles described in, e.g., Creighton (1984) Proteins: Structure and Molecular Properties (2d Ed. 1993), W. H. Freeman and Company. In some instances it can be useful to further characterize substitutions based on two or more of such features (e.g., substitution with a “small polar” residue, such as a Thr residue, can represent a highly conservative substitution in an appropriate context).

Substantial changes in function can be made by selecting substitutions that are less conservative than those shown in the defined groups, above. For example, non-conservative substitutions can be made which more significantly affect the structure of the peptide in the area of the alteration, for example, the alpha-helical, or beta-sheet structure; the charge or hydrophobicity of the molecule at the target site; or the bulk of the side chain. The substitutions which generally are expected to produce the greatest changes in the peptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine. Accordingly, these and other nonconservative substitutions can be introduced into peptide variants where significant changes in function/structure is desired and such changes avoided where conservation of structure/function is desired.

Those skilled in the art will be aware of additional principles useful in the design and selection of peptide variants. For example, residues located in surface positions of a peptide typically a strong preference for hydrophilic amino acids. Steric properties of amino acids can greatly affect the local structures that a protein adopts or favors. Proline, for example, exhibits reduced torsional freedom that can lead to the conformation of the peptide backbone being locked in a turn and with the loss of hydrogen bonding, often further resulting in the residue appearing on a surface loop of a protein. In contrast to Pro, Gly has complete torsional freedom about a main peptide chain, such that it is often associated with tight turns and regions buried in the interior of the protein (e.g., hydrophobic pockets). The features of such residues often limit their involvement in secondary structures. However, residues typically involved in the formation of secondary structures are known. For example, residues such as Ala, Leu, and Glu (amino acids without much bulk and/or polar residues) typically are associated with alpha-helix formation, whereas residues such as Val, Ile, Ser, Asp, and Asn can disrupt alpha helix formation. Residues with propensity for beta-sheet structure formation/inclusion include Val and Ile and residues associated with turn structures include Pro, Asp, and Gly. The skilled artisan can consider these and similar known amino acid properties in the design and selection of suitable peptide variants, such that suitable variants can be prepared with only routine experimentation.

Desirably, conservation in terms of hydropathic/hydrophilic properties also is substantially retained in a variant peptide as compared to a parent peptide (e.g., the weight class, hydropathic score, or both of the sequences are at least about 50%, at least about 60%, at least about 70%, at least about 75%, at least about 80%, at least about 85%, at least about 90%, at least about 95%, or more (e.g., about 65-99%) retained). Methods for assessing the conservation of the hydropathic character of residues/sequences are known in the art and incorporated in available software packages, such as the GREASE program available through the SDSC Biology Workbench (see also, e.g., Kyte and Doolittle et al., J. Mol. Biol. 157:105-132(1982); Pearson and Lipman, PNAS (1988) 85:2444-2448, and Pearson (1990) Methods in Enzymology 183:63-98 for a discussion of the principles incorporated in GREASE and similar programs).

It also is advantageous that structure of the variant peptide is substantially similar to the structure of the parent peptide. Methods for assessing similarity of peptides in terms of conservative substitutions, hydropathic properties, weight conservation, and similar considerations are described in e.g., WO 03/048185, WO 03/070747, and WO 03/027246.

The retention of similar residues also or alternatively can be measured by a similarity score, as determined by use of a BLAST program (e.g., BLAST 2.2.8 available through the NCBI). Suitable variants typically exhibit at least about 45%, such as at least about 55%, at least about 65%, at least about 75%, at least about 85%, at least about 90%, at least about 95%, or more (e.g., about 70-99%) similarity to the parent peptide.

As discussed elsewhere herein, other points of variation/divergence between a variant and a parent can be acceptable (e.g., inclusion of non-naturally-occurring amino acids, derivatized amino acids, insertions, deletions, and extensions to the sequence, etc.) provided that such changes do not substantially impair the ability of the variant to bind γ2 DIII as compared to the parent peptide.

L5G2D3BPs provided by the invention can be characterized on the basis of, among other things, including one or more sequences defined by one or more particular formulas of exemplary CDR, VH, and VL variants set forth elsewhere herein.

In this respect, the invention provides, for example, a L5G2D3BP that comprises a VL CDR1 sequence (which also can be referred to as a CDR-L1 sequence) variant that comprises (and typically consists essentially of) a sequence according to the formula Cys Xaa₂ Xaa₃ Ser Xaa₅ Xaa₆ Xaa₇ Xaa₈ Xaa₉ Xaa₁₀ Xaa₁₁ Xaa₁₂ Xaa₁₃ Xaa₁₄ Tyr Xaa₁₆ Xaa₁₇ Trp Tyr Xaa₂₀ (SEQ ID NO:64), wherein(a) Xaa₂ is Arg or Ser; (b) Xaa₃ is Ser or Ala; Xaa₅ is Lys, Gln, or is missing (i.e., is absent or removed); (c) Xaa₆ is Ser or Gly; (d) Xaa₇ is Leu or missing; (e) Xaa₈ is Leu, Val, or is missing; (f) Xaa₉ is His or Ser; (g) Xaa₁₀ is Asn, Ser, or missing; Xaa₁₁ is Ile, Asn, or is missing; (h) Xaa₁₂ is Gly or missing; (i) Xaa₁₃ is Ile, Asn, or Val; () Xaa₁₄ is Thr or Ser; (k) Xaa₁₆ is Leu or Ile; (I) Xaa₁₇ is Phe or His; and (m) Xaa₂₀ is Leu or Gln (Formula I). Unless otherwise specified, the symbol Xaa refers to any suitable amino acid residue. In another aspect, the invention provides a L5G2D3BP that comprises a VL CDR1 variant that consists essentially of a sequence according to a truncated version of Formula I wherein the N-terminal Cys and/or 1, 2, or 3 of the C-terminal residues (i.e., Trp Tyr Xaa₂₀) are absent from the formula.

In another respect, the invention provides a L5G2D3BP that comprises a CDR-LL that comprises (and typically consists essentially of) a sequence according to the formula Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₄ Xaa₁₅ Xaa₁₆ Xaa₁₇ Xaa₁₈ Xaa₁₉ Xaa₂₀, wherein Xaa₂ is Arg or Ser; Xaa₃ is Ser or Ala; Xaa₅ is Lys, Gln, or is missing; Xaa₆ is Ser or Gly; Xaa₇ is Leu or is missing; Xaa₈ is Leu, Val, or is missing; Xaa₉ is His or Ser; Xaa₁₀ is Asn, Ser, or missing; Xaa₁₁ is Ile, Asn, or is missing; Xaa₁₂ is Gly or is missing; Xaa₁₃ is Ile, Asn, or Val; Xaa₁₄ is Thr or Ser; Xaa₁₆ is Leu or Ile; Xaa₁₇ is Phe or His and the sequence is optionally characterized by (a) one or more of the following Xaal is any suitable residue other than Cys or is missing; Xaa₁₈ is any suitable residue other than Trp or is missing; Xaa₁₉ is any suitable residue other than Tyr or is missing; and Xaa₂₀ is Leu, Gln, or is missing and/or (b) Xaa₄ is any suitable residue other than Ser and Xaa₁₅ is Tyr; Xaa₁₅ is any suitable residue other than Tyr and Xaa₄ is Ser; or Xaa₄ is any suitable residue other than Ser and Xaa₁₅ is any suitable residue other than Tyr (Formula II). In another aspect, the invention provides L5G2D3BPs that comprise a CDR-L1 that consists essentially of a formula that corresponds to a truncated Formula II sequence (or truncated version thereof), wherein (i) Xaa₁ and/or (ii) Xaa₁₈, Xaa₁₉, and/or Xaa₂₀ are absent. In another aspect, a L5G2D3BP can comprise a CDR-L1 consisting essentially of a sequence similar to Formula II, but wherein (I) Xaa₂ is any suitable residue other than Arg or Ser; Xaa₃ is any suitable residue other than Ser or Ala; Xaa₆ is any suitable residue other than Ser or Gly; Xaa₉ is any suitable residue other than His or Ser; Xaa₁₄ is any suitable residue other than Thr or Ser; Xaa₁₆ is any suitable residue other than Leu or Ile; and/or Xaa₁₇ is any suitable residue other than Phe or His and/or (II) Xaa₈ is any suitable residue other than Leu or Val; Xaa₁₀ is any suitable residue other than Asn or Ser; Xaa₁₁ is any suitable residue other than Ile or Asn; and/or Xaa₁₂ is any suitable residue other than Gly.

Another facet of the invention is embodied in various L5G2D3BPs that comprise a CDR-L1 variant sequence that comprises (and typically consists essentially of) a sequence according to the formula Cys Xaa₁ Xaa₂ Ser Xaa₃ Xaa₄ Leu₇ Xaa₅ Xaa₆ Xaa₇ Xaa₈ Gly₁₂ Xaa₉ Xaa₁₀ Try Xaa₁₁ Xaa₁₂ Trp Tyr Xaa₁₃ (SEQ ID NO:48), wherein (I) Xaa₃ is Lys, Gln, or is missing; Xaa₅ is Leu, Val or is missing; Xaa₇ is Asn, Ser, or is missing; Xaa₈ is Ile, Asn, or is missing; Xaa₉ is Ile, Asn, or Val; and Xaal, Xaa₂, Xaa₄, Xaa₆, (II) Xaa₁₀-Xaa₁₃ are defined by the residue from a corresponding position in any one of SEQ ID NOS:24-26 except for one or more of the following:(a) Xaa₁ is any suitable residue other than Arg or Ser; (b) Xaa₂ is any suitable residue other than Ser or Ala; (c) Xaa₄ is any suitable residue other than Ser or Gly (such as a suitable small residue); (d) Xaa₆ is any suitable residue other than His or Ser (such as a suitable polar residue); (e) Xaa₁₀ is any suitable residue other than Thr or Ser (such as a suitable polar residue); (f) Xaa₁₁ is any suitable residue other than Leu or Ile (such as a suitable aliphatic uncharged residue); (g) Xaa₁₂ is any suitable residue other than Phe or His (such as a suitable different cycloalkenyl residue);(h) Xaa₁₃ is any suitable residue other than Leu or Gln; and (III) optionally Leu₇ or Gly₁₂ is missing or replaced by another suitable amino acid residue (Formula III).

In still another aspect, the invention provides a L5G2D3BP that comprises a CDR-L1 sequence that comprises (and typically that consists essentially of) a sequence according to the formula Cys Arg Ser Ser Xaa₅ Ser Leu Leu His Xaa₁₀ Xaa₁₁ Gly Ile Thr Tyr Leu His Trp Tyr Leu (SEQ ID NO:27), wherein (I) (a) Xaa₅ is Lys, Gln, or is missing; (b) Xaa₁₀ is Asn, Ser, or is missing; or (c) Xaa₁₁ is Ile, Asn, or is missing or (II) one or more of Xaa₅, Xaa₁₀, and Xaa₁₁ represent suitable residues other than those defined respectively in (a), (b), and/or (c), the other members of Xaa₅, Xaa₁₀, and Xaa₁₁ being defined according to (a)-(c) (Formula IV). In a variation on this aspect, the invention further provides L5G2D3BPs that comprise a CDR-L1 consisting essentially of a truncated Formula IV sequence, wherein the N-terminal Cys residue and 1, 2, or 3 of the C-terminal residues thereof are absent.

In another particular aspect, the invention provides a L5G2D3BP that comprises a CDR-L1 that comprises (and typically that consists essentially of) a sequence according to the formula Cys Xaa₂ Xaa₃Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Trp Tyr Xaa₂₀, wherein (a) Xaa₁₃ is Ile, Asn, or Val; Xaa₁₄ is Thr or Ser; Xaa₁₆ is Leu or Ile; Xaa₁₇ is Phe or His; Xaa₅ is Lys, Gln, or is missing; Xaa₈ is Leu, Val, or is missing; Xaa₁₀ is Asn, Ser, or is missing; Xaa₁₁ is Ile, Asn, or is missing; Xaa₂ is Arg or Ser; Xaa₃ is Ser or Ala; Xaa₆ is Ser or Gly; Xaa₉ is His or Ser; Xaa₇ is any suitable residue other than Leu or is missing; Xaa₁₂ is any suitable residue other than Gly or is missing; Xaa₂₀ is Leu, Gln, or is missing; and (b) one or both of the following (I) Xaa₄ is any suitable residue other than Ser; (II) Xaa₁₅ is any suitable residue other than Tyr (Formula V). In a variation on this aspect, the invention provides L5G2D3BPs comprising a CDR-L1 that consists essentially of a sequence similar to Formula V but lacking the N-terminal Cys residue and/or 1, 2, or 3 of the C-terminal residues of the formula (i.e., a “truncated” version of Formula V). In another aspect, the invention provides a L5G2D3BP that comprises a CDR-L1 that consists essentially of a sequence defined by Formula V, or a truncated version of Formula V, except for one or more of the following: Xaa₅ is any suitable residue other than Lys or Gln; Xaa₈ is any suitable residue other than Leu or Val; Xaa₁₀ is any suitable residue other than Asn or Ser; and Xaa₁₁ is any suitable residue other than Ile or Asn. Formula V and Formula V-like sequences also or alternatively can be characterized by one or more of the following: Xaa₇ is any suitable residue other than Leu; Xaa₁₂ is any suitable residue other than Gly; and Xaa₂₀ is any suitable residue other than Leu or Gln. In another aspect, Xaa₁₇ in any of the foregoing Formula V and Formula V-like sequences represents any suitable residue other than Phe or His.

In yet a further aspect, the invention provides a L5G2D3BP that comprises a variant VL CDR1 sequence that comprises (and typically that consists essentially of) a sequence according to the formula Xaa₁ Xaa Xaa₃ Xaa₄ Xaa₅ Xaa Xaa Xaa₈ Xaa Xaa₁₀ Xaa₁₁ Xaa₁₂ Xaa₁₃ Xaa₁₄ Xaa₁₅ Xaa₁₆ Xaa Xaa₁₈ Xaa₁₉ Xaa, wherein (a) Xaa represents any suitable amino acid residue; (b) Xaa₃ is Ser, Ala, or Thr; Xaa₅ is Lys, Gln, or is missing/absent; Xaa₈ is Leu, Val, or is missing; Xaa₁₀ is Asn, Ser, or is missing; Xaa₁₁ is Ile, Asn, or is missing; Xaa₁₂ is Gly or missing; Xaa₁₃ is Ile, Asn, or Val; Xaa₁₄ is a hydrophilic uncharged residue; and Xaa₁₆ is an aliphatic uncharged residue, and (c) Xaa₁, Xaa₄, Xaa₁₅, and Xaa₁₈, Xaa₁₉, are defined by the corresponding residues in SEQ ID NO:64, except that the variant VL CDR1 sequence differs from SEQ ID NO:64 in one or more of the following: Xaa₁ is any suitable residue other than Cys; Xaa₄ is any suitable amino acid residue other than Ser; Xaa₁₅ is any suitable residue other than Tyr; Xaa₁₈ is any suitable residue other than Trp; and Xaa₁₉ is any suitable residue other than Tyr (Formula VI).

Desirably, a variant VL CDR1 sequence comprised within the above-described formulas, will exhibit at least about 40% (e.g., about 50% or more (such as about 55-99%), about 65% or more, about 75% or more, or about 90% or more) amino acid sequence identity to a sequence provided under SEQ ID NO:27 and/or the VL CDR1 sequences of mAb 4G1, mAb 5D5, and/or mAb 6C12.

A suitable amino acid residue substitution in the context of a CDR variant is any amino acid residue that permits the CDR to interact with the γ2 DIII epitope to which the parent CDR is selective/specific and to cooperatively associate with other parent CDRs and/or variant CDRs similarly specific/selective for that epitope. Similar principles apply to suitable substitutions, additions, insertions, and deletions in the context of variants of CDR associated sequences, VH sequences, and VL sequences. Factors influencing the selection of a suitable amino acid sequence substitution can include the impact of the residue on the conformation of the CDR (e.g., retention of CDR loop structure and flexibility) and the ability to engage in noncovalent interactions (e.g., Van der Waals interactions, hydrogen bonding interactions, ionic interactions, and/or other interactions characteristic of epitope-variable region binding) with the epitope and/or other similar CDRs in a manner similar to or advantageous over the replaced residue in the parent CDR. Given the relatively few residues that are selected for modification in the CDR formulas described herein, and the number of techniques available for rapid screening of numerous sequences (e.g., phage display techniques), one or more suitable amino acid residue substitutions should be identifiable for any variable residue in the CDR variants described herein using no more than routine experimentation. Applications of similar principles allow variants also or alternatively defined by insertions, deletions, or additions to likewise be identified.

In another aspect, the invention provides a L5G2D3BP comprising a CDR-L2 (VL CDR2) sequence variant that comprises (and typically consists essentially of) a sequence according to the formula Ile Tyr Xaa₃ Xaa₄ Ser Xaa₆ Xaa₇ Xaa₈ Ser (SEQ ID NO:68), wherein Xaa₃ is Gln, Arg, or Asp; Xaa₄ is Met, Val, or Thr; Xaa₆ is Lys or Asn; Xaa₇ is Leu or Arg; and Xaa₈ is Ala or Phe (Formula VIl). In a more particular aspect, the invention provides a L5G2D3BP comprising a CDR-L2 that consists essentially of a Formula VII sequence wherein following the C-terminal Ser is the sequence Gly Val Pro, a portion thereof (i.e., Gly Val or simply Gly), or a variant thereof wherein at least one of these residues are retained (e.g., Gly Xaa Pro, Xaa Val Pro, or Xaa Val Xaa, etc.). Xaa refers to any suitable amino acid residue (typically naturally occurring L-amino acid residue) herein unless otherwise specified.

In another aspect, the invention provides a L5G2D3BP comprising a variant CDR-L2 sequence that comprises (and typically consists essentially of) a sequence according to the formula Xaa₁ Xaa₂ Xaa₃ Xaa₄ Xaa₅ Xaa₆ Xaa₇ Xaa₈ Xaa₉, wherein Xaa₁₀-Xaa₉ are defined according to the residues found in the corresponding positions in SEQ ID NO:68 with the exception that the variant CDR-L2 sequence differs from SEQ ID NO:68 by one or more of the following Xaa₁ is any suitable residue other than Ile; Xaa₂ is any suitable residue other than Tyr; Xaa₃ is Gln, Arg, or Asp; Xaa₄ is Met, Val, or Thr; Xaa₅ is any suitable residue other than Ser; Xaa₆ is any suitable amino acid residue other than Lys and Asn; Xaa₇ is any suitable residue other than Leu and Arg; Xaa₈ is any suitable residue other than Ala and Phe; and Xaa₉ is any suitable residue other than Ser (Formula VIII). In another aspect, a L5G2D3BP can comprise a CDR-L2 that consists essentially of a sequence according to a formula similar to Formula VIII, but wherein Xaa₆ is Lys or Asn; Xaa₇ is Leu or Arg; and/or Xaa₈ is Ala or Phe. In another aspect, a L5G2D3BP can comprise a CDR-L2 that consists essentially of a Formula VIII or Formula VIII-like sequence wherein Xaal and/or Xaa₂ thereof are (a) Ile and/or Tyr, respectively, (b) either or both absent, or (c) characterized by a combination of (a) and (c) (e.g., Xaal is absent and Xaa₂ is Tyr). In a different but related aspect, the invention provides a L5G2D3BP comprising a Formula VIII or Formula VIII-like sequence wherein Xaa₃ and/or Xaa₄ differ from the corresponding residues in any one of SEQ ID NOS:65-67.

In a further aspect, the invention provides a L5G2D3BP that comprises a CDR-L2 (VL CDR2) that comprises (and typically that consists essentially of) a sequence according to the formula Tyr Xaa₁ Met Xaa₂ Lys Leu Ala Xaa₄ Gly Val Pro (SEQ ID NO:49), wherein (a) Xaa₂ and Xaa₄ are either independently or both any suitable residue other than Ser and (b) Xaa₁ is Gln, Arg, or Asp (Formula IX). In a variation on this aspect, the invention provides a L5G2D3BP comprising a sequence that consists essentially of a truncated Formula IX sequence, wherein 1, 2, or 3 of the C-terminal residues of the sequence (e.g., Gly₉ Val₁₀ Pro₁₁) are missing and/or 1 or 2 of the N-terminal residues (i.e., Ile₁ and/or Tyr₂) are missing.

In another aspect, the invention provides a L5G2D3BP that comprises a CDR-L2 that comprises (and typically consists essentially of) a sequence according to the formula Ile-Tyr-Xaa₁-Met-Ser-Lys-Leu-Ala-Ser-Gly-Val-Pro (SEQ ID NO:31), wherein Xaa₁ is Gln, Arg, or Asp (Formula X). In a variation on this aspect, the invention provides a L5G2D3BP comprising a sequence consisting essentially of sequence according to a truncated version of Formula X, wherein 1 or 2 of the N-terminal residues and/or 1, 2, or 3 of the C-terminal residues thereof are missing or substituted with other suitable amino acid residue(s).

In another aspect, the invention provides a L5G2D3BP that comprises a CDR-L3 (VL CDR3) variant sequence that comprises (and typically that consists essentially of) a sequence according to the formula Cys Xaa₂ Gln Xaa₄Xaa₅ Xaa₆ Xaa₇ Pro Xaa₉ Thr Phe Gly Xaa₁₃ Xaa₁₄ (SEQ ID NO:77), wherein Xaa₂ is Ala, Ser, or Gln; Xaa₄ is Asn, Ser, or Trp; Xaa₅ is Leu, Thr, or Ser; Xaa₆ is Glu, His, or Ser; Xaa₇ is Leu, Val, or Ser; Xaa₉ is Pro or Trp; Xaa₁₃ is Ser or Gly; and Xaa₁₄ is Gly or Ser (Formula XI). In an additional aspect, a L5G2D3BP can comprise a CDR-L3 consisting essentially of a truncated Formula XI sequence, wherein the N-terminal Cys and/or Phe₁₁-Xaa₁₄ or Gly₁₂-Xaa₁₄ thereof is/are missing. A Ln-5 γ2 DIII-binding peptide can further be characterized by comprising a CDR-L3 consisting essentially of a sequence similar to Formula XI or truncated Formula XI sequence, wherein Xaa₉ represents any suitable amino acid residue other than Pro or Trp. In another aspect, a L5G2D3BP can comprise a CDR-L3 that consists essentially of a Formula XI-like or truncated Formula XI-like sequence wherein also or alternatively Xaa₁₃ is any suitable residue other than Ser and Gly; Xaa₁₄ is any suitable residue other than Gly or Ser; or Xaa₁₃ and Xaa₁₄ are any suitable residues other than Ser and Gly.

In another aspect, the invention provides a L5G2D3BP that comprises a CDR-L3 that comprises (and typically that consists essentially of) a sequence according to the formula Xaa₁ Xaa₂ Xaa₃ Xaa₄Xaa₅ Xaa₆ Xaa₇ Xaa₈ Xaa₉ Xaa₁₀ Xaa₁₁ Xaa₁₂ Xaa₁₃ Xaa₁₄, wherein Xaa₂ is Ala, Ser, or Gln; Xaa₄ is Asn, Ser, or Trp; Xaa₅ is Leu, Thr, or Ser; Xaa₆ is Glu, His, or Ser; Xaa₇ is Leu, Val, or Ser; Xaa₉ is Pro or Trp; and one the sequence is further characterized by one or more of the following Xaa₃ is any suitable residue other than Gln; Xaa₁₃ is Ser, Gly, or is missing; Xaa₁₄ is Gly, Ser, or is missing; Xaa₁ is any suitable residue other than Cys or is missing; Xaa₁₁ is any suitable residue other than Phe or is missing; and Xaa₁₂ is any suitable residue other than Gly or is missing; Xaa₈ is any suitable residue other than Pro; and Xaa₁₀ is any suitable residue other than Thr (Formula XII).

In still another aspect, the invention provides a L5G2D3BP that comprises a CDR-L3 that comprises (and typically that consists essentially of) a sequence according to the formula Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄,wherein Xaa₂ is Ala, Ser, or Gln; Xaa₄ is Asn, Ser, or Trp; Xaa₅ is Leu, Thr, or Ser; Xaa₆ is Glu, His, or Ser; Xaa₇ is Leu, Val, or Ser; and Xaa₉, Xaa₃, Xaa₈, Xaa₁₀, Xaa₁₃, Xaa₁₄, Xaa₁, Xaa₁₁, and Xaa₁₂ are defined by corresponding residues in any one of SEQ ID NOS:32-34 except for one or more of: Xaa₉ is any suitable residue other than Pro or Trp; Xaa₃ is any suitable residue other than Gln; Xaa₈ is any suitable residue other than Pro; Xaa₁₀ is any suitable residue other than Thr; Xaa₁₃ is any suitable residue other than Ser or Gly or is missing; Xaa₁₄ is any suitable residue other than Gly or Ser or is missing; Xaa₁ is any suitable residue other than Cys or is missing; Xaa₁₁ is any suitable residue other than Phe or is missing; and Xaa₁₂ is any suitable residue other than Gly or is missing (Formula XIII).

Another feature of the invention is a L5G2D3BP that comprises a CDR-L3 variant sequence that comprises (and typically that consists essentially of) a sequence according to the formula Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄, wherein Xaa₂ is Ala, Ser, or Gln; Xaa₄ is Asn, Ser, or Trp; Xaa₅ is Leu, Thr, or Ser; Xaa6 is Glu, His, or Ser; and Xaa₇ is Leu, Val, or Ser and Xaa₁, Xaa₃, and Xaa₈-Xaa₁₄ are defined by the corresponding residues in SEQ ID NO:35 except for one or more of the following: Xaa₁ is any suitable residue other than Cys; Xaa₃ is any suitable residue other than Gln; Xaa₈ is any suitable residue other than Pro; Xaa₉ is any suitable residue other than Pro or Trp; Xaa₁₀ is any suitable residue other than Thr; Xaa₁₁ is any suitable residue other than Phe; Xaa₁₂ is any suitable residue other than Gly; Xaa₁₃ is any suitable residue other than Ser and Gly; and Xaa₁₄ is any suitable residue other than Ser or Gly (Formula XIV).

In another aspect, the invention provides a L5G2D3BP comprising a CDR-L3 that comprises (and typically that consists essentially of) a sequence similar to any one of Formulas XII-XIV, wherein the N-terminus of the sequence is a Cys residue and/or the C-terminus of the sequence is defined by the formula Phe Gly Xaa₁ Xaa₂, wherein Xaa₁ and Xaa₂ typically both represent (independently) Ser or Gly residues. In a similar aspect, the invention provides a L5G2D3BP comprising a CDR-L3 that comprises (and typically that consists essentially of) a sequence similar to Formula XI wherein the C-terminus of the sequence is Phe-Gly and the sequence optionally can be associated with any combination of Ser and Gly residues located adjacent to the C-terminus of the sequence.

The invention also provides a L5G2D3BP that comprises a CDR-L3 (VL CDR3) that comprises (and typically that consists essentially of) a sequence according to the formula Cys Ala Gln Xaa₂ Ser Xaa₃ Leu Xaa₄ Pro Xaa₅ Phe Gly Gly Gly (SEQ ID NO:82), wherein (I) Xaa₂ is Asn, Ser, or Trp and Xaa₃ is Glu, His, or Ser and (II) the formula is characterized by one or more of the following (a) Xaa₄ is any suitable residue other than Pro and Xaa₅ is Thr; (b) Xaa₅ is any suitable residue other than Thr and Xaa₄ is Pro; or (c) Xaa₄ is any suitable residue other than Pro and Xaa₅ is any suitable residue other than Thr (Formula XV).

In another aspect, the invention provides a L5G2D3BP that comprises a CDR-L3 that comprises (and typically that consists essentially of) a sequence according to the formula Cys Ala Gln Xaa₁ Ser Xaa₂ Leu Pro Pro Thr Phe Gly Gly Gly (SEQ ID NO:35), wherein Xaa₁ is Asn, Ser, or Trp and Xaa₂ is Glu, His, or Ser (Formula XVI). In a further variation, the invention provides a L5G2D3BP that comprises a CDR-L3 that consists essentially of a sequence according to a formula identical to Formula XVI or truncated Formula XVI sequence except that one or both of Xaa₁ and Xaa₂ differ from the corresponding position(s) of any one of SEQ ID NOS:32-34.

In another facet, the invention provides a L5G2D3BP that comprises a CDR Hi sequence that comprises (and typically that consists essentially of) a sequence according to the formula Cys Xaa₂ Xaa₃ Xaa₄ Gly Xaa₆ Xaa₇ Xaa₈ Xaa₉ Xaa₁₀ Xaa₁₁ Xaa₁₂ Xaa₁₃ Xaa₁₄ Xaa₁₅ Trp Xaa₁₇, wherein Xaa₂ is Val, Ser, or Thr; Xaa₃ is Ala or Val; Xaa₄ is Ser or Thr; Xaa₆ is Phe, Asp, or Tyr; Xaa₇ is Thr or Ser; Xaa₈ is Phe or Ile; Xaa₉ is Ser or Thr; Xaa₁₀ is Asn or Ser; Xaa₁₁ is Phe, Gly, or Asp; Xaa₁₂ is Trp or Tyr; Xaa₁₃ is Met, Arg, or Ala; Xaa₁₄ is Asn or Trp; Xaa₁₅ is Asn or is absent; and Xaa₁₇ is Val or Ile (Formula XVII).

In a different aspect, the invention provides a L5G2D3BP comprising a CDR H1 variant sequence comprising (and typically consisting essentially of) a sequence according to the formula Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇, wherein X₂ is Val, Ser, or Thr; X₆ is Phe, Asp, or Tyr; Xaa₁₁ is Phe, Gly, or Asp; Xaa₁₃ is Met, Arg, or Ala; and Xaa₁, Xaa₃-Xaa₅, Xaa₇-Xaa₁₂, and Xaa₁₄-Xaa₁₇ are defined according to a corresponding residue in any one of SEQ ID NOS:73-77 with the exception that these residues differ from these sequences in one or more of the following: Xaa₁ is any suitable residue other than Cys; X₃ is any suitable residue other than Ala or Val (typically a small residue and/or a hydrophobic residue); Xaa₄ is any suitable residue other than Ser or Thr (typically a small and/or polar residue); Xaa₅ is any suitable residue other than Gly; Xaa₇ is any suitable residue other than Ser or Thr (typically a small and/or polar residue); Xaa₈ is any suitable residue other than Phe or Ile; Xaa₉ is any suitable residue other than Ser or Thr (typically a small and/or polar residue); Xaa₁₀ is any suitable residue other than Asn or Ser (typically a small residue and/or a residue involved in turn formation); Xaa₁₂ is any suitable residue other than Trp or Tyr (typically a cycloalkenyl-associated residue and/or a hydrophobic residue); Xaa₁₄ is any suitable residue other than Asn or Trp; Xaa₁₅ is any suitable residue other than Asn or is absent/missing; Xaa₁₆ is any suitable residue other than Trp; and Xaa₁₇ is any suitable residue other than Val or Ile (typically an aliphatic and/or hydrophobic residue) (Formula XVIII). In another aspect, the invention provides a L5G2D3BP comprising a CDR H1 sequence consisting essentially of a sequence that is identical to Formula XVIII except in that Xaa₃, Xaa₄, Xaa₇, Xaa₈, Xaa₉, Xaa₁₀, Xaa₁₂, Xaa₁₄, Xaa₁₅, and Xaa₁₇ represent residues found in correspondin positions in one or more of SEQ ID NOS:73-75. In another aspect, the invention provides a L5G2D3BP comprising a CDR H1 sequence consisting essentially of a Formula XVIII sequence or such a Formula XVIII-like sequence wherein Xaa₁₅ represents a suitable amino acid residue other than Asn.

Another facet of the invention is a L5G2D3BP that comprises a VH CDR1 (CDR-H1) that comprises (and typically consists essentially of) a sequence according to the formula Cys Xaa₁ Val Thr Xaa₂ Xaa₃ Ser Ile Thr Ser Xaa₄ Tyr Xaa₅ Asn Xaa6 Trp Ile (SEQ ID NO:51), wherein Xaa₁ is Ser, Val, or Thr; Xaa₃ is a Phe, Asp, or Tyr residue; Xaa₄ Phe, Gly, or Asp; Xaa₅ is a Met, Arg, or Ala; Xaa₂ optionally is any suitable residue other than Gly; and Xaa₆ is Asn, another suitable residue, or is missing (Formula XIX). In another facet, the invention provides a L5G2D3BP that comprises (and typically consists essentially of) a sequence according to the formula Cys Ser Val Thr Gly Phe Ser Ile Thr Ser Xaa₁ Tyr Xaa₂ Asn Xaa₃ Trp Ile (SEQ ID NO:39) (Formula XIXa). In a particular aspect, the invention provides such a L5G2D3BP wherein Xaa₁ is Phe, Gly, or Asp; Xaa₂ is Met, Arg, or Ala; and/or Xaa₃ is Asn, another suitable residue, or is missing (Formula XIXb). In another aspect the invention provides L5G2D3BPs comprising a CDR H1 sequence that consists essentially of a truncated Formula XIX sequence or similar sequence (e.g., a Formula XIXa sequence), wherein 1, 2, 3, or 4 of the N-terminal residues and/or 1, 2, or 3 of the C-terminal residues of the Formula XIX sequence are absent (similar truncations are contemplated with respect to all of the above-described CDR H1 sequences).

Any of the above-described CDR H1 variant sequences typically exhibit at least about 40% amino acid sequence identity (such as about 50% or more, about 60% or more, about 70% or more, about 80% or more, or at least about 90% amino acid sequence identity (e.g., about 65-99% identity)) to a portion of one or more of SEQ ID NOS:73-75.

In another aspect, the invention provides a L5G2D3BP that comprises a CDR H1 variant comprising (and typically consists essentially of) a sequence according to the formula Cys Ser Val Thr Gly Phe Ser Ile Thr Ser Xaa₁ Tyr Xaa₂Asn Xaa₃Trp Ile (SEQ ID NO:76), wherein Xaa₁ is Phe, Gly, or Asp; Xaa₂ is Met, Arg, or Ala; and Xaa₃ is Asn or absent (Formula XX). In another aspect, the invention provides a L5G2D3BP comprising a CDR H1 that comprises a truncated Formula XX sequence wherein 1, 2, 3, or 4 of the N-terminal and/or 1 or 2 of the C-terminal residues in the sequence are absent. In another aspect, the invention provides a L5G2D3BP comprising a CDR H1 sequence consisting essentially of a sequence according to identical to Formula XX or a truncated version thereof (as described in the preceding sentence) but for one or more of the following: Gly₅ is substituted with any suitable amino acid residue (subscripts with respect to residues refer to the position of the indicated residue in the subject sequence, here the sequence of Formula XIII); Phe₆ is substituted with either Asp or Tyr; Ser₇ is substituted with Thr; Ile₈ is substituted with Phe; Thrg is substituted with Ser; Serlo is substituted with Asn; Tyr₁₂ is substituted with Trp; and/or Asn₁₄ is substituted with Trp.

In another aspect, the invention provides a L5G2D3BP that comprises a CDR-H2 that consists essentially of a sequence according to Leu Glu Trp Met Gly Tyr Ile Ser Tyr Lys Gly Xaa₁ Xaa₂ Xaa₃ Ala Thr His Tyr Asn Pro Ser Leu Lys Ser Arg Ile Ser (SEQ ID NO:43), wherein (a) Xaa₁ is Asn or is missing; (b) Xaa₂ is Asn or is missing; and (c) Xaa₃ is Tyr or is missing (Formula XXI). Such a CDR-H2 variant, and other CDR-H2 variants described above, desirably retain at least about 40% amino acid sequence identity, such as about 50% or more, about 60% or more, about 70% or more, about 80% or more, about 90% or more (e.g., about 65-99%) amino acid sequence identity to one or more of SEQ ID NOS:40-42.

In another aspect, the invention provides a binding protein comprising a CDR-H2 that consists essentially of a sequence defined by a formula identical to Formula XXI but for Xaal-Xaa₃ represent one or more amino acids that differ(s) from the sequence Asn-Asn-Tyr by substitution with suitable amino acid residue(s). In another aspect, the invention provides a L5G2D3BP comprising a VH CDR2 sequence that consists essentially of a sequence according to a formula similar to Formula XXI or foregoing Formula XXI-like formula wherein 1, 2, 3, 4, or 5 of the N-terminal residues and/or 1, 2, or 3 of the C-terminal residues of Formula XXI are absent. In a variation on this aspect, the invention provides a L5G2D3BP comprising a VH CDR2 according to a formula essentially identical to Formula XXI or the foregoing (above-described) Formula XXI-like sequence (or truncated version of either thereof) except in that one or more of Xaa₇, Xaa₁₆, Xaa₁₈, Xaa₂₁, Xaa₂₂, Xaa₂₃, and Xaa₂₅ retain the residue found in the corresponding position in SEQ ID NO:43.

Another feature of the invention is L5G2D3BPs that comprise a CDR-H2 sequence that comprises (and typically that consists essentially of) a sequence according to the formula Leu Glu Xaa₃ Xaa₄ Xaa₅ Xaa₆ Ile Xaa₈ Xaa₉ Xaa₁₀ Xaa₁₁ Xaa₁₂ Xaa₁₃ Xaa₁₄ Xaa₁₅ Thr Xaa₁₇ Try Xaa₁₉ Xaa₂₀ Ser Leu Lys Xaa₂₄ Arg Xaa₂₆ Xaa₂₇ (SEQ ID NO:78), wherein Xaa₃ is Trp or Tyr; Xaa₄ is Val or Met; Xaa₅ is Ala or Gly; Xaa₆ is Glu or Tyr; Xaa₈ is Arg, Ser, or Thr; Xaa₉ is Leu or Tyr; Xaa₁₀ is Lys, Arg, or Ser; Xaa₁, is Ser or Gly; Xaa₁₂ is Asn or is missing; Xaa₁₃ is Asn or is missing; Xaa₁₄ is Tyr or is missing; Xaa₁₅ is Ala, Thr, or Gly; Xaa₁₇ is His, Tyr, or Asn; Xaa₁₉ is Ala or Asn; Xaa₂₀ is Glu or Pro; Xaa₂₄ is Gly or Ser; Xaa₂₆ is Phe or Ile; and Xaa₂₇ is Thr or Ser (Formula XXII). Such variants and other CDR-H2 variants typically exhibit at least about 40%, such as about 50% or more, about 60% or more, about 70% or more, about 80% or more, or about 90% or more (e.g., about 65-99%) identity to one or more of SEQ ID NOS:40-42.

In a further aspect, the invention provides a L5G2D3BP that comprises a CDR-H2 that comprises (and typically consists essentially of) a sequence according to the formula Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Xaa₁₈ Xaa₁₉ Xaa₂₀ Xaa₂₁ Xaa₂₂ Xaa₂₃ Xaa₂₄ Xaa₂₅ Xaa₂₆ Xaa₂₇, wherein (I) Xaa₈ is Arg, Ser, or Thr; Xaa₁₀ is Lys, Arg, or Ser; Xaa₁₅ is Ala, Thr, or Gly; Xaa₁₇ is His, Tyr, or Asn; Xaa₁₂ is Asn or is missing; Xaa₁₃ is Asn or is missing; Xaa₁₄ is Tyr or is missing; Xaa₃ is Trp, Tyr, or is missing; Xaa₄ is Val, Met, or is missing; Xaa₅ is Ala or Gly; Xaa₆ is Glu or Tyr; Xaa₉ is Leu or Tyr; Xaa₁₁ is Ser or Gly; Xaa₁₉ is Ala or Asn; Xaa₂₀ is Glu or Pro; Xaa₂₄ is Gly or Ser; Xaa₂₆ is Phe or Ile; Xaa₂₇ is Thr or Ser; and (II) Xaa₁, Xaa₂, Xaa₇, Xaa₁₆, Xaa₁₈, Xaa₂₁, Xaa₂₂, Xaa₂₃, and Xaa₂₅ are defined respectively by a residue in a corresponding position in an alignment of SEQ ID NOS:40-42 except in that one or more of these positions can vary from SEQ ID NOS:40-42 by one or more of the following: Xaa₁ is any suitable residue other than Leu or is missing; Xaa₂ is any suitable residue other than Glu or is missing; Xaa₇ is any suitable residue other than Ile; Xaa₁₆ is any suitable residue other than Thr; Xaa₁₈ is any suitable residue other than Tyr; Xaa₂₁ is any suitable residue other than Ser; Xaa₂₂ is any suitable residue other than Leu; Xaa₂₃ is any suitable residue other than Lys; and Xaa₂₅ is any suitable residue other than Arg or is missing (Formula XXIII). In one aspect, the invention provides a L5G2D3BP comprising a Formula XXIII sequence wherein 1, 2, 3, 4, or 5 of the N-terminal residues and/or 1, 2, or 3 of the C-terminal residues are missing or represent residues not found in corresponding positions in any one of SEQ ID NOS:40-42. In a further aspect, the invention provides a L5G2D3BP comprising a CDR-H2 consisting essentially of a sequence defined by a formula identical to Formula XXIII except in that Xaa₁₂, Xaa₁₃, and/or Xaa₁₄ represent any suitable amino acid residue(s) and differ from residues in corresponding positions in any one of SEQ ID NOS:40-42. In another aspect, the invention provides a L5G2D3BP comprising a CDR-H2 consisting essentially of a sequence identical to the foregoing Formula XXIII-like formula or Formula XXIII except in that Xaa₁ and Xaa₂ are Leu and Glu, respectively.

In an additional aspect, the invention provides a L5G2D3BP that comprises a CDR-H2 that comprises (and typically consists essentially of) a sequence according to Leu Glu Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Xaa₁₈ Xaa₁₉ Xaa₂₀ Xaa₂₁ Xaa₂₂ Xaa₂₃ Xaa₂₄ Xaa₂₅ Xaa₂₆ Xaa₂₇ Xaa₂₈, wherein (I) (a) Xaa₃ is Trp or Tyr, (b) Xaa₄ is Val or Met; (c) Xaa₅ is Ala or Gly; (d) Xaa₆ is Glu or Tyr; (e) Xaa₈ is Arg, Ser, or Thr; (f) Xaa₉ is Leu or Tyr; (g) Xaa₁₀ is Lys, Arg, or Ser; (h) Xaa₁₁ is Ser or Gly; (i) Xaa₁₂ is Asn or is missing; (j) Xaa₁₃ is Asn or is missing; (k) Xaa₁₄ is Tyr or is missing; (I) Xaa₁₅ is Ala, Thr, or Gly; (m) Xaa₁₇ is His, Tyr, or Asn; (n) Xaa₁₉ is Ala or Asn; (o) Xaa₂₀ is Glu or Pro; (p) Xaa₂₄ is Gly or Ser; (q) Xaa₂₆ is Phe or Ile; and (r) Xaa₂₇ is Thr or Ser, and (II) (a) Xaa₇ is a suitable residue other than Ile; (b) Xaa₁₆ is a suitable residue other than Thr; (c) Xaa₁₈ is a suitable residue other than Tyr; (d) Xaa₂₁ is a suitable residue other than Ser; (e) Xaa₂₂ is a suitable residue other than Leu; (f) Xaa₂₃ is a suitable residue other than Lys; (g) Xaa₂₅ is a suitable residue other than Arg; or (h) any combination of (II)(a)-(g) where unsubstituted residues in the group of Xaa₇, Xaa₁₆, Xaa₁₈, Xaa₂₁, Xaa₂₂, Xaa₂₃, and Xaa₂₅ match (are equivalent to) the corresponding position in SEQ ID NO:43 (Formula XXIV).

In another aspect, the invention provides L5G2D3BPs that comprise a CDR-H3 that comprises (and typically consists essentially of) a sequence according to the formula Cys Xaa₂ Xaa₃ Xaa₄ Xaa₅ Xaa₆ Xaa₇ Xaa₈ Xaa₉ Xaa₁₀ Xaa₁₁ Phe Xaa₁₃ Tyr Trp Gly Gln Gly (SEQ ID NO:79), wherein Xaa₂ is Thr or Ala; Xaa₃ is Arg, Gly, or Asn; Xaa₄ is Pro or is missing; Xaa₅ is Tyr or is missing; Xaa₆ is Asp or Asn; Xaa₇ is Tyr or Phe; Xaa₈ is Tyr or Asp; Xaa₉ is Gly or Glu; Xaa₁₀ is Ser, Arg, or Asn; Xaa₁₁ is Ser, Thr, or Phe; and Xaa₁₃ is Ala or Asp (Formula XXV). The invention also provides a similar γ2-binding protein comprising a sequence according to a formula that is essentially identical to Formula XXV except in that Phel₂ is substituted with a different suitable amino acid residue and/or Tyr₁₄ is/are substituted with (respectively and independently) a different suitable amino acid residue. In another aspect, a L5G2BP that comprises a CDR-H3 that comprises (or consists essentially of) a sequence defined by a formula similar to the preceding Formula XXV-like formula or Formula XXV except for one or more of: Xaa₂ is any suitable residue other than Thr or Ala; Xaa₄ is any suitable residue other than Pro; Xaa₅ is any suitable residue other than Tyr; Xaa₆ is any suitable residue other than Asp or Asn; Xaa₇ is any suitable residue other than Tyr or Phe; Xaa₈ is any suitable residue other than Tyr or Asp; Xaa₉ is any suitable residue other than Gly or Glu; and Xaa₁₃ is any suitable residue other than Ala or Asp.

In an additional aspect, the invention provides a L5G2D3BP comprising a VH CDR3 (CDR-H3) that comprises (and typically that consists essentially of) a sequence according to the formula Xaa₁ Xaa₂Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Xaa₁₈, wherein (a) Xaa₃ is Arg, Gly, or Asn; Xaa₁₀ is Ser, Arg, or Asn; Xaa₁₁ is Ser, Thr, or Phe; Xaa₄ is Pro or is missing; Xaa₅ is Tyr or is missing; Xaa₂ is Thr or Ala; Xaa₆ is Asp or Asn; Xaa₇ is Tyr or Phe; Xaa₈ is Tyr or Asp; Xaa₉ is Gly or Glu; Xaa₁₃ is Ala or Asp; Xaa₁ is any suitable residue other than Cys or is missing; Xaa₁₅ is any suitable residue other than Trp or is missing; Xaa₁₆ is any suitable residue other than Gly or is missing; Xaa₁₇ is any suitable residue other than Gln or is missing; and Xaa₁₈ is any suitable residue other than Gly or is missing; and (b) (i) Xaa₁₂ is any suitable residue other than Phe and Xaa₁₄ is Tyr; (ii) Xaa₁₄ is any suitable residue other than Tyr and Xaa₁₂ is Phe; or (iii) Xaa₁₂ and Xaa₁₄ respectively are any suitable residue other than Tyr and Phe (Formula XXVI). In another aspect, the invention provides a L5G2D3BP comprising a CDR-H3 that consists essentially of a sequence according to a formula identical to Formula XXVI except that Xaa₁₂ is Phe and Xaa₁₄ is Tyr. In another aspect, the invention provides a L5G2D3BP comprising a CDR-H3 that consists essentially of a sequence according to a formula that is essentially identical to Formula XXVI except that Xaa₄, Xaa₅, or both Xaa₄ and Xaa₅ represent any suitable residues other than Pro, Tyr, or Pro and Tyr, respectively. In another aspect, a γ2 binding protein can comprise a CDR-H3 that consists essentially of a sequence according to a formula that is identical to the foregoing Formula XXVI-like formula or Formula XXVI except for that one or more of Xaa₃, Xaa₁₀, and Xaa₁ representing suitable residue(s) that differ from residues at corresponding positions in any one of SEQ ID NOS:44-46.

Another facet of the invention is a L5G2D3BP that comprises a VH CDR3 (CDR-H3) that comprises (and typically consists essentially of) a sequence according to the formula Cys Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Trp Gly Gln Gly (SEQ ID NO:52), wherein (I) (a) Xaa₂ is Thr or Ala; (b) Xaa₃ is Arg, Gly, or Asn; (c) Xaa₄ is Pro or missing (absent); (d) Xaa₅ is Tyr or missing; (e) Xaa₆ is Asp or Asn; (f) Xaa₇ is Tyr or Phe; (g) Xaa₈ is Tyr or Asp; (h) Xaa₉ is Gly or Glu; (i) Xaa₁₀ is Ser, Arg, or Asn; 0) Xaa₁₁ is Ser, Thr, or Phe; (k) Xaa₁₃ is Ala or Asp; and (II) (a) Xaa₁₂ is any suitable residue other than Phe and Xaa₁₄ is Tyr; (b) Xaa₁₄ is any suitable residue other than Tyr and Xaa₁₂ is Phe, or (c) Xaa₁₂ is any suitable residue other than Phe and Xaa₁₄ is any suitable residue other than Tyr (Formula XXVII). In another aspect, the invention provides a L5G2D3BP comprising a VH CDR3 consisting essentially of a sequence according to a formula identical to Formula XXVII except for Xaa₁₂ is Phe and Xaa₁₄ is Tyr. In yet another aspect, the invention provides a L5G2D3BP comprising a VH CDR3 that consists essentially of a formula identical to Formula XXVII except that one or more of Xaa₂, Xaa₄, Xaa₅, Xaa₆, Xaa₇, Xaa₈, Xaa₉, Xaa₁₃, and Xaa₁₄ represent suitable amino acid residue(s) that differ from the residues in the corresponding positions in all of SEQ ID NOS:44-46. In a similar aspect, the invention provides a L5G2D3BP comprising a CDR-H3 consisting essentially of a sequence defined by a truncated version of Formula XXVII, wherein 1, 2, or 3 of the N-terminal residues and/or 1, 2, 3, or 4 of the C-terminal residues of Formula XXVII are absent. In a further aspect, a similar CDR-H3 can consist essentially of a sequence defined by a formula that is identical to Formula XXIX or such a truncated Formula XXVII sequence except for one or more of the following: Xaa₄ is any suitable residue other than Pro; Xaa₅ is any suitable residue other than Tyr; Xaa₂ is any suitable residue other than Thr or Ala; Xaa₆ is any suitable residue other than Asp or Asn; Xaa₇ is any suitable residue other than Tyr or Phe; and Xaa₈ is any suitable residue other than Gly or Glu.

In another aspect, the invention provides a L5G2D3BP that comprises a CDR-H3 which comprises (and typically consists essentially of) a sequence according to the formula Cys Ala Xaa₁ Xaa₂ Xaa₃ Asp Tyr Tyr Gly Xaa₄ Ser Phe Ala Tyr Trp Gly Gln Gly (SEQ ID NO:47), wherein Xaa₁ is Arg, Gly, or Asn; Xaa₂ is Pro or is missing; Xaa₃ is Tyr or is missing; and Xaa₄ is Ser, Arg, or Asn (Formula XXVIII). In a further aspect, the invention provides a L5G2D3BP that comprises a Formula XXVIII sequence wherein 1, 2, or 3 N-terminal residues and/or 1, 2, 3, or 4 C-terminal residues of the Formula XXVIII sequence are absent. In a further aspect, the invention provides a L5G2D3BP that comprises a CDR-H3 which consists essentially of a sequence defined by a formula identical to Formula XXVIII except for one or more of the following: Xaa₂ is any suitable residue other than Pro; Xaa₃ is any suitable residue other than Tyr; and Xaa₁ and/or Xaa₄ differ from residue(s) in corresponding position(s) of any one of SEQ ID NOS:44-46.

In general, any of the Xaa residues in the above-described formulas may be substituted with any suitable amino acid residue or deleted by a suitable deletion.

In yet another aspect, the invention provides a L5G2D3BP comprising a variant VL CDR1 consisting essentially of a sequence having at least at least about 40% amino acid sequence identity, such as about 50% amino acid sequence identity or more, typically at least about 60% amino acid sequence identity (e.g., at least about 75% amino acid sequence identity) to one or more of SEQ ID NOS:24-26, wherein (a) the residue of the variant VL CDR1 corresponding to position 2 of SEQ ID NOS:24-26, when these sequences are aligned, is any suitable residue other than Arg; (b) the residue of the variant VL CDR1 corresponding to position 4 of SEQ ID NOS:24-26 is any suitable residue other than Ser; (c) the residue of the variant VL CDR1 corresponding to position 12 of SEQ ID NOS:24-26 is any suitable residue other than Gly; or (d) the variant VL CDR1 differs in the residues corresponding to two or three of positions 2, 4, and/or 12 of SEQ ID NOS:24-26 from SEQ ID NO:27 by substitutions according to (a)-(c).

Identity in the context of amino acid sequences of the invention can be determined by a Needleman-Wunsch alignment analysis (see Needleman and Wunsch, J. Mol. Biol. (1970) 48:443-453), such as by analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs). Further related methods for assessing identity between sequences are described in, e.g., WO 03/048185.

In a further aspect, the invention provides a L5G2D3BP that comprises a variant VL CDR2 that consists essentially of a sequence having about 40% or more, such as at least about 50% amino acid sequence identity, such as at least about 65% amino acid sequence identity with one or more of SEQ ID NOS:28-30, and typically at least about 75% amino acid sequence identity (e.g., about 85% amino acid sequence identity such as about 60-99% amino acid sequence identity) with one or more of SEQ ID NOS:28-30, wherein (a) the residue in the variant VL CDR2 that corresponds to position 5 of SEQ ID NO:31 is any suitable residue other than a Ser; (b) the residue in the variant VL CDR2 that corresponds to position 9 of SEQ ID NO:31 is any suitable residue other than a Ser; or (c) the residues in the variant VL CDR2 that correspond to positions 5 and 9 of SEQ ID NO:31 are both any suitable residue other than a Ser.

In an additional aspect, the invention provides a L5G2D3BP that comprises a VL CDR3 that consists essentially of a sequence having about 40% or more amino acid sequence identity, such as at least about 50% identity, such as at least about 65% sequence identity to one or more of SEQ ID NOS:32-34, typically at least about 70% amino acid sequence identity (e.g., about 75-99% identity) to one or more of SEQ ID NOS:32-34 (e.g., about 80% identity to one or more of SEQ ID NOS:32-34), wherein (a) the residue in the variant VL CDR3 that corresponds to position 3 of SEQ ID NO:35 is any suitable residue other than Gln; (b) the residue in the variant VL CDR3 that corresponds to position 8 of SEQ ID NO:35 is any suitable residue other than Pro; (c) the residue in the variant VL CDR3 that corresponds to position 10 of SEQ ID NO:35 is any suitable residue other than Thr; or (d) two or more of the residues in the variant VL CDR3 that correspond to positions 3, 8, and 10 of SEQ ID NO:35 differ from that sequence according to a combination of (a) and (b); (a) and (c); (b) and (c); or (a), (b), and (c).

In another aspect, the invention provides a L5G2D3BP that comprises a variant VH CDR3 that consists essentially of a sequence having about 40% or more, such as at least about 65% identity (e.g., about 65-95% identity), such as about 70%, about 80% or more (e.g., about 90%) identity to one or more of SEQ ID NOS:44-46, wherein (a) the amino acid residue in the variant VH CDR3 that corresponds to position 12 of SEQ ID NO:47 is any suitable residue other than Phe; (b) the amino acid residue in the variant VH CDR3 that corresponds to position 14 of SEQ ID NO:47 is any suitable residue other than Tyr; or (c) the amino acid residues in the variant VH CDR3 that correspond to positions 12 and 14 of SEQ ID NO:47 are any suitable residues other than Phe and Tyr, respectively.

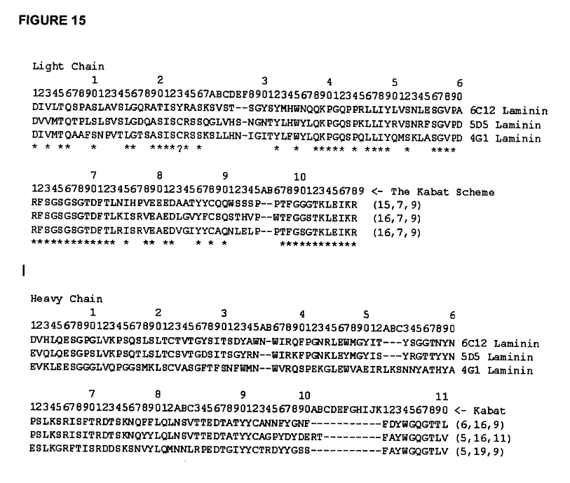

In a further aspect, the invention provides a L5G2D3BP that comprises the VL Fab region of mAb 4G1 as set forth in FIG. 15 (SEQ ID NO:53). In another facet, the invention provides a L5G2D3BP that comprises a variant VL Fab region having more than about 40% amino acid sequence identity, for example at least about 55% amino acid sequence identity (e.g., about 60-99% identity), more typically at least about 70% (e.g., about 75%, about 80%, about 85%, about 90%, about 95% or more (such as about 80-99%)) amino acid sequence identity to SEQ ID NO:53. In another aspect, the invention provides a L5G2D3BP that also or alternatively comprises the VH Fab region of mAb 4G1 (SEQ ID NO:56—see FIG. 15). In a variation of such an aspect, the invention provides a L5G2D3BP that comprises a variant VH Fab region having more than about 40% amino acid sequence identity (e.g., about 50-99% identity), and typically at least about 70% (e.g., about 75%, about 80%, about 85%, about 90%, about 95% or more (e.g., about 80-99%)) amino acid sequence identity to SEQ ID NO:56, optionally in combination with the mAb 4G1 VL region or a suitable variant thereof.

Another feature of the invention is a L5G2D3BP that comprises the VL region of mAb 5D5 (SEQ ID NO:54—see FIG. 15) or a variant VL region having more than about 40% amino acid sequence identity, such as at least about 50% (e.g., about 60-95%), such as at least about 70% (e.g., about 75%, about 80%, about 85%, about 90%, about 95% or more (such as about 80-99%)) amino acid sequence identity to SEQ ID NO:54. The invention additionally provides a L5G2D3BP that also or alternatively comprises the VH region of mAb 5D5 (SEQ ID NO:57—see FIG. 15) or a variant VH region having more than about 40%, such as at least about 55% (e.g., about 60-99%), at least about 70% (e.g., about 75%, about 80%, about 85%, about 90%, about 95% or more (such as about 80-99%)) amino acid sequence identity to SEQ ID NO:57.

In another facet, the invention provides a L5G2D3BP that comprises the VL region of mAb 6C12 (SEQ ID NO:55—see FIG. 15) or a variant VL region having about 40% or more amino acid sequence identity, such as at least about 50% (e.g., 60-95%), and typically at least about 70% (e.g., about 75%, about 80%, about 85%, about 90%, about 95% or more (such as about 80-99%)) amino acid sequence identity to SEQ ID NO:55. Additionally, the invention provides a L5G2D3BP that also or alternatively comprises the VH region of mAb 6C12 (SEQ ID NO:58—see FIG. 15) or a variant VH region having about 40% or more, such as at least about 50% (e.g., about 55-99%), more typically at least about 70% (e.g., about 75%, about 80%, about 85%, about 90%, about 95% or more (such as about 80-99%)) amino acid sequence identity to SEQ ID NO:58.

Any of the above-described VL, VH, and CDR sequences can be combined in any suitable manner to provide new L5G2D3BPs having specificity and/or selectively characteristics similar to mAb 4G1, mAb 5D5, or mAb 6C12, but differing in other characteristics, such as different immunogenicity in a human patient, affinity for antigenic determinant(s)/epitope(s), increased in vivo half-life, etc. In preparing new L5G2D3BPs targeting epitope(s)/antigenic determinant(s) similar to those targeted by mAbs 4G1, 5D5, or 6C12, particular attention typically may be paid to the CDR-H3 region of the peptide, if present, as this region is typically the strongest contributor to epitope binding In antibodies. For this reason as well, L5G2D3BPs that lack one or more CDR regions will usually retain one of the above-described CDR-H3 sequences or a functional variant thereof, typically at least in combination with a functionally and structurally related CDR-L3 sequence.

“CDR” domain sequences specifically described with reference to the foregoing CDR sequence-defined aspects and other aspects of the invention also may include within their description one or more typically CDR-associated residues located at the N— and/or C-terminus of the actual CDR (in other words, certain framework residues may be included in the “CDR” sequences described in such aspects). Thus, for example, in one aspect the invention provides a L5G2D3BP comprising a VL CDR1 consisting essentially of a sequence according to any one of SEQ ID NOS:24-27 and 48 wherein the N-terminal residue (e.g., the N-terminal Cys in the context of SEQ ID NOS:24-26) and/or one, two, or three of the C-terminal amino acid residues are missing. In another similar exemplary aspect, the invention provides a L5G2D3BP comprising a VL CDR2 consisting essentially of a sequence according to any one of SEQ ID NOS:28-31 and 49, wherein one or two of the N-terminal residues and/or one, two, or three of the C-terminal residues are missing. In a further exemplary aspect, the invention provides a L5G2D3BP comprising a VL CDR2 that consists essentially of a sequence according to any one of SEQ ID NOS:32-35 and 50, wherein the N-terminal residue (e.g., the N-terminal Cys in the context of SEQ ID NOS:32-34) and/or one, two, three, or four of the C-terminal residues are missing. In a further aspect, the invention provides a L5G2D3BP comprising a VL CDR3 that consists essentially of a sequence according to any one of SEQ ID NOS:32-35 or a sequence according to Formula XI or Formula XV, wherein the N-terminal Cys and/or 1, 2, 3, or 4 of the C-terminal residues (e.g., any portion of the C-terminal sequence according to the formula Phe-Gly-Xaa-Gly) are removed or otherwise absent (L5G2D3BPs comprising similar formula wherein such residues are removed are another feature of the invention). In another facet, the invention provides a L5G2D3BP comprising a VH CDR1 that consists essentially of a sequence according to any one of SEQ ID NOS:36-39 and 51, Formula XVII, or Formula XXI, wherein one, two, three, or four of the N-terminal residues (e.g., any portion of the N-terminal sequence according to the formula Cys-Xaa-Gln-Xaa in SEQ ID NOS:36-38) and/or one or two C-terminal residues in SEQ ID NOS:36-38) are missing (i.e., are removed or otherwise absent from the sequence). In yet another aspect, the invention provides a L5G2D3BP comprising a VH CDR2 that consists essentially of a sequence according to any one of SEQ ID NOS:40-43 or Formula XXII, wherein one, two, three, four, or five of the N-terminal amino acids thereof and/or one, two, three, four, five, or six of the C-terminal amino acids thereof are missing. In a further aspect, the invention provides a L5G2D3BP that comprises a VH CDR3 that consists essentially of any one of SEQ ID NOS:44-47 and 52 or Formula XXVI, wherein the N-terminal one, two, or three amino acid residues and/or the C-terminal one, two, three, or four amino acid residues are missing. An L5G2D3BP comprising each individual sequence encompassed by these combinations is provided by this paragraph. The invention also provides L5G2D3BPs wherein these “truncated” CDR-associated (or expected “true” CDRs) are combined with each other and/or other CDR sequences described herein.

The invention further provides L5G2D3BPs comprising (a) a VL CDR1 consisting essentially of a sequence according to one of Formulas I-III and V-VI, wherein, if not already so defined, the N-terminal amino acid residue of the sequence is Cys and the C-terminal portion of the sequence is Trp-Tyr-Leu; (b) a VL CDR2 consisting essentially of a sequence according to any one of Formulas VIII-X, wherein the N-terminal portion of the sequence, if not already so defined, is Ile-Tyr; (c) a VL CDR2 consisting essentially of a sequence according to any one of Formulas XI-XVI, wherein the N-terminal residue of the sequence is Cys and the C-terminal portion of the sequence falls within the formula Phe-Gly-Xaa₁-Xaa₂, wherein Xaa₁ and/or Xaa2 typically are independently selected from Gly and Ser but otherwise are any suitable residue(s), if not so already defined; (d) a VH CDR1 consisting essentially of a sequence according to any one of Formulas XVII-XXI, wherein the N-terminal residue is Cys and the C-terminal portion of the sequence is Trp-Xaa1, wherein Xaa1 typically represents Val or Ile but otherwise is any suitable residue, if not already so defined; (e) a VH CDR2 consisting essentially of a sequence according to any one of Formulas XXII-XXV, wherein, if not already so defined, the N-terminal portion of the sequence is defined by the formula Leu-Glu-Xaa₁-Xaa₂, wherein Xaa₁ typically is Trp or Tyr and Xaa₂ typically is Val or Met; and/or (f) a VH CDR3 consisting essentially of a sequence according to any one of Formulas XXVI-XXX, if not already so defined, wherein the N-terminal amino acid of the sequence is Cys and the C-terminal portion of the sequence is according to the formula Trp-Gly-Xaa-Gly, wherein “Xaa” herein and throughout, unless otherwise specified, represents any suitable amino acid residue.

In another aspect, the invention provides a L5G2D3BP comprising a variant of one or more of the CDRs disclosed in FIG. 11 and FIG. 12, wherein one, two, three, four, or more of the amino acid sequence residues of the CDR(s) and/or CDR-associated sequence(s), including those residues that define the N and C terminus of the CDRs, are substituted with a conservative amino acid residue substitution, or the sequence varies by one, two, three, or four deletions, insertions, and/or additions. Such modifications desirably do not cause substantial differences in the variant sequence in terms of structure and hydrophobicity from the parent sequence.

In another aspect, the invention provides a L5G2D3BP that comprises a light chain Fab region that consists essentially of a sequence selected from SEQ ID NOS:60-62. In an alternative aspect, the invention provides a L5G2D3BP that comprises a light chain Fab region variant sequence according to the formula Met Lys Phe Xaa₄ Val Gln Leu Leu Xaa₉ Leu Leu Leu Ile Trp Ile Pro Ala Ser Ser Ser Xaa₂₁ Xaa₂₂ Asp Ile Val Met Thr Gln Ser Pro Xaa₃₁ Ser Leu Ser Val Ser Leu Gly Asp Xaa₄₀ Ala Ser lIe Ser Cys Arg Ser Ser Xaa₄₉ Ser Leu Leu His Xaa54 Xaa₅₅ Gly Ile Thr Tyr Leu His Trp Tyr Leu Gln Lys Pro Gly Gln Ser Pro Lys Leu Leu Ile Tyr Xaa₇₇ Met Ser Lys Leu Ala Ser Gly Val Pro Asp Arg Phe Ser Gly Ser Gly Ser Gly Thr Asp Phe Thr Leu Lys Ile Ser Arg Val Glu Ala Glu Asp Leu Gly Ile Tyr Tyr Cys Ala Gln Xaa₁₁₈ Ser Xaa₁₁₉ Leu Pro Pro Thr Phe Gly Gly Gly Thr Lys Leu Glu Ile Lys Arg (SEQ ID NO:63), wherein Xaa₄ is Ser, Pro, or Gln; Xaa₉ is Gly, Ser, or missing; Xaa₂₁ is Arg or missing; Xaa₂₂ is Gly or missing; Xaa₃₁ is Phe, Leu, or Ala; Xaa₄₀ is Ser, Gln, or Lys; Xaa₄₉ is Lys, Gln, or missing; Xaa54 is Asn, Ser, or missing; Xaa₅₅ is Ile, Val, or Asn; Xaa₇₇ is Arg, Ser, or Gln; Xaa₁₁₈ is Asn, Ser, or Trp; and Xaa₁₁₉ is Glu, His, or Ser (Formula XXIX).

Another feature of the invention is a L5G2D3BP that comprises a variant light chain Fab region sequence comprising (and typically consisting essentially of) an amino acid sequence according to the formula Xaa₁ Xaa₂Xaa₃ Xaa₄Xaa₅ Xaa₆ Xaa₇ Xaa₈ Xaa₉ Xaa₁₀Xaa₁₁ Xaa₁₂ Xaa₁₃ Xaa₁₄ Xaa₁₅ Xaa₁₆ Xaa₁₇ Xaa₁₈ Xaa₁₉ Xaa₂₀ Xaa₂₁ Xaa₂₂ Xaa₂₃ Xaa₂₄ Xaa₂₅ Xaa₂₆ Xaa₂₇ Xaa₂₈ Xaa₂₉ Xaa₃₀ Xaa₃₁ Xaa₃₂ Xaa₃₃ Xaa₃₄ Xaa₃₅ Xaa₃₆ Xaa₃₇ Xaa₃₈ Xaa₃₉ Xaa₄₀ Xaa₄₁ Xaa₄₂ Xaa₄₃ Xaa₄₄ Xaa₄₅ Xaa₄₆ Xaa₄₇ Xaa₄₈ Xaa₄₉ Xaa₅₀ Xaa₅₁ Xaa₅₂ Xaa₅₃ Xaa₅₄ Xaa₅₅ Xaa₅₆ Xaa₅₇ Xaa₅₈ Xaa₅₉ Xaa₆₀ Xaa₆₁ Xaa₆₂ Xaa₆₃ Xaa₆₄ Xaa₆₅ Xaa₆₆ Xaa₆₇ Xaa₆₈ Xaa₆₉ Xaa₇₀ Xaa₇₁ Xaa₇₂ Xaa₇₃ Xaa₇₄ Xaa₇₅ Xaa₇₆ Xaa₇₇ Xaa₇₈ Xaa₇₉ Xaa₈₀ Xaa₈₁ Xaa₈₂ Xaa₈₃ Xaa₈₄ Xaa₈₅ Xaa₈₆ Xaa₈₇ Xaa₈₈ Xaa₈₉ Xaa₉₀ Xaa₉₁ Xaa₉₂ Xaa₉₃ Xaa₉₄ Xaa₉₅ Xaa₉₆ Xaa₉₇ Xaa₉₈ Xaa₉₉ Xaa₁₀₀ Xaa₁₀₁ Xaa₁₀₂ Xaa₁₀₂ Xaa₁₀₃ Xaa₁₀₄ Xaa₁₀₅ Xaa₁₀₆ Xaa₁₀₇ Xaa₁₀₈ Xaa₁₀₉ Xaa₁₁₀ Xaa₁₁₁ Xaa₁₁₂ Xaa₁₁₃ Xaa₁₁₄ Xaa₁₁₅ Xaa₁₁₆ Xaa₁₁₇ Xaa₁₁₈ Xaa₁₁₉ Xaa₁₂₀ Xaa₁₂₁ Xaa₁₂₂ Xaa₁₂₃ Xaa₁₂₄ Xaa₁₂₅Xaa₁₂₆ Xaa₁₂₇ Xaa₁₂₈ Xaa₁₂₉ Xaa₁₃₀ Xaa₁₃₁ Xaa₁₃₂ Xaa₁₃₃ wherein Xaa₂ is Arg, Lys, or Asp; Xaa₃ is Phe or Trp; Xaa₄ is Ser, Pro, or Gln; Xaa₅ is Val or Ala; Xaa₆ is Gln or Arg; Xaa₇ is Leu or Ile; Xaa₈ is Leu or Phe; Xaa₉ is Ser, Gly, or missing; Xaa₁₀ is Leu, Val, or Phe; Xaa₁₂ is Val, met, or Leu; Xaa₁₃ is Ile, Leu, or Phe; Xaa₁₄ is Trp or Ser; Xaa₁₅ is Ala or Ile; Xaa₁₆ is Pro or Ser; Xaa₁₇ is Gly, Ala, or Val; Xaa₁₈ is Ser or Ile; Xaa₁₉ is Thr, Ser, or Ile; Xaa₂₀ is Thr, Ser, or Ile; Xaa₂₁ is Ala or Ser; Xaa₂₂ is missing or Arg; Xaa₂₃ is missing or Gly; Xaa₂₄ is Asp or Gln; Xaa₂₅ is Ile or Val; Xaa₂₇ is Met or Leu; Xaa₃₀ is Ala, Thr, or Ser; Xaa₃₁ is Ala or Pro; Xaa₃₂ is Phe, Leu, or Ala; Xaa₃₃ is Ser or Ile; Xaa₃₄ is Asn, Leu, or Met; Xaa₃₅ is Pro or Ser; Xaa₃₆ is Val or Ala; Xaa₃₇ is Thr or Ser; Xaa₃₈ is Leu or Pro; Xaa₄₀ is Thr, Asp, or Glu; Xaa₄₁ is Ser, Gln, or Lys; Xaa₄₂ is Ala or Val; Xaa₄₃ is Ser or Thr; Xaa₄₄ is Ile or Met; Xaa₄₅ is Ser or Thr; Xaa₄₇ is Arg or Ser; Xaa₄₈ is Ser or Ala; Xaa₅₀ is Lys, Gln, or missing; Xaa₅₁ is Ser or Gly; Xaa₅₂ is Leu or Ser; Xaa₅₃ is Leu, Val, or missing; Xaa₅₄ is His or missing; Xaa₅₅ is Asn, Ser, or missing; Xaa₅₆ is Ile, Asn, or Val; Xaa₅₇ is Gly or Ser; Xaa₅₈ is Ile, Asn, or Tyr; Xaa₅₉ is Thr or Ile; Xaa₆₀ is Tyr or His; Xaa₆₁ is Leu or Trp; Xaa₆₂ is Phe, His or Tyr; Xaa₆₃ is Trp or Gln; Xaa₆₄ is Tyr or Gln; Xaa₆₅ is Leu or Lys; Xaa₆₆ is Gln or Ser; Xaa₆₇ is Gly or Ser; Xaa₆₈ is Gln or Pro; Xaa₆₉ is Ser or Lys; Xaa₇₀ is Pro or Arg; Xaa₇₁ is Gln, Lys, or Trp; Xaa₇₂ is Leu or Ile; Xaa₇₃ is Leu or Tyr; Xaa₇₄ is Ile or Asp; Xaa₇₅ is Tyr or Thr; Xaa₇₆ is Gln, Arg, or Ser; Xaa₇₇ is Met, Val, or Lys; Xaa₇₈ is Ser or Leu; Xaa₇₉ is Lys, Asn, or Ala; Xaa₈₀ is Leu, Arg, or Ser; Xaa₈₁ is Ala, Phe, or Gly; Xaa₈₂ is Ser or Val; Xaa₈₃ is Gly or Pro; Xaa₈₄ is Val or Ala; Xaa₈₅ is Pro or Arg; Xaa₈₆ is Asp or Phe; Xaa₈₇ is Arg or Ser; Xaa₈₈ is Phe or Gly; Xaa₉₃ is Ser or Thr; Xaa₉₄ is Gly or Ser; Xaa₉₅ is Thr or Tyr; Xaa₉₆ is Asp or Ser; Xaa₉₇ is Phe or Leu; Xaa₉₈ is Thr or missing; Xaa₉₉ is Leu or missing; Xaa₁₀₀ is Arg, Lys, or Thr; Xaa₁₀₃ is Arg or Ser; Xaa₁₀₄ is Val or Met; Xaa₁₀₅ is Glu or Gln; Xaa₁₀₉ is Val, Leu, or Ala; Xaa₁₁₀ is Gly or Ala; Xaa₁₁₁ is Ile, Val, or Thr; Xaa₁₁₃ is Tyr or Phe; Xaa₁₁₅ is Ala, Ser, or Gln; Xaa₁₁₇ is Asn, Ser, or Trp; Xaa₁₁₈ is Leu, Thr, or Ser; Xaa₁₁₉ is Glu, His, or Ser; Xaa₁₂₀ is Leu, Val, or Ser; Xaa₁₂₂ is Leu, Val, or Ser; and Xaa₁₂₆ is Ser or Gly; Xaa₁₂₇ is Gly or Ser; and further wherein Xaa₁, Xaa₁₁, Xaa₂₆, Xaa₂₈, Xaa₂₉, Xaa₃₉, Xaa₄₆,Xaa₄₈, Xaa₈₉-Xaa₉₂, Xaa₁₀₁-Xaa₁₀₂, Xaa₁₀₆-Xaa₁₀₈, Xaa₁₁₂, Xaa₁₁₄, Xaa₁₁₆, Xaa₁₂₁, Xaa₁₂₃-Xaa₁₂₅, and Xaa₁₂₈-Xaa₁₃₅ are defined by the corresponding residues at these positions in SEQ ID NO:63 except that the variant light chain Fab sequence differs from SEQ ID NO:63 by one or more of the following: Xaa₁ is any suitable residue other than Met; Xaa₁₁ is any suitable residue other than Leu; Xaa₂₆ is any suitable residue other than Val; Xaa₂₈ is any suitable residue other than Thr; Xaa₂₉ is any suitable residue other than Gln; Xaa₃₉ is any suitable residue other than Gly; Xaa₄₆ is any suitable residue other than Cys; Xaa₄₉ is any suitable residue other than Ser; Xaa₈₉ is any suitable residue other than Ser; Xaa₉₀ is any suitable residue other than Gly; Xaa₉₁ is any suitable residue other than Ser; Xaa₉₂ is any suitable residue other than Gly (optionally with the requirement that Xaa₈₉-Xaa₉₁, forms a flexible sequence similar to a Ser-Gly flexible linker sequence); Xaa₁₀₁, is any suitable residue other than Ile; Xaa₁₀₂ is any suitable residue other than Ser; Xaa₁₀₆ is any suitable residue other than Ala; Xaa₁₀₇ is any suitable residue other than Glu; Xaa₁₀₈ is any suitable residue other than Asp; Xaa₁₁₂ is any suitable residue other than Tyr; Xaa₁₁₄is any suitable residue other than Cys; Xaa₁₁₆ is any suitable residue other than Gln; Xaa₁₂₁ is any suitable residue other than Pro; Xaa₁₂₃ is any suitable residue other than Thr; Xaa₁₂₄ is any suitable residue other than Phe; Xaa₁₂₅ is any suitable residue other than Gly; Xaa₁₂₈ is any suitable residue other than Thr; Xaa₁₂₉ is any suitable residue other than Lys; Xaa₁₃₀ is any suitable residue other than Leu; Xaa₁₃₁ is any suitable residue other than Leu; Xaa₁₃₂ is any suitable residue other than Glu; Xaa₁₃₃ is any suitable residue other than Ile; Xaa₁₃₄ is any suitable residue other than Lys; and Xaa₁₃₅ is any suitable residue other than Arg (Formula XXX). Desirably, such a sequence exhibits at least about 45%, such as about 60% or more, about 75% or more, about 80% or more, about 85% or more, about 90% or more, or about 95% or more (e.g., about 70-99%) identity to one or more of SEQ ID NOS:60-62.

The invention further provides L5G2D3BPs that comprise a variant light chain Fab sequence that comprises (and typically that consists essentially of) a sequence according to the formula Xaa Xaa₂ Xaa Xaa₄ Xaa Xaa Xaa Xaa Xaa₉ Xaa₁₀ Xaa Xaa₁₂ Xaa₁₃ Xaa Xaa Xaa Xaa₁₇ Xaa Xaa₁₉ Xaa Xaa Xaa Xaa Xaa Xaa Xaa Xaa Xaa Xaa₂₉ Xaa Xaa₃₁Xaa Xaa₃₃ Xaa Xaa Xaa Xaa Xaa Xaa₃₉ Xaa₄₀ Xaa Xaa Xaa Xaa Xaa Xaa Xaa Xaa Xaa₄₉ Xaa Xaa Xaa₅₂ Xaa Xaa₅₄ Xaa₅₅ Xaa Xaa₅₇ Xaa Xaa Xaa Xaa₆₁ Xaa Xaa Xaa Xaa Xaa Xaa Xaa Xaa Xaa Xaa Xaa₇₂ Xaa Xaa Xaa Xaa Xaa₇₇ Xaa₇₈ Xaa Xaa₈₀ Xaa₈₁ Xaa₈₂ Xaa Xaa Xaa Xaa Xaa Xaa Xaa Xaa Xaa Xaa Xaa Xaa Xaa Xaa Xaa Xaa Xaa Xaa Xaa₁₀₁, Xaa Xaa Xaa Xaa Xaa Xaa Xaa Xaa Xaa Xaa Xaa₁₁₂ Xaa Xaa Xaa Xaa₁₁₆ Xaa Xaa₁₁₈ Xaa₁₁₉ Xaa₁₂₀ Xaa₁₂₁ Xaa Xaa Xaa Xaa Xaa Xaa Xaa Xaa Xaa Xaa Xaa Xaa Xaa Xaa, wherein Xaa is any suitable amino acid residue; Xaa₂ is Arg, Lys, or Asp; Xaa₄ is Ser, Pro, or Gln; Xaa₉ is Gly, Ser, or missing; Xaa₁₀ is Leu, Val, or Phe; Xaa₁₂ is Val, Met, or Leu; Xaa₁₃ is Leu, Phe, or Ile; Xaa₁₇ is Gly, Ala, or Val; Xaa₁₉ is Thr, Ser, or lIe; Xaa₂₉ is Ala, Thr, or Ser; Xaa₃₁ is Phe, Leu, or Ala; Xaa₃₃ is Asn, Leu, or Met; Xaa₃₉ is Thr, Asp, or Glu; Xaa₄₀ is Ser, Gln, or Lys; Xaa₄₉ is Lys, Gln, or missing/absent; Xaa₅₂ is Leu, Val or missing; Xaa₅₄ is Asn, Ser, or missing; Xaa₅₅ is Ile, Asn, or Val; Xaa₅₇ is Ile, Asn, or Tyr; Xaa₆₁ is Phe, His, or Tyr; Xaa₇₂ is Gln, Lys, or Trp; Xaa₇₇ is Gln, Arg, or Ser; Xaa8o is Lys, Asn, or Ala; Xaa₈₁ is Leu, Arg, or Ser; Xaa₈₂ is Ala, Phe, or Gly; Xaa₁₀₁, is Arg, Lys, or Thr; Xaa₁₁₂ is Ile, Val, or Thr; Xaa₁₁₆ is Ala, Ser, or Gln; Xaa₁₁₈ is Asn, Ser, or Trp; Xaa₁₁₉ is Leu, Thr, or Ser; Xaa₁₂₀ is Glu, His, or Ser; and Xaa₁₂₁ is Leu, Val, or Ser (Formula XXXI). Desirably, such a sequence exhibits at least about 45%, such as about 60% or more, about 75% or more, about 80% or more, about 85% or more, about 90% or more, or about 95% or more (e.g., about 70-99%) identity to one or more of SEQ ID NOS:60-62.

In another aspect, the invention provides an L5G2D3BP that comprises a sequence that corresponds to a portion of a sequence according to any one of formulas XXIX-XXXI (i.e., comprises a span of contiguous amino acids from such a sequence) or a highly similar sequence (e.g., a sequence having at least about 65%, at least about 75%, at least about 80%, at least about 85%, at least about 90%, at least about 95%, or more, such as about 70-99%, identity to such a portion of Formula XXIX, XXX, or XXXI (or any one of SEQ ID NOS:60-62)), wherein the sequence mediates or contributes to γ2 DIII binding by the L5G2D3BP. Typically, such a sequence is at least about 10 amino acids in length, such as about 15 or more, about 20 or more, about 30 or more, about 40 or more, about 50 or more, at least about 60 or more (e.g., about 25-65) amino acids in length.

In another aspect, the invention provides a L5G2D3BP comprising a light chain Fab sequence according to a portion of one of SEQ ID NOS:60-62 wherein about 1-20 of the N-terminal amino acid residues in the light chain sequence of SEQ ID NOS:60-62 are absent from the light chain Fab sequence. In another aspect, the invention provides a light chain variant Fab sequence according to a fragment of any one of Formulas XXIX-XXXI, that lacks about 1-20 amino acid residues from the N-terminus portion of these formulas.

The deletion of N-terminal sequences from the various light chain Fab and heavy chain Fab sequences described herein typically serves to remove or disable the signal sequence portion of light or heavy chain peptides comprising such sequences. Thus, these sequences can be removed, for example, where it is desirable to replace these sequences with exogenous signal sequence(s). Removal of these sequences also can be desirable where these peptides are produced by chemical synthesis (e.g., solid phase peptide synthesis). Removal may further be desirable where such sequence(s) are incorporated into larger peptide sequences, such as in the context of a fusion protein or fusion protein(s) comprising such sequences. Consideration of targeting the endoplasmic reticulum for proper folding and glycosylation, assembly of H2L2 monomers and other multi-chain structures (in the case of antibody and antibody-like molecules) and formation of interchain disulfide bonds, and secretion from the cell (e.g., by vesicle transport from the Golgi apparatus and exocytosis) can be factors considered in making modifications to variant VH and VL sequences lacking such signal sequences when producing L5G2D3BPs comprising such sequences by recombinant techniques. An ordinarily skilled artisan can select appropriate exogenous signal sequences that provide for such processing in a manner similar to that of wild-type antibodies or for different processing, such as in the case of expressing recombinant L5G2D3BPs in a non-chordate cell, using routine experimentation and drawing upon known techniques.

Another feature of the invention is L5G2D3BPs that comprise a heavy chain Fab variant sequence that comprises (and typically that consists essentially of) a sequence according to the formula Xaa₁ Val Xaa₃ Leu Xaa₅ Glu Ser Gly Xaa₉ Xaa₁₀ Leu Val Xaa₁₃ Pro Xaa₁₅ Xaa₁₆ Xaa₁₇ Xaa₁₈ Xaa₁₉ Leu Xaa₂₁ Cys Xaa₂₃ Xaa₂₄ Xaa₂₅ Gly Xaa₂₇ Xaa₂₈ Xaa₂₉ Xaa₃₀ Xaa₃₁ Xaa₃₂ Xaa₃₃ Xaa₃₄ Xaa₃₅ Asn Trp Xaa₃₈ Arg Xaa₄₀ Xaa₄₁ Pro Xaa₄₃ Xaa44 Xaa₄₅ Leu Glu Xaa48 Xaa₄₉ Xaa₅₀ Xaa₅₁ Ile Xaa₅₃ Xaa₅₄ Xaa₅₅ Xaa₅₆ Xaa₅₇ Xaa₅₈ Xaa₅₉ Xaa₆₀ Thr Xaa₆₂ Try Xaa₆₄ Xaa₆₅ Ser Leu Lys Xaa₆₉ Arg Xaa₇₁ Xaa₇₂ Xaa₇₃ Xaa₇₄ Arg Asp Xaa₇₇ Ser Lys Xaa₈₀ Xaa₈₁ Xaa₈₂ Xaa₈₃ leu Gln Xaa₈₆ Asn Xaa₈₈ Xaa₈₉ Xaa₉₀ Xaa₉₁ Glu Asp Thr Xaa₉₅ Xaa₉₆ Tyr Tyr Cys Xaa₁₀₀ Xaa₁₀₁ Xaa₁₀₂ Xaa₁₀₃ Xaa₁₀₄ Xaa₁₀₅ Xaa₁₀₆ Xaa₁₀₇ Xaa₁₀₈ Xaa₁₀₉ Phe Xaa₁₁₁ Tyr Trp Gly Gln Gly Thr Xaa₁₁₈ Xaa₁₁₉ Tyr Val Ser Xaa₁₂₀ (SEQ ID NO:72), wherein the variable residues of the sequence can be defined according to the following table:

TABLE 8

| Pos. | AAs | Pos. | AAs | Pos. | AAs | Pos. | AAs | Pos. | AAs | Pos. | AAs |
|---|---|---|---|---|---|---|---|---|---|---|---|
| 1 | D or E | 3 | H, Q, | 5 | Q or E | 9 | P or G | 10 | G or S | 13 | K or Q |
| | | | or K | | | | | | | | |
| 15 | S or G | 16 | Q or G | 17 | S or T | 18 | L or M | 19 | S or K | 21 | T or S |
| 23 | T, S, | 24 | V or A | 25 | T or S | 27 | Y, D, | 28 | S or T | 29 | I or F |
| | or V | | | | | | or F | | | | |
| 30 | T or S | 31 | S or N | 32 | D, G, | 33 | Y or F | 34 | A, R, | 35 | W, |
| | | | | | or −* | | | | or W | | M, or − |
| 38 | I or V | 40 | Q or K | 41 | F or S | 43 | G or E | 44 | N or K | 45 | R, K, |
| | | | | | | | | | | | or G |
| 48 | W or Y | 49 | M or V | 50 | G or A | 51 | Y or E | 53 | T, S, | 54 | Y or L |
| | | | | | | | | | or R | | |
| 55 | S, R, | 56 | G or S | 57 | N or − | 58 | N or − | 59 | Y or − | 60 | G, T, |
| | or K | | | | | | | | | | or A |
| 62 | N, Y, | 64 | N or A | 65 | P or E | 69 | S or G | 71 | I or F | 72 | S or T |
| | or H | | | | | | | | | | |
| 73 | F or I | 74 | T or S | 77 | T or D | 80 | N or S | 81 | Q or N | 82 | F, Y, |
| | | | | | | | | | | | or V |
| 83 | F or Y | 86 | L or M | 88 | S or N | 89 | V or L | 90 | T or R | 95 | A or G |
| 96 | T or I | 100 | A or T | 101 | N, G, | 102 | P or − | 103 | Y or − | 104 | N or D |
| | | | | | or R | | | | | | |
| 105 | F or Y | 106 | Y or D | 107 | G or E | 108 | N, R, | 109 | F, T, | 111 | D or A |
| | | | | | | | or S | | or S | | |
| 118 | T or L | 119 | L or V | 91 | T or P | 120 | S or A | | | | |

*“−” = missing
AAs = amino acids
Pos. = position

Desirably, such a variant heavy chain Fab sequence exhibits significant identity (e.g., at least about 40% amino acid sequence identity (e.g., about 50-99% identity), such as about 60% or more, about 70% or more, about 80% or more, about 90% or more, about 95% or more (e.g., about 65-99%) AA sequence identity to one or more of SEQ ID NOS:69-71.

In a variation on the aspect described in the preceding paragraph, the invention provides an L5G2D3BP comprising a heavy chain Fab sequence consisting essentially of a sequence according to a formula identical to Formula XXXII but for one or more of Val₂, Leu₄, Glu₆, Ser₇, Gly₈, Leu₁₁, Val₁₂, Pro₁₄, Leu₂₀, Cys₂₂, Gly₂₆, Asn₃₆, Trp₃₇, Arg₃₉, Pro₄₂, Leu₄₆,Glu₄₇,Ile₅₂,Thr₆₁, Ser₆₆, Leu₆₇, Lys₆₈, Arg₇₀, Arg₇₅, Asp₇₆, Ser₇₈, Lys₇₉, Leu₈₄, Gln₈₅, Asn₈₇, Ser₈₈, Glu₉₂,Asp₉₃, Thr₉₄, Tyr₉₇, Tyr₉₈, Cys₉₉, Phe₁₁₀, Tyr₁₁₂, Trp₁₁₃, Gly₁₁₄, Gln₁₁₅, Gly₁₁₆, and Thr₁₁₇ are substituted. Typically most or all of such substitutions are replacements with other naturally occurring amino acids, and commonly most or all such replacements are conservative substitutions (e.g., about ⅔^{rd}, about ¾^{th}, or more, of such substitutions are conservative). Desirably, such substitution(s) increase the affinity of the L5G2D3BP over L5G2D3BPs comprising a Formula XXXII sequence lacking the indicated substitution(s).

As noted above, heavy chain sequences described herein, such as sequences defined by Formula XXXII or by variations on Formula XXXII described in the preceding paragraphs (and any one of SEQ ID NOS:69-71) can be truncated by about 1-20 amino acid residues in the N-terminus. L5G2D3BPs comprising such truncated heavy chain sequences (including heavy chain sequence variants according to the formulas provided above) are additional features of this invention.

The invention further provides a L5G2D3BP that comprises a sequence that corresponds to a portion of a sequence according to Formula XXXII or the above-described Formula XXXII variants (or a span of contiguous amino acid residues therefrom) that mediates or contributes to γ2 binding by the L5G2D3BP or a very similar sequence (e.g., a sequence that is more than about 60% identical, such as at least about 70%, at least about 80%, at least about 90%, at least about 95% or more, such as about 75-99% identical to such a Formula XXXIV sequence or a sequence selected from SEQ ID NOS:69-71). The size of such an amino acid sequence typically is at least about 10 amino acid residues, such as at least about 15, 20, 30, 40, 50, or more amino acid residues (e.g., about 12-60 AAs).

In another aspect, the invention provides L5G2D3BPs that comprise a heavy chain sequence consisting essentially of SEQ ID NO:69, SEQ ID NO:70, and/or SEQ ID NO:71. In a further aspect, the invention provides L5G2D3BPs that comprise a sequence that corresponds to a fragment of any one of SEQ ID NOS:69-71 that mediates or contributes to γ2 DIII binding. Typically, such a fragment is at least about 10, such as about 15, about 20, about 30, about 40, about 50, about 60 or more amino acids in length. In a further facet, the invention provides L5G2D3BPs that comprise a heavy chain sequence consisting essentially of SEQ ID NO:84, SEQ ID NO:85, or SEQ ID NO:86. Also or alternatively, any of these heavy chain Fab variant sequences can be modified by truncation of about 1-20 of the N-terminal residues of the sequence in an L5G2D3BP, as such portion may be proteolytically cleaved in peptide processing in certain expression systems (i.e., such residues may represent a signal sequence).

In another aspect, the invention provides a nucleic acid comprising a sequence that codes for production of a L5G2D3BP. A L5G2D3BP-encoding nucleic acid can have any suitable characteristics and comprise any suitable features or combinations thereof. Thus, for example, a L5G2D3BP-encoding nucleic acid may be in the form of DNA, RNA, or a hybrid thereof, and may include nonnaturally-occurring bases, a modified backbone (e.g., a phosphothioate backbone that promotes stability of the nucleic acid), or both. The nucleic acid advantageously comprises features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

In a further aspect, the invention provides a vector comprising a L5G2BP-encoding nucleic acid (e.g., a nucleic acid comprising a L5G2D3BP-encoding sequence). A vector refers to a delivery vehicle that promotes the expression of a L5G2BP-encoding nucleic acid, the production of a L5G2BP peptide, the transfection/transformation of target cells, the replication of the L5G2BP-encoding nucleic acid, promotes stability of the nucleic acid, promotes detection of the nucleic acid and/or transformed/transfected cells, or otherwise imparts advantageous biological and/or physiochemical function to the L5G2BP-encoding nucleic acid. Unless otherwise stated, a vector in the context of this invention can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one exemplary aspect, a L5G2D3BP-encoding nucleic acid is comprised in a naked DNA or RNA vector, including, for example, a linear expression element (as described in, e.g., Sykes and Johnston (1997) Nat Biotech 17: 355-59), a compacted nucleic acid vector (as described in, e.g., U.S. Pat. No. 6,077, 835 and/or International Patent Application WO 00/70087), a plasmid vector such as pBR322, pUC 19/18, or pUC 118/119, a “midge” minimally-sized nucleic acid vector (as described in, e.g., Schakowski et al. (2001) Mol Ther 3: 793-800), or as a precipitated nucleic acid vector construct, such as a CaPO4-precipitated construct (as described in, e.g., International Patent Application WO 00/46147, Benvenisty and Reshef (1986) Proc Natl Acad Sci USA 83: 9551-55, Wigler et al. (1978), Cell 14:725, and Coraro and Pearson (1981) Somatic Cell Genetics 7:603). Such nucleic acid vectors and the usage thereof are well known in the art (see, e.g., U.S. Pat. Nos. 5,589,466 and 5,973,972).

In one aspect, the vector is suitable for expression of the L5G2D3BP in a bacterial cell. Examples of such vectors include, for example, vectors which direct high level expression of fusion proteins that are readily purified (e.g., multifunctional E. coli cloning and expression vectors such as BLUESCRIPT (Stratagene), pIN vectors (Van Heeke & Schuster, J Biol Chem 264: 5503-5509 (1989); pET vectors (Novagen, Madison Wis.); and the like).

An expression vector also or alternatively can be, for example, a vector suitable for expression in a yeast system. Any vector suitable for expression in a yeast system can be employed. Suitable vectors for use in, e.g., S. cerevisiae include, e.g., vectors comprising constitutive or inducible promoters such as alpha factor, alcohol oxidase and PGH (reviewed in, e.g., Ausubel, supra, and Grant et al., Methods in Enzymol 153: 516-544 (1987)).

A nucleic acid and/or vector can also comprise a nucleic acid sequence encoding a secretion/localization sequence, which can target a polypeptide, such as a nascent polypeptide chain, to a desired cellular compartment, membrane, or organelle, or which directs polypeptide secretion to periplasmic space or into cell culture media. Such sequences are known in the art, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

L5G2BP-encoding nucleic acid vectors can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter), effective poly (A) termination sequences, an origin of replication for plasmid product in E. coli, an antibiotic resistance gene as a selectable marker, and/or a convenient cloning site (e.g., a polylinker). Nucleic acids also can comprise an inducible promoter as opposed to a constitutive promoter such as CMV IE (the skilled artisan will recognize that such terms are actually descriptors of a relative degree of gene expression under certain conditions). In one aspect, the invention provides a nucleic acid comprising a sequence encoding a L5G2BP or related molecule, which sequence is operatively linked to a tissue specific promoter that promotes expression of the sequence in Ln-5-associated tissues such as Ln-5-sensitive cancer cell-associated tissues. Examples of such cancer-related tissue specific promoter systems are described in, e.g., Fukazawa et al., Cancer Res. 2004;64(1):363-9; Latham et al., Cancer Res. 2000 60(2):334-41; and Shirakawa et al., Mol Urol. 2000;4(2):73-82.

In another aspect, the nucleic acid is positioned in and/or delivered to the host cell or host animal via a viral vector. Any suitable viral vector can be used in this respect, and several are known in the art. A viral vector can comprise any number of viral nucleic acid sequences and/or molecules, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), a vector similar to those described in U.S. Pat. No. 5,849,586 and International Patent Application WO 97/04748, or an intact virus particle comprising viral nucleic acids and the nucleic acid of the invention. A viral particle viral vector can comprise a wild-type viral particle or a modified viral particle. The viral vector can be a vector which requires the presence of another vector or wild-type virus for replication and/or expression (i.e., a viral vector can be a helper-dependent virus), such as an adenoviral vector amplicon. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors. Examples of such viruses and viral vectors are in, e.g., Fields et al., eds., VIROLOGY, Raven Press, Ltd., New York (3rd ed., 1996 and 4th ed., 2001); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995), Levine, “Viruses,” Scientific American Library No. 37 (1992), MEDICAL VIROLOGY, D. O. White et al., eds., Acad. Press (2nd ed. 1994), and INTRODUCTION TO MODERN VIROLOGY, Dimock, N. J. et al., eds., Blackwell Scientific Publications, Ltd. (1994).

Viral vectors that can be employed with polynucleotides of the invention and the methods described herein thus include, for example, adenovirus and adeno-associated vectors, as in, e.g., Carter (1992) Curr Opinion Biotech 3: 533-539 (1992) and Muzcyzka (1992) Curr Top Microbiol Immunol 158: 97-129 (1992). Additional types and aspects of AAV vectors are described in, e.g., Buschacher et al., Blood, 5 (8), 2499-504, Carter, Contrib. Microbiol. 4: 85-86 (2000), Smith-Arica, Curr. Cardiol. Rep. 3 (1): 41-49 (2001), Taj, J. Biomed. Sci. 7 (4): 279-91 (2000), Vigna et al., J. Gene Med. 2 (5):308-16 (2000), Klimatcheva et al., Front. Biosci. 4: D481-96 (1999), Lever et al., Biochem. Soc. Trans. 27 (6):841-47 (1999), Snyder, J Gene Med. 1 (3):166-75 (1999), Gerich et al., Knee Surg. Sports Traumatol. Arthrosc. 5 (2): 118-23 (1998), and During, Adv. Drug Deliv. Review 27 (1) :83-94 (1997), and U.S. Pat. Nos. 4,797,368; 5,139,941; 5,173,414; 5,614,404; 5,658,785; 5,858,775; and 5,994,136, as well as other references discussed elsewhere herein). Adeno-associated viral vectors can be constructed and/or purified using the methods set forth, for example, in U.S. Pat. No. 4,797,368 and Laughlin et al., Gene 23:65-73 (1983).

Another type of viral vector that can be employed to deliver polynucleotides and other nucleic acids of the invention is a papillomaviral vector. Suitable papillomaviral vectors are known in the art and described in, e.g., Hewson (1999) Mol Med Today 5 (1): 8, Stephens (1987) Biochem J 248 (1):1-11, and U.S. Pat. No. 5,719,054. Useful papillomaviral vectors are provided in, e.g., International Patent Application WO 99/21979. Alphavirus vectors also can be useful gene delivery vectors. Alphavirus vectors are known in the art and described in, e.g., Carter (1992) Curr Opinion Biotech 3:533-539, Muzcyzka (1992) Curr Top Microbiol Immunol. 158:97-129, Schlesinger Expert Opin Biol Ther. March 2001; 1 (2):177-91, Polo et al. Dev Biol (Basel). 2000; 104:181-5, Wahlfors et al. Gene Ther. March 2000; 7 (6):472-80, Colombage et al. Virology. Oct. 10, 1998; 250 (1):151-63, and International Patent Applications WO 01/81609, WO 00/39318, WO 01/81553, WO 95/07994, and WO 92/10578.

Another advantageous group of viral vectors are herpes viral vectors. Examples of herpes viral vectors are described in, e.g., Lachmann et al., Curr Opin Mol Ther October 1999; 1 (5):622-32, Fraefel et al., Adv Virus Res. 2000; 55:425-51, Huard et al., Neuromuscul July 1997; 7 (5):299-313, Glorioso et al., Annu Rev Microbiol. 1995; 49:675-710, Latchman, Mol Biotechnol. October 1994; 2 (2):179-95, and Frenkel et al., Gene Ther. 1994; 1 Suppl 1:S40-6, as well as U.S. Pat. Nos. 6,261,552 and 5,599,691.

Retroviral vectors, including lentiviral vectors, also can be advantageous gene delivery vehicles in particular contexts. There are numerous retroviral vectors known in the art. Examples of retroviral vectors are described in, e.g., Miller, Curr Top Microbiol Immunol (1992) 158: 1-24; Salmons and Gunzburg (1993) Human Gene Therapy 4: 129-141; Miller et al. (1994) Methods in Enzvmolosv 217: 581-599, Weber et al., Curr Opin Mol Ther. October 2001; 3 (5): 439-53, Hu et al., Pharmacol Rev. December 2000; 52 (4): 493-511, Kim et al., Adv Virus Res. 2000; 55: 545-63, Palu et al., Rev Med Virol. May-Jun. 2000; 10 (3): 185-202, and Takeuchi et al., Adv Exp Med Biol. 2000; 465: 23-35, as well as U.S. Pat. Nos. 6,326,195, 5,888,502, 5,580,766, and 5,672, 510.

Adenoviral vectors also can be suitable viral vectors for gene transfer. Adenoviral vectors are well known in the art and described in, e.g., Graham et al (1995) Mol Biotechnol 33 (3): 207-220, Stephenson (1998) Clin Diagn Virol 10 (2-3): 187-94, Jacobs (1993) Clin Sci (Lond). 85 (2): 117-22, U.S. Pat. Nos. 5,922,576, 5,965,358 and 6,168,941 and Int'l Patent Appns WO 98/22588, WO 98/56937, WO 99/15686, WO 99/54441, and WO 00/32754.

Other suitable viral vectors include pox viral vectors. Examples of such vectors are discussed in, e.g., Berencsi et al., J Infect Dis (2001) 183 (8): 1171-9; Rosenwirth et al., Vaccine February 8, 2001; 19 (13-14):1661-70; Kittlesen et al., J Immunol (2000) 164 (8):4204-11; Brown et al. Gene Ther 2000 7 (19):1680-9; Kanesa-thasan et al., Vaccine (2000) 19 (4-5):483-91; Sten (2000) Drua 60 (2): 249-71. Vaccinia virus vectors are preferred pox virus vectors. Examples of such vectors and uses thereof are provided in, e.g., Venugopal et al. (1994) Res Vet Sci 57 (2): 188-193, Moss (1994) Dev Biol Stand 82: 55-63 (1994), Weisz et al. (1994) Mol Cell Biol 43: 137-159, Mahr and Payne (1992) Immunobioloev 184 (2-3): 126-146, Hruby (1990) Clin Microbiol Rev 3 (2): 153-170, and International Patent Applications WO 92/07944, WO 98/13500, and WO 89/08716.

Other features of the invention include recombinant cells, such as yeast, bacterial, and mammalian cells (e.g., immortalized mammalian cells) comprising such a nucleic acid, vector, or combinations of either or both thereof. For example, in one exemplary aspect the invention provides a cell comprising a nucleic acid stably integrated into the cellular genome that comprises a sequence coding for expression of a L5G2BP (e.g., a L5G2D3BP) of the invention. In another aspect, the invention provides a cell comprising a non-integrated nucleic acid, such as a plasmid, cosmid, phagemid, or linear expression element, which comprises a sequence coding for expression of a L5G2BP.

Immunogenic peptides comprising any of the above-described antigenic determinant regions of Ln-5 y2 (including two or more of such ADRs) are another feature of the invention. Such immunogens can be used to elicit a direct immune response in a method comprising an active immunotherapy regimen.

The invention further provides a fusion protein comprising a γ2 ADR immunogenic portion (comprising one or more of such γ2 ADRs or essentially identical sequences) and a fusion partner sequence that modulates the functional and/or physiochemical properties of the immunogenic peptide by, for example, improving the half-life of the fusion protein (e.g., by inclusion of an immunoglobulin domain sequence); facilitating detection and/or purification of the fusion protein (by comprising, e.g., a fluorescent peptide sequence, a reporter enzyme sequence, an epitope tag, a hexa-histidine sequence, or the like); promoting the targeting of the fusion protein (e.g., by comprising a ligand or portion of a ligand specific for a receptor on a target cell); promoting induction of a distinct immune response (e.g., corresponds to a cancer antigen or an immunogenic fragment thereof); acting as a cytotoxic agent; or achieving any combination thereof (e.g., a heat shock fusion protein partner can increase an immune response generated against a non-similar, heterologous antigen portion of a fusion protein, while also increasing the in vivo half-life of a fusion protein). Fusion proteins also can comprise one or more cleavage sites, particularly between domains. Additional features and characteristics of fusion proteins that can be suitably incorporated in such fusion proteins are discussed with respect to other fusion proteins provided by the invention.

Variants of such immunogenic peptides, and derivatives of such immunogenic peptides and immunogenic peptide variants are additional features of the invention (e.g., such γ2 immunogenic peptide derivatives can be modified by chemical coupling, genetic fusion, non-covalent association, and the like, to other molecular entities such as antibodies, toxins, radioisotope, cytotoxic agents, or cytostatic agents).

Peptide mimitopes, comprising Ln-5 epitope sequences also can, for example, be useful as “cancer vaccine” candidates. Such peptides also can be useful in the purification of L5G2BPs, such as anti-γ2 mAbs. In addition to the B-cell epitope sequences described herein, such peptides can be engineered or selected to also or alternatively comprise one or additional anti-Ln-5 T cell epitopes, such as anti-γ2 T cell epitopes. Such epitopes can be identified by any suitable technique known in the art (e.g., by T cell epitope prediction software applications). In another aspect, a fusion protein comprising antigenic γ2 DIII B cell epitopes and a heterologous sequence comprising anti-cancer associated T cell epitopes is provided. In another aspect, γ2 DIII binding sequences are included in a peptide with Ln-5-associated T cell epitopes.

In another aspect, the invention provides a nucleic acid comprising a sequence encoding such an immunogenic peptide. Such a nucleic acid can be delivered to a host in a suitable vector, such as a replication-deficient and/or targeted vector (e.g., a targeted nucleic acid vector or a replication-deficient and targeted adenovirus vector), or any other suitable vector described herein or known in the art. The invention also provides compositions comprising one or more of such immunogenic peptides and/or immunogenic peptide-encoding nucleic acids (e.g., with one or more pharmaceutically acceptable carriers, excipients, etc.).

In another aspect, the invention provides secondary and anti-idiotypic antibodies related to L5G2BPs.

A secondary antibody refers to an antibody specific for, and typically raised against, another antibody, such as anti-γ2 DIII antibody (although antibodies against other L5G2BPs can be similarly used/incorporated where applicable). An anti-idiotypic (anti-Id) antibody is an antibody which recognizes unique determinants generally associated with the antigen-binding site of an antibody (e.g., a determinant in an anti-γ2 antibody provided by the invention). An anti-idiotypic antibody can be prepared by immunizing an animal of the same species and genetic type (e.g., a mouse strain) as the source of an anti-γ2 DIII mAb with the mAb to which an anti-Id is being prepared (other methods also may be suitable for preparing such antibodies). The immunized animal typically can recognize and respond to the idiotypic determinants of the immunizing antibody by producing an antibody to these idiotypic determinants (the anti-Id antibody). Examples of such antibodies are described in, e.g., U.S. Pat. No. 4,699,880. Such antibodies are further features of the invention.

An anti-Id antibody can also be used as an immunogen to induce an immune response in yet another animal, producing a so-called anti-anti-Id antibody or an anti-Id antibody response. An anti-anti-Id antibody may be epitopically identical (or substantially so) to the original mAb, which induced the anti-Id. Thus, by using antibodies to the idiotypic determinants of a mAb, it is possible to identify clones expressing antibodies of identical or near identical specificity. Anti-Id antibodies can be varied (thereby producing anti-Id antibody variants) and/or derivatized by any suitable technique(s), such as those described elsewhere herein with respect to anti-γ2 antibodies and other L5G2BPs. For example, anti-Id mAbs can be coupled to a carrier such as keyhole limpet hemocyanin (KLH) and used to immunize BALB/c mice. Sera from these mice typically contain anti-anti-Id antibodies that have binding properties similar if not identical to an original/parent γ2 DIII antibody.

Anti-Id and anti-anti-Id antibodies, nucleic acids comprising L5G2BP-encoding sequences, cells and vectors comprising such nucleic acid molecules, immunogenic peptides corresponding to particular γ2 ADRs, and the like can be referred to as “related compounds” (i.e., compounds that are “related” to L5G2BPs).

Two or more L5G2BPs can be combined and one or more L5G2BPs can be combined with other agents to provide new and advantageous compositions. Similarly, combinations of L5G2BPs and combinations of one or more L5G2BPs and one or more other compositions can be advantageously applied to treat γ2 peptide-associated conditions, such as carcinomas. Similarly, one or more L5G2BPs can be delivered to a patient in combination with the application of one or more therapeutic or prophylactic techniques (e.g., application of radiotherapy, application of surgical techniques, application of a specialized diet, etc.).

In one exemplary aspect, the invention provides a composition comprising two or more L5G2BPs, two or more nucleic acids comprising L5G2BP-encoding sequences (e.g., two or more vectors comprising such nucleic acids), or at least one L5G2BP and at least one L5G2BP-encoding nucleic acid. In a further aspect, the invention provides compositions wherein one or more L5G2BPs and/or nucleic acids comprising L5G2BP-encoding sequences are combined with one or more secondary active agents (agents that induce, promote, and/or enhance a physiological response, typically due to the action of the agent on cells, components thereof, and/or products thereof (or reaction therewith), typically due to action on a cellular receptor), optionally in further combination with one or more pharmaceutically acceptable excipients. In another aspect, L5G2BPs or related compositions (L5G2BP-encoding nucleic acids, related vectors, cells, L5G2BP anti-Id antibodies, etc.) are delivered to a patient in connection with the administration of one or more other therapies directed at treating a condition related to γ2-associated conditions, such as cancer and pre-cancerous conditions.

As suggested above, one exemplary feature of the invention is a composition comprising a plurality of L5G2BPs, such as a “cocktail” of anti-γ2 DIII antibodies. In one such aspect, the invention provides a cocktail of L5G2BPs that each have a different specificity and/or selectivity profile with respect to γ2 binding. In another aspect, the invention provides a cocktail of two or more L5G2BPs some or all of which compete for binding a portion of γ2 where the antigenic determinant regions for these antibodies overlap—an example of such a composition is a composition comprising mAb 5D5, mAb6C12, and derivatives, fragments, and/or variants of either thereof.

In another exemplary facet, the invention provides a composition comprising an effective combination of at least one L5G2BP and at least one additional (or “secondary” or “second”) antineoplastic therapeutic agent, such as an antineoplastic immunogenic peptide, antibody, or small molecule drug (particular examples of which are provided further herein). Thus, for example, in one aspect the invention provides a therapeutic regimen for treatment of patients having γ2 laminin-5 secreting tumors that comprises administering a composition comprising an anti-γ2 DIII mAb in combination with another anti-neoplastic therapeutic and/or prophylactic agent to a patient in need thereof and optionally a pharmaceutically acceptable carrier therefor.

In a further aspect, two or more L5G2BPs or one or more L5G2BPs and one or more secondary agents are co-delivered (e.g., co-administered) to a subject separately but in a coordinated manner so as to reach an at least additive effect in the patient.

The terms “coadministration,” “coadminister,” and the like herein refer to both simultaneous (or concurrent) and serial but related administration, unless otherwise indicated. Coadministration of agents can be accomplished in any suitable manner and in any suitable time. In other words, coadministration can refer to administration of a L5G2D3BP before, simultaneously with, or after, the administration of the secondary antineoplastic agent, at any time(s) that result in an enhancement in the anti-cancer response over the administration of solely the antineoplastic agent, L5G2D3BP, or both (i.e., over either independently). Words such as “co-deliver” and “co-delivered” are to be similarly interpreted, except in also encompassing delivery by other routes besides administration (e.g., expression of a protein from an administered nucleic acid, internal targeted production of an active anti-cancer agent from an administrated prodrug, etc.), unless otherwise stated or clearly contradicted by context.

L5G2BPs can be used in the treatment of a large number of conditions, including a wide variety of cancers. Accordingly, L5G2BPs can be combined with a large number of anti-cancer therapeutic and/or prophylactic agents and therapies. Non-limiting examples of such agents include fluoropyrimidiner carbamates, such as capecitabine; non-polyglutamatable thymidylate synthase inhibitors; nucleoside analogs, such as tocladesine; antifolates such as pemetrexed disodium; taxanes and taxane analogs; topoisomerase inhibitors; polyamine analogs; mTOR inhibitors (e.g., rapamcyin ester); alkylating agents (e.g., oxaliplatin); lectin inhibitors; vitamin D analogs (such as seocalcitol); carbohydrate processing inhibitors; antimetabolism folate antagonists; thumidylate synthase inhibitors; other antimetabolites (e.g., raltitrexed); ribonuclease reductase inhibitors; dioxolate nucleoside analogs; thimylate syntase inhibitors; gonadotropin-releasing hormone (GRNH) peptides; human chorionic gonadotropin; chemically modified tetracyclines (e.g., CMT-3; COL-3); cytosine deaminase; thymopentin; DTIC; carmustine; carboplatin; vinblastine; temozolomide; vindesine; thymosin-α; histone deacetylase inhibitors (e.g., phenylbutyrate); DNA repair agents (e.g., DNA repair enzymes and related compositions such as Dimericine™ (T4 endonuclease V-containing liposome)); gastrin peptides (and related compositions such as Gastrimmune™); GMK and related compounds/compositions (see, e.g., Knutson, Curr Opin Investig Drugs. January 2002;3(1):159-64 and Chapman et al., Clin Cancer Res. December 2000;6(12):4658-62); beta-catenin blockers/inhibitors and/or agents that lower the amount of beta-catenin production in preneoplastic or neoplastic cell nuclei (see, e.g., U.S. Pat. No. 6,677,116), agents that upregulate E-cadherin expression (or E-cadherin); agents that reduce slug (beta-catenin-associated) gene expression; agents that block, inhibit, or antagonize PAI-1 or that otherwise modulate urokinase plasminogen activator (uPA) interaction with the uPA receptor; survivins; DNA demethylating agents; “cross-linking” agents such as platinum-related anti-cancer agents (cisplatin, carboplatin, etc.); agents that block antiapoptotic signaling, such as agents that inhibit MAPK and Ras signaling pathways or components thereof (e.g., agents that interfere with the production and/or function of cyclin D); growth suppressive agents, such as an antimetabolite such as Cepecitabine/Xeloda, cytarabine/Ara-C, Cladribine/Leustatin, Fludaraine/Fludara, fluorouracil/5-FU, gemcitabine/Gemzar, mercaptopurine/6-MP, methotrexate/MTX, thioguanine/6-TG, Allopurinol/Zyloprim, etc.; an acylating agent such as Busulfan, Cyclophosphamide, mechlorethamaine, Melphalan, thiotepa, semustine, carboplatin, cisplatin, procarbazine, dacarbazine, Althretamine, Lomustine, Carmustine, Chlorambucil, etc.; a topoisomerase inhibitor such as Camptothecins as Topotecan, Irinotecan; such as Podophyllotoxins as EtoposideNP16, TeniposideNP26, etc.; an inhibitor of microtuble and/or spindle formation, such as Vincristine, Vinblastine, Vinorelbine, or Taxane such as Paxlitaxel, Docetaxel, combrestatin, Epothilone B, etc; RRR-alpha-tocopheryl succinate; anthracyclins as Daunorubicin/Cerubidine and Doxorubicin; idarubicin; mitomycins; plicamycin; retinoic acid analogues such as all trans retinoic acid, 13-cis retinoic acid, etc.; inhibitors of receptor tyrosine kinases; inhibitors of ErbB-1/EGFR such as iressa, Erbitux, etc.; inhibitors of ErbB-2/Her2 such as Herceptin, etc.; inhibitors of c-kit such as Gleevec; inhibitors of VEGF receptors such as ZD6474, SU6668, etc.; Inhibitors of ErbB3, ErbB4, IGF-IR, insulin receptor, PDGFRa, PDGFRbeta, Flk2, Flt4, FGFR1, FGFR2, FGFR3, FGFR4, TRKA, TRKC, c-met, Ron, Sea, Tie, Tie2, Eph, Ret, Ros, Alk, LTK, PTK7, etc.; cancer related enzyme inhibitors such as metalloproteinase inhibitors such as marimastat, Neovastat, etc.; cathepsin B; modulators of cathepsin D dehydrogenase activity; glutathione-S-transferases and related compounds such as glutacylcysteine synthetase and lactate dehydrogenase; proteasome inhibitors (e.g., Bortezomib); tyrosine kinase inhibitors; farnesyl transferase inhibitors; HSP90 inhibitors (e.g., 17-allyl amino geld-anamycin) and other heat shock protein-inhibitors; mycophenolate mofetil; mycophenolic acid; asparaginase; calcineurin-inhibitors; TOR-inhibitors; multikine molecules; enkephalins (see, e.g., U.S. Pat. No. 6,737,397); SU11248 (Pfizer); BAY 43-9006 (Bayer and Onyx); inhibitors of “lymphocyte homing” mechanisms such as FTY720; Tarceva; Iressa; Glivec; thalidomide; and adhesion molecule inhibitors (e.g., anti-LFA, etc.). Additional anti-neoplastic agents that can be used in the combination composition and combination administration methods of the invention include those described in, e.g., U.S. Pat. Nos. 6,660,309, 6,664,377, 6,677,328, 6,680,342, 6,683,059, and 6,680,306, as well as International Patent Application WO 2003070921.

Where appropriate, one or more of such agents also or alternatively can be conjugated to a L5G2BP. Such conjugates are another feature of the invention.

In one aspect, the invention provides combination compositions and combination delivery/administration protocols that include one or more biological response modifiers (BRMs) in addition to one or more L5G2BPs. BRMs generally are products produced by cells that stimulate or restore the ability of the immune system to act against disease agents (e.g., cancer cells), such as cytokines or antibodies.

In yet another aspect, an L5G2BP and/or related compound is delivered in association with a molecule that binds to free γ2 and/or γ2 fragment peptides, such as a Ln-5-interacting integrin or γ2 peptide-binding fragment thereof (other examples of such agents are described herein and/or are known in the art).

Combination compositions and combination delivery methods also or alternatively can include anti-anergic agents (e.g., small molecule compounds, proteins, glycoproteins, or antibodies that break tolerance to tumor and cancer antigens).

Additional exemplary and particular types of compositions and therapies that may be used/applied with delivery of one or more L5G2BPs or related compositions are described in turn below. It will be recognized that these classifications of agents and methods are non-limiting and only used for convenience. Some of the fore mentioned agents may fall into one or more of the categories described below. Some anti-cancer agents also cannot be readily classified. This includes, for example, the relatively new tyrosine kinase inhibitor imatinib mesylate (Gleevec® or Glivec®). Another example of novel anti-cancer agents are agents that induce fusion of tumor cells, such as measles glycoproteins and related nucleic acids (see, e.g., U.S. Pat. No. 6,750,206).

One feature of the invention is embodied in compositions comprising at least one L5G2BP (e.g., an anti-γ2 DIII mAb or other L5G2D3BP) and a suitable secondary antibody (e.g., a suitable secondary anti-cancer mAb). In general, such a composition can comprise any secondary antibody or combination of secondary antibodies that does not significantly interfere with the specificity, selectivity, and/or affinity of the L5G2BP. Typically, a secondary antibody component of a L5G2BP combination composition exhibits no more than about 5%, such as no more than about 10%, and commonly no more than about 20% competition for γ2 binding with the L5G2BP(s) of the composition.

In a particular aspect, the invention provides a combination composition that includes at least one L5G2BP and at least one secondary anti-cancer monoclonal antibody. A number of suitable anti-cancer mAbs are known in the art and similar suitable antibodies can be developed against cancer-associated targets. Particular examples of suitable second anti-cancer mAbs include anti-CD20 mAbs (such as Rituximab and HuMax-CD20), anti-Her2 mAbs (e.g., Trastuzumab), anti-CD52 mAbs (e.g., Alemtuzumab and Capath® 1H), anti-EGFR mAbs (e.g., Cetuximab, HuMax-EGFr, and ABX-EGF), Zamyl, Pertuzumab, anti-A33 antibodies (see U.S. Pat. No. 6,652,853), anti-oncofetal protein mAbs (see U.S. Pat. No. 5,688,505), anti-PSMA mAbs (see, e.g., U.S. Pat. No. 6,649,163 and Milowsky et al., J Clin Oncol. Jul. 1, 2004;22(13):2522-31. Epub 2004 June 01), anti-TAG-72 antibodies (see U.S. Pat. No. 6,207,815), anti-aminophospholipid antibodies (see U.S. Pat. No. 6,406,693), anti-neurotrophin antibodies (U.S. Pat. No. 6,548,062), anti-C3b(i) antibodies (see U.S. Pat. No. 6,572,856), anti-cytokeratin (CK) mAbs, anti-MN antibodies (see, e.g., U.S. Pat. No. 6,051,226), anti-mtsl mAbs (see, e.g., U.S. Pat. No. 6,638,504), anti-PSA antibodies (see, e.g., Donn et al., Andrologia. 1990;22 Suppl 1:44-55; Sinha et al., Anat Rec. August 1996;245(4):652-61; and Katzenwadel et al., Anticancer Res. May-June 2000; 20(3A):1551-5); antibodies against CA125; antibodies against integrins like integrin beta1; antibodies/inhibitors of VCAM; anti-alpha-v/beta-3 integrin mAbs; anti-kininostatin mAbs; anti-aspartyl (asparaginyl) beta-hydroxylase (HAAH) intrabodies (see, e.g., U.S. Pat. No. 6,783,758); anti-CD3 mAbs (see, e.g., U.S. Pat. Nos. 6,706,265 and 6,750,325) and anti-CD3 bispecific antibodies (e.g., anti-CD3/Ep-CAM, anti-CD3/her2, and anti-CD3/EGP-2 antibodies—see, e.g., Kroesen et al., Cancer Immunol Immunother. November-December 1997; 45(3-4):203-6); and anti-VEGF mAbs (e.g., bevacizumab). Other possibly suitable second mAb molecules include alemtuzumab, edrecolomab, tositumomab, ibritumomab tiuxetan, and gemtuzumab ozogamicin. In one aspect, the invention provides combination compositions and combination therapies that comprise one or more antibodies, typically monoclonal antibodies, targeted against angiogenic factors and/or their receptors, such as VEGF, bFGF, and angiopoietin-1; and monoclonal antibodies against other relevant targets (see also, generally, Reisfeld et al., Int Arch Allergy Immunol. March 2004; 133(3):295-304; Mousa et al., Curr Pharm Des. 2004;10(1):1-9; Shibuya, Nippon Yakurigaku Zasshi. December 2003;122(6):498-503; Zhang et al., Mol Biotechnol. October 2003; 25(2):185-200; Kiselev et al., Biochemistry (Mosc). May 2003;68(5):497-513; Shepherd, Lung Cancer. August 2003;41 Suppl 1:S63-72; O'Reilly, Methods Mol Biol. 2003;223:599-634; Zhu et al., Curr Cancer Drug Targets. June 2002;2(2):135-56; and International Patent Application WO 2004/035537).

Where appropriate such antibodies can be conjugated to a cytotoxin, radionuclide, or another anti-cancer agent. Also where appropriate, immunogenic peptide targets of such antibodies can be used to induce an immune response in a combination composition or combination administration method of the invention.

Where appropriate the targets for these secondary antibodies also can be targeted by multispecific L5G2BPs of the invention. Thus, for example, VH, VL, and/or CDR sequences from such mAbs also can be used in the context of bispecific and multispecific L5G2BPs, such as L5G2D3BPs, described elsewhere herein. Likewise, mAbs specific for targets discussed above with respect to bispecific mAbs, cancer antigens, and other molecules discuss with respect to other aspects also can be used in the methods of the invention and incorporated in the compositions of the invention.

Other antibodies developed against lymphomas, leukemia cells, micrometastases, and solid tumors also may be useful in combination methods and/or combination compositions of the invention. Antibodies that inhibit functions vital for tumor cell survival, growth, invasiveness and/or migration; antibodies that induce ADCC or CDC against tumor/cancer cells; antibodies that interrupt key cancer progression-related signaling events; and/or that deliver a toxic payload to preneoplastic and/or neoplastic cells can be particularly useful in such methods and compositions. Death of the tumor cells also might lead to the release of tumor antigens that “vaccinate” the immune system and stimulates it to produce a secondary response that then targets tumor/cancer cells. Thus, in one aspect, the invention provides a method of targeting a particular population of cancer cells or tumor(s) followed by monitoring of the patient for a secondary response, and providing further anti-cancer therapy if such secondary response is deemed unsatisfactory. Over-expressed oncogenes and tumor-specific antigens can be advantageous targets for such antibodies. Tumor antigens, which can be useful in this context or in their own right as immunogenic peptides (“vaccines”) are described in, for example, Stauss et al.: Tumor antigens recognized by T cells and antibodies. Taylor and Frances (2003) and Durrant et al., Expert Opin. Emerging Drugs 8(2):489-500 (2003).

In a further aspect, the invention provides a combination treatment therapy comprising such a combination of anti-γ2 mAbs or other L5G2BPs and one or more anti-cancer secondary mAb(s). It will be understood that variants of such secondary mAbs, derivatives of such secondary mAbs or secondary mAb variants, and functional fragments of any thereof, also or alternatively can be used in the methods of the invention and incorporated into the compositions of the invention.

In another aspect, the invention provides combination compositions and combination therapy methods involving a “chemotherapeutic agent” in addition to an anti-cancer L5G2BP. Chemotherapeutic agents in the context of this invention typically refer to small molecule compounds selectively toxic to cancer and pre-cancer cells and/or exert one or more effects on the cell cycle of at least some cell types that likely include cancer and pre-cancer cells.

Combination of chemotherapeutic agents, which commonly are used in cancer treatment, also can be combined with one or more L5G2BPs, in composition or by co-delivery. For example, in one aspect the invention provides a method of treating cancer comprising delivering to a patient an effective amount of one or more L5G2BPs in association with an effective application of CHOP chemotherapy (chemotherapy comprising a combination of cyclophosphamide, doxorubicin, vincristine, and prednisone), such that an at least additive anti-cancer effect (if not a greater than additive effect) is obtained.

In another facet, a L5G2BP and/or related composition is delivered in association with a chemotherapeutic that acts on the DNA level of cancer progression (non-limiting examples of conventional chemotherapeutic agents at the DNA level include anti-metabolites (such as 6-mercaptopurine, 6-thioguanine, methotrexate, 5-fluorouracil, and hydroxyurea), cytarabine, alkylating agents, procarbazine, topoisomerase inhibitors, platinum derivatives, anthracyclines, and antibiotics)).

In another particular aspect, the L5G2D3BP or related composition is delivered to a subject or comprised in a composition with an “RNA level” chemotherapeutic agent (or combination thereof), nonlimiting examples of which include L-asparaginase, Vinca alkaloid, taxanes, anti-cancer taxane combination compositions (such as docetaxel-plus-prednisone), and topoisomerase inhibitors.

In another particular aspect, an L5G2BP and/or related composition is delivered in association with an effective dose of dacarbazine (DTIC).

In another particular facet, the invention provides combination compositions and combination therapies that involve effective amount(s) of one or more L5G2BPs and one or more chemotherapeutic agents selected from 5-Fluorouracil, actinomycin D (Dactinomycin), amsacrine, arsenic trioxide, asparaginase, azadcytadine (5-azacytidine, 5AZ), busulfan (myleran), capecitabine, carboplatin (paraplatin), carmustine (BiCNU), chlorambucil (Leukeran), cisplatin (Platinol), cyclophosphamide (Cyroxan), cytarabine (Ara-C), Dacarbazine, Dactinomycin, Daunorubicin (Cerubidine), Docetaxel (Taxotere), Doxorubicin (Adriamycin Doxil), Epirubicin (Ellence), Etoposide (VP-16, Vespid), Fludarabine, Fluorouracil, Gemcitabine, Gleevac (Imatinib mesylate, STI 571), Hydroxyurea, Idarubicin, Ifosfamide (Ifex), Irinotecan, Liposomal Doxurubicin, Lomustine, Mechlorechamine (Mustargen), Melphalan, Mercaptopurine (6MP), Methotrexate, Methyl CCNU, Mitomycin (Mutamycin), Mitoxantrone, Nitrogen Mustard, Nitrosoureas, Oxaliplatin, Paclitaxel (Taxol), Pentostatin, Plicamycin, Procarbazine, Streptozocin, Telcyta (alone or in combination with Doxil), Teniposide (Vumon), Thiotepa, Tretinoin, Vinblastine (Velban), Vincristine (Oncovin), and Vinorelbine (Navelbine).

To better illuminate the invention, non-limiting groups of chemotherapeutic agents useful in L5G2BP combination compositions and therapies are described here.

In one aspect, the invention provides combination compositions and combination therapies characterized in comprising one or more alkylating agents. Alkylating agents are so named because of their ability to add alkyl groups to many electronegative groups under conditions present in cells. These agents stop tumor growth by cross-linking guanine bases in DNA double-helix strands—directly attacking DNA. This makes the strands unable to uncoil and separate. As this is necessary in DNA replication, cells affected by such agents can no longer divide. Examples of alkylating agents include Busulfan (Myleran), Busulfan Injection (Busulfex Injection), Carboplatin (Paraplatin), Carmustine Injection (BiCNU Injection), Chlorambucil (Leukeran), Cyclophosphamide Injection (Cytoxan Injection, Neosar), Dacarbazine (DTIC, DTIC-Dome), Ifosfamide (Ifex), Lomustine (CCNU, CeeNU), Mechlorethamine (Mustargen, Nitrogen mustard), Melphalan (Alkeran, L-PAM), Melphalan Injection (Alkeran Injection), Streptozocin (Zanosar), and Thiotepa (Thioplex).

In another facet, the invention provides combination compositions and therapies that comprise one or more L5G2BPs and one or more antimetabolites. Antimetabolites prevent cancer cells from processing nutrients and other substances that are necessary for normal activity in the cancer cells. More particularly, antimetabolites masquerade as purine or pyrimidine, which become the building blocks of DNA. They prevent these substances becoming incorporated in to DNA during the “S” phase of the cell cycle, thereby preventing cell division. There are several different cellular targets for antimetabolites. Some common classes of antimetabolites Folate Antagonists, Purine Antagonists, and Pyrimidine Antagonists.

Folate antagonists, also known as antifolates, inhibit dihydrofolate reductase (DHFR), an enzyme involved in the formation of nucleotides. When this enzyme is blocked, nucleotides are not formed, disrupting DNA replication and cell division. Methotrexate is the primary folate antagonist used as a chemotherapeutic agent.

The purine antagonists function by inhibiting DNA synthesis in two different ways. They can inhibit the production of the purine containing nucleotides, adenine and guanine. If a cell doesn't have sufficient amounts of purines, DNA synthesis is halted and the cell cannot divide. They also may be incorporated into the DNA molecule during DNA synthesis. The presence of the inhibitor is thought to interfere with further cell division. Examples of purine antagonists include 6-Mercaptopurine, Dacarbazine, and Fludarabine.

Pyrimidine antagonists also can be combined or co-delivered with one or more L5G2BPs. Pyrimidine antagonists act to block the synthesis of pyrimidine containing nucleotides. These drugs typically have structures similar to the natural compound that they replace. By acting as ‘decoys’, these drugs can prevent the production of the finished nucleotides. They may exert their effects at different steps in that pathway and may directly inhibit crucial enzymes. Pyrimidine antagonists may also be incorporated into a growing DNA chain and lead to termination of the process. Examples of pyrimidine antagonists include 5-fluorouracil, Arabinosylcytosine, Capecitabine, and Gemcitabine.

In an exemplary aspect, the invention provides combination compositions and therapies characterized by including one or more metabolites selected from Floxuridine (FUDR, Fluorodeoxyuridine), Fludarabine Phosphate (Fludara), Gemcitabine Hydrochloride (Gemzar), Hydroxyurea (Droxia, Hydrea), Mercaptopurine (6-MP, Purinethol), Methotrexate (Rheumatrex, Trexall), Methotrexate Injection (Amethopterin, MTX Injection), Thioguanine (6-TG, TG), and combinations of any thereof.

The invention also relates to combination compositions and therapies including one or more antineoplastic hormones. Antineoplastic hormones interfere at the cellular level with receptors for growth stimulating proteins. By blocking the receptor, the production or release of growth factors is reduced. Examples of such agents include Diethylstilbestrol Injection (Stilphostrol Injection), Megestrol (Megace), and Mitotane (Lysodren).

In a further facet, the invention provides combination compositions and combination delivery methods characterized in including one or more mitotic inhibitors. Mitotic inhibitors generally prevent cell division by interfering with the protein called tubulin. Tubulin is the basic building block of the fibers that are responsible for ensuring that each cell continues to multiply. Examples of such agents include Docetaxel (Taxotere), Etoposide Injection (Toposar, VePesid Injection), Etoposide Oral (VP-16, VePesid Oral), Paclitaxel (Onxol, Taxol), Vinblastine (Velban), and Vincristine (Oncovin, Vincasar).

Another somewhat related class of classic chemotherapeutic agents is plant alkaloids. These alkaloids are derived from plants and block cell division by preventing microtubules being synthesized. These are vital for cell division and without them it can not occur. The main examples are vinca alkaloids such as vincristine.

Another also somewhat related class of classic chemotherapeutic agents is “genotoxic drugs.” Genotoxic drugs are chemotherapy agents that affect nucleic acids and alter their function. These drugs may directly bind to DNA or they may indirectly lead to DNA damage by affecting enzymes involved in DNA replication and possibly apoptosis. Such genotoxic chemotherapy treatments include alkylating agents, intercalating agents (drugs that “wedge” into the spaces between the nucleotides in the DNA double helix, thereby interfering with transcription and replication, and often inducing mutations), and enzyme inhibitors (e.g., agents that inhibit key enzymes, such as topoisomerases, involved in DNA replication inducing DNA damage). Thus, the genotoxic drug class of chemotherapeutic agents overlaps with other classes described elsewhere herein. A goal of treatment with any of these agents includes (i.e., a common mechanism of action associated with such agents is) the induction of DNA damage in the cancer cells. DNA damage, if severe enough, will induce cells to undergo apoptosis. Genotoxic chemotherapy drugs typically affect both normal and cancerous cells. The selectivity of the drug action is based on the sensitivity of rapidly dividing cells, such as cancer cells, to treatments that damage DNA. Examples of agents that can be classified as genotoxic agents include Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolamide, and Topotecan.

Another feature of the invention is embodied in combination methods and compositions that comprise one or more L5G2BPs (or delivery thereof) and one or more nucleic acids that act as anti-cancer agents (or delivery thereof).

In a particular aspect, the invention provides combination compositions and methods, wherein a L5G2BP is combined with or delivered in association with a nucleic acid comprising a sequence encoding a tumor suppressor. In one exemplary facet, one or more L5G2BPs are delivered in association with the delivery of a p53 tumor suppressor gene (see, e.g., Roth et al., Oncology (Huntingt). October 1999;13(10 Suppl 5):148-54) and Nielsen et al., Cancer Gene Ther. January-February 1998;5(1):52-63). Additional tumor suppressor targets include, for example, BRCA1, RB1, BRCA2, DPC4 (Smad4), p21, E2F-1, FUS1 compounds (e.g., INGN 401), MSH2, MLH1, and DCC. Such a nucleic acid can be delivered in the form of a suitable vector, host cell, etc. For example, one or more L5G2BPs can be combined with or delivered in association with a replication-deficient adenovirus encoding human recombinant wild-type p53/SCH58500.

A further feature of the invention is the provision of combination methods and combination compositions that include one or more L5G2BPs and one or more nucleic acids that are able to reduce one or more aspects of expression of particular cancer-associated genes. Such agents include antisense nucleic acids and inhibitory RNA (iRNA) molecules.

In one exemplary aspect, the invention provides combination compositions and methods that involve one or more antisense nucleic acids targeted to oncogenes, mutated, or deregulated genes. In another exemplary aspect, the invention provides combination compositions and methods that involve at least one siRNA molecule targeted to mutated or deregulated genes. Another feature of the invention is combination compositions and methods that include one or more antisense oligonucleotides and/or siRNAs that reduce the expression of oncogenes or other cancer progression-related genes (e.g., Ln-5-targeted or integrin-targeted antisense molecules—which are described in, e.g., U.S. Pat. No. Application 2003224993 and O'Toole et al., Exp Cell Res. Jun. 15, 1997;233(2):330-9, and/or antisense molecules against Ln-5-modulators, such as MT1-MMP antisense oligonucleotides (see, e.g., Giles et al., J Cell Sci. August 2001;114(Pt 16):2967-76)), other inhibitors of Ln-5 production or activity (e.g., p300—see, e.g., Miller et al., J Biol Chem. Mar. 17, 2000;275(11):8176-82), mutated genes, and/or deregulated genes.

For example, a L5G2BP, such as a L5G2D3BP, can be combined with or administered in association with an anti-cancer antisense nucleic acid (e.g., Genasense™ (augmerosen/G3139)), LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCα, clusterin, IGFBPs, protein kinase A, cyclin D1, or Bcl-2—see, e.g., Benimetskaya et al., Clin Prostate Cancer. June 2002;1(1):20-30; Tortora et al., Ann NY Acad Sci. December 2003;1002:236-43; Gleave et al., Ann NY Acad Sci. December 2003;1002:95-104.; Lahn et al., Ann NY Acad Sci. December 2003;1002:263-70; Kim et al., Int J Oncol. January 2004;24(1):5-17; Stahel et al., Lung Cancer. August 2003;41 Suppl 1:S81-8; Stephens et al., Curr Opin Mol Ther. April 2003;5(2):118-22; Cho-Chung, Arch Pharm Res. March 2003;26(3):183-91; and Chen, Methods Mol Med. 2003;75:621-36)). In another exemplary aspect, a L5G2BP is delivered in association with or combined in a composition with an anti-cancer inhibitory RNA molecule (see, e.g., Lin et al., Curr Cancer Drug Targets. November 2001;1(3):241-7, Erratum in: Curr Cancer Drug Targets. June 2003;3(3):237; Lima et al., Cancer Gene Ther. May 2004;11(5):309-16; Grzmil et al., Int J Oncol. January 2004;24(1):97-105; Collis et al., Int J Radiat Oncol Biol Phys. Oct. 1, 2003;57(2 Suppl):S144; Yang et al., Oncogene. Aug. 2003; 22(36):5694-701; and Zhang et al., Biochem Biophys Res Commun. April 18, 2003; 303(4): 1169-78 for discussion relating to such iRNA molecules, related principles, and related methods).

In another facet, the invention provides combination compositions and combination administration methods where a L5G2D3BP is combined with an anti-cancer nucleozyme, such as a ribozyme, examples of which include angiozyme (Ribozyme Pharmaceuticals) (see e.g., Pennati et al., Oncogene. Jan. 15, 2004;23(2):386-94; Tong et al., Clin Lung Cancer. February 2001; 2(3):220-6; Kijima et al., Int J Oncol. March 2004;24(3):559-64; Tong et al., Chin Med J (Engl). October 2003;116(10):1515-8; and Orlandi et al., Prostate. Feb. 1, 2003;54(2):133-43) and herzyme (in a related sense see U.S. Pat. No. 6,617,438). Additional anti-cancer ribozymes are described in, e.g., U.S. Pat. No. Applications 20030195164, 20030050236, and 20030105043 and U.S. Pat. Nos. 6,482,803 and 6,489,163. See also Poliseno et al., Current Pharmaceutical Biotechnology August 2004, vol. 5, no. 4, pp. 361-368(8) for a review of RNA-based drugs.

In yet another aspect, a L5G2BP is combined or co-delivered with an immunostimulatory nucleic acid (in another aspect, a nucleic acid comprising a sequence encoding a L5G2BP and at least one immunostimulatory sequence is provided). Numerous examples of suitable immunostimulatory nucleic acids have been described in the art (see, e.g., Krieg, Trends in Microbiol 7: 64-65 (1999); Wooldridge et al., Curr Opin Oncol. November 2003;15(6):440-5; Jahrsdorfer et al., Semin Oncol. August 2003;30(4):476-82; Jahrsdorfer et al., Curr Opin Investig Drugs. June 2003;4(6):686-90; Carpentier et al., Front Biosci. Jan. 1, 2003; 8:e115-27; U.S. Pat. No. 6,406,705; U.S. Pat. No. 6,218,371; U.S. Patent Application 20040181045; and U.S. Patent Application 20040087538).

In another aspect, the invention provides methods for treating γ2-associated conditions that comprising delivering one or more L5G2BPs and/or combining one or more L5G2BPs (or related compositions) with at least one Ln-5-encoding nucleic acid (in a form associated with normal cell basement membrane attachment/association), at least one nucleic acid that upregulates endogenous Ln-5 production (e.g., by so-called gene activation), and/or one or more cells expressing Ln-5 at levels at least as great as in normal basement membrane-associated epithelial cells. Other functional gene replacement methods also can be used in the context of the methods and reflected in compositions of this invention (e.g., providing a nucleic acid encoding a non-cancer-associated version of a tumor suppressor such as p53). Another use of gene therapy is the introduction of enzymes into these cells that make cancer cells susceptible to particular chemotherapy agents (e.g., introducing thymidine kinase into cancer cells so as to make them susceptible to aciclovir).

In a further aspect, the invention provides combination compositions and administration methods wherein a L5G2BP is combined with or co-administered with a basal lamina-targeted and/or basal lamina-associated factor modulating anti-cancer molecules (e.g., a molecule that inhibits breakdown of the basal lamina in cancer progression), such as ginsenoside-Rb2, anti-MMP-1 antibodies, anti-integrin antibodies, anti-MMP2 antibodies and inhibitors, anti-MT1-MMP antibodies and inhibitors, anti-EGF-R antibodies, anti-BMP-1 inhibitors and antibodies, and inhibitors of urokinase-type plasminogen activator (uPA) and/or plasminogen activation to plasmin (aprotinin, amiloride, EACA, tranexamic acid, anti-uPA antibody). In another aspect, the invention provides such compositions and methods wherein the composition or method comprises an inhibitor of Thymosin beta 4.

In an additional facet, the invention provides combination compositions and combination administration methods wherein a L5G2BP is combined or codelivered with a virus or related molecule (e.g., a virus like particle, a viral nucleic acid, etc.) that acts as an active agent against cancer. In one aspect, the invention provides combination compositions and methods that comprise one or more L5G2BPs (or related compositions) and at least one oncolytic virus. Examples of such viruses include oncolytic adenoviruses and herpes viruses, which may or may not be modified viruses (see, e.g., Teshigahara et al., J Surg Oncol. January 2004;85(1):42-7; Stiles et al., Surgery. August 2003;134(2):357-64; Zwiebel et al., Semin Oncol. August 2001;28(4):336-43; Varghese et al., Cancer Gene Ther. December 2002;9(12):967-78; and Wildner et al., Cancer Res. Jan. 15, 1999;59(2):410-3).

Various viruses, viral proteins, and the like can be used in combination compositions and combination administration methods. Replication-deficient viruses, that generally are capable of one or only a few rounds of replication in vivo, and that are targeted to tumor cells, can, for example, be useful components of such compositions and methods. Such viral agents can comprise or be associated with nucleic acids encoding immunostimulants, such as GM-CSF and/or IL-2. Both naturally oncolytic and such recombinant oncolytic viruses (e.g., HSV-1 viruses; reoviruses; replication-deficient and replication-sensitive adenovirus; etc.) can be useful components of such methods and compositions (see, e.g., Varghese et al., Cancer Gene Ther. December 2002;9(12):967-78; Zwiebel et al., Semin Oncol. August 2001;28(4):336-43; Sunarmura et al., Pancreas. April 2004;28(3):326-9; Shah et al., J Neurooncol. December 2003;65(3):203-26; and Yamanaka, Int J Oncol. April 2004;24(4):919-23).

Additional features of the invention include combination administration methods and combination compositions wherein a L5G2BP is combined or delivered with an anti-cancer immunogen, such as a cancer antigen/tumor-associated antigen (e.g., an epithelial cell adhesion molecule (Ep-CAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, CEA, cyclin-dependent kinase 4, β-catenin, capsase-B, tyrosinase, cancer-associated viral vaccines (e.g., human papillomavirus vaccines), tumor-derived heat shock proteins, and the like, additional examples of which are described elsewhere herein) (see also, e.g., Acres et al., Curr Opin Mol Ther February 2004, 6:40-7; Taylor-Papadimitriou et al., Biochim Biophys Acta. Oct. 8, 1999; 1455(2-3):301-13; Emens et al., Cancer Biol Ther. July-August 2003;2(4 Suppl 1):S161-8; and Ohshima et al., Int J Cancer. Jul. 1, 2001;93(1):91-6). A number of other suitable cancer antigens/tumor-associated antigens described herein (e.g., gp75) and similar molecules known in the art also or alternatively can be used in such combination administration methods or incorporated in such combination compositions.

Anti-cancer immunogenic peptides also include anti-Idiotypic “vaccines” such as BEC2 anti-Idiotypic mAb (Mitumomab—see, e.g., Chapman, Curr Opin Investig Drugs. June 2003; 4(6):710-5 and McCaffery et al., Clin Cancer Res. April 1996;2(4):679-86), CeaVac® and related anti-Idiotypic mAbs (see, e.g., Foon et al., J Clin Oncol. September 1999;17(9):2889-5), anti-idiotypic mAb to MG7 mAb (see, e.g., Fengtian et al., Chin Med Sci J. December 2002;17(4):215-9), and other anti-cancer anti-Idiotypic Abs (see, e.g., Birebent et al., Vaccine. Apr. 2, 2003; 21(15):1601-12, Li et al., Chin Med J (Engl). September 2001;114(9):962-6, Schmitt et al., Hybridoma. October 1994;13(5):389-96, Maloney et al., Hybridoma. 1985 Fall;4(3):191-209, Raychardhuri et al., J Immunol. Sep. 1, 1986;137(5):1743-9, Pohl et al., Int J Cancer. Apr. 1, 1992; 50(6):958-67, Bohlen et al., Cytokines Mol Ther. December 1996;2(4):231-8, and Maruyama, J Immunol Methods. Jun. 1, 2002;264(1-2):121-33). Such anti-Idiotypic Abs can be advantageously optionally conjugated to a carrier, which may be a synthetic (typically inert) molecule carrier, a protein (e.g., keyhole limpet hemocyanin (KLH) (see, e.g., Ochi et al., Eur J Immunol. November 1987;17(11):1645-8)), or a cell (e.g., a red blood cell—see, e.g., Wi et al., J Immunol Methods. Sep. 1, 1989;122(2):227-34)).

Compositions and combination administration methods of the invention also include the inclusion or coadministration of nucleic acid vaccines, such as naked DNA vaccines encoding such cancer antigens/tumor-associated antigens (see, e.g., U.S. Pat. Nos. 5,589,466, 5,593,972, 5,703,057, 5,879,687, 6,235,523, and 6,387,888).

In another aspect, the combination administration method and/or combination composition comprises an autologous vaccine composition. In a further aspect, the combination composition and/or combination administration method comprises a whole cell vaccine or cytokine-expressing cell (e.g., a recombinant IL-2 expressing fibroblast, recombinant cytokine-expressing dendritic cell, and the like) (see, e.g., Kowalczyk et al., Acta Biochim Pol. 2003;50(3):613-24; Reilly et al., Methods Mol Med. 2002;69:233-57; and Tirapu et al., Curr Gene Ther. February 2002;2(1):79-89). Another example of a therapeutic autologous cell method that can be useful in combination methods of this invention is the MyVax® Personalized Immunotherapy method (previously referred to as GTOP-99) (available through Genitope Corporation—Redwood City, Calif., USA) (see U.S. Pat. Nos. 5,972,334 and 5,776,746).

In a further aspect, combination compositions and/or combination administration methods of the invention comprise administration of an immunomodulatory compound or modulator thereof (e.g., an anti-inhibitory immunomodulatory antibody). Examples of such compounds include T cell activating and proliferation-promoting molecules, such as B7 molecules (B7-1, B7-2, variants thereof, and fragments thereof) (see, e.g., Adv Exp Med Biol. 2000;465:381-90 and U.S. Patent Application 20030208058), ICOS (inducible co-stimulator) molecules, and OX40 molecules (see Coyle et al., Springer Semin Immunopathol. February 2004;25(3-4):349-59 and U.S. Pat. No. 6,312,700). Another example of such a molecule is an inhibitor of a negative T cell regulator, such as an antibody against CTLA4, such as MDX-010 (Phan et al. (2003) Proc. Natl. Acad. Sci. U.S.A. 100: 8372). Antibodies against several members of the TNF receptor (TNFR) family have been shown to augment T cell proliferative responses. Antibodies to CD27 have also been shown to enhance T cell proliferation. The interaction of the integrin family member LFA-1 (lymphocyte function-associated antigen 1, or CD18/CD11a) with its ligands intercellular adhesion molecule (ICAM)-1, -2 and -3 is well known to be an important participant in the activation of T cells. SLAM (signaling lymphocyte activation molecule, or CDw150) is another T cell regulator. The heat-stable antigen (HSA or CD24) is a glycophosphatidylinositol (GPI)-linked protein of 27 amino acids found on the surface of hematopoietic and neuronal cells in an extensively glycosylated 38-70 kDa form that enhances T cell proliferation. 4-1BB is a co-stimulatory receptor for T cells. TNFR-associated factors (TRAFs) also are T cell signaling molecules. CD40L also can act as an immunomodulator. The CD2-LFA3 pathway also is important to T cell regulation (and accordingly agents that act on it can be included in combination methods and compositions). NK cell activating and proliferating agents, such as stimulatory KIR molecules also can be included in such combination methods and compositions. Other immunomodulating agents that can also or alternatively be included in such combination compositions and methods are TGF-beta inhibitors.

Cytokines and chemokines, which represent an important subset of immunomodulators, are discussed in detail in the following discussion.

The invention provides combination composition and combination delivery methods comprising at least one L5G2BP and at least one anti-cancer cytokine, chemokine, or combination thereof.

In general, any suitable anti-cancer cytokine and/or chemokine can be used with and/or combined with L5G2BPs in the methods and compositions of this invention. Suitable chemokines and cytokines result in a detectably greater and/or more comprehensive immune response to cancer cells or related tissues (e.g., tumors) in vivo and do not substantially impede the binding of the L5G2D3BP(s) in the composition/method.

Examples of suitable cytokines and growth factors include interferons (e.g., IFNβ, IFNα (e.g., INFα2b), and IFNγ (e.g., IFNγ1b)) and interleukins (e.g., IL-2, IL-4, IL-6, IL-7, IL-10, IL-12, IL-13, IL-15, IL-18, IL-23, IL-24, IL-27, IL-28a, IL-28b, IL-29, etc.). Additional cytokines that can be included in such compositions and methods include KGF, IFNβ, GM-CSF, CD40L, Flt3 ligand, stem cell factor, ancestim, and TNFα (see, e.g., Dranoff, Nat Rev Cancer. January 2004; 4(1):11-22 and Szlosarek, Novartis Found Symp. 2004;256:227-37; discussion 237-40, 259-69).

Suitable chemokines can include Glu-Leu-Arg (ELR)-negative chemokines such as IP-10, MCP-3, MIG, and SDF-1 alpha from the human CXC and C—C chemokine families. Suitable cytokines include cytokine derivatives, cytokine variants, cytokine fragments, and cytokine fusion proteins (see, e.g., Eliason, BioDrugs, 2001;15(11):705-11 (with respect to PEGylated cytokines) and Shibuya et al., Laryngoscope. November 2003;113(11):1870-84 (other cytokine derivatives), and WO 01/79258 (albumin-cytokine fusion proteins)).

These and other methods involving naturally occurring peptide-encoding nucleic acids herein can alternatively or additionally performed by “gene activation” and homologous recombination gene upregulation techniques, such as are described in U.S. Pat. Nos. 5,968,502, 6,063,630, and 6,187,305 and European Patent Publication 0 505 500. Additionally useful cytokines for such combination therapies and compositions are described elsewhere herein.

In another aspect, the invention provides a combination composition or combination administration method comprising a L5G2BP and an adjuvant, typically in further combination with an anti-cancer immunogenic peptide (or surrogate nucleic acid/nucleic acid-encoding molecule). Non-limiting examples of suitable adjuvants are QS21, GM-CSF, SRL-172, histamine dihydrochloride, thymocartin, Tio-TEPA, monophosphoryl-lipid A/micobacteria compositions, alum, incomplete Freund's Adjuvant, Montanide ISA, Ribi Adjuvant System, TiterMax adjuvant, syntex adjuvant formulations, immune-stimulating complexes (ISCOMs), GerbuR adjuvant, CpG oligodeoxynucleotides, lipopolysaccharide, and polyinosinic:polycytidylic acid.

Combination compositions and combination administration methods also can involve “whole cell” and “adoptive” immunotherapy methods and “internal vaccination” techniques. For example, such methods can comprise infusion or re-infusion of immune system cells (e.g., tumor-infiltrating lymphocytes (TILs), such as CD4+ and/or CD8+ T cells (e.g., T cells expanded with tumor-specific antigens and/or genetic enhancements), antibody-expressing B cells or other antibody producing/presenting cells, dendritic cells (e.g., anti-cytokine expressing recombinant dendritic cells, dendritic cells cultured with a DC-expanding agent such as GM-CSF and/or Flt3-L, and/or tumor-associated antigen-loaded dendritic cells), anti-tumor NK cells, so-called hybrid cells, or combinations thereof (see, e.g., Fishman et al., Expert Rev Anticancer Ther. December 2003;3(6):837-49; Whiteside et al., Cancer Immunol Immunother. March 2004; 53(3):240-8; Conrad et al., Curr Opin Mol Ther. August 2003;5(4):405-12; Trefzer et al., Mol Biotechnol. September 2003;25(1):63-9; Reinhard et al., Br J Cancer. May 2002;86(10):1529-33; Korbelik et al., Int J Cancer. Jul. 2001;93(2):269-74; Costa et al., J Immunol. Aug. 15, 2001; 167(4):2379-87; Hanson et al., Immunity. August 2000;13(2):265-76; Matsui et al., Int Immunol. July 2003;15(7):797-805; and Ho et al., Cancer Cell. May 2003;3(5):431-7). Cell lysates also may be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see, e.g., Bystryn et al., Clinical Cancer Research Vol. 7, 1882-1887, July 2001), optionally admixed with adjuvants such as alum, also can be advantageous components in such methods and methods. U.S. Pat. No. 6,699,483 provides another example of a whole cell anti-cancer therapy. Additional examples of such whole cell immunotherapies that can be usefully combined in L5G2BP-related compositions and methods are described elsewhere herein.

In another aspect, one or more L5G2BPs are delivered to a patient in association with the delivery of an effective amount of antigen-pulsed dendritic cells or other anti-cancer immune cells (e.g., NK cells).

In yet another aspect, a L5G2BP can be delivered to a patient in combination with the application of an internal vaccination method. Internal vaccination refers to induced tumor or cancer cell death, such as drug-induced or radiation-induced cell death of tumor cells, in a patient, that typically leads to elicitation of an immune response directed towards (i) the tumor cells as a whole or (ii) parts of the tumor cells including (a) secreted proteins, glycoproteins or other products, (b) membrane-associated proteins or glycoproteins or other components associated with or inserted in membranes, and/or (c) intracellular proteins or other intracellular components. An internal vaccination-induced immune response may be humoral (i.e. antibody—complement-mediated) or cell-mediated (e.g., the development and/or increase of endogenous cytotoxic T lymphocytes that recognize the internally killed tumor cells or parts thereof). In addition to radiotherapy, non-limiting examples of drugs and agents that can be used to induce said tumor cell-death induction and internal vaccination methods include conventional chemotherapeutic agents, cell-cycle inhibitors, anti-angiogenesis drugs, monoclonal antibodies, apoptosis-inducing agents, and signal transduction inhibitors.

In another aspect, the invention provides combination compositions and combination administration methods that involve at least one L5G2BP and one or more cell cycle control/apoptosis regulators (or cell cycle/apoptosis “regulating agents”).

A cell cycle control/apoptosis regulator that can be combined with L5G2BP(s) can include, for example, one or more molecules that target and modulate cell cycle control/apoptosis regulators such as (i) cdc-25 (with NSC 663284 as a non-limiting example (see, e.g., Pu et al (2003) J Biol Chem 278, 46877)), (ii) cyclin-dependent kinases that overstimulate the cell cycle (non-limiting examples of which are flavopiridol (L868275, HMR1275; Aventis), 7-hydroxystaurosporine (UCN-01, KW-2401; Kyowa Hakko Kogyo), and roscovitine (R-roscovitine, CYC202; Cyclacel)—as reviewed by Fischer & Gianella-Borradori (2003) Exp Op Invest Drugs 12, 955-970), and (iii) telomerase modulators (such as BIBR1532 (Damm et al (2001) EMBO J 20, 6958-6968) and SOT-095 (Tauchi et al (2003) Oncogene 22, 5338-5347)). Mycobacterium DNA has been reported to be capable of inducing apoptosis in cancer cells (see, e.g., U.S. Pat. No. 6,794,368).

Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2 (see Igney and Krammer (2002) Nature Rev. Cancer 2, 277-288; Makin; Dive (2003) Trends Mol Med 9, 2519; Smyth et al (2003) Immunity 18, 1-6; and Panaretakis et al. (2003) Oncogene 22, 4543-4556).

In another aspect, the invention provides combination compositions and combination delivery methods comprising a telomerase inhibitor, telomerase vaccine, or combination thereof in addition to at least one L5G2BP or related molecule. Examples of such compositions and related techniques are described in U.S. Pat. Nos. 6,440,735 and 6,713,055.

In yet another aspect, the invention provides combination compositions and combination administration methods that comprise one or more growth factor inhibitors.

A number of antibodies (e.g., mAbs) against growth factors and growth factor receptors are known that can be useful in promoting the treatment of cancer. For example, antibodies against the extracellular ligand binding domain of epidermal growth factor receptor (EGF-R) proteins that are abnormally activated in epithelial tumors can be useful in the treatment of aggressive epithelial cell-derived tumors. Antibodies against low molecular weight molecules and small molecules that inhibit the tyrosine kinase domains of such receptors also can be useful in combination compositions or combination administration methods. Non-limiting examples of such molecules include Herceptin (monoclonal antibody), Cetuximab (monoclonal antibody), Tarceva (small molecule low molecular weight inhibitor), and Iressa (small molecule low molecular weight inhibitor). Additional related and useful antibodies suitable for inclusion in such combination compositions and administration methods are described elsewhere herein.

In a further aspect, the invention provides combination compositions and methods that include one or more L5G2BPs (or related molecule surrogates) and one or more inhibitors of angiogenesis, neovascularization, and/or other vascularization (such agents are referred to by terms such as anti-angoigenesis agents, anti-angiogenic drugs, etc. herein). Nonlimiting examples of such agents include (individually or in combination) endostatin and angiostatin (reviewed in Marx (2003) Science 301, 452-454) and derivatives/analogues thereof; anti-angiogenic heparin derivatives and related molecules (e.g., heperinase III); VEGF-R kinase inhibitors and other anti-angiogenic tyrosine kinase inhibitors (e.g., SU011248—see Rosen et al., Clinical Oncology; May 31-June 3, 2003, Chicago, Ill., USA (abstract 765)); temozolomide; Neovastat™ (Gingras et al., Invest New Drugs. 2004 Jan;22(1):17-26); Angiozyme™ (Weng et al., Curr Oncol Rep. March 2001;3(2):141-6); NK4 (Matsumoto et al., Cancer Sci. April 2003; 94(4):321-7); macrophage migration inhibitory factor (MIF); cyclooxygenase-2 inhibitors; resveratrol (see, e.g., Sala et al., Drugs Exp Clin Res. 2003;29(5-6):263-9); PTK787/ZK 222584 (see, e.g., Klem, Clin Colorectal Cancer. November 2003;3(3):147-9 and Zips et al., Anticancer Res. September-October 2003;23(5A):3869-76); anti-angiogenic soy isoflavones (e.g., Genistein—see, e.g., Sarkar and Li, Cancer Invest. 2003;21 (5):744-57); Oltipraz; thalidomide and thalidomide analogs (e.g., CC-5013—see, e.g., Tohnya et al., Clin Prostate Cancer. March 2004;2(4):241-3); other endothelial cell inhibitors (e.g., Squalamine and 2-methoxyestradiol); fumagillin and analogs thereof; somatostatin analogues; pentosan polysulfate; tecogalan sodium; molecules that block matrix breakdown (such as suramin and analogs thereof (see, e.g., Marchetti et al., Int J Cancer. Mar. 20, 2003;104(2):167-74, Meyers et al., J Surg Res. Jun. 15, 2000;91(2):130-4, Kruger and Figg, Clin Cancer Res. July 2001;7(7):1867-72, and Gradishar et al., Oncology. May 2000; 58(4):324-33)); dalteparin (Scheinowitz et al., Cardiovasc Drugs Ther. July 2002;16(4):303-9); matrix metalloproteinase inhibitors (such as BMS-275291—see Rundhaug, Clin Cancer Res. February 2003;9(2):551-4; see generally, Coussens et al. Science 2002;295:2387-2392); angiocol; anti-PDGF mAbs and other PDGF (platelet derived growth factor) inhibitors; and PEDFs (pigment epithelium derived growth factors).

In another aspect, the invention provides combination compositions and combination administration methods wherein at least one L5G2BP is combined with or delivered in association with a hormonal regulating agent, such as an anti-androgen and/or anti-estrogen therapy agent or regimen (see, e.g., Trachtenberg, Can J Urol. June 1997;4(2 Supp 1):61-64; Ho, J Cell Biochem. February 15, 2004;91(3):491-503), tamoxifen, a progestin, a luteinizing hormone-releasing hormone (or an analog thereof or other LHRH agonist), or an aromatase inhibitor (see, e.g., Dreicer et al., Cancer Invest. 1992;10(1):27-41). Steroids (often dexamethasone) can inhibit tumour growth or the associated edema (brain tumors) and also can be suitable for combination with L5G2BPs (or related compound surrogates thereof). One or more L5G2BPs can be similar provided or combined with an antiandrogene such as Flutaminde/Eulexin; a progestin, such as hydroxyprogesterone caproate, Medroxyprogesterone/Provera, Megestrol acepate/Megace, etc.; an adrenocorticosteroid such as hydrocortisone, prednisone, etc.; a luteinising hormone-releasing hormone (LHRH) analogue such as buserelin, goserelin, etc.; and/or a hormone inhibitor such as octreotide/Sandostatin, etc. In a particular aspect, L5G2BP(s) are provided or combined with an anti-cancer agent that is an estrogen receptor modulator (ERM) such as tamoxifen, idoxifene, fulvestrant, droloxifene, toremifene, raloxifene, diethylstilbestrol, ethinyl estradiol/Estinyl, etc., or a combination of any thereof. Combination compositions and combination administration methods also or alternatively can comprise tamoxifen. Further teachings relevant to cancer immunotherapy are provided in, e.g., Berczi et al., “Combination Immunotherapy of Cancer” in NEUROIMMUNE BIOLOGY, Volume 1: New foundation of Biology, Berczi I, Gorczynski R, Editors, Elsevier, 2001;pp. 417-432.

In a particular aspect, one or more L5G2BPs (or suitable related molecule surrogates therefor—such substitution of L5G2BPs with surrogates is contemplated throughout unless otherwise stated or clearly contradicted) is combined with or delivered in association with one or more aromatase inhibitors, such as anastrazole/Arimidex, aminoglutethimide/Cytraden, Exemestane, etc. Anti-aromatase agents inhibit the cytochrome P-450 component of the aromatase enzyme complex by interfering with the electron transfer from NADPH. Examples of such agents include anastrozole (Arimidex) and letrozole (Femara). These drugs can be also classified into first-generation (e.g. aminoglutethimide), second-generation (e.g. formestane and fadrazole) and third-generation (e.g. anastrozole, letrozole and exemestane) compounds. Anti-aromatase agents may also be divided into Type I and Type II inhibitors. The Type I inhibitors have a steroidal structure similar to androgens and inactivate the enzyme irreversibly by blocking the substrate-binding site, and are therefore known as aromatase inactivators. Examples of such drugs include formestane and exemestane (Aromasin). Type II inhibitors are non-steroidal and their action is reversible. Examples include anastrozole and letrozole. In one aspect, one or more L5G2BPs are provided or combined with one or more of such molecules selected from Formestane, Exemestane, Aminoglutethimide, Anastrozole, and Letrozole.

Prostate cancer is often sensitive to finasteride, an agent that blocks the peripheral conversion of testosterone to 5-hydroxy-testosterone. L5G2BPs can be provided or combined with this agent or provided in association with various forms of androgen deprivation therapy (ADT).

In one aspect, one or more L5G2BPs are combined with or co-delivered with one or more intracellular signaling inhibitors. Examples of such compounds include tyrosine kinase inhibitors (Gleevec®, imatinib mesylate), modulators of the ras signaling pathway, and regulators of protein trafficking. Other examples include serine/threonine kinase inhibitors, protein-tyrosine phosphatases inhibitors, dual-specificity phosphatases inhibitors, and serine/threonine phosphatases inhibitors.

In another aspect, the invention provides combination compositions and combination delivery methods comprising one or more immune system inhibitors and one or more L5G2BPs. Numerous immunosuppressive/immunomodulatory agents are known, examples of which include T lymphocyte homing modulators (e.g., FTY-720—see, e.g., Yangawa et al., J Immunol. Jun. 1, 1998;160(11):5493-9); calcineurin inhibitors (such as valspodar, PSC 833, and other MDR-1 or p-glycoprotein inhibitors); and TOR-inhibitors (e.g., sirolimus, everolimus, and rapamcyin).

Other features the invention are combination compositions and combination delivery methods comprising one or more L5G2BPs and one or more antineoplastic antibiotics. Such antibiotic chemotherapy agents prevent or delay cell replication. There are many differing antitumour antibiotics, but generally they prevent cell division by two ways: (1) binding to DNA making it unable to separate (2) inhibiting ribonucleic acid (RNA), preventing enzyme synthesis. Examples of such agents include Bleomycin (Blenoxane), Dactinomycin (Actinomycin D, Cosmegen), Daunorubicin (Cerubidine), Doxorubicin (Adriamycin, Rubex), Idarubicin (Idamycin), Mitomycin (Mitomycin-C, Mutamycin), Mitoxantrone (Novantrone), Pentostatin (Nipent), Plicamycin (Mithracin, Mithramycin), and combinations thereof.

In other aspects, a L5G2BP (e.g., an anti-γ2 DIII mAb) or related composition is delivered to a host in association with a thrombosis modulating agent such as a low molecular weight heparin, standard heparin, pentasaccharides, thrombin inhibitory agents (melagatran, ximelagatran, etc.), and/or coagulation factors like Factor VII, Factor VIII, etc.

Chemotherapeutic drugs may lack the ability to adequately penetrating tumors to kill them because these cells may be dead or lack a good blood supply. Anaerobic bacteria, such as Clostridium novyi, can consume the interior of oxygen-poor tumors. Such bacteria die when they come in contact with the tumor's oxygenated sides, meaning they are likely harmless to the rest of the body. The application of such bacteria and one or more L5G2BPs represents another feature of the invention. Typically, such methods are practiced in further combination with a chemotherapeutic agent.

As indicated above, various methods effective in the treatment of cancer can be combined with the delivery of an effective amount of one or more L5G2BPs to a subject. Particular examples of such techniques are described in further detail here.

In one aspect, the invention provides a combination method that comprises application of radiation or associated administration of radiopharmaceuticals to a patient in combination with one or more L5G2BPs. In a related facet, the invention provides a composition comprising an effective combination of one or more L5G2BPs and one or more radiopharmaceuticals.

The source of radiation in such methods can be either external or internal to the patient being treated (radiation treatment can, for example, be in the form of external beam radiation therapy (EBRT) or brachytherapy (BT)). Radioactive elements that can be used in practicing such methods and included in such compositions include, e.g., radium, Cesium-137, Iridium-192, Americium-241, Gold-198, Cobalt-57, Copper-67, Technetium-99, Iodide-123, Iodide-131, and Indium-111. Additionally useful radionuclides that can be incorporated in radiopharmaceuticals and used in such methods are discussed elsewhere herein (e.g., in the context of L5G2BP conjugates). In a particular aspect, such methods and compositions also further optionally include one or more radiation protectors. These drugs are designed to protect normal cells from radiation. One example is the intravenous drug amifostine (Ethyol). In another particular aspect, intensity-modulated radiation therapy (IMRT) is applied in combination with the delivery of one or more L5G2BPs. IMRT delivers radiation therapy that is targeted to tumor shape, minimizing damage to healthy tissue. More particularly, IMRT allows radiation beams to be divided up and delivered in different intensities and directions to match the tumor's shape. Another similar technique, three-dimensional conformal radiation therapy, has been demonstrated to be effective in some situations and can be combined with L5G2BP therapeutic and prophylactic regimens. Pulsed delivery (gating) of radiation also or alternatively can be used in such methods, reducing the field of radiation by compensating for natural breathing patterns.

In a further facet, L5G2BPs are provided in association with the application of a radiogenic therapy. One example of such a therapy is the localized production of cytotoxic agents by radiation stimulation/activation of a prodrug or gene associated with a radiation-inducible promoter (such a gene may code for a cytotoxic protein, an enzyme that activates a co-delivered prodrug, etc.). Another example, is targeted auger-emitting radiolabeled molecules. These therapies can control cancer by delivering targeted radiation to specific receptor bearing cells. Auger electrons are emitted by radioactive isotopes (Iodine-125 or Indium-111). The electrons have very short ranges and therefore have the potential to be delivered to specific sets of target cells, sparing healthy cells. In another exemplary method, a nucleic acid comprising a radiation-induced gene sequence that codes for a protein that can be targeted by a cytotoxic agent is delivered in association with one or more L5G2BPs. Radiation is applied to produce the protein and the cytotoxic agent delivered so as to provide a targeted therapy.

In further aspects, L5G2BPs are delivered in connection with application of photodynamic therapy. In general, such therapies involve the delivery of a photosensitizing agent that makes cells more sensitive to light and, by doing so, causes cancer cells to be destroyed when a laser light is directed on a cancerous area. Thus, various prophylactic and therapeutic regimens of the invention also or alternatively can be combined with anti-cancer directed photodynamic therapy (e.g., anti-cancer laser therapy—which optionally can be practiced with the use of photosensitizing agent, see, e.g., Zhang et al., J Control Release. Dec. 5, 2003; 93(2):141-50)). Rhodium compounds, for example, can damage DNA in living cells in a manner similar to platinum classic chemotherapy drugs, while remaining benign until irradiated with light.

Lasers also can be used in the performance of precise anti-cancer surgeries (e.g., where labeled L5G2BPs have identified cancerous tissues and/or precancerous growths). Other forms of surgery also or alternatively can be applied in connection with the delivery of one or more L5G2BPs. Anti-cancer surgical techniques (e.g., colectomy, proctocolectomy, polypectomy, prostatectomy, segmental resection, lobectomy, pneumonectomy, lumpectomy, mastectomy, etc.) are well known in the art and accordingly are not discussed in detail here (see, e.g., CANCER SURGERY, Harvey and Beatie (W.B. Saunders Company 1996); ADVANCED ONCOLOGIC SURGERY, Roh et al. Eds. (Mosby-Year Books, 1^{st} Ed. 1994); CANCER SURGERY, McKenna et al. Eds. (Lippincott Williams & Wilkins 1994); The M.D. ANDERSON SURGICAL ONCOLOGY HANDBOOK, Feig et al. (Lippincott Williams & Wilkins; 3rd Ed. 2002); and SURGICAL ONCOLOGY: CONTEMPORARY PRINCIPLES AND PRACTICE, Bland et al. (McGraw-Hill Professional; 1^{st} Ed. 2001). In a particular aspect, L5G2BP therapy and/or labeled L5G2BP diagnostic techniques is/are combined with anti-cancer cryosurgery. In another aspect, organs (such as the ovaries or testicles) that make the hormones may be removed in connection with L5G2BP anti-cancer therapy.

In a further aspect, L5G2BP therapy is combined with the application of a bone marrow transplant and/or anti-cancer stem cell therapy. Stem cell transplantation (SCT), for example, may advantageously used in cancer treatment. The SCT may be autologous (the person's own cells that were saved earlier), allogeneic (cells donated by another person), or syngeneic (cells donated by an identical twin). SCT methods and related principles are known in the art (see, e.g., Georges et al., Int J Hematol. January 2003;77(1):3-14; Tabbara et al., Anticancer Res. November-December2003 23(6D):5055-67; Bhatia et al., Expert Opin Biol Ther. January 2001;1(1):3-15; Huugen et al., Neth J Med. May 2002;60(4):162-9; Margolin et al., J Urol. April 2003;169(4):1229-33; and U.S. Pat. No. 6,143,292). Bone marrow transplant is an even more well known method used in treatment of certain cancers (see, e.g., Thomas, Ann N Y Acad Sci. Dec. 29, 1995;770:34-41; Kolb and Holler, Stem Cells. 1997;15 Suppl 1:151-8; Thomas, Semin Hematol. October 1999;36(4 Suppl 7):95-103).

L5G2BPs also can be delivered in association with application of other therapeutic methods such as anti-cancer sound-wave and shock-wave therapies (see, e.g., Kambe et al., Hum Cell. March 1997;10(1):87-94); anti-cancer thermotherapy (see, e.g., U.S. Pat. No. 6,690,976), and/or anti-cancer neutraceutical therapy (see, e.g., Roudebush et al., Vet Clin North Am Small Anim Pract. January 2004;34(1):249-69, viii and Rafi, Nutrition. January 2004;20(1):78-82). Other methods include diet therapies (e.g., fasting therapy (which may be aided by anti-obesity agents or anti-appetite agents) or adoption of a high potassium, low sodium (saltless) diet, with no fats or oils, and high in fresh raw fruits and vegetables—see, e.g., A Cancer Therapy: Results of Fifty Cases, Max Gerson, Gerson Inst; 6th edition). Another technique that may be combined with L5G2BP anti-cancer therapy is inuslin potentiation therapy, wherein low-dose insulin is given in conjunction with low-dose chemotherapy and L5G2BP anti-cancer therapy.

L5G2BP anti-cancer methods also can be applied in conjunction with various adjunct therapies designed to ameliorate cancer-associated and cancer treatment-associated conditions, such as treatments for depression, treatments for pain (e.g., by delivery of morphine or a morphine derivative), treatment for incontinence, treatment for impotence, etc.

The inventive methods described herein also or alternatively can be practiced in connection with the delivery of one or more agents that promote access of an L5G2BP, related compound, or combination thereof to the interior of a tumor. Thus, for example, such methods can be performed in association with the delivery of a relaxin, which is capable of relaxing a tumor (see, e.g., U.S. Pat. No. 6,719,977). As another example of such a technique, a L5G2D3BP or related compound (e.g., an anti-Idiotype anti-γ2 DIII mAb or an immunogenic γ2 peptide) can be bonded to a cell penetrating peptide (CPP). Cell penetrating peptides and related peptides (such as engineered cell penetrating antibodies) are described in, e.g., Zhao et al., J Immunol Methods. Aug. 1, 2001;254(1-2):137-45; Hong et al., Cancer Res. Dec. 1, 2000;60(23):6551-6; Lindgren et al., Biochem J. Jan. 1, 2004;377(Pt 1):69-76; Buerger et al., J Cancer Res Clin Oncol. December 2003;129(12):669-75; Pooga et al., FASEB J. January 1998;12(1):67-77; and Tseng et al., Mol Pharmacol. October 2002;62(4):864-72. Intratumoral administration of L5G2D3BPs or vectors comprising L5G2D3BP-encoding or related molecule-encoding nucleic acid sequences also or alternatively can be used to facilitate therapeutic regimen aspects of the invention.

Additional target Ln-5-binding molecules and molecules that are involved with Ln-5-influenced aspects of cancer progression and, accordingly, are advantageous targets for secondary molecules in the context of combination compositions and/or combination delivery methods (or as being one of the targets in a bispecific anti-γ2 DIII antibody as described elsewhere herein) include α6β1 integrin, α3β1 integrin, α2β1 integrin, α6β1 integrin, laminin-6, laminin-7, EGF-R, type VII collagen, fibulin-1, fibulin-2, Rho GTPases, BP180, syndecan-4, nidogen-1, phosphorylated hsp-27, p300, a cytokeratin, and other matrix metalloproteinases (e.g., MMP-1, MMP-2, MMP-9, and MMP-14 (which also known as Membrane-type matrix metalloproteinase 1 (MT1)), tissue inhibitor of matrix metalloproteinase-1 (TIMP-1) and TIMP-2, E-cadherin, bone morphogenic protein-1 (BMP-1), and the 67 kDa laminin receptor. Thus, in one aspect, the invention provides a method of reducing a cancer progression aspect (e.g., cancer cell migration) in a human patient in need thereof comprising delivering a L5G2BP and an antibody specific for one or more of these non-similar molecules to the patient in amounts and under conditions such that cancer progression is detectably reduced in the patient. Additional types of such molecules are discussed elsewhere herein and/or are known in the art.

The invention further provides compositions and kits comprising one or more pharmaceutically acceptable excipients and one or more L5G2BPs and/or related compositions (e.g., a γ2 DIII immunogenic peptide comprising one of the γ2 DIII antigenic determinant regions described herein (which may be modified by, e.g., cyclization), a nucleic acid encoding such an immunogenic peptide and/or a LG2B3, a vector comprising such a nucleic acid, a cell comprising such a nucleic acid or vector, or an anti anti-γ2 DIII antibody).

In general, L5G2BPs and related compositions can be administered in combination with any suitable pharmaceutically acceptable excipient or combination thereof (i.e., any suitable excipient component). A pharmaceutically acceptable excipient refers to any inactive agent that is combined with an active agent to form a pharmaceutically acceptable and active composition. Excipients include inert pharmaceutically acceptable carriers and diluents. Excipients also include compositions that modulate (and typically improve) the physiochemical properties of a pharmaceutical composition. Examples of such excipients include stabilizers, preservatives, solubilizers, solvents, and solutes. Excipients also include flavorants, coloring agents, etc. L5G2BPs and related compounds and combinations described herein can be formulated in any manner suitable for administration to a subject, such as a human patient.

In one such aspect, a L5G2BP can be combined with one or more carriers appropriate a desired route of administration. L5G2BPs may be, for example, admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and optionally further tabletted or encapsulated for conventional administration. Alternatively, an antibody or other L5G2BPs may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier or diluent may include time delay material, such as glyceryl monostearate or glyceryl distearate alone or with a wax, or other materials well known in the art.

Pharmaceutically acceptable carriers generally include any and all suitable solvents, dispersion media, coatings, antibacterial and antifungal agents, isotonic and absorption delaying agents, and the like that are physiologically compatible with an L5G2BP or related composition or combination provided by the invention. Further examples of pharmaceutically acceptable carriers include sterile water, saline, phosphate buffered saline (PBS), dextrose, glycerol, ethanol, and the like, as well as combinations of any thereof.

In many cases, it can be desirable to include isotonic agents, for example, sugars, polyalcohols (such as mannitol), sorbitol, or sodium chloride in a pharmaceutical composition. Pharmaceutically acceptable substances such as wetting agents, emulsifying agents, preservatives, and buffers, which desirably can enhance the shelf life or effectiveness of the L5G2BP and/or related composition active component.

Suitability for carriers and other components of pharmaceutical compositions is typically determined based on the lack of significant negative impact on the desired biological properties of the L5G2BP, related composition, or combination (e.g., less than a substantial impact—such as about 10% or less relative inhibition, about 5% or less relative inhibition, etc. on γ2 binding by the L5G2BP component of the composition). See also, e.g., Powell et al. “Compendium of excipients for parenteral formulations” PDA J Pharm Sci Technol. 52:238-311 (1998) and the citations therein for additional information related to typically suitable excipients well known to pharmaceutical chemists.

L5G2BP compositions also include compositions comprising any suitable combination of a L5G2BP peptide and related salt. Any suitable salt, such as an alkaline earth metal salt in any suitable form (e.g., a buffer salt), can be used in the stabilization of L5G2BPs (preferably the amount of salt is such that oxidation and/or precipitation of the L5G2BP is avoided). Suitable salts typically include sodium chloride, sodium succinate, sodium sulfate, potassium chloride, magnesium chloride, magnesium sulfate, and calcium chloride. In one aspect, an aluminum salt is used to stabilize a L5G2BP in a composition of the invention, which aluminum salt also may serve as an adjuvant when such a composition is administered to a patient. Compositions comprising a base and L5G2BPs also are provided. In other aspects, the invention provides a L5G2BP composition that essentially lacks a tonicifying amount of any salt.

A composition for pharmaceutical use also can include various diluents, fillers, salts, buffers, detergents (e.g., a nonionic detergent, such as Tween-80), stabilizers, stabilizers (e.g., sugars or protein-free amino acids), preservatives, tissue fixatives, solubilizers, and/or other materials suitable for inclusion in a pharmaceutically composition. Examples of suitable components also are described in, e.g., Berge et al., J. Pharm. Sci., 6661), 1-19 (1977); Wang and Hanson, J. Parenteral. Sci. Tech: 42, S4-S6 (1988), U.S. Pat. Nos. 6,165,779 and 6,225, 289, and other documents cited herein. Such a pharmaceutical composition also can include preservatives, antioxidants, or other additives known to those of skill in the art. Additional pharmaceutically acceptable carriers are known in the art and described in, e.g., Urquhart et al., Lancet, 16, 367 (1980), Lieberman et al., PHARMACEUTICAL DOSAGE FORMS—DISPERSE SYSTEMS (2nd ed., vol. 3,1998); Ansel et al., PHARMACEUTICAL DOSAGE FORMS & DRUG DELIVERY SYSTEMS (7th ed. 2000); Martindale, THE EXTRA PHARMACOPEIA (31st edition), Remington's Pharmaceutical Sciences (16th-20th editions); THE PHARMACOLOGICAL BASIS OF THERAPEUTICS, Goodman and Gilman, Eds. (9th ed.-1996); Wilson and Gisvolds' TEXTBOOK OF ORGANIC MEDICINAL AND PHARMACEUTICAL CHEMISTRY, Delgado and Remers, Eds. (10th ed.-1998), and U.S. Pat. Nos. 5,708,025 and 5,994,106. Principles of formulating pharmaceutically acceptable compositions also are described in, e.g., Platt, Clin. Lab Med., 7:289-99 (1987), Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), and “Drug Dosage,” J. Kans. Med. Soc., 70 (I), 30-32 (1969). Additional pharmaceutically acceptable carriers particularly suitable for administration of vectors are described in, for example, International Patent Application WO 98/32859.

L5G2BP compositions, related compositions, and combinations according to the invention may be in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles (see, e.g., Baek et al., Methods Enzymol. 2003;362:240-9; Nigavekar et al., Pharm Res. March 2004;21(3):476-83), microparticles, and suppositories. In one exemplary aspect, the invention provides an effective amount of one or more anti-γ DIII antibodies contained in liposomes formulated for delivery to cancer-associated cells.

Formulations of L5G2BP compositions also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax. Any of the foregoing mixtures may be appropriate in treatments and therapies in accordance with the present invention, provided that the binding of the L5G2BP to γ2 is not significantly inhibited and/or the biological activity of related molecule(s) significantly inhibited by the formulation and the formulation is physiologically compatible and tolerable with the planned route of administration. The optimal form for any L5G2BP-associated composition depends on the intended mode of administration, the nature of the composition or combination, and therapeutic application or other intended use.

Typically, compositions in the form of injectable or infusible solutions, such as compositions similar to those used for passive immunization of humans with other antibodies, are used for delivery of L5G2BPs of the invention. A typical mode for delivery of L5G2BP compositions is by parenteral administration (e.g., intravenous, subcutaneous, intraperitoneal, and/or intramuscular administration). In one aspect, an anti-γ2 DIII antibody is administered to a human patient by intravenous infusion or injection. In another aspect, an anti-γ2 DIII antibody is administered by intramuscular or subcutaneous injection. As already indicated, intratumor administration also may be useful in certain therapeutic regimens.

L5G2BPs, such as anti-γ2 DIII antibodies, antibody fragments, and derivatives thereof, may be formulated in, for example, solid formulations (including, e.g., granules, powders, projectile particles, or suppositories), semisolid forms (gels, creams, etc.), or in liquid forms (e.g., solutions, suspensions, or emulsions).

Antibodies and other L5G2BPs also may be applied in a variety of solutions. Suitable solutions for use in accordance with the invention typically are sterile, dissolve sufficient amounts of the antibody and other components of the composition (e.g., an immunomodulatory cytokine such as GM-CSF, IL-2, and/or KGF), stable under conditions for manufacture and storage, and not harmful to the subject for the proposed application.

A L5G2BP may be subjected to conventional pharmaceutical operations such as sterilization and/or may contain conventional adjuvants, such as preservatives, stabilizers, wetting agents, emulsifiers, buffers, etc. A composition also can be formulated as a solution, microemulsion, dispersion, powder, macroemulsion, liposome, or other ordered structure suitable to high drug concentration. Desirable fluidity properties of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. These and other components of a pharmaceutically acceptable composition of the invention can impart advantageous properties such as improved transfer, delivery, tolerance, and the like.

In one exemplary aspect, the active compound or combination is prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., SUSTAINED AND CONTROLLED RELEASE DRUG DELIVERY SYSTEMS, J. R. Robinson, ed., Marcel Dekker, Inc., New York, 1978. Other teachings relevant to such systems are known in the art. For example, Heller, Biodegradable Polymers in Controlled Drug Delivery, in: CRC Critical Reviews in Therapeutic Drua Carrier Systems, Vol. 1, CRC Press, Boca Raton, Fla., 1987, pp 39-90, describes encapsulation for controlled drug delivery, and Di Colo (1992) Biomaterials 13:850-856 describes controlled drug release from hydrophobic polymers.

In another aspect, compositions of the invention orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

L5G2BPs, related compounds (e.g., L5G2BP-encoding nucleic acids), and related compositions can generally be delivered in any suitable dosage. Determination of precise dosage for optimal effect will vary with a number of factors (condition to be treated, age of patient, health of patient, type(s) of L5G2BP or surrogate used, presence of additional active agents and/or application of related therapies, etc.), such that it is often more useful to describe dosage in terms of an amount sufficient or optimal for inducing, promoting, and/or enhancing a particular effect. In this respect, compositions of the invention can include “therapeutically effective amount,” a “prophylactically effective amount” , or “physiologically effective amount” of a L5G2BP or related composition (or “first” and “second” amounts in the case of a combination composition comprising a L5G2BP and a second element; first, second, and third amounts in the case of three included agents; etc.).

A “therapeutically effective amount” refers to an amount effective, when delivered in appropriate dosages and for appropriate periods of time, to achieve a desired therapeutic result in a host (e.g., the inducement, promotion, and/or enhancement of a physiological response associated with reducing one or more aspects of cancer progression, increasing the likelihood of survival over a period of time (e.g., 18-60 months after initial cancer treatment), reducing the spread of cancer cell-associated growths, and/or reducing the likelihood of recurrence of tumor growth). A therapeutically effective amount of a L5G2D3BP may vary according to factors such as the disease state, age, sex, and weight of the individual, and the ability of the L5G2BP to elicit a desired response in the individual. A therapeutically effective amount is also one in which any toxic or detrimental effects of the antibody or antibody portion are outweighed by the therapeutically beneficial effects.

A “prophylactically effective amount” refers to an amount effective, at dosages and for periods of time necessary, to achieve a desired prophylactic result (e.g., a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc.). Typically, because a prophylactic dose is used in subjects prior to or at an earlier stage of disease, the prophylactically effective amount will be less than the therapeutically effective amount.

Where the phrase “effective amount” is used without a modifier such as “therapeutically” or “prophylactically”, the phrase is intended to mean an amount that is at least as great as the minimum prophylactically effective or therapeutically effective amount and that is appropriate for the indicated use. In one exemplary aspect, the invention relates to a method of treating cancer in a patient comprising delivering to the patient an effective amount of one or more L5G2BPs, a L5G2BP combination composition, a related composition, etc. Such an amount typically ranges from the minimum amount required to induce an effective prophylactic effect in a patient that is not suffering from a cancer condition (but may be at risk of developing such a condition) to an amount sufficient for one or more therapeutic regimens (e.g., significantly reducing tumor burden). In other words, the phrase “effective amount” encompasses both “prophylactically effective” and “therapeutically effective” amounts unless otherwise stated or clearly contradicted by context.

In another aspect, the invention relates to a method of inducing, promoting, and/or enhancing one or more physiological events/activities in a patient (e.g., the reduction of cancer progression). A “physiologically effective” amount refers to an amount that is sufficient to induce, promote, and/or enhance the desired physiological effect(s).

The compositions of the invention can be administered in any suitable dosage regimen. Suitability with respect to dosage regimens refers to the administration of any number of doses of a composition, any number of times in a relevant period (typically a day) that result in a desired physiological effect. Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the L5G2BP, related composition, or combination and (b) the particular therapeutic or prophylactic effect to be achieved. The total time of a course of treatment also can be any suitable time and also is likely to vary with a number of similar factors that will be determinable to skilled practitioners with routine experimentation.

Dosage of anti-cancer agents, such as L5G2BPs, typically is adjusted for the patient's body surface area (BSA), a composite measure of weight and height that mathematically approximates the body volume. Thus, in one aspect, the invention relates to the delivery of a BSA-adjusted amount of a L5G2BP or L5G2BP-comprising composition. The BSA is usually calculated with a mathematical formula or a nomogram, rather than by direct measurement. Such methods are known in the art.

An exemplary, non-limiting range for a therapeutically or prophylactically effective amount of an antibody or antibody portion of the invention is about 0.1-100 mg/kg, such as about 0.1-50 mg/kg, for example about 0.1-20 mg/kg, and more particularly about 1-10 mg/kg (e.g., at about 0.5 mg/kg (such as 0.3 mg/kg), about 1 mg/kg, or about 3 mg/kg). Generally, such an amount is administered once per day or less (e.g., 2-3 times per week, 1 times per week, or 1 time every two weeks).

In the case of combination compositions, the L5G2BP or related compound is coformulated with and/or coadministered with one or more additional therapeutic agents as described elsewhere herein (e.g., an antigenic peptide or an immunostimulatory cytokine). Such combination therapies may require lower dosages of the L5G2BP and/or the co-administered agents, thus avoiding possible toxicities or complications associated with the various monotherapies.

Another aspect of the present invention provides a kit comprising a L5G2BP, related composition, or combination thereof, pharmaceutically excipient component, and optionally other pharmaceutical composition components (e.g., one or more secondary active agents). A kit may include, in addition to one or more L5G2BPs, various diagnostic or therapeutic agents. A kit may also include instructions for use in a diagnostic or therapeutic method. Such instructions can be, for example, provided on a device included in the kit. Advantageously, such a kit includes a L5G2BP and a diagnostic agent that can be used in one or more of the various diagnostic methods described elsewhere herein. In another preferred embodiment, the kit includes a L5G2D3BP, related compound, or combination composition in a highly stable form (such as in a lyophilized form) in combination with pharmaceutically acceptable carrier(s) that can be mixed with the highly stable composition to form an injectable composition for near term administration.

L5G2BPs, related compounds, and combination compositions described above and elsewhere herein, such as the above-described pharmaceutically acceptable compositions, are useful in a variety of therapeutic and prophylactic regimens and diagnostic and prognostic applications.

In one aspect, the invention provides a method of detection, diagnosis, prognosis, monitoring, and therapeutic treatment of cancer and aspects thereof (e.g., the invention provides a method of inhibiting the migration of invasive phenotype cancer cells through the delivery of a L5G2BP to the cancer cells or the vicinity of such cells, such as near the invasive front of a cancer cell population).

Particular aspects of such methods will be discussed in turn below.

In one exemplary aspect, the invention provides a method of reducing cancer progression in a mammalian host, such as a human patient, having a detectable level of cancer cells comprising administering a L5G2BP, a related composition (e.g., a nucleic acid encoding a L5G2BP, an anti-anti-Idiotypic γ2 DIII antibody, etc.), or a combination composition of the invention an amount sufficient to detectably reduce the progression of the cancer in the host.

A cancer cell in the context of this invention is any cell that divides and reproduces abnormally with uncontrolled growth (e.g., by exceeding the “Hayflick limit” of normal cell growth (as described in, e.g., Hayflick, Exp. Cell Res., 37,614 (1965)). “Cancers” generally consist of single or several clones of cells that are capable of partially independent growth in a host (e.g., a benign tumor) or fully independent growth in a host (malignant cancer). Cancer cells arise from host cells via neoplastic transformation (“carcinogenesis”).

Terms such as “preneoplastic,” “premalignant,” and “precancerous” with respect to the description of cells and/or tissues herein refer to cells or tissues having a genetic and/or phenotypic profile that signifies a significant potential of becoming cancerous. Usually such cells can be characterized by one or more differences from their nearest nonneoplastic counterparts that signal the onset of cancer progression or significant risk for the start of cancer progression. Such precancerous changes, if detectable, can usually be treated with excellent results. Accordingly, delivery of L5G2BPs to such cells and tissues as part of a prophylactic regimen is an important aspect of the invention. Some cancers have well defined precancer precursors, others do not. In general, a precancerous state will be associated with the incidence of neoplasm(s) or preneoplastic lesion(s). Examples of known and likely preneoplastic tissues include ductal carcinoma in situ (DCIS) growths in breast cancer, cervical intra-epithelial neoplasia (CIN) in cervical cancer, adenomatous polyps of colon in colorectal cancers, atypical adenomatous hyperplasia in lung cancers, and actinic keratosis (AK) in skin cancers. Preneoplastic phenotypes and genotypes for various cancers, and methods for assessing the existence of a preneoplastic state in cells, have been characterized. See, e.g., Medina, J Mammary Gland Biol Neoplasia. October 2000;5(4):393-407; Krishnamurthy et al., Adv Anat Pathol. May 2002;9(3):185-97; Ponten, Eur J Cancer. October 2001;37 Suppl 8:S97-113; Niklinski et al., Eur J Cancer Prev. June 2001;10(3):213-26; Walch et al., Pathobiology. January-February 2000; 68(1):9-17; and Busch, Cancer Surv. 1998;32:149-79. Gene expression profiles can increasingly be used to differentiate between normal, precancerous, and cancer cells. For example, familial adenomatous polyposis genes prompt close surveillance for colon cancer; mutated p53 tumor-suppressor gene flags cells that are likely to develop into aggressive cancers; osteopontin expression levels are elevated in premalignant cells, and increased telomerase activity also can be a marker of a precancerous condition (e.g., in cancers of the bladder and lung).

“Cancer progression” refers to any event or combination of events that promote, or which are indicative of, the transition of a normal, non-neoplastic cell to a cancerous, neoplastic cell, the migration of such neoplastic cells, and the formation and growth of tumors therefrom (which latter aspect can be referred to as tumor progression). Examples of such events include phenotypic cellular changes associated with the transformation of a normal, non-neoplastic cell to a recognized pre-neoplastic phenotype, and cellular phenotypic changes that indicate transformation of a pre-neoplastic cell to a neoplastic cell.

Aspects of cancer progression (also referred to herein as “cancer progression stages”) include cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, aneuploidy, and focus formation), proliferation of transformed cells, development of metastatic potential, migration and metastasis (e.g., the disassociation of the cell from a location and relocation to another site), new colony formation, tumor formation, tumor growth, neotumorogenesis (formation of new tumors at a location distinguishable and not in contact with the source of the transformed cell(s)), and any combinations thereof.

Carcinogenesis is typically associated with the activation of genes that regulate cell growth via bypassing the host cell's regulatory controls (e.g., bypassing or overcoming a host cell's normally active apoptotic signaling pathway(s)) and the reduced expression of tumor-suppressor genes. Multiple genes typically are deregulated in association with the development of fully malignant tumors.

Cancer progression often is also or alternatively described by the general stages of initiation, promotion, and progression. In tumor-forming cancers, for example, cancer progression often is described in terms of tumor initiation, tumor promotion, malignant conversion, and tumor progression (see, e.g., Cancer Medicine, 5^{th} Edition (2000) B. C. Decker Inc., Hamilton, Ontario, Canada (Blast et al. eds.)). Tumor initiation, which reflects the presence of morphological, genetic, and/or behavioral changes at the cellular or tissue level (e.g., the induction of mitogenesis, compensatory cell proliferation, preneoplasia and hyperplasia, survival of premalignant or malignant cells (immortalization, immunosuppression), and occurrence of cancer-associated effects on metastatic potential, etc.), typically results from irreversible genetic damage, such as carcinogen-induced mutations. The initiation stage typically is characterized by the conversion of a normal cell to an initiated cell in response to DNA-damaging agents. Tumor promotion comprises the selective clonal expansion of initiated cells. Tumor progression comprises the expression of the malignant phenotype and the tendency of already malignant cells to acquire more aggressive characteristics with time. The promotion stage typically is characterized by the transformation of an initiated cell into a population of preneoplastic cells, due to alterations in gene expression and cell proliferation. The progression stage typically is characterized by the transformation of the preneoplastic cells to a neoplastic cell population as a result of additional genetic alterations.

Terms such as neoplastic transformation or neoplastic conversion also can describe a stage of cancer progression. Neoplastic conversion is the transformation of a preneoplastic cell into one that expresses a neoplastic phenotype. Once neoplastic conversion is complete, cells with altered gene structure need to multiply to express the cancer associated gene structure. Cell duplication determines the rate of expression and the associated cancer risk. Epigenetic events in general, and DNA methylation in particular, are associated with modulating changes from a normal to preneoplastic to neoplastic state. Neoplastic transformation also is associated with the activation of growth regulatory genes, such as growth factor receptors (e.g., IGF-I receptor, Erb receptor dimers and components thereof (e.g., Erb-B receptor dimers), wnt receptor, fims, neu); molecules involved in signal transduction (src, abl, ras); and transcription factors (jun, fos, myc), which often are referred to as cellular oncogenes or “(c) oncogenes”. Additional factors involved in neoplastic transformation include genes that inhibit growth (e.g., p53 and Rb) and genes that regulate apoptosis, such as bcl-2. Neoplastic transformation also involves the inappropriate activation of genes that control cell growth.

In another stage of cancer progression, immunogenic tumors typically escape immune-surveillance of the host enabling their growth. Additional related aspects of cancer progression include evasion of apoptosis by the cancer cell, achieving limitless replication potential, achieving self-sufficiency in growth factor expression, achieving abnormal insensitivity to anti-growth signals; achieving sustained angiogenesis, and metastasis.

Metastasis refers to the spread of cancer cells from one site in a medium to another, such as in the tissue(s) of a patient. Metastasis also typically is involved with a number of distinct physiological events, which include the escape of cancer cells from an initial site via lymphatic channels or protease activity; the survival of cancer cells in circulation; arrest in secondary site(s); extravasation into surrounding tissue; initiation and maintenance of growth, and vascularization of metastatic tumor(s). Metastasis also may involve the ability of tumor cells to secrete proteases that allow invasion beyond the immediate location of the primary tumor. A prominent characteristic of malignant phenotype is the propensity for genomic instability and uncontrolled growth.

Metastatic cancer cells typically penetrate the extracellular matrix (ECM) and the basement membrane of the blood vessels to metastasize to a target organ (ectopic site). The EMC consists of proteins embedded in a carbohydrate complex (heparan sulfate peptidoglycans), and proteases surrounding a tumor are active in this breaking down the host tissue. Thus, the penetration of the ECM and basement membranes and breakdown of related host tissues also are relevant aspects of cancer progression. Indeed, there often is a complex mix of the normally consecutive processes of cell attachment, detachment, as well as degradation of extracellular matrix proteins, and migration, which is needed for the locomotion of invasive tumor cells to distant locations. All of these activities are important aspects of cancer progression in the context of the present invention. Thus, for example, delivery of a L5G2BP, related compound, or combination composition can be used as a means of reducing any one of these physiological activities in association with the treatment of cancer in a patient.

Methods for detecting cancers and cancer progression include (a) clinical examination (symptoms can include swelling, palpable lumps, enlarged lymph nodes, bleeding, visible skin lesions, and weight loss); (b) imaging (X-ray techniques, mammography, colonoscopy, computed tomography (CT and/or CAT) scanning, magnetic resonance imaging (MRI), etc.); (c) immunodiagnostic assays (e.g., detection of CEA, AFP, CA125, etc.); (d) antibody-mediated radioimaging; and (e) analyzing cellular/tissue immunohistochemistry.

Delivering L5G2BPs to a subject (either by direct administration or expression from a nucleic acid therein, such as from a pox viral gene transfer vector) and practicing the other methods of the invention can be used to reduce, treat, prevent, or otherwise ameliorate any suitable aspect of cancer progression. The methods of the invention are particularly useful in the reduction and/or amelioration of tumor growth, cancer migration, and cancer cell invasiveness, as described further herein. Methods that reduce, prevent, or otherwise ameliorate such aspects of cancer progression, independently and collectively, are advantageous features of the invention. Additional advantageous aspects of the invention include the reduction of metastases, the reduction of the spread of tumors, the reduction of tumor growth, and combinations of any thereof. Another favorable aspect is the effectiveness of such methods in the treatment of cancers characterized by micrometastases. In another facet, this invention relates to the use of L5G2BPs, related compounds, and/or related compositions, as described herein, in the preparation of a medicament for treatment of cancer.

Different therapeutic regiments involving L5G2BPs, related compounds, and combination compositions can be applied with respect to different aspects of cancer progression. Thus, for example, in one aspect a L5G2BP is delivered to a patient as part of an anti-initiation strategy. Advantageous secondary antineoplastic agents and techniques for administration, delivery, or application in the context of an anti-initiation therapeutic regimen include, for example, DNA repair enzymes, molecules that scavenge for reactive oxygen species and electrophiles, and compositions that enhance carcinogen detoxification. In another aspect, a L5G2BP, related composition, or combination composition is delivered, administered, or applied in the context of an anti-promotion and/or anti-proliferation therapeutic regimen. Advantageous secondary agents and techniques in the context of such a therapeutic regimen include, for example, agents and techniques that induce cancer cell death, agents and techniques that suppress cancer cell proliferation (e.g., chemotherapeutic agents), and agents that alter cancer cell-associated gene expression (e.g., agents that reduce expression of cancer-promoting genes, methods that involve re-introducing functional tumor suppressors, etc.).

The detection of cancer progression can be achieved by any suitable technique, several examples of which are known in the art. Examples of suitable techniques include PCR and RT-PCR (e.g., of cancer cell associated genes or “markers”), biopsy, electron microscopy, positron emission tomography (PET), computed tomography, immunoscintigraphy and other scintegraphic techniques, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques. Examples of these and other suitable techniques are described in, e.g., Rieber et al., Cancer Res., 36 (10), 3568-73 (1976), Brinkley et al., Tex. Rep. Biol. Med., 37,26-44 (1978), Baky et al., Anal. Quant. Cytol., 2 (3), 175-85 (1980), Laurence et al., Cancer Metastasis Rev., 2 (4), 351-74 (1983), Cooke et al., Gut, 25 (7), 748-55 (1984), Kim et al, Yonsei Med. J., 26 (2), 167-74 (1985), Glaves, Prog. Clin. Biol. Res., 212, 151-67 (1986), McCoy et al., Immunol. Ser., 53,171-87 (1990), Jacobsson et al., Med. Oncol. Tumor. Pharmacother., 8 (4), 253-60 (1991), Swierenga et al., IARC Sci. Publ., 165-93 (1992), Hirnle, Lymphology, 27 (3), 111-3 (1994), Laferte et al., J. Cell Biochem., 57 (1), 101-19 (1995), Machiels et al., Eur. J. Cell Biochem., 66 (3), 282-92 (1995), Chaiwun et al., Pathology (Phila), 4 (1), 155-68 (1996), Jacobson et al, Ann. Oncol., 6 (Suppl. 3), S3-8 (1996), Meijer et al., Eur. J. Cancer, 31A (7-8), 1210-11 (1995), Greenman et al., J. Clin. Endocrinol. Metab., 81 (4), 1628-33 (1996), Ogunbiyi et al., Ann. Surg. Oncol., 4 (8), 613-20 (1997), Merritt et al., Arch. Otolaryngol. Head Neck Surg., 123 (2), 149-52 (1997), Bobardieri et al., Q. J. Nucl. Med., 42 (1), 54-65 (1998), Giordano et al., J. Cell Biochem, 70 (1), 1-7 (1998), Siziopikou et al., Breast J., 5 (4), 221-29 (1999), Rasper, Surgery, 126 (5), 827-8 (1999), von Knebel et al. Cancer Metastasis Rev., 18 (1), 43-64 (1999), Britton et al., Recent Results Cancer Res., 157,3-11 (2000), Caraway et al., Cancer, 90 (2), 126-32 (2000), Castillo et al., Am. J. Neuroadiol., 21 (5), 948-53 (2000), Chin et al., Mayo Clin. Proc., 75 (8), 796-801 (2000), Kau et al., J. Ortohinolaryngol. Relat. Spe., 62 (4), 199-203 (2000), Krag, Cancer J. Sci. Am., 6 (Suppl. 2), S121-24 (2000), Pantel et al., Curr. Opin. Oncol., 12 (1), 95-101 (2000), Cook et al., Q. J. Nucl. Med., 45 (1), 47-52 (2001), Gambhir et al., Clin. Nucl. Med., 26 (10), 883-4 (2001), MacManus et al., Int. J. Radiat. Oncol. Biol. Phys., 50 (2), 287-93 (2001), Olilla et al., Cancer Control., 8 (5), 407-14 (2001), Taback et al., Recent Results Cancer Res.,158,78-92 (2001), and references cited therein. Related techniques are described in U.S. Pat. Nos. 6,294,343, 6,245,501, 6,242,186, 6,235,486, 6,232,086, 6,228,596, 6,200,765, 6,187,536, 6,080,584, 6,066,449, 6,027,905, 5,989,815, 5,939,258, 5,882,627, 5,829,437, 5,677,125, and 5,455,159 and WO 01/69199, WO 01/64110, WO 01/60237, WO 01/53835, WO 01/48477, WO 01/04353, WO 98/12564, WO 97/32009, WO 97/09925, and WO 96/15456.

A reduction of cancer progression can include any detectable decrease in (1) the rate of normal cells transforming to neoplastic cells (or any aspect thereof), (2) the rate of proliferation of pre-neoplastic or neoplastic cells, (3) the number of cells exhibiting a pre-neoplastic and/or neoplastic phenotype, (4) the physical area of a cell media (e.g., a cell culture, tissue, or organ (e.g., an organ in a mammalian host)) comprising pre-neoplastic and/or neoplastic cells, (5) the probability that normal cells and/or preneoplastic cells will transform to neoplastic cells, (6) the probability that cancer cells will progress to the next aspect of cancer progression (e.g., a reduction in metastatic potential), or (7) any combination thereof. Such changes can be detected using any of the above-described techniques or suitable counterparts thereof known in the art, which typically are applied at a suitable time prior to the administration of a therapeutic regimen so as to assess its effectiveness. Times and conditions for assaying whether a reduction in cancer potential has occurred will depend on several factors including the type of cancer, type and amount of L5G2BP, related composition, or combination composition being delivered to the host. The ordinarily skilled artisan will be able to make appropriate determinations of times and conditions for performing such assays applying techniques and principles known in the art with routine experimentation.

Other methods useful for diagnosing cancer progression include tumor grading and staging methods, such as the American Joint Commission on Cancer grading system, the National Program of Cancer Registries “General Staging” method (also known as Summary Staging, California Staging, and SEER Staging), and/or commonly used specialized grading systems (e.g., a high Gleason tumor grade score is indicative of an aggressive cancer in the context of prostate cancer; a TNM (Tumor, Nodes, Metastasis) Staging System often is useful in the context of colorectal cancer, and the Scarff-Bloom-Richardson system often is used in the context of breast cancer assessments) (see, e.g., Fawcett and Drew, Prof Nurse. April 2002; 17(8):470-2; Toloza et al., Chest. January 2003;123(1 Suppl):157S-166S; Fischer et al., Lancet Oncol. November 2001;2(11):659-66; and Perrotti et al., Urology. August 1999;54(2):208-14; Zinkin, Dis Colon Rectum. January 1983;26(1):37-43; see also generally Neal, Clinical Oncology (Oxford University Press—3rd Ed. 2003), Price, TREATMENT OF CANCER (Oxford University Press—4th Ed. 2002), Franks, INTRODUCTION TO THE CELLULAR AND MOLECULAR BIOLOGY OF CANCER (Oxford University Press—3rd Ed. 1997); Bast et al., Cancer Medicine, 5th Edition (BC Decker Inc.—2000); Adami, TEXTBOOK OF CANCER EPIDEMIOLOGY (Oxford University Press 2002). Further methods for identifying cancer and/or diagnosing cancer progression include cancer gene-related DNA methylation (see, e.g., Carmen et al., J. Natl. Cancer Inst., 93(22) (2001)), DNA cytometry, mitosis assays (as to frequency, normalcy, or both), pleomorphism evaluations, the presence of autocrine stimulatory loop activity, tubule formation measurements, keritinization assays, intercellular bridge formation assays, epithelial pearl detection, aberrant hormone receptor expression or form production assays (e.g., Her2 overexpression assays), and other cancer-associated gene expression assays (e.g., PRL-3 protein tyrosine phosphatase gene expression assays). Additionally useful diagnostic methods are described in U.S. Pat. No. 6,682,901 and PCT Publication WO 03/033667. The inventive therapeutic regimens of the invention (involving, among other things, the delivery of a L5G2BP or related compound to a patient in need thereof so as to reduce cancer progression in one or more aspects thereof) can be practiced in association with finding an indication of cancer and/or cancer progression in a patient as determined by any one of these or other diagnostic assays described herein or their equivalents. Such methods are additional features of this invention.

In particular aspects, the invention provides a method of treating a cancer graded as greater than T0 (e.g., T1, T2, or T3). In another aspect, the invention provides a method of preventing the progression of a patient's tumor load from T2 or T3 to T4. In another aspect, the invention relates to the treatment of cancer in a patient classified as having a T4 state. In another aspect, the invention also or alternatively relates to the treatment of a patient classified as having an M1 cancer. In a further facet, the invention relates to the treatment of a cancer that is staged as more progressed than a stage I cancer (e.g., a stage II and/or stage III cancer). In a further aspect, the invention provides a method of reducing or halting progression of such stage II and/or stage III cancers to stage IV cancers. In another facet, the invention provides a method of treating a cancer classified as a stage IV cancer.

The reduction of cancer cell migration and invasiveness are particularly advantageous aspects of the invention. Accordingly, the detection of a reduction in cancer progression in one or both of these physiological responses is particularly useful. Methods suitable for assessing these forms of cancer progression include Boyden and Transwell chamber assays (see, e.g., US 20020052307; Hujanen and Terranova (1985) Cancer Res. 45: 3517-3521; and Pelletier, A. J., Kunicki, T. and Quaranta, V. (1996), J. Biol. Chem. 271:364); matrigel migration assays (see, e.g., Zhang et al., Oncogene. Apr. 15, 2004;23(17):3080-8 and Knutson et al., Molecular Biology of the Cell, 7: 383-396, 1996); integrin betal assays (see, e.g., Berry et al., Breast Cancer. 2003;10(3):214-9); beta-catenin and related molecule assays (e.g., E-cadherin and/or “slug” gene expression assays); radiographic assays (such as barium radiographic invasiveness assays); positron emission tomography assessments; magnetic resonance imaging (MRI) techniques (e.g., measurement of tumor diameter and/or volume); DNA cytometry; mammographic measurements; fluorescence in situ hybridization (FISH) analysis methods (e.g., using nucleic acid probes relevant for cancer gene expression, such as HER-2/neu probes); biopsy; angiogenesis assessments; and measuring relevant invasiveness-associated biological markers (including endogenous Ln-5, and particularly forms of Ln-5, Ln-5 gene expression levels/patterns, Ln-5 nucleic acid methylation, and Ln-5 fragments/portions (such as portions of γ2, γ2/β33 heterodimers, and β33 fragments) associated with cancer progression); simultaneous measurements of serum sCEA and TIMP1; etc.). The characterization of invasive cells is well known in the art. A discussion of invasive cell characteristics and related principles can be found in, e.g., King RJB (1996) Cancer Biology (Addison Wesley Longman Ltd., Harlow Essex) and Liolta and Stetler-Stevenson (1991)—Cancer Res 51 :5054s-5059s.

A detailed exemplary protocol for a Transwell assay of cell migration is provided in this paragraph for purposes of illustrating a diagnostic tool that, like other assays described herein, can be used in the context of assessing the suitability of L5G2BPs, related compounds, and combination compositions/administration methods, while also providing another useful feature of the invention. Transwell plates with pore size of 12 μm can be obtained from Costart (Cambridge, Mass., USA). The lower side of the membrane can be coated with about 2.5 μg of EHS type IV collagen overnight (o/n) at room temperature (RT). Suitable cells, for example HSC-3 cells, are removed from cell culture, and incubated with or without test molecules, such as test anti-γ2 DIII mAbs (typically about 25, 50, or 100 μg/ml of the test anti-γ2 DIII mAbs, normal mouse IgG, unspecific mAb, and/or nothing (control) is used in such assays), for a suitable time (e.g., about 30 minutes) at a suitable temperature (typically about 37° C.). Cells can then be transferred to the upper part of the chamber with antibodies and allowed to migrate for a sufficient period of time (typically about 6 hours), with or without addition of FCS and optionally in the presence of a low concentration (e.g., 0.1%) of BSA. A sufficient amount of FN is used as a chemoattractant in the lower part of the chamber (e.g., 2.5 μg/ml). Cells from the upper side of the membrane can be removed and cells that have migrated through the membrane can be stained and the number of cells calculated under a microscope by field (typically the view is divided into a circular area of 10 fields diameter). Such assays, using for example, HSC-3 cells, have been used to demonstrate the motility-inducing effects of γ2 peptides.

The methods of the invention can be used to reduce the cancer progression of a number of cancer types and the therapeutic protocols of the invention are generally not restricted to any particular type or types of cancer. Advantageously, methods of the invention can be used to, for example, reduce the cancer progression in prostate cancer cells, melanoma cells (e.g., cutaneous melanoma cells, ocular melanoma cells, and/or lymph node-associated melanoma cells), breast cancer cells, colon cancer cells, and lung cancer cells. Methods of the invention also can be used to reduce cancer progression in both tumorigenic and non-tumorigenic cancers (e.g., non-tumor-forming hematopoietic cancers). Methods of the invention are particularly useful in the treatment of epithelial cancers (e.g., carcinomas). Methods of the invention also or alternatively are advantageous in the treatment of colorectal cancers, breast cancers, lung cancers, vaginal cancers, cervical cancers, and/or squamous cell carcinomas (e.g., of the head and neck). Additional potential targets for therapeutic uses of L5G2BPs, related compounds, and related compositions include sarcomas and lymphomas. Advantageous targets also include solid tumors and/or disseminated tumors (e.g., myeloid and lymphoid tumors, which can be acute or chronic).

Additional features of the inventive methods include the reduction in the size and/or number of and/or prevention of the formation of tubular networks associated with Ln-5. Another feature of the invention is a method of preventing poorly aggressive neoplastic cells from developing a vasculogenic phenotype. Such aspects can be combined with the general feature of reducing the invasive potential (aggressiveness) and/or metastatic potential of cancers by such methods. Reduction of neoplastic and/or preneoplastic cell migration, reduction of cell division, and/or reduction of cell migration by administration of compositions of the invention are additional physiological endpoints that can be achieved by application of inventive methods described herein.

In another exemplary aspect, the invention provides a method of increasing the ratio of quiescent to invasive neoplastic cells in a mammalian host (e.g., a human patient) comprising administering a therapeutically effective amount of a L5G2BP (e.g., an anti-γ2 DIII mAb such as a cytotoxin-conjugated or radionuclide-conjugated mAb), related molecule, or related composition (e.g., a combination composition) of the invention so as to increase the ratio of quiescent to invasive cells in the host.

In a further aspect, the invention provides a method of preventing the formation of cancer associated tubular networks in a mammalian host (e.g., a human patient) comprising administering a physiologically effective amount of a L5G2BP (e.g., a therapeutically effective amount of an anti-γ2 DIII mAb), a related compound, or a combination thereof so as to detectably reduce the risk of developing cancer associated tubular networks, prolong the onset of cancer associated tubular networks, and/or reduce the number of expected cancer associated tubular networks formed in the host.

In yet another aspect, the invention provides a method of reducing the invasive potential of a population of cancer cells in a mammalian host (e.g., in a human patient) comprising administering a physiologically effective amount of a L5G2BP (e.g., a therapeutically effective amount of an anti-γ2 DIII mAb, a variant thereof, a fragment of either thereof, or a derivative of any thereof), related compound, or combination composition so as to detectably reduce the invasive potential of the cancer cells.

In still another aspect, the invention provides a method of reducing cell migration, reducing tumor growth, reducing neoplastic and/or pre-neoplastic cell division, or any combination thereof in a mammalian host (e.g., a human patient in need thereof) comprising administering a therapeutically effective amount of a L5G2BP (e.g., a L5G2D3BP), related compound, or combination composition of the invention to the host so as to achieve the desired outcome(s).

In a further aspect, the invention provides a method of promoting remission of a cancer in a mammalian host, such as a human patient, comprising administering a composition comprising a L5G2BP, such as an anti-γ2 DIII mAb, that competes with mAb 5D5 and/or mAb 6C12 (e.g., relatively inhibits at a level of at least about 10% as determined by ELISA, such as inhibits binding by about 15% or more, about 20% or more, about 25% or more, etc.) to the host, so as to promote cancer remission in the host. In a particular aspect, the invention provides a method for treating a local recurrence of a cancer in a human patient. In another aspect, the invention provides a method of treating a distant recurrence of a cancer in a human patient.

In yet another aspect, the invention provides a method for modulating MAP kinase activity in neoplastic or preneoplastic cells of a mammalian host, such as a human cancer patient, comprising contacting the cells with a physiologically effective amount of a composition comprising a L5G2BP (e.g., a therapeutically effective amount of an anti-γ2 DIII antibody), related compound, or combination composition, so as to detectably modulate MAP kinase activity in the cells (which method may be practiced in vivo, in vitro, ex vivo, etc.).

In an even further aspect, the invention provides a method for reducing the risk of developing a cancerous condition, reducing the time to onset of a cancerous condition, reducing the severity of a cancer diagnosed in the early stages, and/or reducing the affected area of a cancer upon development thereof in a mammalian host (e.g., a human patient), comprising administering to a host a prophylactically effective amount of a L5G2BP, related compound, or combination composition of the invention so as to achieve the desired physiological effect(s).

In another facet, the invention provides a method of treating a condition associated with γ2-associated peptide activity in a patient in need of such treatment or at substantial risk of developing a disease, disorder, or condition wherein such treatment is beneficial. Exemplary prophylactic applications of L5G2BPs, related compounds, and related compositions include reducing the severity of an imminent cancer, reducing the spread of an imminent cancer, reducing the likelihood of developing cancer, reducing the effects of an imminent cancer, or a combination of any thereof, etc.

Unless otherwise stated or clearly contradicted by context, the term “treatment” refers to the delivery of an effective amount of a therapeutically active compound of the invention with the purpose of preventing any symptoms or disease state to develop or with the purpose of easing, ameliorating, or eradicating (curing) such symptoms or disease states already developed. The term “treatment” is thus meant to include prophylactic treatment. Terms similar to “treatment” (e.g., “treating”) should be similar construed unless otherwise stated or clearly contradicted. However, it will be understood that “therapeutic regimens” and “prophylactic regimens” provided by the invention can be considered separate and independent aspects of this invention (e.g., such regimens may differ in terms of dosage, dosage regimen, etc.).

In another aspect, the invention provides methods for inhibiting tumor growth and/or metastasis in an individual in need thereof, comprising contacting the tumor with an amount of a L5G2BP, related compound, or related composition (e.g., a combination composition) of the invention, so as to inhibit tumor growth and/or metastasis. The invention similarly relates to the use of L5G2BPs or such related compounds or related compositions in the preparation of medicaments for inhibiting tumor growth and metastasis.

In one aspect, the method is applied to reduce the growth of tumor(s) that secrete detectable amounts of Ln-5 or Ln-5 subunit(s), such as an abnormally high level of a Ln-5 (e.g., a Ln-5 form associated with cancer), a β3/γ2 heterotrimer peptide, a γ2 monomer peptide, or combination thereof, as compared to non-neoplastic epithelial cells. The phrase “laminin-5 secreting tumor” refers to a tumor that expresses detectable amounts of Ln-5 or a fragment thereof, unless otherwise stated (e.g., where the tumor produces an abnormally high amount of a Ln-5 peptide or subunit). In these and other aspects, subjects treated by delivery of L5G2BPs, related compounds, or related compositions typically are mammals and commonly is a human. Ln-5 secreting tumors include, but are not limited to, carcinomas. Ln-5 secreting carcinomas include, but are not limited to squamous cell carcinomas (including but not limited to squamous cell carcinoma of skin, hypopharynx, cervix, and vulva), gastric carcinomas, colon adenocarcinomas, colorectal carcinomas, and cervical carcinomas. Other carcinomas that can be treated by inventive methods described herein include ductal mammary carcinomas. The invention provides methods of treating these types of cancers and relates to the use of L5G2BPs, related compounds, and related compositions for the preparation of medicaments to treat such conditions. Other common cancers that can be treated by inventive methods described herein include malignant melanomas.

Inhibiting tumor growth generally means causing a reduction in the amount of tumor growth that would occur in the absence of treatment and/or substantially complete cessation of detectable tumor growth, and includes decreases in tumor size and/or decrease in the rate of tumor growth. Inhibiting metastases means to reduce the amount of tumor metastasis that would occur in the absence of treatment, and includes a relative decrease in the number and/or size of metastases.

In another aspect, the invention provides a method for inhibiting migration of one or more laminin-5 secreting tumor(s), cancer cells/cancer cell populations, preneoplastic cells/cell populations, or population of other epithelial cells in a human patient comprising delivering a L5G2BP, such as an anti-γ2 DIII of the invention, a related compound (e.g., a DNA vaccine encoding a γ2 immunogenic peptide corresponding to one of the epitopes described herein), or a related composition of the invention to the tumor(s), cell population(s), etc. or to an area sufficiently near the tumor(s), cell population(s), etc., and in an amount and under conditions such that migration of the Ln-5 secreting tumor(s) or cell(s) is/are detectably inhibited (as determined by cessation of movement of the tumor in the patient or by comparison of tumor migration after administration of the L5G2BP against tumor migration at a similar stage in cancer progression in a suitably sized population of similar patients not treated with the L5G2BP, related compound, or related composition, such as may be determined through clinical trials involving administration of such compositions).

In one aspect, “migration” of cells means persistent migration (i.e., in such an aspect, the invention provides a method of reducing persistent migration in cancer cells). In another aspect, the invention provides a method of reducing neoplastic and/or preneoplastic cell polarization in a mammalian host, such as human, by delivery of a L5G2D3BP, related compound, or related composition of the invention.

In another aspect, such methods can be used to reduce the invasive/migratory potential of tumor cells. In still a different aspect, the inventive methods can provide means for eliciting, promoting, and/or enhancing an anti-tumor effect by slowing the growth, spread, or growth and spread of the front of a tumor into surrounding tissues, or the expected growth, spread, or growth and spread of a tumor. Tumor cell growth inhibition can be measured by any suitable standard and technique using, e.g., other methods described herein and/or inhibition assays such as are described in WO 89/06692.

In a further aspect, inventive methods described herein provide a mechanism for detectably retarding the invasive/migratory potential of laminin-5 positive cancer cells (e.g., by administering an anti-γ2 DIII mAb of the invention alone or in combination with one or more additional anti-cancer agents). Inventive methods further provide a mechanism for detectably retarding the invasive and/or migration potential of a laminin-5 positive cancer cell by exposing said cell to a monoclonal antibody specific to the γ2 chain of laminin-5. Inventive methods provided herein additionally can be applied to detectably disrupt contacts and/or associations between cancer cells, such as invading malignant cells, and an associated matrix, such as a provisional matrix of the type found in the immediate surroundings of cancer cells.

The invention further provides a method for reducing the scattering of tumor cells in a mammalian cell population, such as a population of tumor cells in a human patient, an animal model, or in culture, comprising delivering a L5G2BP, a related compound, or a related composition (e.g., a combination composition) of the invention to the cells under conditions and in an amount such that tumor cell scattering is detectably reduced. Again, in this and other aspects of the invention, the term delivery means delivery by any suitable technique including, e.g., by expression from a gene transfer vector, by direct administration (e.g., injection in solution or biolistic delivery), or any other suitable method.

Another feature of the invention is a method for inhibiting the formation of budding tumor cells and/or reducing tumor cell budding in a tumor comprising delivering a L5G2BP of the invention, related compound, or related composition of the invention in an amount and under conditions suitable for detectably inhibiting budding tumor cell formation and/or generally reducing tumor cell budding a tumor. Tumor cell budding in, e.g., colorectal carcinoma has been associated with the presence of intracellular laminin-5 (See, e.g., Sordat, et al., J. Pathol. 185: 44-52, 1998). As with other aspects of the invention directed to inhibition or reduction, such measurements can be made with respect to an individual, if appropriate (e.g., a detection that ongoing tumor cell budding has ceased in an individual would be one measure of the successful application of this method) and/or in the context of a patient population wherein successful application of the method is measured against the typical biological phenomena observed in a control group of a substantially similar pool of patients (e.g., patients suffering from a carcinoma associated with budding tumor cells).

The inventive methods also can be used to reduce the mimicry of embryonic vasculogenesis by aggressive cancer cells. Vasculogenic mimicry is described in, e.g., Seftor et al., Cancer Res. Sep. 1, 200161(17):6322-7. Inventive methods provided here also or alternatively can be used to reduce the formation of neoplastic tubules and/or the dissociation of cells from neoplastic tubules.

An additional aspect of the invention is to provide a method for inhibiting or slowing the growth and/or spreading of a tumor into surrounding tissue by delivering to a patient in need thereof an anti-γ2 DIII mAb or other effective L5G2BP, related compound, or combination composition.

In one aspect of the invention, a therapeutically effective amount of a L5G2BP, related compound, and/or related composition is administered or otherwise delivered (e.g., by gene expression, gene activation, or combination thereof) to cells or tissues associated with degraded, fragmented, and/or cleaved γ2 peptides and/or β3 peptides which are associated with epithelial cell migration, such as epithelial-derived tumor cell migration (such peptides may be referred to as promigratory γ2 fragments, β3 fragments, and/or γ2/β3 heterodimer peptides—see, e.g., Udayakumar et al., Cancer Res. May 1, 2003;63(9):2292-9; Seftor et al., Mol Cancer Ther. November 2002;1(13):1173-9; Katayama et al., J Mol Histol. March 2004;35(3):277-86; and Giles et al., J Cell Sci. August 2001;114(Pt 16):2967-76 for a description of such promigratory γ2 and β3 peptide fragments and Sordat et al., J Pathol. May 1998;185(1):44-52 with respect to promigratory γ2/β3 heterodimers). In another aspect, a therapeutically effective amount of a L5G2BP, related compound, and/or combination composition is delivered to cells or tissues associated with detectable levels of aberrant Ln-5 production and/or defectively-processed Ln-5 peptides (see, e.g., Tunggal et al., Am J Pathol. February 2002;160(2):459-68, and Sordat et al., J Pathol. 1998, supra). In a further aspect, a L5G2D3BP, related compound, or combination composition, is delivered in an amount effective to reduce and/or impede cell migration in tissues associated with defective basement membrane and/or hemidesmosome structures. In yet another aspect, the invention provides a method of reducing and/or impeding cell migration through the delivery of an effective amount of a L5G2BP, related compound, or combination composition to tissues associated with a decrease in alpha 3 integrin chain production, alpha 6 chain production, and/or beta 4 chain production. In a further aspect, the invention provides a method of reducing epithelial cell migration (e.g., epithelial-derived tumor cell migration) in a patient comprising delivering an effective amount of a L5G2BP, related compound, and/or combination composition to tissues or cells associated with silencing of Ln-5 genes through, e.g., mutation and/or aberrant Ln-5 promoter methylation (see, e.g., Sathyanarayana et al., Clin Cancer Res. July 2003;9(7):2665-72 and Sathyanarayana et al., Clin Cancer Res. Dec. 15, 2003; 9(17):6389-94, for a description of such physiological events).

In another aspect, the invention provides a method for modulating (e.g., interfering, such as inhibiting) tumor cell-basement membrane interaction and/or adhesion comprising administering or otherwise delivering a L5G2BP of the invention, a related compound, or a combination composition in an amount effective to reduce tumor cell-basement membrane interaction and/or adhesion. Thus, in one aspect, inventive methods described herein can be applied to reduce tumor cell/basement membrane adhesion. Such adhesion is important, if not crucial, for the invasion of non-malignant tissues by epithelial cancer cells.

Another feature of the inventive methods described herein is the ability to apply such methods to interfere with interactions between aberrant γ2 chain peptides or fragments and surrounding tissues or cells.

Yet another feature of inventive methods provided herein is the ability to inhibit the conversion of carcinoma cell phenotype from epithelial to spindle-shaped (e.g., reduce the rate of such conversion, reduce the total number of converted cells, etc., with respect to the human patient and/or a population of substantially similar patients). Thus, in an exemplary aspect, the invention provides a method for reducing the conversion of such a phenotypic change which method comprises delivering an effective amount of a L5G2BP to precancerous epithelial cells so as to reduce such conversion.

Inventive methods provided herein also can be particular advantageous in the context of inducing, promoting, and/or enhancing a physiological response associated with the treatment of cancer and/or reducing at least one aspect of cancer progression in cancer cells classified as poorly differentiated (see, e.g., Sordat et al., J. Pathol., 185:44-52 (1998) for an example of research discussing such poorly differentiated cancer cells).

Inventive methods provided herein also can be particularly advantageous with respect to the elimination (partial or total) of micrometastases and/or for the prevention of a recurrence of cancer in a patient previously diagnosed with cancer but currently in a state of remission. Methods for assessing recurrence and/or the risk of recurrence are known in the art (see, e.g., U.S. Pat. No. 6,656,684) and can include application of other cancer diagnostic methods described herein. Additional aspects related to recurrence and remission are discussed elsewhere herein.

Delivering a L5G2BP, related compound, and/or related composition of the invention to a mammalian host, such as a human patient, also provides a method for reducing the migration of epithelial cells generally, including noncancerous epithelial cells (the role of Ln-5 in epithelial cell migration is discussed in, e.g., Verrando, et al., Lab Invest. 71: 567-74, 1994; Kikkawa, et al., J. Biochem. (Tokyo). 116: 862-9,1994; Zhang, et al., Exp. Cell. Res. 227: 30922, 1996; O'Toole, et al., Exp. Cell. Res. 233: 330-9,1997; Tani, et al., Am. J. Pathol. 151: 1289-302, 1997; and Salo, et al., Matrix Biology, in press, 1999). Ln-5 is associated with epithelial cell migration in, for example, wound healing as well as cancer. For this and other reasons, the method of this aspect can be applied either to inhibit epithelial cell migration in both malignant and nonmalignant cells. Thus, for example, in one aspect, the invention relates to the use of L5G2BPs, related compounds, and/or related compositions in the preparation of a medicament for modulating wound healing or for a method of modulating wound healing activities (e.g., in a patient in need thereof), for example by modulating (e.g., reducing) epithelial cell migration (e.g., keratinocyte migration) in the context of wound healing in a mammalian host.

In another aspect, inventive methods provided herein can be used as a technique for modulating cell differentiation and/or cell proliferation. As indicated above, inventive methods also can be used to reduce cell migration. Cell migration assays are described in U.S. patent application Ser. No. 10/695,559. Thus, for example, the invention provides a method for blocking migration of epithelial cells comprising delivering to such cells an inhibitory amount of a composition comprising one or more antibodies against γ2 DIII.

A further aspect of the invention is to provide a method for promoting an immune response against a tumor by administering a L5G2BP, such as a monoclonal antibody against γ2 DIII, to a patient in need thereof in an amount and under conditions sufficient to induce a detectable immune response against the tumor. Inducement of an immune response can be measured by any suitable result, such as the inducement, promotion, and/or enhancement of antibody-mediated opsonization, complement-mediated response, etc.

In a particular aspect, inventive methods described here provide a mechanism for inhibiting the formation of interactions between γ2 DIII and other Ln-5 binding molecules or Ln-5 chains involving Cys442 comprising administering a L5G2D3BP, such as an anti-γ2 DIII mAb, which is specific for a portion of γ2 DIII that comprises Cys442 or that is near enough to Cys442 to cause steric hindrance that prevents peptide binding to γ2 DIII at Cys442, such that access to Cys442 is effectively blocked by the binding of the L5G2D3BP.

In further aspects, the invention provides a method for inhibiting or blocking Ln-5 heterotrimer formation comprising administering a L5G2BP that is specific and/or selective for a region of γ2 that is essentially for heterotrimer formation. In another aspect, the invention provides a method for intermolecular interactions associated γ2 comprising administering a L5G2BP that binds to at least a portion of γ2 or γ2-associated portion of Ln-5 (e.g., DIII or the hinge region of Ln-5), such that γ2-associated intermolecular interactions are reduced or inhibited.

In another aspect, the invention provides a mechanism for reducing the processing of the γ2 chain comprising administering or otherwise delivering an effective amount of an anti-γ2 DIII antibody or other L5G2BP that is selective and/or specific for a region comprising at least some of the Ln-5 region overlapping the cleavage site so as to reduce processing of the γ2 chain in a chordate, such as a mammalian host (e.g., a human patient). Such a therapeutic strategy also can be used to reduce one or more aspects of cancer progression in a human patient having a condition associated with γ2-peptide-associated cancer or pre-cancer growth.

In another exemplary aspect, the invention provides a method for reducing the adhesion between cancer cells and matrix structures such as the basement membrane. Cell adhesion assays for Ln-5 associated cells are described in, e.g., U.S. Patent Application 10/695,559. Moreover, the adhesion properties of laminin-5 have been demonstrated in several cell attachment studies (see, e.g., Carter et al., (1991) Cell 65: 599-610; Rousselle et al., (1991 ) J. Cell Biol. 114: 567:576; Sonnenberg et al., (1991) J. Cell Biol. 113: 907-917; Niessen, et al., (1994) Exp. Cell. Res. 211: 360-367; and Rousselle et al. (1994) J. Cell Biol. 125:205214)). Thus, in an exemplary aspect, the invention provides a method of reducing cancer cell-matrix adhesion comprising administering or otherwise delivering an effective amount of a L5G2BP to a mammalian host so as to detectably reduce such adhesion.

The adhesive function of laminin-5 has been shown to be mediated through, e.g., α3β1 and α6β4 integrins (see, e.g., Carteret al. (1991) Cell 65: 599-610; Sonnenberg (1991) J. Cell Biol. 113: 907-917; and Rousselle (1994) J. Cell Biol. 125: 205214)). Thus, in one variation of the aspect of the preceding paragraph, a L5G2BP, such as an anti-γ2 DIII antibody, can be administered with a molecule that targets and binds α3β1 integrin, α6β4 integrin, α6β1 integrin, or combination of any thereof, such as antibodies specific for these integrins, thereby further blocking Ln-5-mediated association of cells and the surrounding matrix, such as the ECM or lamina lucida.

In another similar aspect, the invention provides a method of reducing tumor cell invasiveness in a mammal, such as a human patient, comprising delivering a L5G2BP to the mammal and a protein that binds (and desirably inhibits) a matrix metalloproteinase-2 (MMP-2), such as an anti-MMP2, under conditions and in respective amounts such that tumor cell invasiveness is reduced (surrogate nucleic acid(s) also can be used in such a method). The invention also provides combination compositions characterized by inclusion of effective amounts of such molecules. The role of Ln-5 and MMP-2 in tumor cell budding and invasiveness is explored in, e.g., Masaki et al., Anticancer Res. September-October 2003;23(5b):4113-9. In this and other suitable combination aspects of the invention, a bispecific antibody comprising a set of VH and VL sequences specific for γ2 DIII and another set of VL and VL sequences specific for a second molecule, such as MMP-2, can be used as an alternative to delivery of two separate molecules (bispecific antibodies and combination compositions that comprise similar combinations that may likewise be suitable basis for multispecific antibodies are discussed elsewhere herein). In this aspect and other combination administration method aspects, a single administration of a combination compound of the invention can be employed in place of a separate delivery strategy (here, e.g., a composition comprising an anti-γ2 DIII antibody and a anti-MMP-2 antibody can be used to so inhibit cell invasiveness, rather than requiring both antibodies to be administered to a host).

In another sense, the invention provides a method of increasing the likelihood of survival over a relevant period in a human patient diagnosed with cancer. For example, the invention provides a method of increasing the likelihood of survival about six months, about nine months, about one year, about three years, about five years, about seven years, about ten years, or more, after treatment with a L5G2BP or L5G2BP composition of the invention, as compared to not receiving treatment with the L5G2BP or related composition (survival rates can be determined by studies on a population of similar patients, such as in the context of a clinical trial).

In another aspect, the invention provides a method for improving the quality of life of a cancer patient comprising administering to the patient a composition of the invention in an amount effective to improve the quality of life thereof. Methods for assessing patient quality of life in cancer treatment are well known in the art (see, e.g., Movass and Scott, Hematol Oncol Clin North Am. February 2004;18(1):161-86; Dunn et al., Aust NZ J Public Health. 2003;27(1):41-53; Morton and Izzard, World J Surg. July 2003;27(7):884-9; Okamato et al., Breast Cancer. 2003;10(3):204-13; Conroy et al., Expert Rev Anticancer Ther. August 2003;3(4):493-504; List et al., Cancer Treat Res. 2003;114:331-51; and Shimozuma et al., Breast Cancer. 2002;9(3):196-202).

In a further aspect, molecules, compositions, and methods of the invention can be characterized by not substantially provoking, and preferably not detectably provoking, a blistering or other type of undesirable autoimmune response (e.g., an autoimmune disease condition) upon administration of an effective amount thereof.

In a more particular aspect, any one of the methods described herein, such as the reduction of invasive cancer cells, is practiced by a method that comprises delivering a L5G2BP to a tissue that is characterized by a lack of mature hemidesmosomes as compared to healthy (nonmalignant) basement membrane-associated tissues. In this and other aspects, the delivery of a L5G2BP can be considered to provide a mechanism for modulating the architecture of the basement membrane in a mammalian host, such as a human cancer patient (e.g., for increasing the amount of mature hemidesmosome-containing tissue in a patient). However, in another aspect, administration of a L5G2BP can provide a mechanism for inhibiting hemidesmosome assembly.

In a further aspect, the inventive methods described herein also or alternatively provide a method for impeding branching morphogenesis of epithelial and epithelial-derived cells. In still another aspect, such methods can be used to modulate epithelial cell polarization.

Inventive methods also can be used as a means to interfere with Ln-5 interaction with interacting molecules (examples of which are discussed elsewhere herein), such as Ln-5 interacting integrins (e.g., administration of a L5G2BP can be used to interfere with Ln-5:α6β1 integrin interactions).

In a further aspect, the inventive methods can be used to decrease the rate of angiogenesis and/or neovascularization, such as ePTFE-associated neovascularization/angiogenesis, in a host, such as in a human cancer patient. In yet another aspect, the inventive methods can be used to delay, reduce, and/or prevent the loss of normal basement membrane barrier structures in the course of cancer progression and/or angiogenesis/neovascularization.

In a further aspect, the invention provides a method of modulating a Ln-5-associated signaling pathway(s), such as in the context of regulating apoptosis in a cancer cell, comprising delivering a L5G2BP to the cell population in an amount and under conditions such that γ2 DIII is bound by the L5G2BP in a manner resulting in modulation of a Ln-5-associated signaling pathway. For example, such methods can be used to modulate RAC and/or NFκB activation, so as to, for example, impede RAC-mediated sustenance of tumor cell viability.

In a further aspect, inventive methods taught herein can be used to reduce hematogenous metastasis and/or tumor cell arrest (e.g., in the context of pulmonary metastases) in a human patient.

In yet another facet, various methods of the invention can be used as a method of modulating Ln-5 associated protein kinase C (PKC), phosphoinositide 3-OH kinase (PI3-K), Akt, and/or MAP kinase activities/pathway(s). In another aspect, the invention provides combination compositions and methods involving inhibitors of one or more of these pathways. In a specific example, the invention provides combination compositions and methods that involve at least one L5G2BP and LY294002 and/or Wortmannin (these compounds and their use are known in the art—see, e.g., Fukuchi et al., Biochim Biophys Acta. Apr. 17, 2000;1496(2-3):207-20; Yu et al., Mol Carcinog. October 2004;41(2):85).

L5G2BPs, related molecules, and/or related compositions (e.g., combination compositions) of the invention can be administered to achieve any combination of the aforementioned physiological responses and promote any of the above-described therapeutic and/or prophylactic regimens. Thus, for example, in one aspect the invention provides a method of reducing the migration and invasiveness of epithelial-derived cancer cells in a human patient in need thereof by, among other things, delivering an amount of a L5G2BP, a combination composition (any of which such compositions may be delivered by, e.g., expression of multiple nucleic acid sequences encoding a L5G2D3BP and other anti-cancer peptide(s)), a L5G2BP related compound, or a combination thereof, such that migration and invasiveness of epithelial-derived cancer cells is detectably reduced.

In another aspect, the invention provides a method of reducing the risk of a start of cancer progression, reducing the risk of further cancer progression in a cell population that has undergone initiation, and/or providing a therapeutic regimen for reducing cancer progression in a human patient, which comprises administered an amount of a L5G2BP, a related compound, or combination composition (or applying a combination administration method) to a patient that has been diagnosed as having cells exhibiting preneoplastic and/or neoplastic cell-like levels and/or types of gene expression, such as cancer-associated patterns of erbB2 (Her2/neu) gene expression; p53 gene expression; BRCA1 and/or BRCA2 gene expression; PTEN gene expression; ras family gene expression (k-ras, h-ras, m-ras, RAB2, RAP2A, etc.), c-MYC gene expression or cancer-like expression of one or more of the following Exol, ASPP2, C/EBPD, p16(INK4a) CDKN2A, R24P, P81L, V126D, BNIP3, MYH, PTCH, B-ras, A-ras, PPAR (α, γ, and Δ), MC1R, TP16p14/ARF, SMAD3, SMAD4, CDK4, p73, p15, AXIN1, raf, CHEK2, SHIP, HFE, p21(CIP1/WAF1), FAS, TSG101, MEN1, GSTPI, P2X7, BRAF, HPV type 16 E7, P27 Cyclin E, Cyclin D, Rb, P300, Mdm2, Fos, Jun, N-Ras, Ki-Ras, Raf-1, AbI, Bcl-2, Bcl-6, Bax, APC (Accession No: M74088), Beta catenin, E-cadherin, PI3-kinase, TGFα, TGFβ, TGFβ receptor, Src, Met, Akt, Alk, Grb2, Shc, and E2F 1-5. In a particular exemplary aspect, the invention provides a method of inhibiting cancer progression (either before or after detection of any aspect thereof) in a human exhibiting cancer-like upregulation/expression of Ras and Myc; expression of Ras with loss of regular p53 gene activity; expression of Ras with loss of regular Rb activity; expression of Ras with loss of regular NFKβ activity; Expression of Ras with loss of regular APC activity; expression of Ras with loss of regular Arf activity; expression of Ras with E7; etc.

Additional benefits of inventive methods include the reduction or prevention of cancer progression in a pre-invasive lesion or growth; inducing, promoting, and/or enhancing tumor regression; enhancing a patient's immune system attach against cancer tumors; enhancement of the effectiveness of an anti-cancer agent; treating a pre-neoplastic or neoplastic disease characterized by abnormal expression of Ln-5 and/or producing promigratory γ2-associated peptides; reducing cancer progression in cell exhibiting marked nuclear atypia; and treating a human for a condition associated with an undesirable epithelial and/or epithelial-derived cell proliferation.

In one aspect, a L5G2BP (or related compound surrogate) is administered or otherwise delivered, in an effective amount, to or near an intraepithelial neoplasia/lesion so as to reduce cancer progression therein, for example in a squamous intraepithelial lesion. In another aspect, inventive methods described herein provide a method of treating cancer in premalignant phase/preinvasive phase cells.

In a further aspect, inventive methods described herein can be applied to significantly reduce the number of cancer cells in a vertebrate host, such that, for example, the total number and/or size of tumors is/are reduced. Such methods can be applied to treat any suitable type of tumor including chemoresistant tumors, solid tumors, and/or metastasized tumors. In a related sense, the invention provides a method for killing preneoplastic and/or neoplastic cells in a vertebrate, such as a human cancer patient.

Inventive methods provided herein can be used to reduce the number, spread, and/or development of metastases in a chordate, such as in a human cancer patient. In a particular aspect, the invention provides a method of reducing the number of metastases in the tissue(s) of a chordate host, such as a mammal, for example a human patient, by at least about 30%, such as about 40% or more, about 50% or more, about 60% or more, or about 70% or more (e.g., about 35-75%) as compared to non-treatment in a substantially similar host over a period of about 3 or more days, such as about one week or more (e.g., about 2, 3, 4, 6, 8, 10, or 12 weeks; about 4, 5, 6, 9, or 12 months; or longer).

Another advantageous aspect of the invention is the reduction of cancer progression in a patient having a cancer staged by the presence of a significant number of poorly differentiated and/or undifferentiated cancer cells (highly anaplastic cells). L5G2BPs, such as conjugated L5G2D3BPs, may be useful in reducing cancer progressions in such patients where other methods have been ineffective.

In another aspect, the invention provides a method wherein a L5G2BP is used to target other molecules to migrating cells, such as migrating and invasive cancer cells, or to other molecules, cells, and/or tissues associated with γ2-associated proteins.

In one such aspect, a L5G2BP is a conjugated peptide, as described elsewhere herein, wherein, for example, the conjugate comprises a radioisotope, a toxin, an apoptotic domain/peptide, or other cytotoxic and/or anti-cancer agent. Such methods are especially useful in targeting the invasion front of a human carcinoma.

Several immunoconjugates, particularly those that incorporate internalizing antibodies and tumor-selective linkers, may exhibit impressive activity against cancer cells in preclinical models. Immunoconjugates that deliver doxorubicin, maytansine, and calicheamicin are examples of such molecules. Gemtuzumab ozogamicin, a calicheamicin conjugate that targets CD33, is an immunoconjugated molecule that has recently been approved by the US Food and Drug Administration (FDA) for treatment of acute myelogenous leukemia (AML). Such immunoconjugates are described in, e.g., Trail et al., Cancer Immunol Immunother. May 2003; 52(5):328-37 and Payne, Cancer Cell. March 2003;3(3):207-12. A number of other conjugated molecules are described elsewhere herein as are combination partners that can also make conjugate partners and/or fusion partners in the context of L5G2BPs to the extent such molecules are suitably able to bind γ2 or an associated portion of Ln-5.

In one aspect, the invention combines a L5G2BP that is conjugated to a molecule that can be used as a reporter or label, such as a green fluorescent protein (GFP) domain, or a radionuclide, and used to identify the location of tumors. In one exemplary aspect, the invention provides a method for identifying a tumor associated with an identified metastases or other identified cancer cell population in a human patient comprising administering such a composition and identifying locations where the L5G2BP has gathered, indicating the presence of migrating epithelial or epithelial-derived cells, such as invasive carcinoma cells, and/or the presence of γ2-associated peptides (e.g., γ2-associated peptides secreted from cancerous or precancerous cells). Using such information, targeted application of radiation therapy, chemotherapy, or application of surgical techniques (e.g., a convention technique such as a biopsy, preferably a minimally invasive technique such as laser-assisted surgery, or an alternative surgical technique such as cryosurgery) can be used to efficiently reduce, isolate, or destroy a cancerous growth associated with Ln-5 or a fragment thereof. Surgery in such contexts can include primary surgery for removing one or more tumors, secondary cytoreductive surgery, and palliative secondary surgery.

In another aspect, L5G2BPs can be used in a pre-targeting protocol. In such protocols, primary molecules (here one or more L5G2BPs) are allowed to bind targets (e.g., γ2-associated peptides) and anti-L5G2BP antibody conjugates are thereafter used to deliver payload(s) to the vicinity of the targets.

In another aspect, the invention provides a method of delivering a prodrug to target cells for targeted therapy. Many therapeutic agents are administered as prodrugs. A prodrug typically is a chemically modified form of the therapeutic agent designed to improve either its pharmacokinetic, pharmacological, or toxicological profiles. A prodrug is typically administered in a masked state. A chemical reaction, which typically is enzymatically facilitated, is usually required for prodrug activation.

In one aspect of the invention, L5G2BPs are used to deliver a conjugated prodrug that can be activated by an exogenous enzyme that is subsequently or simultaneously administered to a patient. In another aspect, L5G2BPs are used to deliver a conjugated enzyme that can be used to activate a subsequently or simultaneously delivered prodrug. Antibody-directed enzyme prodrug therapy (ADEPT) methods, such as described in the foregoing sentence, are known in the art (see, e.g., Bagshawe et al., Br. J. Cancer 58, 700-703, 1988; Senter et al., Proc. Natl. Acad. Sci. USA 85, 4842-4846,1988; Niculescu-Duvaz, et al., Adv. Drug Delivery Rev. 26,151-172, 1997; and International Patent Application WO 93/02703).

Clinically useful prodrug activation systems, such as anti-cancer prodrug systems, can be delivered as masked L5G2BP-prodrug conjugates in combination with exogenous activating enzymes, by which the masked conjugated prodrugs are unmasked and thereby allowed to effect pharmacological effects, such as cytotoxic effects, on target cells. Prodrugs should typically not be activated by endogenous enzymes and are composed accordingly. Thus, for example, in ADEPT therapies bacterial enzymes may be used. In view of the immunogenicity of such enzymes, ADAPT therapy involving “human” or “humanized” catalytic L5G2BP antibodies can be more effective and/or less associated with negative side effects in therapeutic application.

In another aspect, a catalytic L5G2BP antibody is administered in association with a masked prodrug. Such catalytic antibodies typically are selected to catalyze the reaction that is not catalyzed by endogenous enzymes. Prodrug activation by catalytic antibodies (abzymes) specific for a target cell (here, an NK cell and/or another STM-associated cell, such as an NK target cell) is commonly referred to as antibody-directed abzyme prodrug therapy (ADAPT), ADAPT methods can be performed with catalytic L5G2BP antibodies and such antibodies are another feature of the invention. In one aspect, the invention provides a catalytic antibody L5G2BP that is specific for both γ2 and an enzymatic substrate site.

Masked prodrugs can include prodrug versions of anti-cancer agents such as doxorubicin and camptothecin (see, e.g., Barbas et al., Proc. Natl. Acad. Sci USA 96, 6925-6930, 1999), and phenolic N-mustards. Additional methods and principles relevant to such prodrug-associated therapies are provided in, e.g., U.S. Pat. Nos. 5,807,688 and 6,268,488; Miyashita, et al., Proc. Natl. Acad. Sci. USA 90: 5337-5340 (1993); Campbell, et al., J. Am. Chem. Soc. 116: 2165-2166 (1994); Wentworth, et al., Proc. Natl. Acad. Sci. USA 93: 799-803 (1996); and Kakinuma et al., J Immunol Methods. Nov. 1, 2002;269(1-2):269-81.

As indicated already herein, L5G2BPs can be delivered in association or combination with one or more active agents or therapeutic methods. In this respect, generally any combination composition described herein is to be understood as providing support for co-delivery methods involving the various secondary compounds.

In one aspect, the invention provides a method of delivering one or more L5G2BPs in association with application of anti-cancer chemotherapy and/or radiation therapy methods. One benefit of such combination methods is that use of a L5G2BP may permit a reduction in the chemotherapy and/or radiation dosage necessary to inhibit tumor growth and/or metastasis. As used herein, “radiotherapy” includes but is not limited to the use of radio-labeled compounds targeting tumor cells. Any reduction in chemotherapeutic or radiation dosage may benefit a patient by resulting in fewer and decreased side effects relative to standard chemotherapy and/or radiation therapy treatment (accordingly, quality of life may be higher where dosages of such compounds are reduced in cancer treatment). In this aspect, a L5G2BP (e.g., an anti-γ2 DIII antibody) may be administered or otherwise delivered prior to, at the time of, or shortly after a given round of treatment with chemotherapeutic and/or radiation therapy. Typically, an anti-γ2 DIII antibody is administered prior to or simultaneously with a given round of chemotherapy and/or radiation therapy in practicing such methods, which may be all rounds or less than all rounds. The exact timing of antibody administration typically will be determined by an attending physician based on a number of factors. In one exemplary aspect, an anti-γ2 mAb can be administered or otherwise delivered about 24 hours before a given round of chemotherapy and/or radiation therapy and simultaneously with a given round of chemotherapy and/or radiation therapy.

Methods of the invention can be appropriate for application in conjunction with chemotherapy regiments using one or more anti-cancer cytotoxic agents/chemotherapeutic agents, including, but not limited to, cyclophosphamide, taxol (and other taxanes, such as docetaxel, paclitaxel, and the like), 5-fluorouracil, adriamycin, cisplatin, methotrexate, oxaliplatin, irinotecan, topotecan, leucovorin, carmustine, streptozocin, cytosine arabinoside, mitomycin C, prednisone, vindesine, carbaplatinum, and vincristine. Additional chemotherapeutic and cytotoxic agents are described elsewhere herein and further suitable chemotherapeutic and cytotoxic agents are known in the art. For a general discussion of cytotoxic agents used in chemotherapy, see, e.g., Sathe, M. et al., CANCER CHEMOTHERAPEUTIC AGENTS: HANDBOOK OF CLINICAL DATA (1978) and the second edition thereof (Preston—1982), and CANCER CHEMOTHERAPEUTIC AGENTS (Acs Professional Reference Book) (William Foye, Ed. 1995).

Application of therapeutic methods of the invention also can be particularly suitable for those patients in need of repeated or high doses of chemotherapy and/or radiation therapy under current therapeutic regimens. Delivery of L5G2BPs, alone or in combination with such agents, can provide a mechanism for reaching an at least as therapeutically effective outcome in a cancer patient with substantially lower amounts of chemotherapy and/or radiation therapy.

In general, combination administration methods of the invention can comprise any suitable administration scheme, including coadministration (as separate compositions or a single composition wherein the ingredients are mixed or separated) or stepwise administration of the various active agents.

In another aspect, a L5G2BP is delivered in association with inhibitors, binding molecules, or antibodies against Ln-6 and/or antibodies that specifically target Ln-5B, over Ln-5A, or visa versa (or fragments thereof). Ln-5B and related protein fragments are described in, e.g., Kariya et al., J Biol Chem. Jun. 4, 2004;279(23):24774-84. Epub Mar. 23, 2004). The invention further provides a method that also or alternatively comprises delivering antibodies against the P3 chain of Ln-5 to a host, so as to reduce cell migration, invasiveness, etc. In a further aspect, inventive methods described herein can further include the delivery of an antibody against matrilysin, an antibody against CD44, or both.

Various other combinations with L5G2BPs are described elsewhere herein (typically in the context of combination compositions) that can be similarly used in combination therapies of the invention and visa versa.

In delivering L5G2BPs, the amount or dosage range of the L5G2BP employed typically is one that effectively inhibits tumor growth, tumor cell invasiveness, and/or metastasis. An inhibiting amount of antibody that can be employed in such methods, for example, can range from generally between about 0.01 μg/kg body weight to about 15 mg/kg body weight, such as between about 0.05 μg/kg and about 10 mg/kg body weight, more specifically between about 1 μg/kg and about 10 mg/kg body weight, and even more particularly between about 10 μg/kg and about 5 mg/kg body weight.

Usually a daily dosage of active ingredient can be about 0.01 to 100 milligrams per kilogram of body weight. Ordinarily, about 1 to about 5 or about 1 to about 10 milligrams per kilogram per day given in divided doses of about 1 to about 6 times a day or in sustained release form may be effective to obtain desired results.

As a non-limiting example, treatment of Ln-5-related pathologies in humans or animals can be provided as a daily dosage of L5G2BP(s), such as monoclonal, chimeric, and/or murine antibodies, in an amount of about 0.1-100 mg/kg, such as 0.5, 0.9, 1.0,1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every 24, 12, 8, 6, 4, or 2 hours, or any combination thereof.

Other principles relevant to dosage are described elsewhere herein.

In general, L5G2BPs, related compounds, and related compositions can be administered via any suitable route, such as an oral, mucosal, buccal, intranasal, inhalable, intravenous, subcutaneous, intramuscular, parenteral, intertumor, intratumor, or topical route. Such peptides, related compounds, and compositions may also be administered continuously via a minipump or other suitable device. An anti-γ2 antibody or other L5G2BP may be administered parenterally in dosage unit formulations containing conventional pharmaceutically acceptable carriers, adjuvants, and the like (e.g., stabilizers, disintegrating agents, anti-oxidants, etc. as described elsewhere herein). The term “parenteral” as used herein includes, subcutaneous, intravenous, intraarterial, intramuscular, intrasternal, intratendinous, intraspinal, intracranial, intrathoracic, infusion techniques and intraperitoneal delivery. Most commonly, a γ2 DIII antibody will be administered intravenously or subcutaneously, in practicing therapeutic methods of the invention. Routes of injection also include injection into the muscle (intramuscular IM); injection under the skin (subcutaneous (s.c.)); injection into a vein (intravenous (IV)); injection into the abdominal cavity (intraperitoneal (IP)); and other delivery into/through the skin (intradermal delivery, usually by multiple injections, which may include biolistic injections).

An anti-γ2 antibody or other L5G2BP generally will be administered for as long as the disease condition is present, provided that the antibody causes the condition to stop worsening or to improve. An anti-γ2 antibody or other L5G2BP typically is administered as part of a pharmaceutically acceptable composition as described elsewhere herein.

An anti-γ2 antibody or other L5G2BP (or related surrogate/composition) may also be administered or otherwise delivered prophylactically in order to reduce the risk of developing cancer, delay the onset of the occurrence of an event in cancer progression, and/or reduce the risk of recurrence when a cancer is in remission. This type of method may be especially useful in patients wherein it is difficult to locate a tumor that is known to be present due to other biological factors.

In one aspect, a L5G2BP (e.g., a cytotoxic L5G2BP conjugate) or related composition (e.g., a combination composition) is administered by regional perfusion therapy (wherein the agent or composition is delivered directly to target organs or areas affected by or at risk of being affected by cancer).

For therapy, L5G2BPs may be administered topically or parenterally, e.g. by injection at a particular site, for example, subcutaneously, intraperitoneally, intravascularly, intranasally, transdermally, or the like. Formulations for injection can comprise any suitable excipients, and typically will comprise (or be substantially composed of) a physiologically-acceptable medium, such as water, saline, PBS, aqueous ethanol, aqueous ethylene glycols, or the like. Water soluble preservatives which may be employed in such formulations include sodium bisulfite, sodium thiosulfate, ascorbate, benzalkonium chloride, chlorobutanol, thimerosal, phenylmercuric borate, parabens, benzyl alcohol, and phenylethanol. These agents may be present in individual amounts of from about 0.001 to about 5% by weight and preferably about 0.01 to about 2%. Suitable water soluble buffering agents that may be employed in these and other formulations described herein include alkali or alkaline earth carbonates, phosphates, bicarbonates, citrates, borates, acetates, succinates and the like, such as sodium phosphate, citrate, borate, acetate, bicarbonate and carbonate. Additives such as carboxymethylcellulose may be used as a carrier in amounts of from about 0.01 to about 5% by weight. Formulation can vary depending upon the purpose of the formulation, the particular mode employed for modulating the receptor activity, the intended treatment, and the like. The formulation may involve patches, capsules, liposomes, time delayed coatings, pills, or may be formulated in pumps for continuous administration. The specific dosage can be determined empirically in accordance with known ways. See, for example Harrison's, PRINCIPLES OF INTERNAL MEDICINE, 11th ed. Braunwald et al. ed, McGraw Hill Book Co., New York, 1987.

In another aspect, two or more L5G2BPs are delivered to a host, such as a human patient, to induce, promote, and/or enhance a beneficial physiological response, such as the reduction of cancer progression. As described elsewhere herein, in one aspect the invention provides compositions comprising a plurality (e.g., a “cocktail”) of L5G2BPs. This and other combination compositions can be used in various inventive methods provided here and to prepare medicaments for the treatment of diseases, disorders, and conditions described here. Delivery of such combinations of L5G2BPs is another feature of the invention.

In one aspect, inventive methods provided herein can comprise administering or otherwise delivering two different L5G2BPs over a period of time, wherein the delivery of such different L5G2BPs overlap or do not overlap. For example, the invention provides a method of delivering two or more L5G2BPs over a period of one month, the beginning of the therapy involving the second L5G2D3BP starting about 1-3 weeks (e.g., about 10 days) after the first delivery of the first L5G2BP or at any time when a significant immune response to the first L5G2BP develops in the host, such that the continued use of the first L5G2BP has become detrimental to the patient or ineffective. Such methods can be particularly advantageous when using humanized antibodies in the inventive therapeutic regimens described herein.

The invention also provides methods for the identification of, and diagnosis of invasive cells and tissues, and other cells targeted by L5G2BPs, and for the monitoring of the progress of therapeutic treatments, status after treatment, risk of developing cancer, cancer progression, and the like.

In one example of such a diagnostic assay, the invention provides a method of diagnosing the level of invasive cells in a tissue comprising forming an immunocomplex between a L5G2BP and potential Ln-5 containing tissues or components thereof, and detecting formation of the immunocomplex, wherein the formation of the immunocomplex correlates with the presence of invasive cells in the tissue. Such “contacting” can be performed in vivo, using labeled isolated antibodies and standard imaging techniques, or can be performed in vitro on tissue samples.

L5G2BPs can be used to detect γ2-containing peptides and peptide fragments in any suitable biological sample (e.g., a tissue sample from a human patient, a cell culture, etc.) or other composition by any suitable technique. Examples of conventional immunoassays provided by the invention include, without limitation, an ELISA, an RIA, FACS assays, plasmon resonance assays, chromatographic assays, tissue immunohistochemistry, Western blot, and/or immunoprecipitation using a L5G2BP. Thus, in one aspect, the invention provides a method of detecting/assaying γ2-containing peptides in a composition comprising adding to the composition one or more L5G2BPs and thereafter adding labeled or detectable secondary antibodies (e.g., anti-human antibody antibodies or anti-γ2 Ab antibodies) to the composition so as to detect whether any proteins or structures in the composition are bound by the secondary antibody. In another aspect, a L5G2BP conjugated with a detection-facilitating agent (a fluor, an enzyme, a radionuclide, etc.) may be used for such diagnostic assays. Anti-γ2 antibodies of the invention may be used to detect γ2 and γ2-containing peptides in biological samples obtained from humans or in human tissues in vivo. Suitable labels for the antibody and/or secondary antibodies used in such techniques have been disclosed supra, and include various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive materials include ¹²⁵I, ¹³¹I, ³⁵S, and ³H.

Ln-5 γ2 peptides also can be assayed in a biological sample by a competition immunoassay utilizing γ2 peptide standard(s) labeled with a detectable substance and an unlabeled anti-γ2 DIII antibody, for example. In such an assay, the biological sample, a labeled γ2 peptide standard(s) and an anti-γ2 DIII antibody are combined and the amount of labeled γ2 standard bound to the unlabeled antibody is determined. The amount of γ2 peptide in the biological sample typically is inversely proportional to the amount of labeled γ2 standard bound to the anti-γ2 DIII antibody.

The L5G2BPs are particularly useful in the in vivo imaging of tumors, as briefly mentioned elsewhere herein. In vivo imaging of tumors associated with γ2 can be performed by any suitable technique. For example, ⁹⁹Tc or another gamma-ray emitting isotope can be used to label γ2 DIII antibodies in tumors or secondary labeled (e.g., FITC labeled) antibody:γ2 DIII complexes associated with tumors and the labeled antibodies imaged with a gamma scintillation camera (e.g., an Elscint Apex 409ECT device), typically using a low-energy, high resolution collimator or a low-energy all-purpose collimator. Stained tissues can then be assessed for radioactivity counting as an indicator of the amount of Ln-5 γ2-associated peptides in the tumor. The images obtained by the use of such techniques can be used to assess biodistribution of γ2-associated peptides in a patient, mammal, or tissue, for example in the context of using γ2 or γ2-containing peptides as a biomarker for differentiation of epithelial cells (e.g., in the context of cancer progression toward malignant growth) or the presence of invasive cancer cells. Variations on such techniques can include the use of magnetic resonance imaging (MRI) to improve imaging over such gamma scintillation camera techniques. Similar immunoscintigraphy methods and principles useful in performing such techniques are described in, e.g., Srivastava (ed.), RADIOLABELED MONOCLONAL ANTIBODIES FOR IMAGING AND THERAPY (Plenum Press 1988), Chase, “Medical Applications of Radioisotopes,” in REMINGTON'S PHARMACEUTICAL SCIENCES, 18th Edition, Gennaro et al. (eds.), pp. 624-652 (Mack Publishing Co., 1990), and Brown, “Clinical Use of Monoclonal Antibodies,” in BIOTECHNOLOGY AND PHARMACY 227-49, Pezzuto et al. (eds.) (Chapman & Hall 1993) (incorporated in its entirety). Such images also can be used for targeted delivery of other anti-cancer agents, examples of which are described herein (e.g., apoptotic agents, toxins, or CHOP chemotherapy compositions). Moreover, such images also or alternatively serve as the basis for targeting surgical techniques to remove tumors. Furthermore, such in vivo imaging techniques may allow for the identification and localization of a tumor in a situation where a patient is identified as having a tumor (due to the presence of other biomarkers, metastases, etc.), but the tumor cannot be identified by traditional analytical techniques. All of these methods are important features of the invention.

The in vivo imaging and other diagnostic methods provided by this invention can be particularly useful in the detection of micrometastases in a human patient (e.g., a patient not previously diagnosed with cancer or a patient in a period of recovery/remission from a cancer). Carcinoma cancer cells, which may make up to about 90% of all cancer cells, for example, have been demonstrated to stain very well with anti-γ2 DIII antibody conjugate compositions. Detection with anti-γ2 DIII mAbs and other L5G2BPs described herein can be indicative of the presence of carcinomas that are aggressive/invasive and also or alternatively provide an indication of the feasibility of using related anti-γ2 mAb, other L5G2BP, or related composition treatments against such micrometastases. Moreover, anti-γ2 mAbs that are associated with cancer cells are advantageously able to distinguish such cancer-associated tissues and cells from normal cells that associate with other forms of Ln-5.

In another aspect, the invention provides an in vivo imaging method wherein an anti-γ2 antibody is conjugated to a detection-promoting radio-opaque agent, the conjugated antibody is administered to a host, such as by injection into the bloodstream, and the presence and location of the labeled antibody in the host is assayed. This imaging technique can be useful in, among other things, the staging and treatment of Ln-5-associated neoplasms. Through this technique and any other diagnostic method provided herein, the invention provides a method for screening for the presence of disease-related cells in a human patient or a biological sample taken from a human patient.

For diagnostic imaging, radioisotopes may be bound to a L5G2BP either directly, or indirectly by using an intermediary functional group. Useful intermediary functional groups include chelators, such as ethylenediaminetetraacetic acid and diethylenetriaminepentaacetic acid (see, e.g., U.S. Pat. No. 5,057,313). In such diagnostic assays involving radioisotope-conjugated L5G2BPs, the dosage of conjugated peptide delivered to the patient typically is maintained at as low a level as possible through the choice of isotope for the best combination of minimum half-life, minimum retention in the body, and minimum quantity of isotope, which permit detection and accurate measurement.

In addition to radioisotopes and radio-opaque agents, diagnostic methods can be performed using L5G2BPs that are conjugated to dyes (such as with the biotin-streptavidin complex), contrast agents, fluorescent compounds, or molecules and enhancing agents (e.g. paramagnetic ions) for magnetic resonance imaging (MRI) (see, e.g., U.S. Pat. No. 6,331,175, which describes MRI techniques and the preparation of antibodies conjugated to a MRI enhancing agent). Typically, such diagnostic/detection agents are selected from agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., porphyrins, polyamines, crown ethers, bisthiosemicarbazones, polyoximes, and like groups known to be useful for this purpose. Chelates can be coupled to L5G2BPs using standard chemistries. A chelate is normally linked to a L5G2BP, such as an anti-γ2 DIII mAB, by a group, which enables formation of a bond to the molecule with minimal loss of immunoreactivity and minimal aggregation and/or internal cross-linking. Other, more unusual, methods and reagents for conjugating chelates to antibodies are disclosed in, e.g., U.S. Pat. No. 4,824,659. Examples of potentially useful metal-chelate combinations include 2-benzyl-DTPA and its monomethyl and cyclohexyl analogs, used with diagnostic isotopes in the general energy range of about 60 to about 4,000 keV, such as ¹²⁵I, ¹²³I, ¹²⁴I, ⁶²Cu, ⁶⁴Cu, ¹⁸F, ¹¹¹In, ⁶⁷Ga, ⁹⁹Tc, ⁹⁴Tc, ¹¹C, ¹³N, ¹⁵O, and ⁷⁶BR, for radio-imaging. These and similar chelates, when complexed with non-radioactive metals, such as manganese, iron, and gadolinium can be useful for MRI diagnostic methods in connection with L5G2BPs. Macrocyclic chelates such as NOTA, DOTA, and TETA are of use with a variety of metals and radiometals, most particularly with radionuclides of gallium, yttrium, and copper, respectively. Such metal-chelate complexes can be made very stable by tailoring the ring size to the metal of interest. Other ring-type chelates such as macrocyclic polyethers, which are of interest for stably binding nuclides, such as ²²³Ra for RAIT also can be used suitable in diagnostic methods.

Thus, the invention provides diagnostic L5G2BP conjugates, wherein the L5G2BP is conjugated to a contrast agent (such as for magnetic resonance imaging, computed tomography, or ultrasound contrast-enhancing agent) or a radionuclide that can be, for example, a gamma-, beta-, alpha-, Auger electron-, or positron-emitting isotope. Additional useful conjugated L5G2BPs are described elsewhere herein, which also can be useful in diagnostic methods and compositions (e.g., diagnostic kits) provided by this invention. These and other diagnostic techniques can be combined with application of a prophylactic or therapeutic regimen of the invention. For example, in one aspect, the invention provides a method of reducing cancer progression in a human patient having tissue that has been identified as comprising γ2-associated peptides (e.g., promigratory γ2 fragments) associated with cancer progression.

The diagnostic methods of the invention can be advantageously combined with the use of L5G2BPs for inhibiting migration and/or invasiveness of tumor cells to detect localized tumors or populations of cancer cells. Thus, for example, labeled anti-γ2 DIII mAbs can be administered to a patient so as to inhibit migration and invasiveness of cancer cells while also providing a means for visualizing such localized cells. Thereafter, focused and local delivery of anti-cancer therapeutic agents and/or application of anti-cancer surgical techniques can be applied to these areas to effectively remove the detected cancer cells. Kits of labeled anti-γ2 antibodies provided in unit dosage form for such purposes are another exemplary feature of the invention.

In another aspect, the invention provides a kit for diagnosis of cancer progression comprising a container comprising a L5G2BP, such as an anti-γ2 DIII mAB, and one or more reagents for detecting binding of the L5G2BP to a γ2-associated peptide. Reagents can include, for example, fluorescent tags, enzymatic tags, or other detectable tags. The reagents can also include secondary or tertiary antibodies or reagents for enzymatic reactions, wherein the enzymatic reactions produce a product that can be visualized. The invention in an exemplary aspect provides a diagnostic kit comprising one or more anti-γ2 antibodies in labeled or unlabeled form in suitable container(s), reagents for the incubations for an indirect assay, and substrates or derivatizing agents for detection in such an assay, depending on the nature of the label. Control reagent(s) and/or instructions for use (e.g., according to any of the methods described herein) also may be included.

Diagnostic kits can also be supplied for use with a L5G2BP, such as a conjugated/labeled anti-γ2 mAB, for the detection of a cellular activity (e.g., epithelial cell migration, carcinoma invasiveness, etc.) or for detecting the presence of γ2 peptides in a tissue sample or host. In such diagnostic kits, as well as in kits for therapeutic uses described elsewhere herein, a L5G2BP typically is provided in a lyophilized form in a container, either alone or in conjunction with additional antibodies specific for a target cell or peptide. Typically, a pharmaceutical acceptable carrier (e.g., an inert diluent) and/or components thereof, such as a Tris, phosphate, or carbonate buffer, stabilizers, preservatives, biocides, biocides, inert proteins, e.g., serum albumin, or the like, also are included (typically in a separate container for mixing) and additional reagents (also typically in separate container(s)). In certain kits, a secondary antibody capable of binding to the anti-γ2 DIII antibody or other L5G2BP, which typically is present in a separate container, also is included. The second antibody is typically conjugated to a label and formulated in manner similar to the anti-γ2 DIII antibody or other L5G2BP.

In another aspect, any one of the diagnostic methods of the invention can be used as a prognostic indicator of survival of a cancer patient. High levels of γ2 expression in cancer patients have been found to reflect, for example, as much as an about 50% decrease (e.g., about 30-60% increase) in survival 18-60 months after tumor removal or other anti-cancer surgery.

Using the methods described above and elsewhere herein L5G2BPs can be used to define subsets of cancer/tumor cells and characterize such cells and related tissues/growths.

In another example, a L5G2BP, such as an anti-γ2 DIII antibody, can be added to nitrocellulose, or other solid support which is capable of immobilizing cells, cell particles, or soluble proteins. The support can then be washed with suitable buffers followed by treatment with the detectably labeled γ2 peptide or antibody. The solid phase support can then be washed with the buffer a second time to remove unbound peptide or antibody. The amount of bound label on the solid support can then be detected by known method steps.

Linked enzymes that react with an exposed substrate can be used to generate a chemical moiety which can be detected, for example, by spectrophotometric, fluorometric or by visual means, in the context of a L5G2BP conjugate and/or fusion protein. Enzymes which can be used to detectably label γ2 DIII antibodies include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholinesterase. It is also possible to label a L5G2BP with a fluorescent compound. When the fluorescent labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde, and fluorescamine.

Anti-γ2 antibodies can also be detectably labeled using fluorescence-emitting metals such as ¹⁵²Eu, or others of the lanthanide series. These metals can be attached to an anti-γ2 DIII antibody, for example, using such metal chelating groups as diethylenetriaminepentaacetic acid (DTPA) or ethylenediamine-tetraacetic acid (EDTA).

L5G2BPs, such as anti-γ2 DIII antibodies, also can be detectably labeled by coupling to a chemiluminescent compound. The presence of the chemiluminescently labeled antibody is then determined by detecting the presence of luminescence that arises during the course of a chemical reaction. Examples of particularly useful chemiluminescent labeling compounds are luminol, isoluminol, theromatic acridinium ester, imidazole, acridinium salt, and oxalate ester.

Likewise, a bioluminescent compound can be used to label a L5G2BP. Bioluminescence is a type of chemiluminescence found in biological systems in which a catalytic protein increases the efficiency of the chemiluminescent reaction. Presence of a bioluminescent protein is determined by detecting luminescence. Bioluminescent compounds for purposes of labeling include luciferin, luciferase, and aequorin.

Detection of a labeled peptide or antibody, antibody fragment, antibody derivative, or other suitable peptide can be accomplished by a scintillation counter, for example, if the detectable label is a radioactive gamma emitter, or by a fluorometer, for example, if the label is a fluorescent material. In the case of an enzyme label, the detection can be accomplished by colorimetric methods which employ a substrate for the enzyme. Detection can also be accomplished by visual comparison of the extent of enzymatic reaction of a substrate in comparison with similarly prepared standards.

These and other diagnostic techniques can be used to screen any suitable material for γ2 peptides or γ2-containing peptides. Examples of materials that may be screened include, for example, blood, serum, lymph, urine, inflammatory exudate, cerebrospinal fluid, amniotic fluid, a tissue extract or homogenate, and the like. However, the invention is not limited to assays using only these samples, it being possible for one of ordinary skill in the art to determine suitable conditions which allow the use of other samples.

In situ detection can be accomplished by removing a histological specimen from a patient, and providing the combination of labeled antibodies to such a specimen. An antibody (or antibody fragment or other suitable peptide) is preferably provided by applying or by overlaying the labeled antibody (or fragment) to a biological sample. Through the use of such a procedure, it is possible to determine not only the presence of γ2 or γ2-containing peptide (e.g., a γ2/β3 heterodimer) but also the distribution of such peptides in the examined tissue (e.g., in the context of assessing cancer cell spreading). Given the present disclosure, ordinary artisans will readily perceive that any of a wide variety of histological methods (e.g., staining procedures) can be modified in order to achieve such in situ detection.

An antibody, fragment, or derivative of the present invention can be adapted for utilization in an immunometric assay, also known as a “two-site” or “sandwich” assay. In a typical immunometric assay, a quantity of unlabeled antibody (or fragment of antibody) is bound to a solid support that is insoluble in the fluid being tested and a quantity of detectably labeled soluble antibody is added to permit detection and/or quantification of the ternary complex formed between solid-phase antibody, antigen, and labeled antibody.

Typical immunometric assays include “forward” assays in which the antibody bound to the solid phase is first contacted with the sample being tested to extract the γ2 antigen from the sample by formation of a binary solid phase antibody-γ2 peptide complex. After a suitable incubation period, the solid support is washed to remove the residue of the fluid sample, including unreacted γ2 peptide, if any, and then contacted with the solution containing a known quantity of labeled antibody (which functions as a “reporter molecule”). After a second incubation period to permit the labeled antibody to complex with the γ2 peptide bound to the solid support through the unlabeled antibody, the solid support is washed a second time to remove the unreacted labeled antibody. This type of forward sandwich assay can be a simple “yes/no” assay to determine whether γ2 is present or can be made quantitative by comparing the measure of labeled antibody with that obtained for a standard sample containing known quantities of γ peptide. Such “two-site” or “sandwich” assays are described by Wide (RADIOIMMUNE ASSAY METHOD, Kirkham, ed., Livingstone, Edinburgh, 1970, pp. 199-206).

Other types of “sandwich” assays, which can also be useful with γ2 peptides, are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In a “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one embodiment, a combination of antibodies of the present invention specific for separate epitopes can be used to construct a sensitive three-site immunoradiometric assay.

In contrast, a “double-determinant” ELISA, also known as a “two-site ELISA” or “sandwich assay,” requires small amounts of antigen, and such an assay does not require extensive purification of the antigen. Thus, a double-determinant ELISA may be preferred to the direct competitive ELISA for the detection of an antigen in a clinical sample. See, for example, Field et al., Oncogene 4: 1463 (1989) and Spandidos et al., AntiCancer Res. 9: 821(1989) for a discussion of such techniques and related principles.

In a double-determinant ELISA, a quantity of unlabeled mAb, antibody fragment, or similar peptide (which may be referred to as the “capture antibody”) is bound to a solid support, the test sample is brought into contact with the capture antibody, and a quantity of detectably labeled soluble antibody (or antibody fragment) is added to permit detection and/or quantitation of the ternary complex formed between the capture antibody, antigen, and labeled antibody. Methods of performing a double-determinant ELISA are well-known. See, for example, Field et al., supra. See also, generally, IMMUNOLOGICAL METHODS, Vols. I and II, Lefkovits and Pernis, eds., Academic Press, New York, N.Y. (1979 and 1981).

The antibodies and other L5G2BPs of the invention also can be used to at least substantially (e.g., completely) isolate and/or purify Ln-5, γ2, or a γ2 DIII containing peptide or fragment thereof by way of a suitable technique such as chromatography, Western blotting, etc. Antibodies also can be used in other suitable applications where binding of an antigen is desirable (e.g., FACS analysis).

In another aspect, the invention provides a method for isolating γ2 and/or Ln-5, or a Ln-5 associated structure (e.g., protein complex; protein-small molecule (e.g., heparin:Ln-5) complex); or Ln-5 associated cell and, where suitable for removing γ2, Ln-5, or such a Ln-5-associated molecule, structure, or cell from a solution. Various methods such as affinity chromatography techniques can be used for such purification methods.

In another aspect, the invention provides a mechanism for enhancing the isolation of γ2 peptide-associated cells, such as γ2 DIII-associated invasive cancer cells, from a host or host tissue sample by contacting tissue comprising such cells with a medium comprising suitable L5G2BP(s) under conditions such that the cells are at least substantially isolated. Such a method can be useful in, e.g., developing autologous vaccines; diagnosing the profile of such cells with respect to cancer progression; to enrich a cell population for in vitro experiments; or as a technique for removing metastatic cells prior to establishment of additional tumors in a human cancer patient.

Although anti-γ2 antibodies, antibody fragments, related peptides (e.g., fusion proteins comprising VH and/or VL sequences of or derived from anti-γ2 DIII antibodies), and related derivatives and/or variants thereof, are preferred forms of L5G2BPs in the compositions and methods described herein, it should be understood that other suitable peptides that bind to γ2-associated peptides also can be included in such methods and compositions and that sequences from such γ2-binding peptides and variants of such sequences can form part of L5G2BPs, unless otherwise noted. The use and inclusion of such heterologous LG2BPs and peptides comprising such heterologous sequences in inventive methods and compositions described herein represent additional aspects of the invention. Examples of such γ2-binding peptides are known in the art and described elsewhere herein. Variants and derivatives of such peptides and fusion proteins comprising sequences from such peptides or that are functionally and chemically similar to such sequences can be prepared by techniques described elsewhere herein.

The invention provides additional useful compositions and methods and various aspects thereof can be amenable to other forms of description beyond those already provided here. For example, in one aspect, the invention relates to a method of treating a cancer or pre-cancer condition in a human patient comprising a step for delivering an effective amount of a L5G2BP thereto so as treat the cancer or pre-cancer condition. In another aspect, the invention provides a composition comprising a L5G2BP and means for delivery thereof. Various other additional methods are described below.

The invention further provides method of promoting the sale and/or use of a compound according to any of the preceding aspects, or otherwise described herein, comprising distributing information (e.g., by printed materials that are handed out, mailed, etc.; by advertising signage; by television programs and advertisements; by radio programs and advertisements; by internet site postings; by email; by telemarketing; by door-to-door or person-to-person marketing; by funding and/or hosting conferences, panels, forums, etc., by employing and/or contracting for the services of salespeople and/or medical/scientific liaisons, by funding and/or hosting scientific research and publications related to such uses, etc.) related to the use of the compound in the prevention or treatment of any condition or combination of conditions recited in any of the foregoing aspects or described elsewhere herein to any persons or entities of potential interest (e.g., pharmaceutical chains, formulary managers, insurance companies, HMOs, hospitals and hospital chains, other health care companies, pharmacy benefit managers, potential patients, cancer patients, former cancer patients, patients in remission, primary care physicians, nurses, doctors of pharmacy, and/or key opinion leaders).

Ln-5 ADRs and L5G2DBPs provided by aspects of the invention described herein can be used as targets for molecular modeling techniques to identify either the structure of the disclosed compositions or to identify potential or actual molecules, such as small molecules, which interact in a desired way with the disclosed compositions. The nucleic acids, peptides, and related molecules disclosed herein generally can be used as targets in any molecular modeling program or approach. When using the disclosed compositions in modeling techniques, molecules, such as macromolecular molecules, will typically be identified that have particular desired properties such as inhibition or stimulation or the target molecule's function. One way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user. Examples of molecular modeling systems are the CHARMm and QUANTA programs, Polygen Corporation, Waltham, Mass. Various other modeling methods are described elsewhere herein. Related methods and principles are described in, e.g., Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: QUANTITATIVE STRUCTURE-ACTIVITY RELATIONSHIPS IN DRUG DESIGN pp. 189-193 (Alan R. Liss, Inc. 1989); Lewis and Dean, 1989 Proc. R. Soc. Lond. 236,125-140 and 141-162; and, with respect to a model enzyme for nucleic acid components, Askew, et al., 1989 J. Am. Chem. Soc. 111, 1082-1090. Although described above with reference to design and generation of compounds which could alter binding, one could also screen libraries of known compounds, including natural products or synthetic chemicals, and biologically active materials, including proteins, for compounds which alter substrate binding or enzymatic activity.

In another aspect, the invention provides a computer or computer readable medium comprising a database comprising a sequence record comprising at least one character string corresponding to at least one sequence specifically described herein (e.g., an ADR sequence, the sequence of a CDR, a VH region sequence, etc.), a specific sequence encompassed by a formula provided herein, or one of the formulas provided herein. The invention also includes an integrated system comprising a computer or computer readable medium comprising a database comprising at least one such sequence record, the integrated system typically further comprising a user input interface allowing a user to selectively view said at least one sequence record.

Also provided is a method of using a computer system to present information pertaining to at least one of a plurality of sequence records stored in a database, selected from such sequences, formulas, and sequences encompassed in such formulas, the method comprising (a) determining a list of at least one character string corresponding to such a sequence or a subsequence thereof; (b) determining which said at least one character string of the list is selected by a user; and (c) displaying each selected character string, or aligning each selected character string with an additional character string.

In a further aspect, the invention provides a method of identifying an ADR or ADR component in γ2 that comprises subjecting the structure of γ1 or a portion thereof to computer modeling (e.g., simulated docking studies) to identify likely antigenic amino acid residues and finding corresponding residues in γ2 based on a comparison of the sequences of γ1 and γ2.

In another facet, the invention provides methods of identifying small molecule compounds (non-peptide compounds having a molecular weight of about 1000 or less and typically that can be classified as organic compounds) that bind to one or more of the ADRs described herein and that optionally modulate functions associated therewith (e.g., inhibition of γ2 peptide-associated migration of epithelial and epithelial derived precancer and/or cancer cells). A number of suitable assays for identifying small molecule compounds given such targets are known in the art. Competition assays can, for example, be performed to identify small molecules that bind to ADRs or other portions of γ2, Ln-5, etc. Such methods may be advantageously applied when an antibody or other L5G2BP binds to a Ln-5 ADR that has been identified with a particular function, wherein the nature of the ADR, the function, and/or other factors suggest that a small molecule capable of binding to or near this region may be able to modulate Ln-5 target functions.

Potential small molecule binding agents typically are organic molecules having a molecular weight of about 100-2,500 daltons (e.g., about 200-200 daltons, such as about 300-1500 daltons, for example about 500 daltons). Candidate small molecule compounds for such binding assays typically comprise functional groups necessary for structural interaction with proteins, particularly hydrogen bonding, and commonly include at least an amine, carbonyl, hydroxyl or carboxyl group, and often will comprise at least two of such functional chemical groups. Candidate small molecules often comprise cyclical carbon or heterocyclic structures and/or aromatic or polyaromatic structures substituted with one or more of the above functional groups. Candidate small molecule compounds are also found among biomolecules including saccharides, fatty acids, steroids, purines, pyrimidines, derivatives, structural analogs of any thereof, and/or combinations of any thereof. Candidate small molecules can be obtained from a wide variety of sources including libraries of synthetic or natural compounds (such as are commercially available for such screening). For example, libraries of natural compounds in the form of bacterial, fungal, plant, and animal extracts are available or readily produced. Additionally, natural or synthetically produced libraries and compounds may be readily modified through conventional chemical, physical and biochemical means. Known pharmacological agents may also be subjected to directed or random chemical modifications, such as acylation, alkylation, esterification, amidification to produce structural analogs. The invention provides methods for the screening of compounds characterized by any one or more of these characteristics in suitable methods (e.g., a competition assay, a functional assay (e.g., an inhibition of γ2 peptide-associated cell migration), or both) so as to identify molecules that bind to Ln-5 and typically modulate Ln-5-associated physiological responses.

Disclosed compositions (L5G2BPs, γ2 ADRs, etc.) can be used as targets for combinatorial techniques to identify molecules or macromolecular molecules that interact with the disclosed compositions, such as an ADR of γ2 DIII bound by mAb 5D5 and/or mAb 6C12, in a desired way. Combinatorial chemistry generally refers to screening sets of molecules for a desired activity whether based on small organic libraries or other types of molecule libraries. Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636. Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514), or combinations thereof, can be generated and used in such screening methods.

The following documents provide additional compositions, methods, and guidance that can be useful in carrying out the inventive methods described herein and/or with respect to preparing compositions of the invention: Pyke et al., Am J Pathol. October 1994;145(4):782-91; U.S. Provisional Patent Application 60/523,895; U.S. Patent Application 2004/0014665; Abbas et al., 2000 Cellular and Molecular immunology, W.B. Saunders Co.); Lenander et al., Mol Pathol. December 2003;56(6):342-6; Giannelli et al., Clin Cancer Res. Sep. 1, 2003;9(10 Pt 1):3684-91; Lundgren et al., Med Oncol. 2003;20(2):147-56; Masaki, et al., Dig Dis Sci. February 2003;48(2):272-8; Kohlberger et al., Anticancer Res. November-December 2002;22(6B):3541-4; Aoki, Dis Colon Rectum. November 2002;45(11):1520-7; Nordstrom et al., Int J Gynecol Cancer. January-February 2002;12(1):105-9.; Lenander et al., Anal Cell Pathol. 2001;22(4):201-9; Haas et al., J Histochem Cytochem. October 2001; 49(10):1261-8; Yamamoto et al., Clin Cancer Res. April 2001;7(4):896-900; Skyldberg et al., J Natl Cancer Inst. Nov. 3, 1999;91(21):1882-7; Henning et al., Histopathology. April 1999; 34(4):305-9; Nguyen et al., J. Biol. Chem. 275(41):31896-31907 (2000); U.S. Pat. Nos. 5,660,982, 6,120,991, and 6,693,169; U.S. Pat. No. Applications 20020052334 and 20040019004; European Patent Document 0 278 781; International Patent Applications WO 00/26342 and WO 03/016907; Amano et al., J Immunol Methods. Apr. 22, 1999;224(1-2):161-9; and Sordat et al., J Pathol. May 1998;185(1):44-52.

### EXAMPLES

The following examples of experiments related to various aspects of the invention are provided so as to further illustrate the invention but, of course, should not be construed as in any way limiting its scope.

### Example 1

This Example illustrates a competition assay between anti-γ2 DIII monoclonal antibodies.

The following compositions were used in this and other experiments described herein:

ABTS-peroxidase substrate concentrate (10×=1 mg/ml): ABTS (2,2′-azino-bis(3-ethylbenzthiazoline-6-sulfonic acid) was diluted in 0.1 M Na-citrate, pH 5; and was further diluted for use in the competition assays just before use, from 1 ml to 10 ml with Na-citrate buffer and the further diluted solution was mixed with 2 μl 30% hydrogen peroxidase

Citrate buffer: 0.1 M Sodium citrate, pH 5.0;

ELISA plate: Nunc-Immuno Plate MaxiSorp™;

GST-γ2III fusion protein (antigen, human laminin γ2 amino acid (“AA” or “a.a.” residues #392-567);

PBS: 10 mM Potassium phosphate, 150 mM NaCl, pH 7.5;

PBST: 10 mM Potassium phosphate, 150 mM NaCl, pH 7.5, 0.05% Tween-20

AB complex: biotin/avidin system, Elite Standard Kit, cat. PK-1600, Vector Laboratories Inc, Burlingame, Calif., USA)

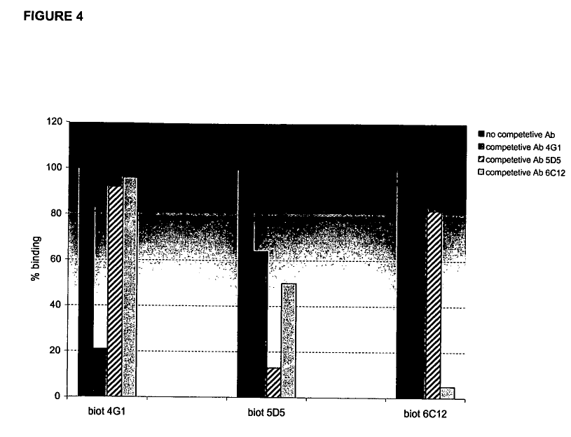

ELISA plates were obtained and coated with GST-γ2III (SEQ ID NO:22), a glutathione S-transferase:γ2 DIII fragment fusion protein, in a concentration of 0.25 μg/well and allowed to incubate overnight at +4° C. Thereafter, the ELISA plates were washed with PBST (3×200 μl/well). Blocking was performed by contacting the wells with a solution of 1% BSA in PBS for 2 hours at room temperature (200 μl/well). A mixture of (a) potentially competitive antibody and (b) biotinylated reference/standard antibody was added to each well (5 μg competitive mAb/well (50 μg/ml, 100 μl/well) and 0.05 μg/well (0.5 μg/ml, 100 μl/well) of biotinylated reference/test mAb (except in the case of control wells which were used to set a 100% mark for relative inhibition measurements). The plates were incubated with the antibody mixtures for 30 minutes at room temperature and then washed 3× with PBST at a concentration of 200 μl well. Antibodies were allowed to complex for 30 minutes at room temperature and thereafter the plates were again washed in PBST (3×200 μl/well). Thereafter the wells were contacted with ABTS substrate for a period of 30 minutes at room temperature, after which absorbance at 405 nm was measured and the measurements used to calculate relative competitive inhibition. The results of these experiments are set forth in FIG. 4.

As shown in FIG. 4, mAbs 5D5 and 6C12 do not significantly compete with mAb 4G1. However, mAb 6C12 was able to significantly compete with 5D5, suggesting that these antibodies may be specific for closely located antigenic determinant sequences in γ2 DIII.

This Experiment demonstrates that competition assays provide a convenient and feasible method for distinguishing anti-γ2 DIII antibodies with respect to ADRs.

### Example 2

This example provides a further illustration of a competition analysis of anti-γ2 DIII antibodies with respect to binding a γ2 DIII peptide.

The same buffers, reagents, plates, and anti-γ2 DIII antigen were used in these assays as is described in Example 1.

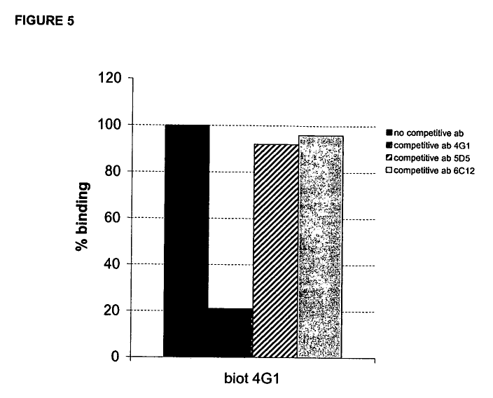

Competition assays using mAb 4G1, 5D5, and 6C12, separately (with no test antibody for a control) against biotinylated mAb 4G1 were performed as described above in Example 1 (competitive mAb 5 μg/well and biotinylated reference mAb 4G1 0.05 μg/well). The results of these experiments, in terms of relative inhibition (% binding) against the no-antibody control (set as 100%) are set forth in FIG. 5. As is shown in FIG. 5, mAb 4G1 clearly recognizes different epitope(s) than mAb 5D5 and mAb 6C12. This data also support the finding that mAb 5D5 and mAb 6C12 may bind different epitopes.

This Example further demonstrates how competition assays can be used to determine relative inhibition between mAb specific for different regions of γ2 DIII.

### Example 3

This Example demonstrates the mapping of antigenic determinant regions in γ2 DIII by direct ELISA by using a series of recombinant GST-γ2III-chain fragments to measure the binding capabilities of individual anti-γ2 DIII antibodies to such fragments.

The following recombinant GST-γ2III-chain fragments were tested in a direct ELISA:

GST-γ2III: the original antigen used for immunizations, a.a.
residues 392-567
GST-γ2III-2: a.a. residues 392-555
GST-γ2III-4: a.a. residues 392-494
GST-γ2III-7: a.a. residues 461-653
GST-γ2III-20: a.a. residues 461-534
(used in second set of experiments only)
(all “a.a. residue” references are with respect to positions in Ln-5 γ2)

The stepwise protocol set forth below was used for the ELISAs, which were performed with biotinylated mAb 4G1, mAb 5D5, and mAb 6C12 (GST, BSA, and plastic used as controls in the first experiment; GST and BSA only in the second experiment) and the γ2 DIII antigens individually (GST-γIII-20 was only tested in the second experiment; nonspecific biotinylated Ig was used as a control for the antibodies in both experiments). All incubations were performed at room temperature except step 1, below. 100 μl of reagents were used per well for incubations and 200 μl of PBST for washings unless otherwise mentioned. In addition, shaking the plate can be helpful for incubations.

ELISA plates were coated with 100 μl GST-γ2III chain fragments at concentration of 1 μg/ml in 0.1 M carbonate/bicarbonate buffer, pH 9, o/n at +4° C. (100 ng/well). The coated plates were washed three times with PBST. Non-specific binding was blocked with BSA-PBS incubation for 90 minutes (200 μl/well). Dilutions of reference standards, negative controls (quality controls), and sample were diluted in BSA-PBS and thereafter incubated for 1 h at room temperature. The plates were again washed three times with PBST. HRP-conjugated anti-mouse IgG secondary antibody was added to the wells (diluted 1:5000 in PBS) and allowed to incubate for 30 minutes. The plates were washed again three times with PBST. ABTS-peroxidase substrate was added. Substrates were prepared immediately before use: ABTS-stock (10×) was diluted with Citrate buffer (see Example 1); 2 μl of 30% hydrogen peroxidase was added to each well and mixed. Substrate was added to the wells and incubated in dark conditions for 30 minutes. Absorbance was read with a microtitre plate reader at 405 nm after 30 min and after 60 min.

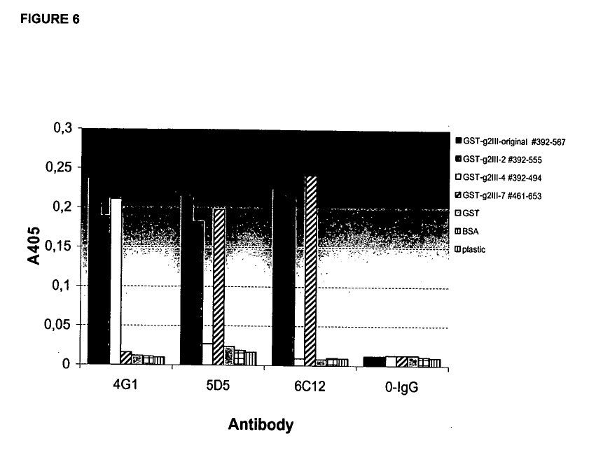

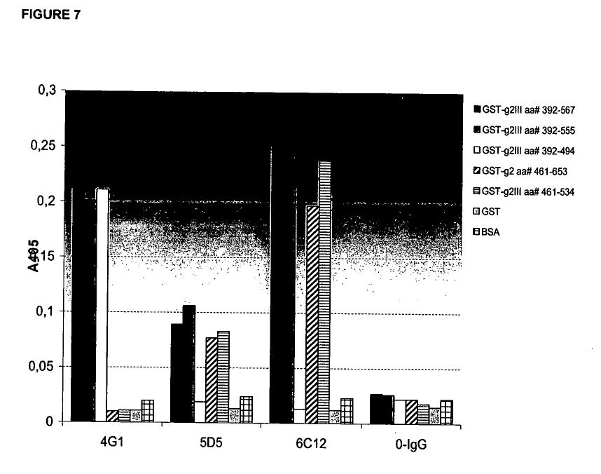

The results for the first experiment and second experiment, respectively, are shown in FIG. 6 and FIG. 7. The results of these experiments serve to further confirm that mAb 4G1 has significantly different properties with respect to binding γ2 DIII than mAbs 5D5 and 6C12.

Specifically, mAb 5D5 showed little binding, and mAb 6C12 showed even less binding, of GST-γ2III fusion proteins comprising Ln-5 residues 392-494, but did exhibit binding against fusion proteins comprising Ln-5 residues 392-555 and 392-567, suggesting that a region between residues 494-555 comprises an epitope or part of an epitope for these mAbs. Monoclonal antibody 4G1, in contrast, showed significant binding to fusion proteins comprising 392-494, but lacked binding to 461-653 and 461-534, which were bound well by both mAb 5D5 and mAb 6C12. Both mAb 5D5 and mAb 6C12 bound fragments comprising residues C-terminal to residues 392-494, as well as the 461-534 fusion protein. This may reflect the presence of one or more epitopes in the region of γ2 residues 461-494 to which these mAbs bind. In contrast, mAb 4G1 bound well to all of the fusion proteins comprising γ2 DIII sequences contained within the region of residues 392-567.

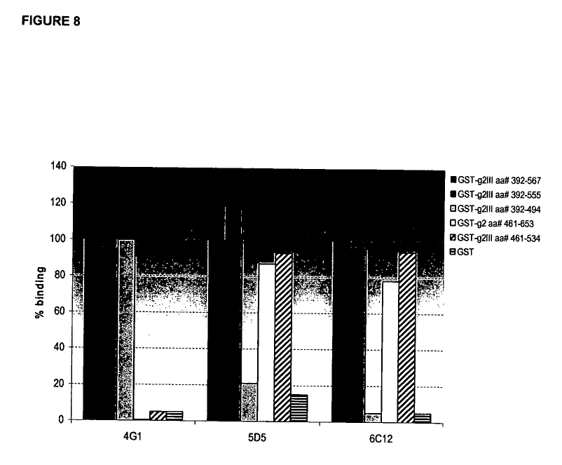

To further compare the antibodies, the binding of the each antibody for the original γ2 DIII fusion protein was set at 100% and the relative binding (% binding) of each antibody with respect to fusion proteins comprising the sequences 392-555, 392-494, 461-653, and 461-534. The results of this analysis are set forth in FIG. 8. These data further confirm the unique profile of mAb 4G1 as opposed to mAb 5D5 and mAb 6C12. All of the data together suggest that mAb 5D5 and mAb 6C12 may share similar, probably overlapping, but somewhat different epitopes.

This Experiment generally demonstrates how antigen fragment mapping can be uses to identify antigenic determinant regions for an antibody. Similar techniques can be used to characterize and design γ2 DIII antibodies and L5G2D3BPs in accordance with the invention.

### Example 4

In order to more specifically characterize an epitope for mAb 4G1, five cyclic peptides, corresponding to predicted antigenic sequences in the region of residues 394-552 of Ln-5 were constructed (reference numbers X252-X256). All of the peptides were allowed to form Cys-Cys disulfide bonds prior to being subjected to ELISA analysis. In all cases except with respect to X253 an extra amino acid residue was added to ensure solubility in water in order that the peptides could readily be analyzed by ELISA.

These sequences and some of their prominent features are presented in Table 9:

TABLE 9

| | | | Ln-5 |
|---|---|---|---|
| Ref. | Sequence | MW | Residues |
| X252 | QDCASGYKRDSARLGPFGTCIPR | 2498 | 394-415 + R |
| | (SEQ ID NO:87) | | |
| X253 | DPDTGDCYSGDENPDIECAD | 2130 | 425-444 |
| | (SEQ ID NO:88) | | |
| X254 | ELCADGYFGDPFGEHGPVRPCQPE | 2620 | 494-516, +E |
| | (SEQ ID NO:89) | | |
| X255 | RQCNNNVDPSASGNCDRLTGR | 2276 | R+, 518-537 |
| | (SEQ ID NO:90) | | |
| X256 | LKCIHNTAGIYCDQR | 1734 | 539-552 + R |
| | (SEQ ID NO:91) | | |

The identity of these peptides was verified by mass spectrometry. Solutions containing the five peptides, in an unpurified state, were individually subjected to direct ELISA analysis using standard techniques. Briefly, NUNC-IMMUNO plates were coated with 100 μl/well of 50 μg/ml peptide in carbonate buffer and allowed to sit overnight at +4° C. Control wells were loaded with 5 μg/ml bovine serum albumin (BSA), 5 μg/ml GST-LN5 (a GST-laminin-γ2-III fusion protein that contains human laminin-γ2-chain amino acid residues 391-567 and is specifically described in US Patent Application 20020062307), or carbonate buffer without protein. The plates were washed five times with PBS to remove unbound peptide and subjected to one hour blocking at room temperature by exposure with a washing/blocking buffer (0.05% Tween-20 in PBS (PBST)), which was thereafter removed. 100 ng of antibody (100 μl of solution containing an antibody solution at a concentration of 1 μg antibody/ml) for each of the three mAbs (4G1, 5D5, and 6C12) was added to the test wells. Dilution buffer was added to the control wells. The plates were incubated at room temperature for one hour, and thereafter washed four times in PBST.

100 μl HRP-anti-mouse secondary Ab diluted in a dilution buffer at a ratio of 1:2000 was added to each well and the plates were incubated for one hour at room temperature, and thereafter washed 4 times with PBST. The plates were then developed with 100 μl TMB substrate for a period of 15-30 minutes in the dark (during which time the plates were monitored for color development). At an appropriate time, the reaction was stopped in each well with the addition of 4M H3PO4. Absorbance measurements were taken at 450 nm and 620 nm, ratios of absorbance determined, and the data evaluated. These experiments were performed in triplicate.

The initial results of these experiments showed that only X252 was bound by mAb 4G1 (data not shown).

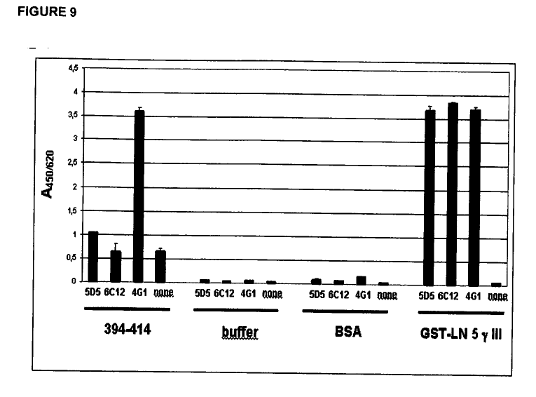

The ratio of 450 nm/620 nm absorbance measurements for each of the mAbs 4G1, 5D5, and 6C12; and control wells; were obtained and evaluated. The results of this aspect of the Experiment are shown in FIG. 9. Specifically, FIG. 9 illustrates a graph of the mean value of three independent absorbance ratio measurements for each 4G1 well and each control well ±SD.

These results show binding of the 4G1 antibody to amino acid residues 394-414 (“epitope X252”), suggesting that a γ2 epitope is represented in or by this sequence or that significant contributing portions of an epitope are comprised within this sequence. As indicated above, two other mAbs specific for γ2, 6C12 and 5D5, do not recognize this epitope. Thus, this data further demonstrates that mAb 4G1 has a unique immunological profile with respect to mAbs 5D5 and 6C12.

This experiment generally illustrates a technique that can be used to identify antigenic determinants in Ln-5 γ2 as a means for characterizing anti-Ln-5 antibodies.

### Example 5

This Example demonstrates the use of peptide fragments representing a number of regions of γ2 DIII in a competition ELISA measuring whether these peptide fragments can disrupt the binding of mAbs 4G1, 5D5, and 6C12 to GST-γIII.

GSTγIII (100 μl/well) was diluted in PBS to a concentration of 5 μg/ml and immobilized on NUNC-IMMUNO plates overnight at +4° C. Unbound protein was removed by washing five times with PBS. The plates were blocked with 0.05% Tween-20 in PBS (PBST) for 1 hour at room temperature. 50 ng of each anti-LN-5 antibody (50 μl 1 μg/ml) was added simultaneously to individual wells and mixed with 50 μl of a test γ2 region peptide in increasing amounts of peptides (0-1-3-5-7-10-20-30-50 μg) in a total volume of 100 μl. The plates were thereafter incubated for 1 hour at room temperature. Unbound proteins were washed out with PBST (repeated five times).

HRP-conjugated anti-mouse antibody in standard dilution buffer (ratio of 1:2000) was added to each well and incubated for 1 hour. The plates were washed four times with PBST. Detection was performed using chromogenic TMB substrate for 15-30 min in dark conditions (color develop monitored during this period). The reaction was stopped at an appropriate endpoint for each well by adding 4M H₃PO₄. Absorbance readings were taken at 450 nm and 620 nm and the ratios of absorbance calculated and evaluated.

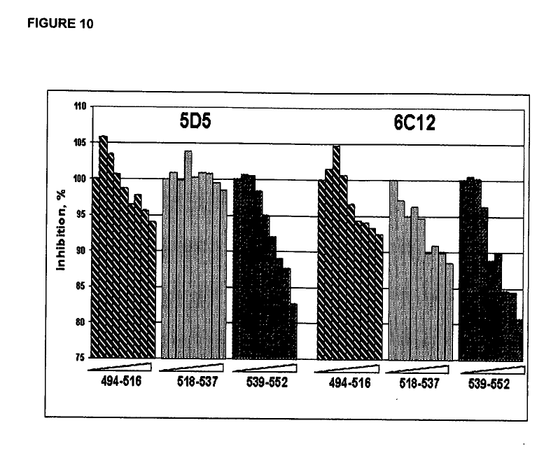

Absorbance measurements obtained by these experiments are set forth as a relative percentage compared to binding of the antibody to full length GST-γ2III (100%) in FIG. 10 (increasing concentrations of γ2 peptides ranging from 0 to 50 μg are indicated with a triangle). As illustrated in FIG. 10, the data obtained by these experiments demonstrate that both 5D5 and 6C12 recognize a complex structural epitope. All three peptides can to some degree inhibit binding of the antibody to GST-LN5γIII. However, the epitopes are somewhat different for 5D5 and 6C12 antibodies, inasmuch as a peptide corresponding to γ2 residues 518-537 does not interfere with binding of 5D5, while this peptide inhibits 6C12 binding.

The results of these experiments demonstrate that conformational epitopes present in γ2 DIII can be bound by Ln-5 γ2 binding proteins having a similar antigen determinant binding profile as mAb 5D5 and/or mAb 6C12. Moreover, this data also helps to confirm the distinct nature of the epitopes identified by these three mAbs. Additionally, this data illustrates that anti-γ2 antibodies exhibit a dose-dependent response across a relevant range of amounts (about 5-50 μg). Finally, these experiments serve to demonstrate how competition analysis can be used to pinpoint antigenic determining regions for Ln-5 γ2 binding peptides.

### Example 6

This example demonstrates the cloning of cDNAs encoding the variable light and heavy regions of anti-γ2 DIII mAbs and determination of CDRs therein by sequence analysis.

In order to clone the variable regions, without having a known variable sequence, the 5′-RACE (Rapid Amplification of cDNA ends) (BD Clontech) was applied to γ2 Ig cDNA, followed by standard polymerase chain reaction (PCR) amplification. An extended 5′ cDNA tail by means of a pre-designed oligonucleotide (SMART II), allowed for reverse transcription of the 5′ end beyond the ATG or initiation of translation signal, serving as a template for a PCR primer that would anneal to this extended 5′ end. The 3′ primers were designed to anneal to areas within the constant regions of the immunoglobulin. Thus, PCR using the 5′ and 3′ primers should result in amplification of cDNAs encompassing the variable regions, and including some sequences regarded as part of the constant regions. This method was used to clone the cDNAs encoding the variable regions of the light and heavy chains of anti-laminin 5 gamma 2 antibodies.

Isolation of RNA

Hybridoma cell lines 5D5, 4G1, and 6C12 were obtained as cell pellets with approximately 4×106 cells. Cells were resuspended in PBS and total RNA was purified using the RNeasy kit following the manufacturer's instructions (Qiagen).

Reverse-Transcription

First strand cDNA synthesis was achieved using 1 microgram of each sample RNA and a BD SMART Race cDNA amplification kit (BD Clontech) to obtain 5′-RACE-ready cDNA. To amplify cDNAs encoding the variable regions of light (VL) and heavy chains (VH) of IgGs using PCR, 2.5 μl of 5′-RACE-ready cDNA from each cell clone was mixed with either universal primer mix UPM and light-chain cDNA specific primers or universal primer mix UPM and heavy chain cDNA-specific primers, following the kit's protocol (BD Clontech). The light chain specific primer used was KK47 (sequence below) and the heavy chain specific primers consisted of a mixture of four different primers KK43, KK44, KK45 and KK46 (sequences below). The PCR run conditions were as follows: 2 min denaturing at 95° C. (initial cycle), followed by 5 sec denaturing at 94° C. and 3 min annealing-extension at 72° C. during 5 cycles, followed by 5 sec denaturing at 94° C., 10 sec annealing at 70° C. and 3 min extension at 72° C. during 5 cycles, and finally 5 sec denaturing at 94° C., 10 sec annealing at 68° C., and 1 min extension at 72° C. during 28 cycles. Samples (5 microliters) were then run on a 0.8% agarose gel in 1× TAE buffer to observe fragment amplification. The expected size of fragments amplified are within 800-900 bp for light chains and 500-600 bp for heavy chains. Once DNA amplification was detected, the fragments were purified using a QIAquick PCR purification kit (Qiagen).

Primers:
KK43:

(SEQ ID NO:53)

5′ ACTAGTTTTGGCTGAGGAGACGGTGACCGTGG 3′
KK44:

(SEQ ID NO:54)

5′ ACTAGTTTTGGCTGAGGAGACTGTGAGAGTGG 3′
KK45:

(SEQ ID NO:55)

5′ ACTAGTTTTGGCTGCAGAGACAGTGACCAGAG 3′
KK46:

(SEQ ID NO:56)

5′ ACTAGTTTTGGCTGAGGAGACGGTGACTGAGG 3′
KK47:

(SEQ ID NO:57)

5′ TCATCAACACTCATTCCTGTTGAAGCTCTTGA 3′

Ligation and Transformation into Competent Cells

The eluted material from the QIAquick purification column was used directly in a ligation reaction together with the bacterial expression vector pCR2.1-TOPO using the TOPO TA Cloning kit (Invitrogen). Two microliters of the TOPO cloning reaction were used to transform chemically competent TOP10 E. coli cells according to the manufacturer's instructions (Invitrogen). Cells are plated out onto LB plates containing 100 microgram/ml ampicillin (selection) and 80 microliters X-Gal (20 mg/ml stock solution). Cells that have been transformed with a pCR2.1-TOPO plasmid containing a cloned fragment would appear as white colonies, whereas cells without a cloned fragment would appear as blue colonies. To confirm that the fragments were cloned successfully, a colony PCR protocol was undertaken.

Colony PCR

White bacteria colonies containing candidate plasmids were removed one by one from the plates using sterile inoculation loops and inoculated onto a new back-up LB plate containing ampicillin, and into 0.2 ml PCR tubes containing 2 microliters of 10 micromolar primer M13 forward (see below), 2 microliters of 10 micromolar primer M13 Reverse (see below), 1 microliter 5 mM dNTP mix, 2.5 microliters of 10× Thermo Pol buffer and 0.5 microliters Taq polymerase (New England Biolabs), in a total reaction volume of 25 microliters. The PCR run conditions were as follows: an initial denaturing step at 94° C. during 2 min, followed by 30 sec denaturing at 94° C., 30 sec annealing at 55° C. and 1 min extension at 72° C. during 25 cycles, and finally a 3 min extension step at 72° C. Samples (5 microliters) were run on a 0.8% agarose gel in 1× TAE buffer to observe if the fragments were amplified. Once candidate fragments show the approximate size expected, approximately 1000 bp for VL (variable light) chains and 800 bp for VH (variable heavy) chains, then samples of bacteria from the back-up plate were used for plasmid minipreparations.

Primers:
M13 Forward:

(SEQ ID NO:58)

5′ GTAAAACGACGGCCAG 3′
M13 Reverse:

(SEQ ID NO:59)

5′ CAGGAAACAGCTATGAC 3′

Plasmid Minipreparations

Selected bacteria were inoculated into culture tubes containing 5 ml of TY2 media containing 100 microgram/ml carbencillin and cultured overnight at 37° C. A plasmid isolation procedure was carried out using the Qiaprep Spin Miniprep kit (Qiagen). Reasonably pure plasmid preparations were obtained and the concentrations were determined.

Sequencing and Computer Analyses

The DNA sequencing was performed by a commercial service supplier (Agowa) and the obtained sequences were analyzed using the Vector NTI Suite 9 software from Informax (Invitrogen). Open-reading frames encoding putative amino acid sequences of either light or heavy chains of immunoglobulins were selected and assembled together with previously known constant regions from light and heavy chains of IgG1 immunoglobulin. See FIG. 15. The primary structures of 5D5, 4G1, and 6C12 antibodies were then used in protein modeling studies to analyze the antibody-antigen interface and to predict the structure of the whole antibody-antigen complex.

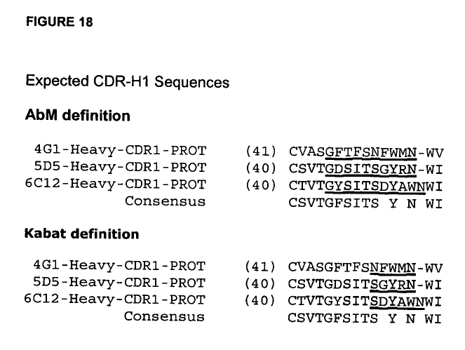

The cloning of the antibodies reveals the CDR regions that determine the specificity of the individual antibody. CDR regions can be aligned in different ways, such as, for example according to the Kabat Scheme for the alignment (see, e.g., http://sapc34.rdg.ac.uk/abeng/ and http://www.biochem.ucl.ac.uk/˜martin/abs/ Generallnfo.html). The CDR sequences identified through this analysis are provided in FIG. 11 and FIG. 12. Alternative rules for CDR alignment (such as the AbM standards) are provided at http://www.bioinf.org.uk/abs/. Using this set of rules, a distinct, alternative set of CDR-H1 sequences were identified. These sequences are presented in FIG. 18. It should be noted that CDR sequences may be identified that meet only some of the features associated with CDR sequences and reflected in these rules (indeed, some of the CDR sequences of mAbs specifically identified herein fall outside of the classical definitions provided by these rules, but were identified by the presence of a number of features reflected in such rules).

This Example demonstrates techniques that can be applied to identify CDR sequences in anti-γ2 DIII antibodies. Variations of these techniques can be, for example, applied to determine similar information about other anti-γ2 DIII antibodies.

### Example 7

Commercially available human laminin gamma2 antibodies (GB3 from Novus Biologicals and D4B5 from Chemicon) were also tested in competition ELISA assays with either 4G1, 5D5 or 6C12 binding to GST-γ2III as described above. Through such an assay it was determined that neither mAb GB3 nor mAb D4B5 have any effect on the mAb 4G1, 5D5 or 6C12 binding to the full length GST-γ2 DIII fusion protein described above (see Example 1). However, it seems that the GB3 and D4B5 do not bind properly, if at all, to the GST-γ2-III fusion protein that was used for coating the ELISA plates. It may be the case that these antibodies do not work well in ELISA (manufacturers did not give any information or instructions for ELISA) or they just do not bind our fusion protein. Therefore it is difficult to make decisions about specificity and/or selectivity of these mAbs. However, what can be said is that these mAbs do not compete with mAb 4G1, mAb 5D5, and/or mAb 6C12 in terms of binding this portion of γ2 DIII.

### Example 8

The following experiment was performed to evaluate the expression of γ2 in a sampling of tumor cell lines.

Subconfluent B16F10 melanoma cells, W4T1 breast carcinoma cells, and Lewis lung carcinoma cells were harvested, washed, and resuspended in PBS. Tumor cell lines were plated, in triplicate, in 96-well microtiter plates and placed in a dry incubator with the cap removed at 37° C. Twenty-four hours later, the incubation resulted in cell lysis and absorption of cellular protein to the wells of the plates. The plates were washed with PBS and blocked with a solution of 1% BSA in PBS (so as to block non-specific binding). After blocking, the wells were again washed.

Monoclonal antibody 5D5 (1.0 μg ml in a solution of 1% BSA in PBS) was added to wells in a total volume of 100 μl. After an appropriate period, plates were washed and incubated with horse radish peroxidase (HRP)-labeled secondary antibody. In control experiments, mAb 5D5 was allowed to bind to BSA-coated wells. Optical density measurements were taken at 490 nm according to normal dry down ELISA procedures. The ELISA data was corrected for non-specific binding of secondary antibody.

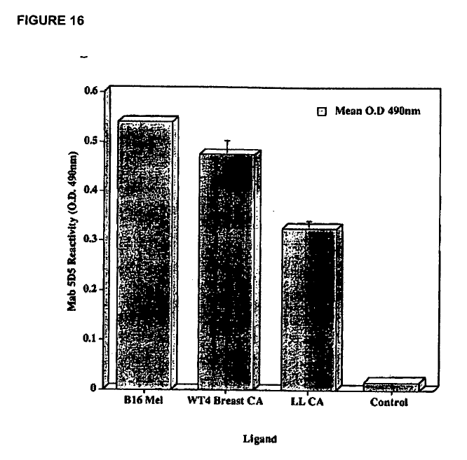

The results of the dry down ELISA, which are set forth in FIG. 16, suggest that mAb 5D5 reacts with proteins expressed in these cancer cells. Accordingly, these results also suggest that these tumor cells produce γ2 peptides that are bound by antibodies provided by the invention.

### Example 9

This experiment was performed to assess the anti-metastatic effects of anti-γ2 DIII mAbs in a chick embryo metastasis assay.

Twelve-day old chick embryos were obtained and each injected with approximately 350,000 B16F10 melanoma cells in the presence of absence of mAb 5D5, mAb 6C12, or non-specific control antibody at a concentration of 100 μg antibody/embryo. The experiments were performed with 5-10 embryos per condition. The embryos were allowed to incubate for a total of 7 days, after which time they were sacrificed and the lungs removed for analysis.

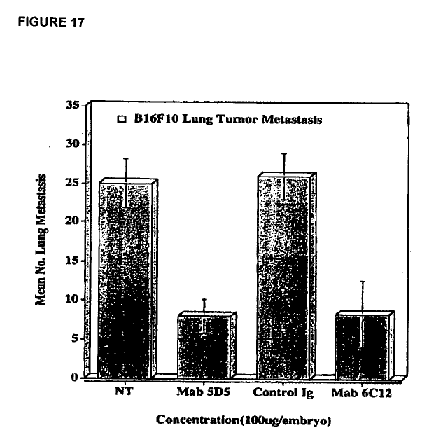

The results of these experiments are presented in FIG. 17. B16F10 melanoma cells readily metastasized in the lungs of the chick embryos. Both mAb 5D5 and mAb 6C12 showed potent inhibition of B16F10 cell metastasis, by approximately 70% as compared to controls.

The results of this Experiment suggest that both mAb 5D5 and mAb 6C12 can inhibit metastasis in chordate cells in vivo.

### Example 10

This example demonstrates the high affinity of anti-γ2 DIII mAbs for γ2 DIII peptides and a method for determining such affinities in other antibodies

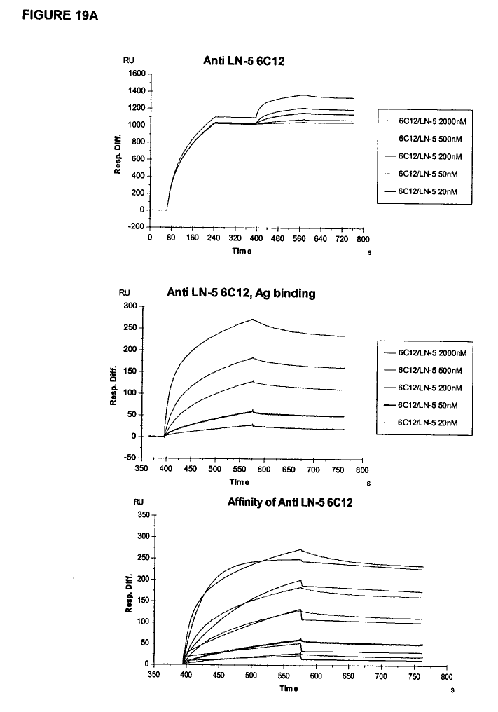

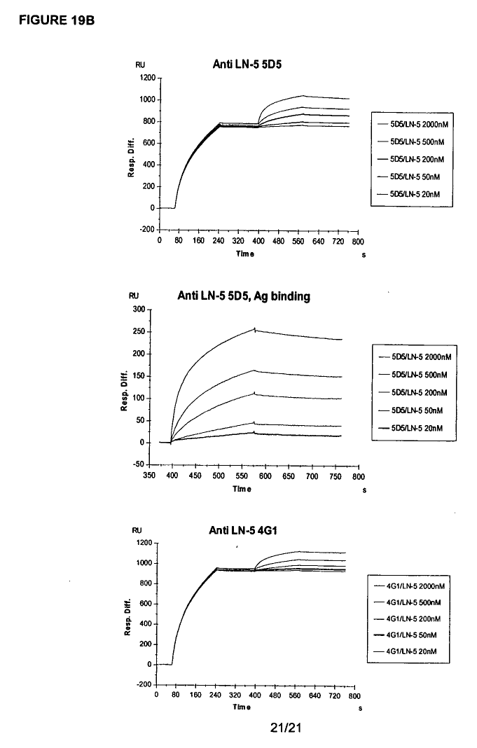

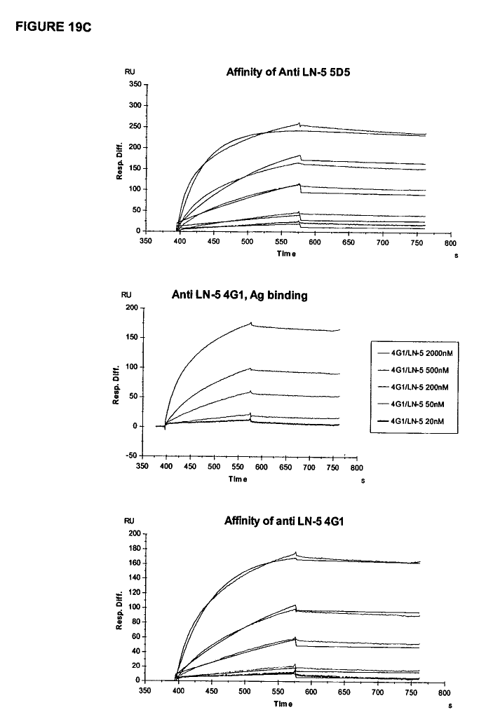

GST-γ2DIII fusion protein (see above) was immobilized to a RAMFc BIAcore chip according to standard techniques and allowed to interact in a surface plasmon resonance affinity assay with compositions containing mAb 4G1, mAb 5D5, and mAb 6C12 individually (in a concentration of 3.4 mg/ml, 2.0 mg/ml, and 2.0 mg/ml, respectively, in HBS-EP buffer (flow 20 μl/min)); affinities for the antigen were determined. Resulting reflection/refractive index data obtained from these analyses (at 200 nM, 500 nM, 200 nM, 50 nM, and 20 nM) are presented individually in FIGS. 19A-19C. Antibody-affinity data calculated from these measurements by standard techniques are presented in Table 10:

TABLE 10

| | ka (1/Ms) | kd (1/s) | KD (M) | Chi2 |
|---|---|---|---|---|
| 6C12 | 1.66E+04 | 3.87E−04 | 2.33E−08 | 115 |
| 5D5 | 1.36E+04 | 2.30E−04 | 1.69E−08 | 81.9 |
| 4G1 | 9.28E+03 | 1.06E−04 | 1.14E−08 | 6.57 |

The results of this experiment demonstrate that mAb 4G1, mAb 5D5, and mAb 6C12 have significant levels of affinity for γ2 DIII peptides. Antibodies and other L5G2D3BPs having similar properties are expected to exhibit similar levels of affinity for γ2 DIII.

Humanized antibodies, fully human antibodies, variant antibodies, as well as fragments thereof, derivatives of any thereof, and other L5G2D3BPs provided by this invention are expected to exhibit substantially similar affinities for similar portions of γ2 DIII (at least about 50%, at least about 70%, at least about 80%, at least about 90%, at least about 95% of such affinities, etc., such as about 60-99% of such affinity), if not essentially similar affinities for γ2 DIII (e.g., such affinity ± about 10%, 15%, 20%, 25%, etc.), or even greater affinity for γ2 DIII (e.g., about 10-9 or about 10-10, etc.). Such affinities are another attribute of some of the anti-γ2 DIII antibodies and other L5G2D3BPs provided by this invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention are to be construed to cover both the singular and the plural, unless otherwise indicated herein or clearly contradicted by context.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXEMPLARY ASPECTS AND FEATURES OF THE INVENTION

To better illustrate the invention, a non-limiting list of exemplary aspects and features of the invention is presented here:

1. A peptide which competes with mAb 5D5 for binding to laminin-5 γ2 domain III.

2. A peptide according to aspect 1, wherein the Ln-5 γ2 domain III binding peptide competes with mAb 5D5 for binding to a region of Ln-5 spanning from about residue 518 to about residue 537 of SEQ ID NO:1.

3. A peptide which competes with mAb 6C12 for binding to laminin-5 γ2 domain III.

4. A peptide according to aspect 3, wherein the Ln-5 γ2 domain III binding peptide competes with mAb 6C12 for binding to a region of Ln-5 spanning from about residue 495 to about residue 555 of SEQ ID NO:1.

5. A peptide according to aspect 4, wherein the Ln-5 γ2 domain III binding peptide competes with mAb 6C12 for binding to a region of Ln-5 spanning from about residue 500 to about residue 548 of SEQ ID NO:1.

6. A peptide according to aspect 5, wherein the Ln-5 γ2 domain III binding peptide competes with mAb 6C12 for binding to a region of Ln-5 spanning from about residue 508 to about residue 543 of SEQ ID NO:1.

7. A peptide according to aspect 6, wherein the Ln-5 γ2 domain III binding peptide competes with mAb 6C12 for binding to a region of Ln-5 spanning from about residue 516 to about residue 533 of SEQ ID NO:1.

8. A peptide according to aspect 3, wherein the Ln-5 γ2 domain III binding peptide competes with mAb 6C12 for binding to a region of Ln-5 consisting essentially of residues 494-516 of SEQ ID NO:1.

9. A peptide according to aspect 3, wherein the Ln-5 γ2 domain III binding peptide competes with mAb 6C12 for binding to a region of Ln-5 consisting essentially of residues 539-552 of SEQ ID NO:1.

10. A peptide according to aspect 3, wherein the Ln-5 γ2 domain III binding peptide competes with mAb 6C12 for binding to a region of Ln-5 consisting essentially of residues 494-516 of SEQ ID NO:1 and to a region consisting essentially of residues 539-552 of SEQ ID NO:1.

11. A peptide according to any of aspects 8 to 10, wherein the Ln-5 γ2 domain III binding peptide does not compete with mAb 6C12 for a region of Ln-5 consisting essentially of residues 518-537 of SEQ ID NO:1.

12. A peptide which cross-competes with both mAb 6C12 and mAb 5D5 for binding to laminin-5 γ2 domain III.

13. A peptide which competes with mAb 4G1 for binding to laminin-5 γ2 domain III.

14. A peptide according to any of aspects 1 to 13, wherein the competition is determined by use of an ELISA as described in the Examples section.

15. A peptide that specifically binds to a Ln-5 γ2 epitope, which epitope is also specifically bound by mAb 6C12.

16. A peptide that specifically binds to a Ln-5 γ2 epitope, which epitope is also specifically bound by mAb 5D5.

17. A peptide that specifically binds to a Ln-5 γ2 epitope, which epitope is also specifically bound by mAb 4G1.

18. A peptide having substantially the same specific binding characteristics of one or more mAbs selected from 4G1, 5D5, and 6C12.

19. A peptide selective for a region of γ2 located within the region from about residue 380 to about residue 400 of SEQ ID NO:1.

20. A peptide according to aspect 19, wherein the Ln-5 γ2 domain III binding peptide is selective for a region of γ2 located within the region from residue 385 to residue 400 of SEQ ID NO:1.

21. A peptide according to aspect 19, wherein the Ln-5 γ2 domain III binding peptide is selective for a region of γ2 located within the region from residue 380 to residue 399 of SEQ ID NO:1.

22. A peptide selective for a region of γ2 located within the region from about residue 440 to about residue 465 of SEQ ID NO:1.

23. A peptide selective for a region of γ2 located within the region from about residue 420 to about residue 460 of SEQ ID NO:1.

24. A peptide selective for a region of γ2 located within the region from about residue 494 to about residue 515 of SEQ ID NO:1.

25. A peptide selective for a region of γ2 located within the region from about residue 517 to about residue 572 of SEQ ID NO:1.

26. A peptide selective for a region of γ2 located within the region from about residue 520 to about residue 550 of SEQ ID NO:1.

27. A peptide selective for a region of γ2 located within the region from about residue 535 to about residue 560 of SEQ ID NO:1.

28. A peptide selective for a region of γ2 located within the region from about residue 539 to about residue 552 of SEQ ID NO:1.

29. A peptide selective for a region of γ2 located within the region from about residue 540 to about residue 550 of SEQ ID NO:1.

30. A peptide selective for a region of γ2 located within the region from about residue 560 to about residue 580 of SEQ ID NO:1.

31. A peptide selective for a region of γ2 located within the region from about residue 560 to about residue 590 of SEQ ID NO:1.

32. A peptide selective for a region of γ2 located within the region from about residue 573 to about residue 602 of SEQ ID NO:1.

33. A peptide which specifically binds to a region of Ln-5 corresponding to from about residue 494 to about residue 534 of SEQ ID NO:1.

34. A peptide which specifically binds to a region of Ln-5 corresponding to from about residue 495 to about residue 530 of SEQ ID NO:1.

35. A peptide which specifically binds to a region of Ln-5 corresponding to from about residue 495 to about residue 545 of SEQ ID NO:1.

36. A peptide which specifically binds to a region of Ln-5 corresponding to from about residue 495 to about residue 550 of SEQ ID NO:1.

37. A peptide which specifically binds to a region of Ln-5 corresponding to from about residue 495 to about residue 570 of SEQ ID NO:1.

38. A peptide which specifically binds to a region of Ln-5 corresponding to from about residue 495 to about residue 590 of SEQ ID NO:1.

39. A peptide which specifically binds to a region of Ln-5 corresponding to from about residue 500 to about residue 550 of SEQ ID NO:1.

40. A peptide which specifically binds to a region of Ln-5 corresponding to from about residue 510 to about residue 520 of SEQ ID NO:1.

41. A peptide which specifically binds to a region of Ln-5 corresponding to from about residue 510 to about residue 535 of SEQ ID NO:1.

42. A peptide which specifically binds to a region of Ln-5 corresponding to from about residue 510 to about residue 545 of SEQ ID NO:1.

43. A peptide which specifically binds to a region of Ln-5 corresponding to from about residue 525 to about residue 535 of SEQ ID NO:1.

44. A peptide which specifically binds to a region of Ln-5 corresponding to from about residue 525 to about residue 545 of SEQ ID NO:1.

45. A peptide which specifically binds to a region of Ln-5 corresponding to from about residue 535 to about residue 545 of SEQ ID NO:1.

46. A peptide which specifically binds to a region of Ln-5 corresponding to from about residue 500 to about residue 550 of SEQ ID NO:1.

47. A peptide which specifically binds to a region of Ln-5 corresponding to from about residue 520 to about residue 550 of SEQ ID NO:1.

48. A peptide that specifically binds a human laminin-5 γ2 conformation-dependent epitope comprising Tyr500 of SEQ ID NO:1.

49. A peptide that specifically binds a human laminin-5 γ2 conformation-dependent epitope, which epitope comprises Tyr500 of Ln-5 γ2 and one, two, three, four, five, six, or seven additional Ln-5 γ2 amino acids located within about 15 Å of Tyr500.

50. A peptide that specifically binds a human laminin-5 γ2 conformation-dependent epitope comprising His508 of SEQ ID NO:1.

51. A peptide that specifically binds a human laminin-5 γ2 conformation-dependent epitope, which epitope comprises His508 of Ln-5 γ2 and one, two, three, four, five, six, or seven additional Ln-5 γ2 amino acids located within about 15 Å thereof.

52. A peptide according to aspect 50 or aspect 51, wherein the conformation-dependent epitope comprises Glu507 of SEQ ID NO:1.

53. A peptide according to any one of aspects 50 to 52, wherein the conformation-dependent epitope comprises Leu534 of SEQ ID NO:1.

54. A peptide that specifically binds a human laminin-5 γ2 conformation-dependent epitope comprising Pro516 of SEQ ID NO:1.

55. A peptide that specifically binds a human laminin-5 γ2 conformation-dependent epitope comprising Ser526 of SEQ ID NO:1.

56. A peptide according to aspect 55, wherein the structural epitope comprises Ala527 of SEQ ID NO:1.

57. A peptide that specifically binds a human laminin-5 γ2 conformation-dependent epitope comprising Arg533 of SEQ ID NO:1.

58. A peptide that specifically binds a human laminin-5 γ2 conformation-dependent epitope, which epitope comprises Arg533 of Ln-5 γ2 and one, two, three, four, five, six, or seven additional Ln-5 γ2 amino acids located within about 15 Å thereof.

59. A peptide that specifically binds a human laminin-5 γ2 conformation-dependent epitope comprising Tyr500 and His508 of SEQ ID NO:1.

60. A peptide according to aspect 59, wherein the structural epitope comprises Arg533 of SEQ ID NO:1.

61. A peptide of aspect 59 or aspect 60, wherein the structural epitope comprises Ser526 of SEQ ID NO:1.

62. A peptide according to any one of aspects 59 to 61, wherein the structural epitope comprises Pro516 of SEQ ID NO:1.

63. A peptide that specifically binds a human laminin-5 γ2 conformation-dependent epitope comprising Tyr500 and Pro516 of SEQ ID NO:1.

64. A peptide according to aspect 63, wherein the structural epitope comprises Arg533 of SEQ ID NO:1.

65. A peptide according to aspect 63 or aspect 64, wherein the structural epitope comprises Ser526 of SEQ ID NO:1.

66. A peptide according to any one of aspects 63 to 65, wherein the structural epitope comprises His508 of SEQ ID NO:1.

67. A peptide that specifically binds a human laminin-5 γ2 conformation-dependent epitope comprising Tyr500 and Ser526 of SEQ ID NO:1.

68. A peptide according to aspect 67, wherein the structural epitope comprises Arg533 of SEQ ID NO:1.

69. A peptide of aspect 67 or aspect 68, wherein the structural epitope comprises His508 of SEQ ID NO:1.

70. A peptide according to any one of aspects 67 to 69, wherein the structural epitope comprises Pro516 of SEQ ID NO:1.

71. A peptide that specifically binds a human laminin-5 γ2 conformation-dependent epitope comprising Tyr500 and Arg533 of SEQ ID NO:1.

72. A peptide according to aspect 71, wherein the structural epitope comprises His508 of SEQ ID NO:1.

73. A peptide of aspect 71 or aspect 72, wherein the structural epitope comprises Ser526 of SEQ ID NO:1.

74. A peptide according to any one of aspects 71 to 73, wherein the structural epitope comprises Pro516 of SEQ ID NO:1.

75. A peptide that specifically binds a human laminin-5 γ2 conformation-dependent epitope comprising His508 and Pro516 of SEQ ID NO:1.

76. A peptide according to aspect 75, wherein the structural epitope comprises Tyr500 of SEQ ID NO:1.

77. A peptide of aspect 75 or aspect 76, wherein the structural epitope comprises Ser526 of SEQ ID NO:1.

78. A peptide according to any one of aspects 75 to 77, wherein the structural epitope comprises Arg533 of SEQ ID NO:1.

79. A peptide that specifically binds a human laminin-5 γ2 conformation-dependent epitope comprising His508 and Ser526 of SEQ ID NO:1.

80. A peptide according to aspect 79, wherein the structural epitope comprises Tyr500 of SEQ ID NO:1.

81. A peptide of aspect 79 or aspect 80, wherein the structural epitope comprises Pro516 of SEQ ID NO:1.

82. A peptide according to any one of aspects 79 to 81, wherein the structural epitope comprises Arg533 of SEQ ID NO:1.

83. A peptide that specifically binds a human laminin-5 γ2 conformation-dependent epitope comprising His508 and Arg533 of SEQ ID NO:1.

84. A peptide according to aspect 83, wherein the structural epitope comprises Tyr500 of SEQ ID NO:1.

85. A peptide of aspect 83 or aspect 84, wherein the structural epitope comprises Pro516 of SEQ ID NO:1.

86. A peptide according to any one of aspects 83 to 85, wherein the structural epitope comprises Ser526 of SEQ ID NO:1.

87. A peptide according to any one of aspects 83 to 86, wherein the structural epitope comprises Glu507 of SEQ ID NO:1.

88. A peptide according to any one of aspects 83 to 87, wherein the structural epitope comprises Leu534 of SEQ ID NO:1.

89. A peptide that specifically binds a human laminin-5 γ2 conformation-dependent epitope comprising Pro516 and Ser526 of SEQ ID NO:1.

90. A peptide according to aspect 89, wherein the structural epitope comprises Tyr500 of SEQ ID NO:1.

91. A peptide of aspect 89 or aspect 90, wherein the structural epitope comprises His508 of SEQ ID NO:1.

92. A peptide according to any one of aspects 89 to 91, wherein the structural epitope comprises Arg533 of SEQ ID NO:1.

93. A peptide that specifically binds a human laminin-5 γ2 conformation-dependent epitope comprising Pro516 and Arg533 of SEQ ID NO:1.

94. A peptide according to aspect 93, wherein the structural epitope comprises Tyr500 of SEQ ID NO:1.

95. A peptide of aspect 93 or aspect 94, wherein the structural epitope comprises His508 of SEQ ID NO:1.

96. A peptide according to any one of aspects 93 to 95, wherein the structural epitope comprises Ser526 of SEQ ID NO:1.

97. A peptide that specifically binds a human laminin-5 γ2 conformation-dependent epitope comprising Ser526 and Arg533 of SEQ ID NO:1.

98. A peptide according to aspect 97, wherein the structural epitope comprises Tyr500 of SEQ ID NO:1.

99. A peptide of aspect 97 or aspect 98, wherein the structural epitope comprises His508 of SEQ ID NO:1.

100. A peptide according to any one of aspects 97 to 99, wherein the structural epitope comprises Pro516 of SEQ ID NO:1.

101. A peptide according to any one of aspects 33 to 100, wherein said peptide binds a Ln-5 γ2 peptide consisting essentially of at least one of SEQ ID NOS:2 to 21.

102. A peptide according to any one of aspects 33 to 101, wherein said peptide comprises a complementarity determining region (CDR) that binds a peptide consisting essentially of at least one of SEQ ID NOS:2 to 21.

103. A peptide that specifically binds a human laminin-5 γ2 conformation-dependent epitope comprising His543 of SEQ ID NO:1.

104. A peptide that specifically binds a human laminin-5 γ2 conformation-dependent epitope, which epitope comprises His543 of Ln-5 γ2 and one, two, three, four, five, six, or seven additional Ln-5 γ2 amino acids located within about 15 Å thereof.

105. A peptide that specifically binds a human laminin-5 γ2 conformation-dependent epitope comprising Ile548 of SEQ ID NO:1.

106. A peptide that specifically binds a human laminin-5 γ2 conformation-dependent epitope, which epitope comprises Ile548 of Ln-5 γ2 and one, two, three, four, five, six, or seven additional Ln-5 γ2 amino acids located within about 15 Å thereof.

107. A peptide that specifically binds a human laminin-5 γ2 conformation-dependent epitope comprising His543 and Ile548 of SEQ ID NO:1.

108. A peptide according to aspect 107, wherein the structural epitope comprises Ser526 of SEQ ID NO:1.

109. A peptide of aspect 107 or aspect 108, wherein the structural epitope comprises Arg533 of SEQ ID NO:1.

110. A peptide that specifically binds a human laminin-5 γ2 conformation-dependent epitope comprising His543 and Ser526 of SEQ ID NO:1.

111. A peptide according to aspect 110, wherein the structural epitope comprises Arg533 of SEQ ID NO:1.

112. A peptide of aspect 110 or aspect 111, wherein the structural epitope comprises Ile548 of SEQ ID NO:1.

113. A peptide that specifically binds a human laminin-5 γ2 conformation-dependent epitope comprising His543 and Arg533 of SEQ ID NO:1.

114. A peptide according to aspect 113, wherein the structural epitope comprises Ser526 of SEQ ID NO:1.

115. A peptide of aspect 113 or aspect 114, wherein the structural epitope comprises Ile548 of SEQ ID NO:1.

116. A peptide that specifically binds a human laminin-5 γ2 conformation-dependent epitope comprising Ile548 and Ser526 of SEQ ID NO:1.

117. A peptide according to aspect 116, wherein the structural epitope comprises Arg533 of SEQ ID NO:1.

118. A peptide of aspect 116 or aspect 117, wherein the structural epitope comprises His543 of SEQ ID NO:1.

119. A peptide that specifically binds a human laminin-5 γ2 conformation-dependent epitope comprising Ile548 and Arg533 of SEQ ID NO:1.

120. A peptide according to aspect 119, wherein the structural epitope comprises Ser526 of SEQ ID NO:1.

121. A peptide of aspect 119 or aspect 120, wherein the structural epitope comprises His543 of SEQ ID NO:1.

122. A peptide which specifically binds to a region of Ln-5 corresponding to from about residue 380 to about residue 400 of SEQ ID NO:1.

123. A peptide which specifically binds to a region of Ln-5 corresponding to from about residue 380 to about residue 420 of SEQ ID NO:1.

124. A peptide which specifically binds to a region of Ln-5 corresponding to from about residue 382 to about residue 407 of SEQ ID NO:1.

125. A peptide which specifically binds to a region of Ln-5 corresponding to from about residue 385 to about residue 395 of SEQ ID NO:1.

126. A peptide which specifically binds to a region of Ln-5 corresponding to from about residue 385 to about residue 399 of SEQ ID NO:1.

127. A peptide which specifically binds to a region of Ln-5 corresponding to from about residue 385 to about residue 400 of SEQ ID NO:1.

128. A peptide which specifically binds to a region of Ln-5 corresponding to from about residue 385 to about residue 442 of SEQ ID NO:1.

129. A peptide which specifically binds to a region of Ln-5 corresponding to from about residue 386 to about residue 400 of SEQ ID NO:1.

130. A peptide which specifically binds to a region of Ln-5 corresponding to from about residue 390 to about residue 460 of SEQ ID NO:1.

131. A peptide which specifically binds to a region of Ln-5 corresponding to from about residue 391 to about residue 460 of SEQ ID NO:1.

132. A peptide which specifically binds to a region of Ln-5 corresponding to from about residue 391 to about residue 461 of SEQ ID NO:1.

133. A peptide which specifically binds to a region of Ln-5 corresponding to from about residue 395 to about residue 414 of SEQ ID NO:1.

134. A peptide which specifically binds to a region of Ln-5 corresponding to from about residue 395 to about residue 420 of SEQ ID NO:1.

135. A peptide which specifically binds to a region of Ln-5 corresponding to from about residue 395 to about residue 442 of SEQ ID NO:1.

136. A peptide which specifically binds to a region of Ln-5 corresponding to from about residue 395 to about residue 445 of SEQ ID NO:1.

137. A peptide which specifically binds to a region of Ln-5 corresponding to from about residue 400 to about residue 440 of SEQ ID NO:1.

138. A peptide which specifically binds to a region of Ln-5 corresponding to from about residue 400 to about residue 420 of SEQ ID NO:1.

139. A peptide which specifically binds to a region of Ln-5 corresponding to from about residue 409 to about residue 418 of SEQ ID NO:1.

140. A peptide which specifically binds to a region of Ln-5 corresponding to from about residue 411 to about residue 420 of SEQ ID NO:1.

141. A peptide which specifically binds to a region of Ln-5 corresponding to from about residue 412 to about residue 414 of SEQ ID NO:1.

142. A peptide which specifically binds to a region of Ln-5 corresponding to from about residue 412 to about residue 435 of SEQ ID NO:1.

143. A peptide which specifically binds to a region of Ln-5 corresponding to from about residue 412 to about residue 442 of SEQ ID NO:1.

144. A peptide which specifically binds to a region of Ln-5 corresponding to from about residue 414 to about residue 435 of SEQ ID NO:1.

145. A peptide which specifically binds to a region of Ln-5 corresponding to from about residue 414 to about residue 442 of SEQ ID NO:1.

146. A peptide which specifically binds to a region of Ln-5 corresponding to from about residue 415 to about residue 440 of SEQ ID NO:1.

147. A peptide which specifically binds to a region of Ln-5 corresponding to from about residue 415 to about residue 445 of SEQ ID NO:1.

148. A peptide which specifically binds to a region of Ln-5 corresponding to from about residue 415 to about residue 460 of SEQ ID NO:1.

149. A peptide which specifically binds to a region of Ln-5 corresponding to from about residue 420 to about residue 435 of SEQ ID NO:1.

150. A peptide which specifically binds to a region of Ln-5 corresponding to from about residue 420 to about residue 442 of SEQ ID NO:1.

151. A peptide which specifically binds to a region of Ln-5 corresponding to from about residue 420 to about residue 460 of SEQ ID NO:1.

152. A peptide which specifically binds to a region of Ln-5 corresponding to from about residue 425 to about residue 440 of SEQ ID NO:1.

153. A peptide which specifically binds to a region of Ln-5 corresponding to from about residue 430 to about residue 442 of SEQ ID NO:1.

154. A peptide which specifically binds to a region of Ln-5 corresponding to from about residue 430 to about residue 460 of SEQ ID NO:1.

155. A peptide which specifically binds to a region of Ln-5 corresponding to from about residue 432 to about residue 443 of SEQ ID NO:1.

156. A peptide which specifically binds to a region of Ln-5 corresponding to from about residue 433 to about residue 442 of SEQ ID NO:1.

157. A peptide which specifically binds to a region of Ln-5 corresponding to from about residue 435 to about residue 455 of SEQ ID NO:1.

158. A peptide which specifically binds to a region of Ln-5 corresponding to from about residue 435 to about residue 460 of SEQ ID NO:1.

159. A peptide which specifically binds to a region of Ln-5 corresponding to from about residue 439 to about residue 447 of SEQ ID NO:1.

160. A peptide which specifically binds to a region of Ln-5 corresponding to from about residue 441 to about residue 449 of SEQ ID NO:1.

161. A peptide which specifically binds to a region of Ln-5 corresponding to from about residue 442 to about residue 460 of SEQ ID NO:1.

162. A peptide which specifically binds to a region of Ln-5 corresponding to from about residue 450 to about residue 460 of SEQ ID NO:1.

163. A peptide that specifically binds a human laminin-5 γ2 conformation-dependent epitope comprising Asp395 of SEQ ID NO:1.

164. A peptide that specifically binds a human laminin-5 γ2 conformation-dependent epitope, which epitope comprises Asp395 of Ln-5 γ2 and one, two, three, four, five, six, or seven additional Ln-5 amino acids located within about 15 Å thereof.

165. A peptide that specifically binds a human laminin-5 γ2 conformation-dependent epitope comprising Thr412 of SEQ ID NO:1.

166. A peptide that specifically binds a human laminin-5 γ2 conformation-dependent epitope, which epitope comprises Thr412 of Ln-5 γ2 and one, two, three, four, five, six, or seven additional Ln-5 amino acids located within about 15 Å thereof.

167. A peptide that specifically binds a human laminin-5 γ2 conformation-dependent epitope comprising Ile414 of SEQ ID NO:1.

168. A peptide that specifically binds a human laminin-5 γ2 conformation-dependent epitope, which epitope comprises Ile414 of Ln-5 γ2 and one, two, three, four, five, six, or seven additional Ln-5 amino acids located within about 15 Å thereof.

169. A peptide that specifically binds a human laminin-5 γ2 conformation-dependent epitope comprising Cys442 of SEQ ID NO:1.

170. A peptide that specifically binds a human laminin-5 γ2 conformation-dependent epitope, which epitope comprises Cys442 of Ln-5 γ2 and one, two, three, four, five, six, or seven additional Ln-5 amino acids located within about 15 Å thereof.

171. A peptide that specifically binds a human laminin-5 γ2 conformation-dependent epitope comprising Asp395 and Thr412 of SEQ ID NO:1.

172. A peptide according to aspect 171, wherein the structural epitope comprises Ile414 of SEQ ID NO:1.

173. A peptide of aspect 171 or aspect 172, wherein the structural epitope comprises Cys442 of SEQ ID NO:1.

174. A peptide that specifically binds a human laminin-5 γ2 conformation-dependent epitope comprising Asp395 and Ile414 of SEQ ID NO:1.

175. A peptide according to aspect 174, wherein the structural epitope comprises Thr412 of SEQ ID NO:1.

176. A peptide of aspect 174 or aspect 175, wherein the structural epitope comprises Cys442 of SEQ ID NO:1.

177. A peptide that specifically binds a human laminin-5 γ2 conformation-dependent epitope comprising Asp395 and Cys442 of SEQ ID NO:1.

178. A peptide according to aspect 177, wherein the structural epitope comprises Thr412 of SEQ ID NO:1.

179. A peptide of aspect 177 or aspect 178, wherein the structural epitope comprises Ile414 of SEQ ID NO:1.

180. A peptide that specifically binds a human laminin-5 γ2 conformation-dependent epitope comprising Thr412 and Ile414 of SEQ ID NO:1.

181. A peptide that specifically binds a human laminin-5 γ2 conformation-dependent epitope, which epitope comprises Thr412 and Ile414 and one, two, three, four, five, six, or more Ln-5 amino acid residues located within about 15 Å of Ile414 and/or Thr412.

182. A peptide according to aspect 180 or aspect 181, wherein the structural epitope comprises Asp395 of SEQ ID NO:1.

183. A peptide of any of the aspects 180 to 182, wherein the structural epitope comprises Cys442 of SEQ ID NO:1.

184. A peptide that specifically binds a human laminin-5 γ2 conformation-dependent epitope comprising Thr412 and Cys442 of SEQ ID NO:1.

185. A peptide according to aspect 184, wherein the structural epitope comprises Asp395 of SEQ ID NO:1.

186. A peptide of aspect 184 or aspect 185, wherein the structural epitope comprises Ile414 of SEQ ID NO:1.

187. A peptide that specifically binds a human laminin-5 γ2 conformation-dependent epitope comprising Ile414 and Cys442 of SEQ ID NO:1.

188. A peptide according to aspect 187, wherein the structural epitope comprises Asp395 of SEQ ID NO:1.

189. A peptide of aspect 187 or aspect 188, wherein the structural epitope comprises Thr412 of SEQ ID NO:1.

190. A peptide which specifically binds to a region of Ln-5 corresponding to from about residue 380 to about residue 460 of SEQ ID NO:1.

191. A peptide which specifically binds to a region of Ln-5 corresponding to from about residue 380 to about residue 570 of SEQ ID NO:1.

192. A peptide which specifically binds to a region of Ln-5 corresponding to from about residue 382 to about residue 407 of SEQ ID NO:1.

193. A peptide which specifically binds to a region of Ln-5 corresponding to from about residue 385 to about residue 395 of SEQ ID NO:1.

194. A peptide which specifically binds to a region of Ln-5 corresponding to from about residue 385 to about residue 440 of SEQ ID NO:1.

195. A peptide which specifically binds to a region of Ln-5 corresponding to from about residue 385 to about residue 445 of SEQ ID NO:1.

196. A peptide which specifically binds to a region of Ln-5 corresponding to from about residue 390 to about residue 570 of SEQ ID NO:1.

197. A peptide which specifically binds to a region of Ln-5 corresponding to from about residue 395 to about residue 415 of SEQ ID NO:1.

198. A peptide which specifically binds to a region of Ln-5 corresponding to from about residue 395 to about residue 445 of SEQ ID NO:1.

199. A peptide which specifically binds to a region of Ln-5 corresponding to from about residue 395 to about residue 450 of SEQ ID NO:1.

200. A peptide which specifically binds to a region of Ln-5 corresponding to from about residue 400 to about residue 420 of SEQ ID NO:1.

201. A peptide which specifically binds to a region of Ln-5 corresponding to from about residue 410 to about residue 445 of SEQ ID NO:1.

202. A peptide which specifically binds to a region of Ln-5 corresponding to from about residue 435 to about residue 535 of SEQ ID NO:1.

203. A peptide which specifically binds to a region of Ln-5 corresponding to from about residue 435 to about residue 550 of SEQ ID NO:1.

204. A peptide which specifically binds to a region of Ln-5 corresponding to from about residue 435 to about residue 590 of SEQ ID NO:1.

205. A peptide which specifically binds to a region of Ln-5 corresponding to from about residue 435 to about residue 608 of SEQ ID NO:1.

206. A peptide which specifically binds to a region of Ln-5 corresponding to from about residue 455 to about residue 465 of SEQ ID NO:1.

207. A peptide which specifically binds to a region of Ln-5 corresponding to from about residue 460 to about residue 500 of SEQ ID NO:1.

208. A peptide which specifically binds to a region of Ln-5 corresponding to from about residue 460 to about residue 515 of SEQ ID NO:1.

209. A peptide which specifically binds to a region of Ln-5 corresponding to from about residue 460 to about residue 525 of SEQ ID NO:1.

210. A peptide which specifically binds to a region of Ln-5 corresponding to from about residue 460 to about residue 530 of SEQ ID NO:1.

211. A peptide which specifically binds to a region of Ln-5 corresponding to from about residue 460 to about residue 550 of SEQ ID NO:1.

212. A peptide which specifically binds to a region of Ln-5 corresponding to from about residue 460 to about residue 570 of SEQ ID NO:1.

213. A peptide which specifically binds to a region of Ln-5 corresponding to from about residue 460 to about residue 590 of SEQ ID NO:1.

214. A peptide which specifically binds to a region of Ln-5 corresponding to from about residue 460 to about residue 600 of SEQ ID NO:1.

215. A peptide which specifically binds to a region of Ln-5 corresponding to from about residue 460 to about residue 610 of SEQ ID NO:1.

216. A peptide which specifically binds to a region of Ln-5 corresponding to from about residue 462 to about residue 516 of SEQ ID NO:1.

217. A peptide which specifically binds to a region of Ln-5 corresponding to from about residue 520 to about residue 550 of SEQ ID NO:1.

218. A peptide which specifically binds to a region of Ln-5 corresponding to from about residue 520 to about residue 570 of SEQ ID NO:1.

219. A peptide which specifically binds to a region of Ln-5 corresponding to from about residue 525 to about residue 535 of SEQ ID NO:1.

220. A peptide which specifically binds to a region of Ln-5 corresponding to from about residue 525 to about residue 545 of SEQ ID NO:1.

221. A peptide which specifically binds to a region of Ln-5 corresponding to from about residue 535 to about residue 545 of SEQ ID NO:1.

222. A peptide which specifically binds to a region of Ln-5 corresponding to from about residue 550 to about residue 570 of SEQ ID NO:1.

223. A peptide which specifically binds to a region of Ln-5 corresponding to from about residue 550 to about residue 590 of SEQ ID NO:1.

224. A peptide which specifically binds to a region of Ln-5 corresponding to from about residue 560 to about residue 570 of SEQ ID NO:1.

225. A peptide which specifically binds to a region of Ln-5 corresponding to from about residue 560 to about residue 590 of SEQ ID NO:1.

226. A peptide which specifically binds to a region of Ln-5 corresponding to from about residue 560 to about residue 600 of SEQ ID NO:1.

227. A peptide according to any of aspects 222 to 226, wherein the N-terminal of the region is Ile548.

228. A peptide according to any of aspects 222 to 226, wherein the N-terminal of the region is within 3 amino acids of Ile548.

229. A peptide according to aspect 228, wherein the N-terminal of the region is within 2 amino acids of Ile548.

230. A peptide which specifically binds to a region of Ln-5 corresponding to from about residue 575 to about residue 600 of SEQ ID NO:1.

231. A peptide which specifically binds to a region of Ln-5 corresponding to from about residue 600 to about residue 620 of SEQ ID NO:1.

232. A peptide which specifically binds to a conformation-dependent epitope, which epitope comprises one or more amino acid residues located within a region defined by about position 415 to about position 516 of Ln-5 γ2 and one or more amino acid residues located within a region defined by about position 539 to about position 555 of Ln-5 γ2.

233. A peptide according to aspect 232, wherein the peptide binds residues 494-516 of Ln-5 γ2 and 539-552 of Ln-5 γ2 with significantly higher affinity and/or avidity than the peptide binds residues 517-540 of Ln-5 γ2.

234. A peptide comprising a VL CDR consisting essentially of SEQ ID NO:24, 25, 26, 28, 29, 30, 32, 33, or 34.

235. A peptide according to aspect 234, wherein the peptide comprises a VL CDR1 consisting essentially of SEQ ID NO:24.

236. A peptide according to aspect 234, wherein the peptide comprises a VL CDR1 consisting essentially of SEQ ID NO:25.

237. A peptide according to aspect 234, wherein the peptide comprises a VL CDR1 consisting essentially of SEQ ID NO:26.

238. A peptide according to aspect 234, wherein the peptide comprises a VL CDR2 consisting essentially of SEQ ID NO:28.

239. A peptide according to aspect 234, wherein the peptide comprises a VL CDR2 consisting essentially of SEQ ID NO:29.

240. A peptide according to aspect 234, wherein the peptide comprises a VL CDR2 consisting essentially of SEQ ID NO:30.

241. A peptide according to aspect 234, wherein the peptide comprises a VL CDR3 consisting essentially of SEQ ID NO:32.

242. A peptide according to aspect 234, wherein the peptide comprises a VL CDR3 consisting essentially of SEQ ID NO:33.

243. A peptide according to aspect 234, wherein the peptide comprises a VL CDR3 consisting essentially of SEQ ID NO:34.

244. A peptide comprising a VH CDR consisting essentially of SEQ ID NO:36, 37, 38, 40, 41, 42, 44, 45 or 46.

245. A peptide according to aspect 244, wherein the peptide comprises a VH CDR1 consisting essentially of SEQ ID NO:36.

246. A peptide according to aspect 244, wherein the peptide comprises a VH CDR1 consisting essentially of SEQ ID NO:37.

247. A peptide according to aspect 244, wherein the peptide comprises a VH CDR1 consisting essentially of SEQ ID NO:38.

248. A peptide according to aspect 244, wherein the peptide comprises a VH CDR2 consisting essentially of SEQ ID NO:40.

249. A peptide according to aspect 244, wherein the peptide comprises a VH CDR2 consisting essentially of SEQ ID NO:41.

250. A peptide according to aspect 244, wherein the peptide comprises a VH CDR2 consisting essentially of SEQ ID NO:42.

251. A peptide according to aspect 244, wherein the peptide comprises a VH CDR3 consisting essentially of SEQ ID NO:44.

252. A peptide according to aspect 244, wherein the peptide comprises a VH CDR2 consisting essentially of SEQ ID NO:45.

253. A peptide according to aspect 244, wherein the peptide comprises a VH CDR2 consisting essentially of SEQ ID NO:46.

254. A peptide comprising (a) a first VL region comprising three VL CDRs, which independently of each other consist essentially of SEQ ID NOS:24, 28, and 32 and (b) a first VH region comprising three VH CDRs, which independently of each other consist essentially of SEQ ID NOS:36, 40, and 44.

255. A peptide comprising (a) a first VL region comprising three VL CDRs, which independently of each other consist essentially of SEQ ID NOS:25, 29, and 33 and (b) a first VH region comprising three VH CDRs, which independently of each other consist essentially of SEQ ID NOS:37, 41, and 45.

256. A peptide comprising (a) a first VL region comprising three VL CDRs, which independently of each other consist essentially of SEQ ID NOS:26, 30, and 34 and (b) a first VH region comprising three VH CDRs, which independently of each other consist essentially of SEQ ID NOS:38, 42, and 46.

257. A peptide according to any of aspects 254 to 256, wherein the VL region and the VH region are present on the same chain in the peptide.

258. A peptide according to aspect 257, wherein the VL region and the VH region are separated by a flexible linker.

259. A peptide according to any of aspects 254 to 256, wherein the VL region and the VH region are present on the separate chains in the peptide.

260. A peptide according to aspect 259, wherein the VL region and the VH region are present on the separate chains in the peptide in the context of an immunoglobulin fold protein.

261. A peptide according to any of aspects 254 to 260, wherein the first VL region and the first VH region are oriented such that the three CDRs in the VL region and the three CDRs in the VH region cooperatively associate to contribute in selectively and/or specifically bind an antigenic determinant on γ2 DIII.

262. A peptide according to aspect 261, wherein the peptide comprises a second VL region identical to the first VL region and a second VH region identical to the first VH region, where the second VL region and the second VH region cooperatively associate to contribute in selectively and/or specifically bind an antigenic determinant on γ2 DIII.

263. A peptide comprising a complementarity determining region (CDR) consisting essentially of the CDR region of mAb 4G1.

264. A peptide comprising a complementarity determining region (CDR) that is a functional variant of the CDR region of mAb 4G1.

265. A peptide according to aspect 264, which has at least about 50% of the affinity, avidity or specificity of mAb 4G1.

266. A peptide according to aspect 265, which has at least about 60% of the affinity, avidity or specificity of mAb 4G1.

267. A peptide according to aspect 266, which has at least about 70% of the affinity, avidity or specificity of mAb 4G1.

268. A peptide according to aspect 267, which has at least about 80% of the affinity, avidity or specificity of mAb 4G1.

269. A peptide according to aspect 268, which has at least about 90% of the affinity, avidity or specificity of mAb 4G1.

270. A peptide comprising a CDR consisting essentially of the CDR region of mAb 5D5.

271. A peptide comprising a CDR that is a functional variant of the CDR region of mAb 5D5.

272. A peptide according to aspect 271, which has at least about 50% of the affinity, avidity or specificity of mAb 5D5.

273. A peptide according to aspect 272, which has at least about 60% of the affinity, avidity or specificity of mAb 5D5.

274. A peptide according to aspect 273, which has at least about 70% of the affinity, avidity or specificity of mAb 5D5.

275. A peptide according to aspect 274, which has at least about 80% of the affinity, avidity or specificity of mAb 5D5.

276. A peptide according to aspect 275, which has at least about 90% of the affinity, avidity or specificity of mAb 5D5.

277. A peptide comprising a CDR consisting essentially of the CDR region of mAb 6C12.

278. A peptide comprising a CDR that is a functional variant of the CDR region of mAb 6C12.

279. A peptide according to aspect 278, which has at least about 50% of the affinity, avidity or specificity of mAb 6C12.

280. A peptide according to aspect 279, which has at least about 60% of the affinity, avidity or specificity of mAb 6C12.

281. A peptide according to aspect 280, which has at least about 70% of the affinity, avidity or specificity of mAb 6C12.

282. A peptide according to aspect 281, which has at least about 80% of the affinity, avidity or specificity of mAb 6C12.

283. A peptide according to aspect 282, which has at least about 90% of the affinity, avidity or specificity of mAb 6C12.

284. A peptide comprising a VL CDR1 sequence consisting essentially of a sequence according to Formula III.

285. A peptide according to aspect 284, wherein Xaa₁ is Arg.

286. A peptide according to aspect 284, wherein Xaa₁ is a suitable amino acid residue other than Arg.

287. A peptide according to any of aspects 284 to 286, wherein Xaa₂ is Ser.

288. A peptide according to any of aspects 284 to 286, wherein Xaa₂ a suitable amino acid residue other than Ser.

289. A peptide according to any of aspects 284 to 288, wherein Xaa₃ is Lys or Gln.

290. A peptide according to any of aspects 284 to 288, wherein Xaa₃ is a suitable amino acid residue other than Lys or Gln.

291. A peptide according to any of aspects 284 to 290, wherein Xaa₄ is Ser.

292. A peptide according to any of aspects 284 to 290, wherein Xaa₄ is a suitable amino acid residue other than Ser.

293. A peptide according to any of aspects 284 to 292, wherein Xaa₅ is Leu or Val.

294. A peptide according to any of aspects 284 to 292, wherein Xaa₅ is a suitable amino acid residue other than Leu or Val.

295. A peptide according to any of aspects 284 to 294, wherein Xaa₆ is His or Ser.

296. A peptide according to any of aspects 284 to 294, wherein Xaa₆ is a suitable amino acid residue other than His or Ser.

297. A peptide according to any of aspects 284 to 296, wherein Xaa₇ is Asn or Ser.

298. A peptide according to any of aspects 284 to 296, wherein Xaa₇ is a suitable amino acid residue other than Asn or Ser.

299. A peptide according to any of aspects 284 to 298, wherein Xaa₈ is Ile or Asn.

300. A peptide according to any of aspects 284 to 298, wherein Xaa₈ is a suitable amino acid residue other than Ile or Asn.

301. A peptide according to aspect 284 comprising a VL CDR1 sequence consisting essentially of a sequence according to Formula IV.

302. A peptide comprising a CDR-L2 sequence consisting essentially of a sequence according to Formula IX.

304. A peptide according to aspect 302, wherein Xaa₁ is a suitable amino acid residue other than Gln, Arg, or Asp.

305. A peptide according to any of aspects 302 to 304, wherein Xaa₂ is Ser.

306. A peptide according to any of aspects 302 to 304, wherein Xaa₂ is a suitable amino acid residue other than Ser.

307. A peptide according to any of aspects 302 to 306, wherein Xaa₄ is Ser.

308. A peptide according to any of aspects 302 to 306, wherein Xaa₄ is a suitable amino acid residue other than Ser.

309. A peptide according to aspect 302 comprising a CDR-L2 sequence consisting essentially of a sequence according to Formula X.

310. A peptide comprising a CDR-L3 sequence consisting essentially of a sequence according to Formula XV.

311. A peptide according to aspect 312, wherein Xaa₁ is Gln.

312. A peptide according to aspect 310, wherein Xaa₁ is a suitable amino acid residue other than Gln.

313. A peptide according to any of aspects 310 to 312, wherein Xaa₂ is Asn, Ser, or Trp.

314. A peptide according to any of aspects 310 to 312, wherein Xaa₂ is a suitable amino acid residue other than Asn, Ser, or Trp.

315. A peptide according to any of aspects 310 to 314, wherein Xaa₃ is Glu, His, or Ser.

316. A peptide according to any of aspects 310 to 314, wherein Xaa₃ is a suitable amino acid residue other than Glu, His, or Ser.

317. A peptide according to any of aspects 310 to 316, wherein Xaa₄ is Pro.

318. A peptide according to any of aspects 310 to 316, wherein Xaa₄ is a suitable amino acid residue other than Pro.

319. A peptide according to any of aspects 310 to 318, wherein Xaa₅ is Thr.

320. A peptide according to any of aspects 310 to 318, wherein Xaa₅ is a suitable amino acid residue other than Thr.

321. A peptide according to aspect 310 comprising a CDR-L3 sequence consisting essentially of a sequence according to Formula XVI.

322. A peptide comprising a CDR-H1 sequence consisting essentially of a sequence according to Formula XIX.

323. A peptide according to aspect 322 wherein Xaa₁ is Ser.

324. A peptide according to aspect 322 wherein Xaa₁ is a suitable amino acid residue other than Ser.

325. A peptide according to aspect 322 wherein Xaa₁ is Thr.

326. A peptide according to aspect 322 wherein Xaa₁ is a suitable amino acid residue other than Thr.

327. A peptide according to any of aspects 322 to 326, wherein Xaa₂ is Gly.

328. A peptide according to any of aspects 322 to 326, wherein Xaa₂ is a suitable amino acid residue other than Gly.

329. A peptide according to any of aspects 322 to 328, wherein Xaa₃ is Asp or Tyr.

330. A peptide according to any of aspects 322 to 328, wherein Xaa₃ is a suitable amino acid residue other than Asp or Tyr.

331. A peptide according to any of aspects 322 to 328, wherein Xaa₃ is Phe.

332. A peptide according to any of aspects 322 to 328, wherein Xaa₃ is a suitable amino acid residue other than Phe.

333. A peptide according to any of aspects 322 to 328, wherein Xaa₄ is Gly, or Asp.

334. A peptide according to any of aspects 322 to 332, wherein Xaa₄ is a suitable amino acid residue other than Gly, or Asp.

335. A peptide according to any of aspects 322 to 332, wherein Xaa₄ is Phe.

336. A peptide according to any of aspects 322 to 332, wherein Xaa₄ is a suitable amino acid residue other than Phe.

337. A peptide according to any of aspects 322 to 336, wherein Xaa₅ is Arg, or Ala.

338. A peptide according to any of aspects 322 to 336, wherein Xaa₅ is a suitable amino acid residue other than Arg or Ala.

339. A peptide according to any of aspects 322 to 336, wherein Xaa₅ is Met.

340. A peptide according to any of aspects 322 to 336, wherein Xaa₅ is a suitable amino acid residue other than Met.

341. A peptide according to any of aspects 322 to 340, wherein Xaa₆ is Asn.

342. A peptide according to any of aspects 322 to 340, wherein Xaa₆ is a suitable amino acid residue other than Asn.

343. A peptide according to aspect 322 comprising a CDR-H1 sequence consisting essentially of a sequence according to Formula XX.

344. A peptide according to aspect 343, wherein Xaa₁ is Phe, Gly, or Asp.

345. A peptide according to aspect 343 or 344, wherein Xaa₂ is Met, Arg, or Ala.

346. A peptide according to any of aspects 343 to 345, wherein Xaa₃ is Asn or a covalent bond.

347. A peptide comprising a CDR-H2 sequence consisting essentially of a sequence according to Formula XXV.

348. A peptide according to aspect 347, wherein Xaa₁ is Asn or a covalent bond;

349. A peptide according to aspect 347 or aspect 348, wherein Xaa₂ is Asn or a covalent bond.

350. A peptide according to any of aspects 347 to 349, wherein Xaa₃ is Tyr or a covalent bond.

351. A peptide comprising a CDR-H3 sequence consisting essentially of a sequence according to Formula XXVIII.

352. A peptide according to aspect 351, wherein Xaa₂ is Thr or Ala.

353. A peptide according to aspect 351 or 352, wherein Xaa₃ is Arg, Gly, or Asn.

354. A peptide according to any of aspects 351 to 353, wherein Xaa₄ is Pro or missing.

355. A peptide according to any of aspects 351 to 354, wherein Xaa₅ is Tyr or a covalent bond.

356. A peptide according to any of aspects 351 to 355, wherein Xaa₆ is Asp or Asn.

357. A peptide according to any of aspects 351 to 356, wherein Xaa₇ is Tyr or Phe.

358. A peptide according to any of aspects 351 to 357, wherein Xaa₈ is Tyr or Asp.

359. A peptide according to any of aspects 351 to 358, wherein Xaa₉ is Gly or Glu.

360. A peptide according to any of aspects 351 to 359, wherein Xaa₁₀ is Ser, Arg, or Asn.

361. A peptide according to any of aspects 351 to 360, wherein Xaa₁₁ is Ser, Thr, or Phe.

362. A peptide according to any of aspects 351 to 361, wherein Xaa₁₃ is Ala or Asp.

363. A peptide according to any of aspects 351 to 362, wherein Xaa₁₂ is any suitable amino acid residue other than Phe, and Xaa₁₄ is Tyr.

364. A peptide according to any of aspects 351 to 362, wherein Xaa₁₄ is any suitable amino acid residue other than Tyr, and Xaa₁₂ is Phe.

365. A peptide according to any of aspects 351 to 362, wherein Xaa₁₂ is any suitable residue other than Phe and Xaa₁₄ is any suitable residue other than Tyr.

366. A peptide according to aspect 351 comprising a CDR-H3 sequence consisting essentially of a sequence according to Formula XXX.

367. A peptide according to aspect 366, wherein Xaa₁ is Arg, Gly, or Asn.

368. A peptide according to aspect 366 or 367, wherein Xaa₂ is missing or Pro.

369. A peptide according to any of the aspects 366 to 368, wherein Xaa₃ is Tyr or missing.

370. A peptide according to any of the aspects 366 to 369, wherein Xaa₄ is Ser, Arg, or Asn.

371. A peptide comprising a variant VL CDR1 sequence consisting essentially of a sequence having at least about 60% amino acid sequence identity to one or more of SEQ ID NOS:24-26, wherein

(a) the amino acid residue of the variant VL CDR1 sequence corresponding to position 2 of SEQ ID NOS:24-26, when these sequences are aligned, is any suitable amino acid residue other than Arg;

(b) the amino acid residue of the variant VL CDR1 sequence corresponding to position 4 of SEQ ID NOS:24-26 is any suitable residue other than Ser;

(c) the amino acid residue of the variant VL CDR1 sequence corresponding to position 12 of SEQ ID NOS:24-26 is any suitable amino acid residue other than Gly; or

(d) the variant VL CDR1 sequence differs in the amino acid residues corresponding to two or three of positions 2, 4, and/or 12 of SEQ ID NOS:24-26 from SEQ ID NO:27 by substitutions according to (a)-(c).

372. A peptide according to aspect 371, wherein the peptide comprising the variant VL CDR1 sequence consists essentially of a sequence having at least about 70% amino acid sequence identity to one or more of SEQ ID NOS:24-26.

373. A peptide comprising a variant VL CDR2 sequence consisting essentially of a sequence having at least about 65% amino acid sequence identity with one or more of SEQ ID NOS:28-30, wherein

(a) the residue in the variant VL CDR2 that corresponds to position 5 of SEQ ID NO:31 is any suitable residue other than a Ser;

(b) the residue in the variant VL CDR2 that corresponds to position 9 of SEQ ID NO:31 is any suitable residue other than a Ser; or (c) the residues in the variant VL CDR2 that correspond to positions 5 and 9 of SEQ ID NO:31 are both any suitable residue other than a Ser.

374. A peptide according to aspect 373, wherein the peptide comprising the variant VL CDR2 sequence consists essentially of a sequence having at least about 75% amino acid sequence identity with one or more of SEQ ID NOS:28-30.

375. A peptide comprising a variant VL CDR3 sequence consisting essentially of a sequence

having at least about 65% sequence identity to one or more of SEQ ID NOS:32-34, wherein

(a) the residue in the variant VL CDR3 that corresponds to position 3 of SEQ ID NO:35 is any suitable residue other than Gln;

(b) the residue in the variant VL CDR3 that corresponds to position 8 of SEQ ID NO:35 is any suitable residue other than Pro;

(c) the residue in the variant VL CDR3 that corresponds to position 10 of SEQ ID NO:35 is any suitable residue other than Thr; or

(d) two or more of the residues in the variant VL CDR3 that correspond to positions 3, 8, and 10 of SEQ ID NO:35 differ from that sequence according to a combination of (a) and (b); (a) and (c); (b) and (c); or (a), (b), and (c).

376. A peptide according to aspect 375, wherein the peptide comprising the variant VL CDR3 sequence consists essentially of a sequence having at least about 70% amino acid sequence identity to one or more of SEQ ID NOS:32-34.

377. A peptide comprising a variant VH CDR3 sequence consisting essentially of a sequence having at least about 65% amino acid sequence identity to one or more of SEQ ID NOS:44-46, wherein

(a) the amino acid residue in the variant VH CDR3 that corresponds to position 12 of SEQ ID NO:47 is any suitable residue other than Phe;

(b) the amino acid residue in the variant VH CDR3 that corresponds to position 14 of SEQ ID NO:47 is any suitable residue other than Tyr; or

(c) the amino acid residues in the variant VH CDR3 that correspond to positions 12 and 14 of SEQ ID NO:47 are any suitable residues other than Phe and Tyr, respectively.

378. A peptide according to aspect 386, wherein the peptide comprising the variant VH CDR3 sequence consists essentially of a sequence having at least about 70% amino acid sequence identity to one or more of SEQ ID NOS:44-46.

379. A peptide according to aspect 378, wherein the peptide comprising the variant VH CDR3 sequence consists essentially of a sequence having at least about 80% amino acid sequence identity to one or more of SEQ ID NOS:44-46.

380. A peptide according to aspect 379, wherein the peptide comprising the variant VH CDR3 sequence consists essentially of a sequence having at least about 90% amino acid sequence identity to one or more of SEQ ID NOS:44-46.

381. A peptide comprising SEQ ID NO:60.

382. A peptide comprising a variant VL region having at least about 70% amino acid sequence identity to SEQ ID NO:60.

383. A peptide according to aspect 382 comprising a variant VL region having at least about 75% amino acid sequence identity to SEQ ID NO:60.

384. A peptide according to aspect 383 comprising a variant VL region having at least about 80% amino acid sequence identity to SEQ ID NO:60.

385. A peptide according to aspect 384 comprising a variant VL region having at least about 85% amino acid sequence identity to SEQ ID NO:60.

386. A peptide according to aspect 385 comprising a variant VL region having at least about 90% amino acid sequence identity to SEQ ID NO:60.

387. A peptide according to aspect 386 comprising a variant VL region having at least about 95% amino acid sequence identity to SEQ ID NO:60.

388. A peptide comprising SEQ ID NO:69.

389. A peptide comprising a variant VH region having at least about 70% amino acid sequence identity to SEQ ID NO:69.

390. A peptide according to aspect 389 comprising a variant VH region having at least about 75% amino acid sequence identity to SEQ ID NO:69.

391. A peptide according to aspect 390 comprising a variant VH region having at least about 80% amino acid sequence identity to SEQ ID NO:69.

392. A peptide according to aspect 391 comprising a variant VH region having at least about 85% amino acid sequence identity to SEQ ID NO:69.

393. A peptide according to aspect 392 comprising a variant VH region having at least about 90% amino acid sequence identity to SEQ ID NO:69.

394. A peptide according to aspect 393 comprising a variant VH region having at least about 95% amino acid sequence identity to SEQ ID NO:69.

395. A peptide comprising SEQ ID NO:61.

396. A peptide comprising a variant VL region having at least about 70% amino acid sequence identity to SEQ ID NO:61.

397. A peptide according to aspect 396 comprising a variant VL region having at least about 75% amino acid sequence identity to SEQ ID NO:61.

398. A peptide according to aspect 397 comprising a variant VL region having at least about 80% amino acid sequence identity to SEQ ID NO:61.

399. A peptide according to aspect 398 comprising a variant VL region having at least about 85% amino acid sequence identity to SEQ ID NO:61.

400. A peptide according to aspect 399 comprising a variant VL region having at least about 90% amino acid sequence identity to SEQ ID NO:61.

401. A peptide according to aspect 400 comprising a variant VL region having at least about 95% amino acid sequence identity to SEQ ID NO:61.

402. A peptide comprising SEQ ID NO:70.

403. A peptide comprising a variant VH region having at least about 70% amino acid sequence identity to SEQ ID NO:70.

404. A peptide according to aspect 403 comprising a variant VH region having at least about 75% amino acid sequence identity to SEQ ID NO:70.

405. A peptide according to aspect 404 comprising a variant VH region having at least about 80% amino acid sequence identity to SEQ ID NO:70.

406. A peptide according to aspect 411 comprising a variant VH region having at least about 85% amino acid sequence identity to SEQ ID NO:70.

407. A peptide according to aspect 406 comprising a variant VH region having at least about 90% amino acid sequence identity to SEQ ID NO:70.

408. A peptide according to aspect 407 comprising a variant VH region having at least about 95% amino acid sequence identity to SEQ ID NO:70.

409. A peptide comprising SEQ ID NO:62.

410. A peptide comprising a variant VL region having at least about 70% amino acid sequence identity to SEQ ID NO:62.

411. A peptide according to aspect 410 comprising a variant VL region having at least about 75% amino acid sequence identity to SEQ ID NO:62.

412. A peptide according to aspect 411 comprising a variant VL region having at least about 80% amino acid sequence identity to SEQ ID NO:62.

413. A peptide according to aspect 412 comprising a variant VL region having at least about 85% amino acid sequence identity to SEQ ID NO:62.

414. A peptide according to aspect 413 comprising a variant VL region having at least about 90% amino acid sequence identity to SEQ ID NO:62.

414. A peptide according to aspect 414 comprising a variant VL region having at least about 95% amino acid sequence identity to SEQ ID NO:62.

415. A peptide comprising SEQ ID NO:71.

416. A peptide comprising a variant VH region having at least about 70% amino acid sequence identity to SEQ ID NO:71.

417. A peptide according to aspect 416 comprising a variant VH region having at least about 75% amino acid sequence identity to SEQ ID NO:71.

418. A peptide according to aspect 417 comprising a variant VH region having at least about 80% amino acid sequence identity to SEQ ID NO:71.

419. A peptide according to aspect 418 comprising a variant VH region having at least about 85% amino acid sequence identity to SEQ ID NO:71.

420. A peptide according to aspect 419 comprising a variant VH region having at least about 90% amino acid sequence identity to SEQ ID NO:71.

421. A peptide according to aspect 420 comprising a variant VH region having at least about 95% amino acid sequence identity to SEQ ID NO:71.

422. A peptide according to any of aspects 234 to 421, wherein the peptide specifically binds Ln-5 γ2.

423. A peptide according to aspect 422, wherein the peptide specifically binds Ln-5 γ2 domain III.

424. A peptide according to any one of aspects 15 to 423, wherein the peptide competes with mAb 5D5 for binding to laminin-5 γ2 domain III.

425. A peptide according to any one of aspects 15 to 423, wherein the peptide competes with mAb 6C12 for binding to laminin-5 γ2 domain III.

426. A peptide according to any one of aspects 15 to 423, wherein the peptide competes with mAb 5D5 and mAb 6C12 for binding to laminin-5 γ2 domain III.

427. A peptide according to any one of aspects 424 to 426, wherein the peptide binds to human laminin-5 γ2 with greater affinity than monoclonal antibodies 5D5 and 6C12.

428. A peptide according to any one of aspects 15 to 423, wherein the peptide competes with mAb 4G1 for binding to laminin-5 γ2 domain III.

429. A peptide according to aspect 428, wherein the peptide binds to human laminin-5 γ2 with greater affinity than monoclonal antibody 4G1.

430. A peptide according to any one of aspects 1 to 4429, wherein the Ln-5 γ2 domain III binding peptide does not detectably bind to the α3 chain of Ln-5.

431. A peptide according to any one of aspects 1 to 430, wherein the Ln-5 γ2 domain III binding peptide does not detectably bind to the β3 chain of Ln-5.

432. A peptide according to any one of aspects 1 to 431, wherein the Ln-5 γ2 domain III binding peptide is selective for a portion of γ2 in the context of a human epithelial cell

433. A peptide according to any one of aspects 1 to 431, wherein the Ln-5 γ2 domain III binding peptide is selective for a portion of γ2 with respect to the basal lamina of a human being.

434. A peptide according to any one of aspects 1 to 433, wherein the Ln-5 γ2 domain III binding peptide is specific for regions only found within laminin-5 γ2 domain III.

435. A peptide according to any one of aspects 1 to 434, wherein the Ln-5 γ2 domain III binding peptide is an antibody.

436. A peptide according to aspect 435, wherein the Ln-5 γ2 domain III binding peptide is a monoclonal antibody.

437. A peptide according to aspect 435 or aspect 436, with the proviso that the antibody is not GB3, D4B5, 4G1, 5D5, and/or 6C12.

438. A peptide according to any one of aspects 435 to 437, wherein the Ln-5 γ2 domain III binding peptide is a human antibody.

439. A peptide according to any one of aspects 435 to 437, wherein the Ln-5 γ2 domain III binding peptide is a humanized antibody.

440. A peptide according to any one of aspects 435 to 437, wherein the Ln-5 γ2 domain III binding peptide is a chimeric antibody.

441. A fragment of a peptide according to any one of aspects 1 to 440,wherein the fragment binds to the γ2 chain of laminin-5 in invasive cancer cells.

442. A peptide according to any one of aspects 1 to 441, wherein the Ln-5 γ2 domain III binding peptide is substantially free of other Ln-5 γ2 domain III binding peptides.

443. A pharmaceutical formulation comprising an Ln-5 γ2 domain III binding peptide according to any one of aspects 1 to 442.

444. A pharmaceutical formulation according to aspect 443 comprising more than one Ln-5 γ2 domain III binding peptide.

445. A composition comprising an amount of a peptide according to any one of aspects 1 to 442 in an amount effective to reduces the movement of invasive cancer cells and a pharmaceutically acceptable carrier.

446. A method of reducing one or more aspects of cancer progression in a mammal comprising delivering a peptide according to any one of aspects 1 to 442 to the human in an amount and under conditions such that one or more aspects of cancer progression are detectably reduced in the mammal.

447. The method of aspect 446, wherein performing the method reduces the movement of invasive cancer cells in the mammal.

448. The method of aspect 446 or aspect 447, wherein delivery of the peptide to the mammal comprises administering a vector comprising a nucleic acid sequence coding for production of the peptide in the mammal.

449. The method of any one of aspects 446 to 448, wherein the delivery of the peptide is performed in combination with the delivery of a second therapeutic agent to the mammal.

450. The method of aspect 449, wherein the peptide and second therapeutic agent (a) primarily reduce different aspects of cancer progression, (b) reduce at least one aspect of cancer progression at a level greater than the predicted additive effect of the peptide and second cancer agent, or (c) accomplish both (a) and (b).

451. The method of aspect 449 or aspect 450, wherein the second therapeutic agent is a laminin-5 modulator.

452. The method of aspect 451, wherein the laminin-5 modulator is an antibody specific for the α3 chain or the β3 chain of laminin-5.

1. An isolated peptide that comprises an antibody or an antibody fragment and that competes with mAb 5D5, mAb 6C12, or cross-competes with both 5D5 and 6C12, in specifically binding to at least a portion of domain III of the γ2 chain of human laminin-5 (Ln-5 γ2 DIII).

2. The peptide of claim 1, wherein the peptide competes with mAb 5D5 more than 6C12 in binding to Ln-5 γ2 DIII.

3. The peptide according to claim 1, wherein the peptide competes with mAb 6C12 more than 5D5 in binding to Ln-5 γ2 DIII.

4. The peptide of claim 1, wherein the peptide is a monoclonal antibody.

5. The peptide of claim 2, wherein the peptide is a monoclonal antibody.

6. The peptide of claim 3, wherein the peptide is a monoclonal antibody.

7. The peptide of claim 4, wherein the monoclonal antibody is a humanized antibody.

8. The peptide of claim 4, wherein the monoclonal antibody binds a protein that is secreted from human cancer cells and comprises at least a portion of Ln-5 γ2 DIII.

9. The peptide of claim 1, wherein the peptide is conjugated to a compound that kills cancer cells.

10. The peptide of claim 1, wherein the peptide is conjugated to a detection agent.

11. A composition comprising a therapeutically effective amount of an isolated peptide according to claim 1 and a pharmaceutically acceptable carrier.

12. The composition of claim 11, wherein the composition comprises a therapeutically effective amount of a monoclonal antibody according to claim 4.

13. The composition of claim 12, wherein the composition further comprises a therapeutically effective amount of at least one secondary anti-cancer agent.

14. An assay for assessing the γ2-associated peptide content of a composition comprising administering a peptide according to claim 1 to the composition and determining whether the peptide binds any γ2-associated peptides in the composition.

15. The assay of claim 14, wherein the composition is the tissue of a human patient.

16. The method of claim 15, wherein the assay is used to assist in directing (a) an anti-cancer surgical procedure, (b) administration of an anti-cancer drug, (c) administration of an anti-cancer radiation therapy, or (d) any combination of any thereof.

17. A method of reducing cancer progression in a patient comprising delivering to the patient a physiologically effective amount of a peptide according to claim 1 so as to reduce cancer progression therein.

18. The method of claim 17, wherein the method comprises administering to the patient a therapeutically effective amount of a composition according to claim 10 so as to treat cancer in the patient.

19. The method of claim 18, wherein the composition comprises an effective amount of a monoclonal antibody according to claim 8.

20. The method of claim 19, wherein the method comprises administering the composition in association with administering an anti-cancer agent, applying an anti-cancer therapy, or both.
